(19) United States
(12) Patent Application Publication
(10) Pub. No.: US 2024/0131187 A1
(43) Pub. Date: Apr. 25, 2024

(54) EFFECTOR PROTEINS, EFFECTOR PARTNERS, COMPOSITIONS, SYSTEMS AND METHODS OF USE THEREOF

(71) Applicant: Mammoth Biosciences, Inc., Brisbane, CA (US)

(72) Inventors: Timothy Robert ABBOTT, South San Francisco, CA (US); Aaron DELOUGHERY, San Francisco, CA (US); David PAEZ-ESPINO, Concord, CA (US); Benjamin Julius RAUCH, San Francisco, CA (US)

(21) Appl. No.: 18/469,512

(22) Filed: Sep. 17, 2023

Related U.S. Application Data
(60) Provisional application No. 63/376,419, filed on Sep. 20, 2022.

(51) Int. Cl. A61K 48/00; C12N 9/12; C12N 15/11

(52) U.S. Cl. CPC A61K 48/005; C12N 9/1241; C12N 15/11

(57) ABSTRACT

Provided herein are compositions, systems, and methods comprising effector proteins, effector partners, and uses thereof. These effector proteins may be characterized as CRISPR-associated (Cas) proteins. Various compositions, systems, and methods of the present disclosure may leverage the activities of these effector proteins for the modification, detection, and/or engineering of nucleic acids.

### CROSS-REFERENCE

This application claims benefit of U.S. Provisional Application No. 63/376,419, filed on Sep. 20, 2022, which is incorporated herein by reference in its entirety.

### INCORPORATION BY REFERENCE OF SEQUENCE LISTING

The instant application contains a Sequence Listing, which has been submitted via Patent Center. The Sequence Listing titled 203477-764201_SL.xml, which was created on Sep. 18, 2023, and is 1,221,105 bytes in size, is hereby incorporated by reference in its entirety.

### FIELD

The present disclosure relates generally to polypeptides, such as effector proteins and/or effector partners, compositions of such polypeptides and guide nucleic acids, systems and methods of using such polypeptides and compositions, including detecting and modifying target nucleic acids.

### BACKGROUND

Clustered Regularly Interspaced Short Palindromic Repeats (CRISPR) and associated proteins (Cas proteins), sometimes referred to as a CRISPR/Cas system, were first identified in certain bacterial species and are now understood to form part of a prokaryotic acquired immune system. CRISPR/Cas systems provide immunity in bacteria and archaea against viruses and plasmids by targeting the nucleic acids of the viruses and plasmids in a sequence-specific manner.

### SUMMARY

The present disclosure provides for polypeptides, such as effector proteins, compositions, methods and systems comprising the same, and uses thereof. In some instances, compositions, systems, and methods comprise effector partners and uses thereof. In some instances, compositions, systems, and methods comprise guide nucleic acids or uses thereof. Compositions, systems, and methods disclosed herein may leverage nucleic acid modifying activities. Nucleic acid modifying activities may include cis cleavage activity, trans cleavage activity, nicking activity, nuclease activity, and/or nucleobase modifying activity. In some instances, compositions, systems and methods are useful for the detection of target nucleic acids. In some instances, compositions, systems and methods are useful for the treatment of a disease or disorder. The disease or disorder may be associated with a target nucleic acid. The disease or disorder may be associated with one or more mutations in the target nucleic acid.

### I. CERTAIN EMBODIMENTS

Provided herein are compositions comprising an isolated polypeptide, or a recombinant nucleic acid encoding the isolated polypeptide, wherein the isolated polypeptide comprises an amino acid sequence that is at least 75% identical to any one of the sequences set forth in TABLE 1.

Also provided herein, are compositions comprising: a polypeptide, or a nucleic acid encoding the polypeptide, wherein the polypeptide comprises an amino acid sequence that is at least 75% identical to any one of the sequences set forth in TABLE 1; and an engineered guide nucleic acid or a DNA molecule that encodes the engineered guide nucleic acid.

Also provided herein, are compositions comprising: a polypeptide, or a nucleic acid encoding the polypeptide, wherein the polypeptide comprises an amino acid sequence that is at least 75% identical to any one of the sequences set forth in TABLE 1; a donor nucleic acid; and an engineered guide nucleic acid or a nucleic acid that encodes the engineered guide nucleic acid.

Also provided herein, are compositions comprising: an isolated polypeptide, or a recombinant nucleic acid encoding the isolated polypeptide, wherein the isolated polypeptide comprises an amino acid sequence that is at least 75% identical to any one of the sequences set forth in TABLE 1; and one or more partner polypeptides or isolated partner polypeptides, or one or more recombinant nucleic acids encoding the one or more partner polypeptides or isolated partner polypeptides.

Also provided herein, are compositions comprising: a polypeptide, or a nucleic acid encoding the polypeptide, wherein the polypeptide comprises an amino acid sequence that is at least 75% identical to any one of the sequences set forth in TABLE 1; one or more partner polypeptides, or one or more nucleic acids encoding the one or more partner polypeptides; and a nucleic acid, wherein the nucleic acid is a donor nucleic acid, or an engineered guide nucleic acid or a nucleic acid that encodes the engineered guide nucleic acid.

Also provided herein, are compositions comprising: a polypeptide, or a nucleic acid encoding the polypeptide, wherein the polypeptide comprises an amino acid sequence that is at least 75% identical to any one of the sequences set forth in TABLE 1; one or more partner polypeptides, or one or more nucleic acids encoding the one or more partner polypeptides; a donor nucleic acid; and an engineered guide nucleic acid or a nucleic acid that encodes the engineered guide nucleic acid.

In some embodiments, the one or more partner polypeptide comprises an amino acid sequence that is at least 75% identical to any one of the sequences set forth in TABLE 1.1.

Also provided herein, are compositions comprising one or more isolated partner polypeptides or one or more recombinant nucleic acids encoding the one or more isolated partner polypeptides wherein the one or more isolated partner polypeptide comprises an amino acid sequence that is at least 75% identical to any one of the sequences set forth in TABLE 1.1.

Also provided herein, are compositions comprising: one or more partner polypeptides, or one or more nucleic acids encoding the one or more partner polypeptides, wherein the one or more partner polypeptide comprises an amino acid sequence that is at least 75% identical to any one of the sequences set forth in TABLE 1.1; and a nucleic acid, wherein the nucleic acid is a donor nucleic acid, or an engineered guide nucleic acid or a nucleic acid that encodes the engineered guide nucleic acid. In some embodiments, the nucleic acid is a donor nucleic acid. In some embodiments, the nucleic acid is an engineered guide nucleic acid or a nucleic acid that encodes the engineered guide nucleic acid.

Also provided herein, are compositions comprising: one or more partner polypeptides, or one or more nucleic acids encoding the one or more partner polypeptides, wherein the one or more partner polypeptide comprises an amino acid sequence that is at least 75% identical to any one of the sequences set forth in TABLE 1.1; and a donor nucleic acid and an engineered guide nucleic acid or a nucleic acid that encodes the engineered guide nucleic acid.

In some embodiments, the polypeptide disclosed herein comprises an amino acid sequence that is at least 80% identical, at least 85% identical, at least 90% identical, at least 95% identical, or at least 100% identical to any one of the sequences set forth in TABLE 1.

In some embodiments, the composition disclosed herein comprises one or more, two or more, three or more, four or more, five or more partner polypeptides, or one or more nucleic acids encoding the one or more, two or more, three or more, four or more, five or more partner polypeptides. In some embodiments, the one or more partner polypeptides comprises an amino acid sequence that is at least 75% identical, at least 80% identical, at least 85% identical, at least 90% identical, at least 95% identical, or at least 100% identical to any one of the sequences set forth in TABLE 1.1.

In some embodiments, the composition disclosed herein comprises two or more partner polypeptides, or one or more nucleic acids encoding the two or more partner polypeptides.

Also provided herein, are compositions comprising one or more partner polypeptides, wherein each partner polypeptide independently comprises an amino acid sequence. In some embodiments, each partner polypeptide independently comprises an amino acid sequence that is 75% identical, at least 80% identical, at least 85% identical, at least 90% identical, at least 95% identical, or at least 100% identical to any one of the sequences set forth in TABLE 1.1. In some embodiments, each partner polypeptide independently comprises an amino acid sequence that is identical to any one of the sequences set forth in TABLE 1.1.

Also provided herein, are compositions comprising a polypeptide and a partner polypeptide combination as described in TABLE 6.

Also provided herein, are compositions comprising a nucleic acid that is a donor nucleic acid.

In some embodiments, the composition disclosed herein comprises modifies a target sequence in a target nucleic acid. In some embodiments, the target sequence is downstream to a protospacer adjacent motif (PAM). In some embodiments, the target nucleic acid comprises an insertion site, and optionally wherein the insertion site is recognized by a polypeptide or partner polypeptide.

Also provided herein, are compositions comprising a nucleic acid encoding an engineered guide nucleic acid or the engineered guide nucleic acid, wherein the engineered guide nucleic acid comprises a first region and a second region comprising a nucleic acid sequence that is complementary to the target sequence in the target nucleic acid, wherein the first region and the second region are heterologous to each other. In some embodiments, the first region, at least partially, interacts with the polypeptide, or partner polypeptide, or both. In some embodiments, the engineered guide nucleic acid comprises at least 10 contiguous nucleotides that are complementary to a eukaryotic sequence. In some embodiments, the engineered guide nucleic acid comprises one or more phosphorothioate (PS) backbone modifications, 2′-fluoro (2′-F) sugar modifications, or 2′-O-Methyl (2′OMe) sugar modifications.

Also provided herein, are compositions further comprising an additional engineered guide nucleic acid that binds a different loci of the target nucleic acid than the engineered guide nucleic acid.

Also provided herein, are compositions comprising one or more polypeptides, or one or more partner polypeptides, wherein the polypeptide or the partner polypeptide, or both, is fused to one or more heterologous polypeptide, and optionally wherein the heterologous polypeptide is a nuclear localization signal (NLS).

Also provided herein, are compositions comprising one or more polypeptides, wherein the polypeptide comprises a RuvC domain that is capable of cleaving a target nucleic acid. In some embodiments, the polypeptide is a nuclease that is capable of cleaving at least one strand of a target nucleic acid or the polypeptide is a nuclease that is capable of modification of at least one strand of a target nucleic acid. In some embodiments, the modification of the target nucleic acid comprises cleaving the target nucleic acid, deleting a nucleic acid of the target nucleic acid, inserting a nucleic acid into the target nucleic acid, substituting a nucleic acid of the target nucleic acid with an alternative nucleic acid, more than one of the foregoing, or combinations thereof. In some embodiments, the modification of the target nucleic acid comprises insertion of a nucleic acid into the target nucleic acid. In some embodiments, the modification of the target nucleic acid comprises insertion of a donor nucleic acid, deletion of a target nucleic acid, insertion of a donor nucleic acid fragment, deletion of a target nucleic acid fragment, or combinations thereof. In some embodiments, the donor nucleic acid can be a nucleotide, a nucleotide sequence, a coding sequence, a gene, a gene fragment, an exon, an intron, an exon fragment, an intron fragment, a gene regulatory region, a gene regulatory region fragment, coding sequences thereof, or combinations thereof. In some embodiments, the modification of the target nucleic acid comprises site-specific recombinase activity. In some embodiments, the modification of the target nucleic acid comprises transposase or transposase-like activity. In some embodiments, the modification of the target nucleic acid comprises modification of a length of about 100 base pairs to about 500 base pairs of the target nucleic acid. In some embodiments, the target sequence is within a human gene.

Also provided herein is a nucleic acid expression vector that encodes a polypeptide, wherein the polypeptide comprises an amino acid sequence that is at least 75% identical to any one of the sequences set forth in TABLE 1. In some embodiments, any of the nucleic acid expression vectors provided herein, encoding the polypeptide and/or the partner polypeptide further encodes a donor nucleic acid. In some embodiments, any of the nucleic acid expression vectors provided herein, encoding the polypeptide, the partner polypeptide, and/or a donor nucleic acid further encodes a target nucleic acid or wherein the library further comprises a separate nucleic acid expression vector encoding the target nucleic acid. In some embodiments, any of the nucleic acid expression vectors provided herein, wherein at least one nucleic acid expression vector is a viral vector. In some embodiments, the viral vector is an adeno associated viral (AAV) vector. In some embodiments, at least one of the nucleic acid expression vectors provided herein is a lipid or a lipid nanoparticle.

Also provided herein is a library of nucleic acid expression vectors comprising the any of the nucleic acid expression vectors provided herein, wherein the nucleic acid expression vector encoding the polypeptide further encodes a partner polypeptide or wherein the library further comprises a separate nucleic acid expression vector encoding the partner polypeptide, and wherein the partner polypeptide comprises an amino acid sequence that is at least 75% identical to any one of the sequences set forth in TABLE 1.1. In some embodiments, a library of any of the nucleic acid expression vectors provided herein, wherein the polypeptide and/or the partner polypeptide further encodes a donor nucleic acid. In some embodiments, a library of any of the nucleic acid expression vectors provided herein encoding the polypeptide, the partner polypeptide, and/or a donor nucleic acid. In some embodiments, a library of any of the nucleic acid expression vectors further encoding a target nucleic acid. In some embodiments, a library further comprising a separate nucleic acid expression vector encoding the donor nucleic acid. In some embodiments, a library further comprising a separate nucleic acid expression vector encoding the target nucleic acid. In some embodiments, a library further comprising a viral vector is an adeno associated viral (AAV) vector. In some embodiments, a library further comprising at least one nucleic acid expression vector is a lipid or a lipid nanoparticle.

Also provided herein is a pharmaceutical composition, comprising any one of the compositions provided herein, or any one of the nucleic acid expression vectors provided herein, or any one of the library of nucleic acid expression vectors provided herein, and a pharmaceutically acceptable excipient.

Also provided herein is a system comprising any one of the compositions provided herein, or any one of the nucleic acid expression vectors provided herein, or any one of the library of nucleic acid expression vectors provided herein. In some embodiments, a system comprising at least one detection reagent for detecting a target nucleic acid. In some embodiments, the at least one detection reagent is selected from a reporter nucleic acid, a detection moiety, an additional polypeptide, or a combination thereof, optionally wherein the reporter nucleic acid comprises a fluorophore, a quencher, or a combination thereof. In some embodiments, the at least one detection reagent is operably linked to a polypeptide or partner polypeptide, such that a detection event occurs upon contacting the system with a target nucleic acid. In some embodiments, any of the systems provided herein, further comprising at least one amplification reagent for amplifying a target nucleic acid. In some embodiments, the at least one amplification reagent is selected from the group consisting of a primer, an activator, a dNTP, an rNTP, and combinations thereof.

Also provided herein is a method of modifying a target nucleic acid comprising contacting the target nucleic acid with any one of the compositions provided herein, any one of the nucleic acid expression vectors provided herein, any one of the library of nucleic acid expression vectors provided herein, any one of the pharmaceutical compositions provided herein, or any one of the systems provided herein. In some embodiments, the method of modifying a target nucleic acid within a human gene. In some embodiments, the method of modifying a target nucleic acid associated with expression of a human gene. In some embodiments, the method of modifying wherein contacting thereby modifies the target nucleic acid. In some embodiments, modifying of the target nucleic acid comprises insertion or deletion of an exon, intron, exon fragment, intron fragment, gene regulatory region, gene regulatory region fragment, or any combinations thereof. In some embodiments, the method further comprising contacting the target nucleic acid with a guide nucleic acid. In some embodiments, the method is performed in a cell. In some embodiments, the method is performed in vivo. In some embodiments, the target nucleic acid comprises a mutation associated with a disease, and optionally wherein the disease is any one of the diseases recited in TABLE 5 and/or wherein the target nucleic acid is encoded by a gene recited in TABLE 4.

Also provided herein is a cell comprising any one of the compositions provided herein, any one of the nucleic acid expression vectors provided herein, any one of the library of nucleic acid expression vectors provided herein, or any one of the systems provided herein. In some embodiments, the cell comprises a target nucleic acid modified by any one of the compositions provided herein, any one of the nucleic acid expression vectors provided herein, any one of the library of nucleic acid expression vectors provided herein.

Also provided herein is a population of cells comprising at least one of the cells provided herein.

Also provided herein is a method of treating a disease associated with a mutation or aberrant expression of a human gene in a subject in need thereof, the method comprising administering to the subject the any one of the pharmaceutical compositions provided herein, and optionally wherein the disease is any one of the diseases recited in TABLE 5 and/or wherein the human gene is a gene recited in TABLE 4.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION OF THE INVENTION

It is to be understood that both the foregoing general description and the following detailed description are exemplary, and explanatory only, and are not restrictive of the disclosure.

The section headings used herein are for organizational purposes only and are not to be construed as limiting the subject matter described.

All documents, or portions of documents, cited in this application, including, but not limited to, patents, patent applications, articles, books, and treatises, are hereby expressly incorporated by reference in their entirety for any purpose.

### II. DEFINITIONS

Unless otherwise indicated, all technical terms used herein have the same meaning as commonly understood by one of ordinary skill in the art to which this invention belongs. Unless otherwise indicated or obvious from context, the following terms have the following meanings:

As used in this specification and the appended claims, the singular forms “a,” “an,” and “the” include plural references unless the context clearly dictates otherwise.

Any reference to “or” herein is intended to encompass “and/or” unless otherwise stated. As used herein, the term “and/or” includes any and all combinations of one or more of the associated listed items.

Use of the term “including” as well as other forms, such as “includes” and “included,” is not limiting.

As used herein, the term “comprise” and its grammatical equivalents specifies the presence of stated features, integers, steps, operations, elements, and/or components, but does not preclude the presence or addition of one or more other features, integers, steps, operations, elements, components, and/or groups thereof.

As used herein, the term “about” in reference to a number or range of numbers is understood to mean the stated number and numbers+/−10% thereof, or 10% below the lower listed limit and 10% above the higher listed limit for the values listed for a range.

The terms “% identical,” “% identity,” and “percent identity,” or grammatical equivalents thereof, with reference to an amino acid sequence or nucleotide sequence, refer to the percent of residues that are identical between respective positions of two sequences when the two sequences are aligned for maximum sequence identity. The % identity is calculated by dividing the total number of the aligned residues by the number of the residues that are identical between the respective positions of the at least two sequences, and multiplying by 100. Generally, computer programs can be employed for such calculations. Illustrative programs that compare and align pairs of sequences, include ALIGN (Myers and Miller, Comput Appl Biosci. 1988 March; 4(1):11-7), FASTA (Pearson and Lipman, Proc Natl Acad Sci USA. 1988 April; 85(8):2444-8; Pearson, Methods Enzymol. 1990; 183:63-98) and gapped BLAST (Altschul et al., Nucleic Acids Res. 1997 Sep. 1; 25(17):3389-40), BLASTP, BLASTN, or GCG (Devereux et al., Nucleic Acids Res. 1984 Jan. 11; 12(1 Pt 1):387-95).

The terms “% complementary”, “% complementarity”, “percent complementary”, and “percent complementarity” or grammatical equivalents thereof, as used interchangeably herein, with reference to two or more nucleic acid molecules refers to the percent of nucleotides in two nucleotide sequences in said nucleic acid molecules of equal length that can undergo cumulative base pairing at two or more individual corresponding positions in an antiparallel orientation. The % complementary is calculated by dividing the total number of the complementary residues by the total number of the nucleotides in one of the equal length sequences, and multiplying by 100. Complete or total complementarity describes nucleotide sequences in 100% of the residues of a nucleotide sequence are complementary to residues in a reference nucleotide sequence. “Partial” complementarity describes nucleotide sequences in which at least 20%, but less than 100%, of the residues of a nucleotide sequence are complementary to residues in a reference nucleotide sequence. In some embodiments, at least 50%, but less than 100%, of the residues of a nucleotide sequence are complementary to residues in a reference nucleotide sequence. In some embodiments, at least 70%, 80%, 90% or 95%, but less than 100%, of the residues of a nucleotide sequence are complementary to residues in a reference nucleotide sequence. Sequences are said to be “substantially complementary” when at least 85% of the residues of a nucleotide sequence are complementary to residues in a reference nucleotide sequence. “Noncomplementary” describes nucleotide sequences in which less than 20% of the residues of a nucleotide sequence are complementary to residues in a reference nucleotide sequence.

The terms, “amplification” and “amplifying,” or grammatical equivalents thereof, as used herein, refers to a process by which a nucleic acid molecule is enzymatically copied to generate a plurality of nucleic acid molecules containing the same sequence as the original nucleic acid molecule or a distinguishable portion thereof.

The term “ATPase activity” refers to catalytic activity that results in the decomposition of ATP as an energy source to help power an enzymatic reaction.

The terms, “bind” or “binding,” as used herein, refer to a non-covalent interaction between macromolecules (e.g., between two polypeptides, between a polypeptide and a nucleic acid; between a polypeptide/guide nucleic acid complex and a target nucleic acid; and the like). While in a state of noncovalent interaction, the macromolecules are said to be “associated” or “interacting” or “binding” (e.g., when a molecule X is said to interact with a molecule Y, it is meant the molecule X binds to molecule Y in a non-covalent manner). Non-limiting examples of non-covalent interactions are ionic bonds, hydrogen bonds, van der Waals and hydrophobic interactions. Not all components of a binding interaction need be sequence-specific (e.g., contacts with phosphate residues in a DNA backbone), but some portions of a binding interaction may be sequence-specific.

The term, “catalytically inactive effector protein,” as used herein, refers to an effector protein that is modified relative to a naturally-occurring effector protein to have a reduced or eliminated catalytic activity relative to that of the naturally-occurring effector protein, but retains its ability to interact with a guide nucleic acid. The catalytic activity that is reduced or eliminated is often a nuclease activity. The naturally-occurring effector protein may be a wildtype protein. In some instances, the catalytically inactive effector protein is referred to as a catalytically inactive variant of an effector protein, e.g., a Cas effector protein.

The term, “cis cleavage,” as used herein, refers to cleavage (hydrolysis of a phosphodiester bond) of a target nucleic acid by a complex of an effector protein and a guide nucleic acid, wherein at least a portion of the guide nucleic acid is hybridized to at least a portion of the target nucleic acid. Cleavage may occur within or directly adjacent to the portion of the target nucleic acid that is hybridized to the portion of the guide nucleic acid.

The term “codon optimized” as used herein refers to a mutation of a nucleotide sequence encoding a polypeptide, such as a nucleotide sequence encoding an effector protein, to mimic the codon preferences of the intended host organism or cell while encoding the same polypeptide. Thus, the codons can be changed, but the encoded polypeptide remains unchanged. For example, if the intended target cell was a human cell, a human codon-optimized nucleotide sequence encoding an effector protein could be used. As another non-limiting example, if the intended host cell were a mouse cell, then a mouse codon-optimized nucleotide sequence encoding an effector protein could be generated. As another non-limiting example, if the intended host cell were a eukaryotic cell, then an eukaryote codon-optimized nucleotide sequence encoding an effector protein could be generated. As another non-limiting example, if the intended host cell were a prokaryotic cell, then a prokaryote codon-optimized nucleotide sequence encoding an effector protein could be generated. Codon usage tables are readily available, for example, at the “Codon Usage Database” available at www.kazusa.or.jp/codon.

The terms, “complementary” and “complementarity,” as used herein, with reference to a nucleic acid molecule or nucleotide sequence, refers to the characteristic of a polynucleotide having nucleotides that can undergo cumulative base pairing with a reference nucleic acid at two or more individual corresponding positions in antiparallel orientation. For example, when every nucleotide in a polynucleotide forms a base pair with every nucleotide in a reference nucleic acid, that polynucleotide is said to be 100% complementary to the reference nucleic acid. However, the term “complementary” by itself can include nucleic acid sequences that are not completely complementary over their entire length. Accordingly, the term “complementary” includes one or more mismatches. A “mismatch” is present at any position in the two opposed nucleotides are not complementary. In a double stranded DNA or RNA sequence, the upper (sense) strand sequence is, in general, understood as going in the direction from its 5′- to 3′-end, and the complementary sequence is thus understood as the sequence of the lower (antisense) strand in the same direction as the upper strand. Following the same logic, the reverse sequence is understood as the sequence of the upper strand in the direction from its 3′- to its 5′-end, while the ‘reverse complement’ sequence or the ‘reverse complementary’ sequence is understood as the sequence of the lower strand in the direction of its 5′- to its 3′-end. Each nucleotide in a nucleic acid that can be paired with its Watson-Crick counterpart (e.g, C with G; A with T/U) is called its complementary nucleotide. The complementarity of modified or artificial base pairs can be based on other types of hydrogen bonding and/or hydrophobicity of bases and/or shape complementarity between bases.

The term, “cleavage assay,” as used herein, refers to an assay designed to visualize, quantitate or identify cleavage of a nucleic acid. In some instances, the cleavage activity may be cis-cleavage activity. In some instances, the cleavage activity may be trans-cleavage activity. A non-limiting example of a cleavage assay is provided in Example 3.

The terms, “cleave,” “cleaving,” and “cleavage,” as used herein, with reference to a nucleic acid molecule or nuclease activity of an effector protein, refer to the hydrolysis of a phosphodiester bond of a nucleic acid molecule that results in breakage of that bond. The result of this breakage can be a nick (hydrolysis of a single phosphodiester bond on one side of a double-stranded molecule), single strand break (hydrolysis of a single phosphodiester bond on a single-stranded molecule) or double strand break (hydrolysis of two phosphodiester bonds on both sides of a double-stranded molecule) depending upon whether the nucleic acid molecule is single-stranded (e.g., ssDNA or ssRNA) or double-stranded (e.g., dsDNA) and the type of nuclease activity being catalyzed by the effector protein.

The term, “clustered regularly interspaced short palindromic repeats (CRISPR),” as used herein, refers to a segment of DNA found in the genomes of certain prokaryotic organisms, including some bacteria and archaea, that includes repeated short sequences of nucleotides interspersed at regular intervals between unique sequences of nucleotides derived from another organism.

The term “cointegrase activity” as used herein refers to catalytic activity that results in the transposition recombination of a first nucleic acid into a second nucleic acid.

The term, “conservative substitution” as used herein refers to the replacement of one amino acid for another such that the replacement takes place within a family of amino acids that are related in their side chains. Conversely, the term “non-conservative substitution” as used herein refers to the replacement of one amino acid residue for another that does not have a related side chain. Genetically encoded amino acids can be divided into four families having related side chains: (1) acidic (negatively charged): Asp (D), Glu (E); (2) basic (positively charged): Lys (K), Arg (R), His (H); (3) non-polar (hydrophobic): Cys (C), Ala (A), Val (V), Leu (L), Ile (I), Pro (P), Phe (F), Met (M), Trp (W), Gly (G), Tyr (Y), with non-polar also being subdivided into: (i) strongly hydrophobic: Ala (A), Val (V), Leu (L), Ile (I), Met (M), Phe (F); and (ii) moderately hydrophobic: Gly (G), Pro (P), Cys (C), Tyr (Y), Trp (W); and (4) uncharged polar: Asn (N), Gln (Q), Ser (S), Thr (T). Amino acids may be related by aliphatic side chains: Gly (G), Ala (A), Val (V), Leu (L), Ile (I), Ser (S), Thr (T), with Ser (S) and Thr (T) optionally being grouped separately as aliphatic-hydroxyl; Amino acids may be related by aromatic side chains: Phe (F), Tyr (Y), Trp (W). Amino acids may be related by amide side chains: Asn (N), Gln (Q). Amino acids may be related by sulfur-containing side chains: Cys (C) and Met (M).

The terms, “CRISPR RNA” or “crRNA,” as used herein, refers to a type of guide nucleic acid which is capable of interacting with an effector protein and/or to a target sequence of a target nucleic acid.

The term, “detectable signal,” as used herein, refers to a signal that can be detected using optical, fluorescent, chemiluminescent, electrochemical or other detection methods known in the art.

The term, “donor nucleic acid,” as used herein, refers to a nucleic acid that is (designed or intended to be) incorporated into a target nucleic acid or target sequence.

The term “dual nucleic acid system” as used herein refers to a system of a transactivated or transactivating guide nucleic acid-tracrRNA duplex that is complexed with one or more polypeptides described herein and imparts sequence selectivity to the complex when interacting with a target nucleic acid

The term, “effector protein,” as used herein, refers to a protein, polypeptide, or peptide that is capable of interacting with a nucleic acid, such as a guide nucleic acid, to form a complex, wherein the complex interacts with a target nucleic acid.

The term, “effector partner” or “partner polypeptide” as used herein, refers to a polypeptide that does not have 100% sequence identity with an effector protein described herein. In some instances, an effector partner described herein may be found in a homologous genome as an effector protein described herein.

The term, “engineered modification,” as used herein refers to a modification of one or more nucleic acid residues of a nucleotide sequence or one or more amino acid residue of an amino acid sequence, such as chemical modification of one or more nucleobases; or chemical modifications to the phosphate backbone, a nucleotide, a nucleobase, or a nucleoside. Such modifications can be made to an effector protein amino acid sequence or guide nucleic acid nucleotide sequence, or any sequence disclosed herein (e.g., a nucleic acid encoding an effector protein or a nucleic acid that encodes a guide nucleic acid). Methods of modifying a nucleic acid or amino acid sequence are known. One of ordinary skill in the art will appreciate that the engineered modification(s) may be located at any position(s) of a nucleic acid such that the function of the nucleic acid, protein, composition or system is not substantially decreased. Nucleic acids provided herein can be prepared according to any available technique including, but not limited to chemical synthesis, enzymatic synthesis, which is generally termed in vitro-transcription, cloning, enzymatic, or chemical cleavage, etc. In some instances, the nucleic acids provided herein are not uniformly modified along the entire length of the molecule. Different nucleotide modifications and/or backbone structures can exist at various positions within the nucleic acid.

The term, “functional fragment,” as used herein, refers to a fragment of a protein that retains some function relative to the entire protein. Non-limiting examples of functions are nucleic acid binding, protein binding, nuclease activity, nickase activity, deaminase activity, demethylase activity, or acetylation activity. A functional fragment may be a recognized functional domain, e.g., a catalytic domain such as, but not limited to, a RuvC domain.

The term, “functional fragment,” as used herein, refers to a fragment of a protein that retains some function relative to the entire protein. Non-limiting examples of functions are nucleic acid binding, protein binding, nuclease activity, nickase activity, transposase activity, cointegrase activity, ATPase activity, deaminase activity, demethylase activity, or acetylation activity. A functional fragment may be a recognized functional domain, e.g., a catalytic domain such as, but not limited to, a RuvC domain.

The term “fusion protein,” as used herein refers to a heterologous protein comprising at least two polypeptides. A fusion protein may comprise one or more of an effector protein and an effector partner, and a fusion partner.

The term “fusion partner,” as used herein, refers to a protein, polypeptide or peptide that is fused, or linked via a linker, to one or more of an effector protein and an effector partner. The fusion partner can impart some function to the fusion protein that is not provided by the effector protein or the effector partner.

The term, “genetic disease,” as used herein, refers to a disease, disorder, condition, or syndrome associated with or caused by one or more mutations in the DNA of an organism having the genetic disease.

The term, “guide nucleic acid,” as used herein, refers to a nucleic acid that, when in a complex with one or more polypeptides described herein can impart sequence selectivity to the complex when the complex interacts with a target nucleic acid. A guide nucleic acid may be referred to interchangeably as a guide RNA, however it is understood that guide nucleic acids may comprise deoxyribonucleotides (DNA), ribonucleotides (RNA), a combination thereof (e.g., RNA with a thymine base), biochemically or chemically modified nucleobases (e.g., one or more engineered modifications described herein), or combinations thereof.

The term, “handle sequence” as used herein, refers to a sequence of nucleotides in a single guide RNA (sgRNA), that is: 1) capable of being non-covalently bound by an effector protein and 2) connects the portion of the sgRNA capable of being non-covalently bound by an effector protein to a nucleotide sequence that is hybridizable to a target nucleic acid.

The term, “heterologous,” as used herein, with reference to at least two different polypeptide sequences, means that the two different polypeptide sequences are not found similarly connected to one another in a native nucleic acid or protein. A protein that is heterologous to the effector protein is a protein that is not covalently linked via an amide bond to the effector protein in nature. In some instances, a heterologous protein is not encoded by a species that encodes the effector protein. A guide nucleic acid may comprise a first sequence and a second sequence, wherein the first sequence and the second sequence are not found covalently linked via a phosphodiester bond in nature. Thus, the first sequence is considered to be heterologous with the second sequence, and the guide nucleic acid may be referred to as a heterologous guide nucleic acid.

The term, “hybridize,” “hybridizable,” or grammatical equivalents thereof refers to a sequence of nucleotides that is able to noncovalently bind, i.e. form Watson-Crick base pairs and/or G/U base pairs, or anneal, to another nucleotide sequence in a sequence-specific, antiparallel, manner (i.e., a nucleotide sequence specifically binds to a complementary nucleotide sequence) under the appropriate in vitro and/or in vivo conditions of temperature and solution ionic strength. Standard Watson-Crick base-pairing includes: adenine (A) pairing with thymidine (T), adenine (A) pairing with uracil (U), and guanine (G) pairing with cytosine (C) for both DNA and RNA. In addition, for hybridization between two RNA molecules (e.g., dsRNA), and for hybridization of a DNA molecule with an RNA molecule (e.g., when a DNA target nucleic acid base pairs with a guide RNA, etc.): guanine (G) can also base pair with uracil (U). For example, G/U base-pairing is at least partially responsible for the degeneracy (i.e., redundancy) of the genetic code in the context of tRNA anti-codon base-pairing with codons in mRNA. Thus, a guanine (G) can be considered complementary to both an uracil (U) and to an adenine (A). Accordingly, when a G/U base-pair can be made at a given nucleotide position, the position is not considered to be non-complementary, but is instead considered to be complementary.

While hybridization typically occurs between two nucleotide sequences that are complementary, mismatches between bases are possible. It is understood that two nucleotide sequences need not be 100% complementary to be specifically hybridizable, hybridizable, partially hybridizable, or for hybridization to occur. Moreover, a nucleotide sequence may hybridize over one or more segments such that intervening or adjacent segments are not involved in the hybridization event (e.g., a bulge, a loop structure or hairpin structure, etc.).

The term “intermediary sequence” or “intermediary RNA sequence” as used herein in a context of a single nucleic acid system, refers to a sequence of nucleotides in a guide nucleic acid that is capable of, at least partially, being non-covalently bound by an effector protein to form a complex. An intermediary RNA sequence is not a transactivating nucleic acid in systems, methods, and compositions described herein.

The term, “in vitro,” as used herein, is used to describe something outside an organism. An in vitro system, composition or method may take place in a container for holding laboratory reagents such that it is separated from the biological source from which a material in the container is obtained. In vitro assays can encompass cell-based assays in which living or dead cells are employed. In vitro assays can also encompass a cell-free assay in which no intact cells are employed. The term “in vivo” is used to describe an event that takes place within an organism. The term “ex vivo” is used to describe an event that takes place in a cell that has been obtained from an organism. An ex vivo assay is not performed on a subject. Rather, it is performed upon a sample separate from a subject.

The term “insertion site” as used herein refers to a location on a target nucleic acid into which a donor nucleic acid may be inserted.

The term “length” as it applies to a nucleic acid (polynucleotide) or polypeptide may be expressed as “kilobases” (kb) or “base pairs (bp),” and may be used interchangeably with the term, “linked nucleosides.” Thus, a length of 1 kb refers to a length of 1000 linked nucleosides, and a length of 500 bp refers to a length of 500 linked nucleosides. Similarly, a protein having a length of 500 linked amino acids may also be simply described as having a length of 500 amino acids.

The term, “linker,” as used herein, refers to a covalent bond or molecule that links a first polypeptide to a second polypeptide or a first nucleic acid to a second nucleic acid.

The term, “modified target nucleic acid,” as used herein, refers to a target nucleic acid, wherein the target nucleic acid has undergone a modification, for example, after contact with an effector protein. In some instances, the modification is an alteration in the sequence of the target nucleic acid. In some instances, the modified target nucleic acid comprises an insertion, deletion, or substitution of one or more nucleotides compared to the unmodified target nucleic acid.

The term, “mutation” as used herein when describing an alteration or modification that changes an amino acid residue or a nucleotide as described herein, such a change or changes can include, for example, deletions, insertions, and/or substitutions. The mutation can refer to a change in structure of an amino acid residue or nucleotide relative to the starting or reference residue or nucleotide. A mutation of an amino acid residue includes, for example, deletions, insertions and substituting one amino acid residue for a structurally different amino acid residue. Such substitutions can be a conservative substitution, a non-conservative substitution, a substitution to a specific sub-class of amino acids, or a combination thereof as described herein. A mutation of a nucleotide includes, for example, changing one naturally occurring base for a different naturally occurring base, such as changing an adenine to a thymine or a guanine to a cytosine or an adenine to a cytosine or a guanine to a thymine. A mutation of a nucleotide base may result in a structural and/or functional alteration of the encoding peptide, polypeptide or protein by changing the encoded amino acid residue of the peptide, polypeptide or protein. A mutation of a nucleotide base may not result in an alteration of the amino acid sequence or function of encoded peptide, polypeptide or protein, also known as a silent mutation. Methods of mutating an amino acid residue or a nucleotide are well known.

The terms, “mutation associated with a disease,” and “mutation associated with a genetic disorder,” as used herein, refers to the co-occurrence of a mutation and the phenotype of a disease. The mutation may occur in a gene, wherein transcription or translation products from the gene occur at a significantly abnormal level or in an abnormal form in a cell or subject harboring the mutation as compared to a non-disease control subject not having the mutation.

The term, “nickase” as used herein refers to an enzyme that possess catalytic activity for single stranded nucleic acid cleavage of a double stranded nucleic acid.

The term “nickase activity” as used herein refers to catalytic activity that results in single stranded nucleic acid cleavage of a double stranded nucleic acid.

The terms, “non-naturally occurring” and “engineered,” as used herein, are used interchangeably and indicate the involvement of the hand of man. The terms, when referring to a nucleic acid, nucleotide, protein, polypeptide, peptide or amino acid, refer to a molecule, such as but not limited to, a nucleic acid, nucleotide, protein, polypeptide, peptide or amino acid refers to a modification of that molecule (e.g., chemical modification, nucleotide sequence, or amino acid sequence) that is not present in the naturally molecule. The terms, when referring to a composition or system described herein, refer to a composition or system having at least one component that is not naturally associated with the other components of the composition or system. By way of a non-limiting example, a composition may include an effector protein and a guide nucleic acid that do not naturally occur together. Conversely, and as a non-limiting further clarifying example, an effector protein or guide nucleic acid that is “natural,” “naturally-occurring,” or “found in nature” includes an effector protein and a guide nucleic acid from a cell or organism that have not been genetically modified by the hand of man.

The terms, “nuclease” and “endonuclease” are used interchangeably herein, refer to an enzyme which possesses catalytic activity for nucleic acid cleavage.

The term, “nuclease activity,” is used herein, refers to catalytic activity that results in nucleic acid cleavage (e.g., ribonuclease activity (ribonucleic acid cleavage), or deoxyribonuclease activity (deoxyribonucleic acid cleavage), etc.).

The term, “nucleic acid,” as used herein refers to a polymer of nucleotides. A nucleic acid may comprise ribonucleotides, deoxyribonucleotides, combinations thereof, and modified versions of the same. A nucleic acid may be single-stranded or double-stranded, unless specified. Non-limiting examples of nucleic acids are double stranded DNA (dsDNA), single stranded (ssDNA), messenger RNA, genomic DNA, cDNA, DNA-RNA hybrids, and a polymer comprising purine and pyrimidine bases or other natural, chemically or biochemically modified, non-natural, or derivatized nucleotide bases. Accordingly, nucleic acids as described herein may comprise one or more mutations, one or more engineered modifications, or both.

The term, “nucleic acid expression vector,” as used herein, refers to a plasmid that can be used to express a nucleic acid of interest.

The term, “nuclear localization signal (NLS),” as used herein, refers to an entity (e.g., peptide) that facilitates localization of a nucleic acid, protein, or small molecule to the nucleus, when present in a cell that contains a nuclear compartment.

A person of ordinary skill in the art would appreciate that referring to a “nucleotide(s)”, and/or “nucleoside(s)”, in the context of a nucleic acid molecule having multiple residues, is interchangeable and describe the sugar and base of the residue contained in the nucleic acid molecule. Similarly, a skilled artisan could understand that linked nucleotides and/or linked nucleosides, as used in the context of a nucleic acid having multiple linked residues, are interchangeable and describe linked sugars and bases of residues contained in a nucleic acid molecule. When referring to a “nucleobase(s)”, or linked nucleobase, as used in the context of a nucleic acid molecule, it can be understood as describing the base of the residue contained in the nucleic acid molecule, for example, the base of a nucleotide, nucleosides, or linked nucleotides or linked nucleosides. A person of ordinary skill in the art when referring to nucleotides, nucleosides, and/or nucleobases would also understand the differences between RNA and DNA (generally the exchange of uridine for thymidine or vice versa) and the presence of nucleoside analogs, such as modified uridines, do not contribute to differences in identity or complementarity among polynucleotides as long as the relevant nucleotides (such as thymidine, uridine, or modified uridine) have the same complement (e.g., adenosine for all of thymidine, uridine, or modified uridine; another example is cytosine and 5-methylcytosine, both of which have guanosine or modified guanosine as a complement). Thus, for example, the sequence 5′-AXG where X is any modified uridine, such as pseudouridine, NI-methyl pseudouridine, or 5-methoxyuridine, is considered 100% identical to AUG in that both are perfectly complementary to the same sequence (5′-CAU).

The term “pharmaceutically acceptable excipient, carrier or diluent,” as used herein, refers to any substance formulated alongside the active ingredient of a pharmaceutical composition that allows the active ingredient to retain biological activity and is non-reactive with the subject's immune system. Such a substance can be included for the purpose of long-term stabilization, bulking up solid formulations that contain potent active ingredients in small amounts, or to confer a therapeutic enhancement on the active ingredient in the final dosage form, such as facilitating absorption, reducing viscosity, or enhancing solubility. The selection of appropriate substance can depend upon the route of administration and the dosage form, as well as the active ingredient and other factors. Compositions having such substances can be formulated by suitable methods (see, e.g., Remington's Pharmaceutical Sciences, 18th edition, A. Gennaro, ed., Mack Publishing Co., Easton, Pa., 1990; and Remington, The Science and Practice of Pharmacy 21st Ed. Mack Publishing, 2005).

The terms, “polypeptide” and “protein” which are used interchangeably herein, refer to a polymeric form of amino acids. A polypeptide may include coded and non-coded amino acids, chemically or biochemically modified or derivatized amino acids, and polypeptides having modified peptide backbones. Accordingly, polypeptides as described herein may comprise one or more mutations, one or more engineered modifications, or both. A peptide generally has a length of 100 or fewer linked amino acids.

The term, “promoter” or “promoter sequence,” is a DNA regulatory region capable of binding RNA polymerase and initiating transcription of a downstream (3′ direction) coding or non-coding sequence. A transcription initiation site, as well as protein binding domains responsible for the binding of RNA polymerase, can also be found in a promoter region. Eukaryotic promoters will often, but not always, contain “TATA” boxes and “CAT” boxes. Various promoters, including inducible promoters, may be used to drive expression by the various vectors of the present disclosure.

The term, “protospacer adjacent motif (PAM),” as used herein, refers to a nucleotide sequence found in a target nucleic acid that directs an effector protein to modify the target nucleic acid at a specific location. In some instances, a PAM is required for a complex of an effector protein and a guide nucleic acid to hybridize to and modify the target nucleic acid. In some instances, the complex does not require a PAM to modify the target nucleic acid.

The term, “recombinant,” as used herein, as applied to proteins, polypeptides, peptides and nucleic acids, refers to proteins, polypeptides, peptides and nucleic acids that are products of various combinations of cloning, restriction, and/or ligation steps resulting in a construct having a structural coding or non-coding sequence distinguishable from endogenous nucleic acids found in natural systems.

The term, “regulatory element,” used herein, refers to transcriptional and translational control sequences, such as promoters, enhancers, polyadenylation signals, terminators, protein degradation signals, and the like, that provide for and/or regulate transcription of a non-coding sequence (e.g., a guide nucleic acid) or a coding sequence (e.g., effector proteins, fusion proteins, and the like) and/or regulate translation of an encoded polypeptide.

The term “repeat hybridization sequence” as used herein refers to a sequence of nucleotides that is, at least, partially complementary to a repeat sequence.

The term “repeat sequence” as used herein refers to a sequence of nucleotides in a guide nucleic acid that is capable of, at least partially, interacting with an effector protein.

The terms, “reporter” and “reporter nucleic acid” are used interchangeably herein to refer to a non-target nucleic acid molecule that can provide a detectable signal upon cleavage by an effector protein. Examples of detectable signals and detectable moieties that generate detectable signals are provided herein.

The term “ribonucleotide protein complex” or “RNP” as used herein refers to a complex of one or more nucleic acids and one or more polypeptides. While the term utilizes “ribonucleotides” it is understood that the one or more nucleic acid may comprise deoxyribonucleotides (DNA), ribonucleotides (RNA), a combination thereof (e.g., RNA with a thymine base), biochemically or chemically modified nucleobases (e.g., one or more enginereed modifications described herein), or combinations thereof.

The term, “sample,” as used herein, generally refers to something comprising a target nucleic acid. In some instances, the sample is a biological sample, such as a biological fluid or tissue sample. In some instances, the sample is an environmental sample. The sample may be a biological sample or environmental sample that is modified or manipulated. By way of non-limiting example, samples may be modified or manipulated with purification techniques, heat, nucleic acid amplification, salts and buffers.

The terms “single guide nucleic acid”, “single guide RNA” and “sgRNA” as used interchangeably herein refers to a type of guide nucleic acid in a single nucleic acid system that interacts with one or more polypeptides described herein to form a complex and imparts sequence selectivity to said complex.

The term “single nucleic acid system” as used herein refers to a guide nucleic acid wherein the guide nucleic acid is a single polynucleotide chain having all the required sequences for a functional complex with an effector protein (e.g., being bound by an effector protein, including in some instances activating the effector protein, and hybridizing to a target nucleic acid, without the need for a second nucleic acid molecule). For example, an sgRNA can have two or more linked guide nucleic acid components (e.g., a repeat sequence and a spacer sequence or a handle sequence and a spacer sequence).

The term “spacer sequence” as used herein, refers to a sequence of nucleotides in guide nucleic acid, which is, at least partially, hybridizable to an equal length of a sequence (e.g., a target sequence) of a target nucleic acid.

The term, “subject,” as used herein, refers to an animal. The subject may be a mammal. The subject may be a human. The subject may be diagnosed or at risk for a disease.

A “syndrome”, as used herein, refers to a group of symptoms which, taken together, characterize a condition.

The term, “target nucleic acid,” as used herein, refers to a nucleic acid that is selected as the nucleic acid for modification, binding, hybridization or any other activity of or interaction with a nucleic acid, protein, polypeptide, or peptide described herein. A target nucleic acid may comprise RNA, DNA, or a combination thereof. A target nucleic acid may be single-stranded (e.g., single-stranded RNA or single-stranded DNA) or double-stranded (e.g., double-stranded DNA).

The term, “target sequence,” as used herein, when used in reference to a target nucleic acid, refers to a sequence of nucleotides found within a target nucleic acid. Such a sequence of nucleotides can, for example, hybridize to a respective length portion of a guide nucleic acid.

The term “trans-activating RNA”, “transactivating RNA” or “tracrRNA” refers to a transactivating or transactivated nucleic acid in a dual nucleic acid system that interacts with an effector protein and hybridizes, at least partially, to a guide nucleic acid to form a guide nucleic acid-tracrRNA duplex.

The term “transactivating” or “trans-activating” as used herein, refers to an outcome of a dual nucleic acid system wherein the two nucleic acids are required to hybridize for the system to have activity.

The term “trans cleavage,” as used herein, in reference to cleavage (e.g, hydrolysis of a phosphodiester bond) of one or more non-target nucleic acids by an effector protein that is complexed with a guide nucleic acid and a target nucleic acid. Trans cleavage activity may be triggered by the hybridization of a guide nucleic acid to a target nucleic acid. The effector protein may cleave the target nucleic acid as well as non-target nucleic acids.

The term “transcriptional activator,” as used herein, refers to a polypeptide or a fragment thereof that can activate or increase transcription of a target nucleic acid molecule.

The term “transcriptional repressor,” as used herein, refers to a polypeptide or a fragment thereof that is capable of arresting, preventing, or reducing transcription of a target nucleic acid.

The term “transgene” as used herein refers to a nucleotide sequence that is inserted into a cell for expression of said nucleotide sequence in the cell. A transgene is meant to include (1) a nucleotide sequence that is not naturally found in the cell (e.g., a heterologous nucleotide sequence); (2) a nucleotide sequence that is a mutant form of a nucleotide sequence naturally found in the cell into which it has been introduced; (3) a nucleotide sequence that serves to add additional copies of the same (e.g., exogenous or homologous) or a similar nucleotide sequence naturally occurring in the cell into which it has been introduced; or (4) a silent naturally occurring or homologous nucleotide sequence whose expression is induced in the cell into which it has been introduced. A donor nucleic acid can comprise a transgene. The cell in which transgene expression occurs can be a target cell, such as a host cell.

The terms, “treatment” or “treating,” are used herein in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying, or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The term “transposase activity” as used herein refers to catalytic activity that results in the transposition of a first nucleic acid into a second nucleic acid.

The term, “variant,” is intended to mean a form or version of a protein that differs from the wild-type protein. A variant may have a different function or activity relative to the wild-type protein.

The term “viral vector,” as used herein, refers to a nucleic acid to be delivered into a host cell via a recombinantly produced virus or viral particle.

### III. INTRODUCTION

Disclosed herein are compositions, systems and methods comprising at least one of:
a) a polypeptide or a nucleic acid encoding the polypeptide;
b) a partner polypeptide or a nucleic acid encoding the partner polypeptide; and
c) a guide nucleic acid or a nucleic acid encoding the guide nucleic acid.

Polypeptides described herein may recognize a desired nucleic acid, such as a target nucleic acid, and cleave a desired nucleic acid by either cis or trans cleavage. In some embodiments, a polypeptide described herein may bind to a target sequence of a target nucleic acid and cleave a desired nucleic acid by either cis or trans cleavage. In some embodiments, a polypeptide is activated when it binds a target sequence of a target nucleic acid to cleave a region of the target nucleic acid that is near, but not adjacent to the target sequence. A polypeptide may be an effector protein, such as a CRISPR-associated (Cas) protein, which may be coupled to a guide nucleic acid that imparts activity or sequence selectivity to the polypeptide.

Also described herein is a partner polypeptide or use thereof. In general, a partner polypeptide comprises an amino acid sequence that is not 100% identical to a polypeptide (i.e., effector protein) described above. Partner polypeptides described herein may have biological activity that is synergistic, complementary, or additive to the activity of polypeptides (i.e., effector proteins) described above. A partner polypeptide may be an effector partner.

In general, guide nucleic acids comprise a first sequence, at least a portion of which interacts with a polypeptide. In some embodiments, guide nucleic acids may further comprise a second sequence that is at least partially complementary to a target nucleic acid.

In some embodiments, effector proteins and/or effector partners described herein are encoded by a codon optimized nucleic acid. In some embodiments, a nucleic acid sequence encoding an effector protein and/or effector partner described herein, is codon optimized. In some embodiments, effector proteins and/or effector partners described herein may be codon optimized for expression in a specific cell, for example, a bacterial cell, a plant cell, a eukaryotic cell, an animal cell, a mammalian cell, or a human cell. In some embodiments, the effector protein and/or effector partner is codon optimized for a human cell. It is understood that when describing coding sequences of polypeptides described herein, said coding sequences do not necessarily require a codon encoding a N-terminal Methionine (M) or a Valine (V) as described for the effector proteins described herein. One skilled in the art would understand that a start codon could be replaced or substituted with a start codon that encodes for an amino acid residue sufficient for initiating translation in a host cell. In some embodiments, when a modifying heterologous peptide, such as a fusion partner protein, protein tag or NLS, is located at the N terminus of the effector protein, a start codon for the heterologous peptide serves as a start codon for the effector protein as well. Thus, the natural start codon encoding an amino acid residue sufficient for initiating translation (e.g., Methionine (M) or a Valine (V)) of the effector protein may be removed or absent.

The compositions, systems and methods described herein are non-naturally occurring. In some embodiments, compositions, systems and methods comprise an engineered guide nucleic acid or a use thereof. In some embodiments, compositions, systems and methods comprise an engineered polypeptide or a use thereof. In some embodiments, compositions, systems and methods comprise an isolated polypeptide or a use thereof. In general, compositions, methods and systems described herein are not found in nature. In some embodiments, compositions, methods and systems described herein comprise at least one non-naturally occurring component. For example, disclosed compositions, methods and systems may comprise a guide nucleic acid, wherein the sequence of the guide nucleic acid is different or modified from that of a naturally-occurring guide nucleic acid.

In some embodiments, compositions, systems and methods comprise at least two components that do not naturally occur together. For example, disclosed compositions, methods and systems may comprise a guide nucleic acid comprising a repeat region and a spacer region which do not naturally occur together. Also, by way of non-limiting example, disclosed compositions, methods and systems may comprise a guide nucleic acid and an effector protein that do not naturally occur together. Similarly, disclosed compositions, methods and systems may comprise a guide nucleic acid and an effector partner that do not naturally occur together. Conversely, and for clarity, an effector protein, effector partner, or guide nucleic acid that is “natural,” “naturally-occurring,” or “found in nature” includes effector proteins, effector partners and guide nucleic acids from cells or organisms that have not been genetically modified by a human or machine.

In some embodiments, the guide nucleic acid comprises a non-natural nucleotide sequence. In some embodiments, the non-natural nucleotide sequence is a nucleotide sequence that is not found in nature. The non-natural nucleotide sequence may comprise a portion of a naturally-occurring sequence, wherein the portion of the naturally-occurring sequence is not present in nature absent the remainder of the naturally-occurring sequence. In some embodiments, the guide nucleic acid comprises two naturally-occurring sequences arranged in an order or proximity that is not observed in nature. Engineered guide nucleic acids may comprise a first sequence and a second sequence that do not occur naturally together. For example, a guide nucleic acid may comprise a sequence of a naturally-occurring repeat region and a spacer region that is complementary to a naturally-occurring eukaryotic sequence. The guide nucleic acid may comprise a sequence of a repeat region that occurs naturally in an organism and a spacer region that does not occur naturally in that organism. A guide nucleic acid may comprise a first sequence that occurs in a first organism and a second sequence that occurs in a second organism, wherein the first organism and the second organism are different. The guide nucleic acid may comprise a third sequence disposed at a 3′ or 5′ end of the guide nucleic acid, or between the first and second sequences of the guide nucleic acid. In some embodiments, the guide nucleic acid comprises two heterologous sequences arranged in an order or proximity that is not observed in nature. In some embodiments, compositions, methods and systems described herein comprise a ribonucleotide complex comprising an effector protein and a guide nucleic acid that do not occur together in nature. Therefore, compositions, methods and systems described herein are not naturally occurring.

In some embodiments, compositions, methods and systems described herein comprise an effector protein that is similar to a naturally occurring effector protein. The effector protein may lack a portion of the naturally occurring effector protein. The effector protein may comprise a mutation relative to the naturally-occurring effector protein, wherein the mutation is not found in nature. The effector protein may also comprise at least one additional amino acid relative to the naturally-occurring effector protein. In some embodiments, the effector protein may comprise a heterologous polypeptide. For example, the effector protein may comprise an addition of a nuclear localization signal relative to the natural occurring effector protein. In some embodiments, a nucleotide sequence encoding the effector protein is codon optimized (e.g., for expression in a eukaryotic cell) relative to the naturally occurring sequence.

In some embodiments, compositions and systems described herein comprise an effector partner that is similar to a naturally occurring effector partner. The effector partner may lack a portion of the naturally occurring effector partner. The effector partner may comprise a mutation relative to the naturally-occurring effector partner, wherein the mutation is not found in nature. The effector partner may also comprise at least one additional amino acid relative to the naturally-occurring effector partner. In some embodiments, the effector partner may comprise a heterologous polypeptide. For example, the effector partner may comprise an addition of a nuclear localization signal relative to the natural occurring effector partner. In some embodiments, a nucleotide sequence encoding the effector partner is codon optimized (e.g., for expression in a eukaryotic cell) relative to the naturally occurring sequence.

### IV. POLYPEPTIDE SYSTEMS

Effector Proteins

Provided herein are compositions, systems, and methods comprising an effector protein or a use thereof. Effector proteins disclosed herein may cleave nucleic acids, including single stranded RNA (ssRNA), double stranded DNA (dsDNA), and single-stranded DNA (ssDNA). Effector proteins disclosed herein may provide cis cleavage activity, trans cleavage activity, nickase activity, nuclease activity, integrase activity, cointegrase activity or a combination thereof.

An effector protein provided herein interacts with a guide nucleic acid to form a complex. In some embodiments, the complex interacts with a target nucleic acid. In some embodiments, an interaction between the complex and the target nucleic acid comprises recognition of a PAM sequence by the effector protein, hybridization of the guide nucleic acid to the target nucleic acid, and/or modification of the target nucleic acid by the effector protein. In some embodiments, an ability of an effector protein to modify a target nucleic acid may be dependent upon the effector protein being bound to a guide nucleic acid and the guide nucleic acid being hybridized to a target nucleic acid. An effector protein may also recognize a protospacer adjacent motif (PAM) sequence present in the target nucleic acid. An effector protein may modify the target nucleic acid by cis cleavage and/or trans cleavage. The modification of the target nucleic acid generated by an effector protein may, as a non-limiting example, result in modulation of the expression of the target nucleic acid (e.g., increasing or decreasing expression of the nucleic acid) or modulation of the activity of a translation product of the target nucleic acid (e.g., inactivation of a protein binding to an RNA molecule or hybridization). An effector protein used herein may be a CRISPR-associated (“Cas”) protein.

An effector protein may function as a single protein, including a single protein that is capable of binding to a guide nucleic acid and modifying a target nucleic acid. Alternatively, an effector protein may function as part of a multiprotein complex, including, for example, a complex having two or more effector proteins, including two or more of the same effector proteins (e.g., dimer or multimer). An effector protein, when functioning in a multiprotein complex, may have only one functional activity (e.g., binding to a guide nucleic acid), while other effector proteins present in the multiprotein complex are capable of the other functional activity (e.g., modifying a target nucleic acid). In some embodiments, an effector protein, when functioning in a multiprotein complex, may have differing and/or complementary functional activity (e.g., transposase activity) to other effector proteins in the multiprotein complex (e.g., cointegrate activity). Multimeric complexes, and functions thereof, are described in further detail below. An effector protein may be a modified effector protein having increased modification activity and/or increased substrate binding activity (e.g., substrate selectivity, specificity, and/or affinity). Alternatively, or in addition, an effector protein may be a catalytically inactive effector protein having reduced modification activity or no modification activity.

TABLE 1 provides illustrative amino acid sequences of effector proteins that are useful in the compositions, systems and methods described herein.

In some embodiments, compositions, systems and methods described herein provided herein comprise an effector protein, wherein the amino acid sequence of the effector protein comprises at least about 200 contiguous amino acids or more of any one of the sequences recited in TABLE 1. In some embodiments, the amino acid sequence of an effector protein provided herein comprises at least about 200, at least about 220, at least about 240, at least about 260, at least about 280, at least about 300, at least about 320, at least about 340, at least about 360, at least about 380, at least about 400 contiguous amino acids, at least about 420 contiguous amino acids, at least about 440 contiguous amino acids, at least about 460 contiguous amino acids, at least about 480 contiguous amino acids, at least about 500 contiguous amino acids, at least about 520 contiguous amino acids, at least about 540 contiguous amino acids, at least about 560 contiguous amino acids, at least about 580 contiguous amino acids, at least about 600 contiguous amino acids, or more of any one of the sequences of TABLE 1.

In some embodiments, other than a truncation of the first 10 amino acids, 20 amino acids, 30 amino acids, 40 amino acids, 50 amino acids, 60 amino acids, 70 amino acids, 80 amino acids, 90 amino acids, or 100 amino acids, and/or a truncation of the last 10 amino acids, 20 amino acids, 30 amino acids, 40 amino acids, 50 amino acids, 60 amino acids, 70 amino acids, 80 amino acids, 90 amino acids, or 100 amino acids, the amino acid sequence of an effector protein provided herein comprises any one of the sequences of TABLE 1.

In some embodiments, compositions, systems, and methods provided herein comprise an effector protein, wherein the effector protein comprises an amino acid sequence that is at least 65% identical to any one of the sequences as set forth in TABLE 1. In some embodiments, an effector protein provided herein is at least 65%, at least 70%, at least 75%, at least 80%, at least 85%, at least 90%, at least 95%, at least 97%, or at least 98%, at least 99%, or 100% identical to any one of the sequences as set forth in TABLE 1. In some embodiments, compositions, systems, and methods provided herein comprise an effector protein and an engineered guide nucleic acid, wherein the effector protein comprises an amino acid sequence that is at least 65%, at least 70%, at least 75%, at least 80%, at least 85%, at least 90%, at least 95%, at least 98%, at least 99%, or 100% identical to any one of the sequences as set forth in TABLE 1.

In some embodiments, an effector protein provided herein comprises an amino acid sequence that is at least 65% identical to any one of the sequences as set forth in TABLE 1. In some embodiments, an effector protein provided herein comprises an amino acid sequence that is at least 70% identical to any one of the sequences as set forth in TABLE 1. In some embodiments, an effector protein provided herein comprises an amino acid sequence that is at least 75% identical to any one of the sequences as set forth in TABLE 1. In some embodiments, an effector protein provided herein comprises an amino acid sequence that is at least 80% identical to any one of the sequences as set forth in TABLE 1. In some embodiments, an effector protein provided herein comprises an amino acid sequence that is at least 85% identical to any one of the sequences as set forth in TABLE 1. In some embodiments, an effector protein provided herein comprises an amino acid sequence that is at least 90% identical to any one of the sequences as set forth in TABLE 1. In some embodiments, an effector protein provided herein comprises an amino acid sequence that is at least 95% identical to any one of the sequences as set forth in TABLE 1. In some embodiments, an effector protein provided herein comprises an amino acid sequence that is at least 98% identical to any one of the sequences as set forth in TABLE 1. In some embodiments, an effector protein provided herein comprises an amino acid sequence that is at least 99% identical to any one of the sequences as set forth in TABLE 1. In some embodiments, an effector protein provided herein comprises an amino acid sequence that is identical to any one of the sequences as set forth in TABLE 1.

In some embodiments, compositions, systems, and methods provided herein comprise an effector protein, wherein the effector protein comprises one or more amino acid alteration relative to any one of the sequences of TABLE 1 wherein the effector protein comprising one or more amino acid alterations is a variant of an effector protein described herein. It is understood that any reference to an effector protein herein also refers to an effector protein variant as described herein. In some embodiments, an effector protein provided herein comprises: 1 amino acid alteration, 2 amino acid alterations, 3 amino acid alterations, 4 amino acid alterations, 5 amino acid alterations, 6 amino acid alterations, 7 amino acid alterations, 8 amino acid alterations, 9 amino acid alterations, 10 amino acid alterations or more relative to any one of the sequences of TABLE 1.

In some embodiments, the one or more amino acid alteration is a conservative or non-conservative substitution. In some embodiments, an effector protein provided herein comprises: 1 conservative amino acid substitution, 2 conservative amino acid substitutions, 3 conservative amino acid substitutions, 4 conservative amino acid substitutions, 5 conservative amino acid substitutions, 6 conservative amino acid substitutions, 7 conservative amino acid substitutions, 8 conservative amino acid substitutions, 9 conservative amino acid substitutions, 10 conservative amino acid substitutions or more relative to any one of the sequences of TABLE 1. In some embodiments, an effector protein provided herein comprises: 1 non-conservative amino acid substitution, 2 non-conservative amino acid substitutions, 3 non-conservative amino acid substitutions, 4 non-conservative amino acid substitutions, 5 non-conservative amino acid substitutions, 6 non-conservative amino acid substitutions, 7 non-conservative amino acid substitutions, 8 non-conservative amino acid substitutions, 9 non-conservative amino acid substitutions, 10 non-conservative amino acid substitutions or more relative to any one of the sequences of TABLE 1.

In some embodiments, the one or more amino acid alterations may result in a change in activity of the effector protein relative to a naturally-occurring counterpart. For example, and as described in further detail below, the one or more amino acid alteration increases or decreases catalytic activity of the effector protein relative to a naturally-occurring counterpart. In some embodiments, the one or more amino acid alterations results in a catalytically inactive effector protein variant.

In some embodiments, described herein are methods and systems for identifying effector proteins for use herein. For example, in some embodiments, such methods for identifying an effector protein suitable for use herein may comprise the steps of identifying a CRISPR array in a database and selecting a sequence within about 150 bp, or about 150 bp to about 3.5 kb of a CRISPR Cas locus, wherein the identified sequence encodes an effector protein and one or more effector partners. In some embodiments, an identified sequence comprises one or more Open Reading Frames (ORFs) of which a first ORF encodes an effector protein and in some embodiments a second ORF encodes one or more effector partner. In embodiments where the identified sequence comprises two ORFs. In some embodiments where the identified sequence comprises two ORFs, the ORFs may overlap. In some embodiments, an effector partner is downstream of an effector protein. In some embodiments, an effector partner is upstream of an effector protein. In some embodiments, an identified sequence encodes an effector protein and two or more effector partners wherein a first effector partner is downstream of the effector protein and the second effector partner is upstream of the effector protein.

Effector Partners

Provided herein, are compositions, systems, and methods comprising an effector partner (e.g., partner polypeptide) or a use thereof. In some embodiments, an effector partner as described herein is referred to as a partner polypeptide. In some embodiments, effector partners described herein may have an activity that is synergistic, complementary, and/or additive to the activity of an effector protein. In some embodiments, effector partners described herein may have cointegrase activity or ATPase activity. In some embodiments, an effector partner comprises an amino acid sequence that is not 100% identical to an amino acid sequence of an effector protein described herein.

In some embodiments, compositions, systems, and methods described herein comprise one or more effector partners. In some embodiments, compositions, systems, and methods described herein comprise 1, 2, 3, 4, 5, 6, 7, 8, 9, 10 or more effector partners. In some embodiments, a genomic sequence encoding a naturally-occurring effector partner provided herein is found downstream of a genomic sequence encoding a naturally-occurring effector protein described herein. In some embodiments, an ORF of a genomic sequence encoding a naturally-occurring effector partner overlaps with an ORF of a genomic sequence encoding a naturally-occurring effector protein. In some embodiments, an ORF of a genomic sequence encoding a naturally-occurring effector partner does not overlap with an ORF of a genomic sequence encoding a naturally-occurring effector protein. In some embodiments, a genomic sequence encoding a naturally-occurring effector partner provided herein is found about 1 bp to about 50 bp downstream of a genomic sequence encoding a naturally-occurring effector protein described herein.

In some embodiments, a genomic sequence encoding a first naturally-occurring effector partner is found downstream of a genomic sequence encoding a naturally-occurring effector protein, and a genomic sequence encoding a second naturally-occurring effector partner is found downstream of the genomic sequence of the first naturally-occurring effector partner. In some embodiments, a genomic sequence encoding a second naturally-occurring effector partner provided herein is found about 1 bp to about 3.5 kb downstream of a genomic sequence encoding a first naturally-occurring effector partner described herein. In some embodiments, a genomic sequence encoding a first naturally-occurring effector partner is found downstream of a genomic sequence encoding a naturally-occurring effector protein, and a genomic sequence encoding a second naturally-occurring effector partner is found upstream of the genomic sequence of the naturally-occurring effector protein. In some embodiments, a genomic sequence encoding a second naturally-occurring effector partner provided herein is found about 1 bp to about 50 bp upstream of a genomic sequence encoding a first naturally-occurring effector protein described herein.

In some embodiments, compositions, systems, and methods described herein comprise one or more, two or more, three or more, four or more, five or more effector partners, or one or more nucleic acids encoding the one or more, two or more, three or more, four or more, five or more effector partners. In some embodiments, compositions, systems, and methods described herein comprise two or more effector partners, or one or more nucleic acids encoding the two or more effector partners. In some embodiments, compositions, systems, and methods described herein comprise three or more effector partners, or one or more nucleic acids encoding the three or more effector partners. In some embodiments, compositions, systems, and methods described herein comprise four or more effector partners, or one or more nucleic acids encoding the four or more effector partners. In some embodiments, compositions, systems, and methods described herein comprise five or more effector partners, or one or more nucleic acids encoding the five or more effector partners. In some embodiments, the one or more effector partners comprise an amino acid sequence that is at least 75% identical, at least 80% identical, at least 85% identical, at least 90% identical, at least 95% identical, or at least 100% identical to any one of the sequences set forth in TABLE 1.1.

In some embodiments, compositions, systems, and methods described herein comprise any one of the effector partners (e.g., partner polypeptide) described herein. In some embodiments, compositions described herein independently comprise any one of the effector partners described herein. In some embodiments, compositions comprise an effector partner which functions as a single protein. In some embodiments, a composition comprising an effector partner as described herein may be independently administered from a composition comprising an effector protein as described herein. In some embodiments, an independently administered composition comprising one or more effector partners as described herein, wherein each effector partner independently comprises an amino acid sequence that is at least 75% identical, at least 80% identical, at least 85% identical, at least 90% identical, at least 95% identical, or at least 100% identical to any one of the sequences set forth in TABLE 1.1. In some embodiments, an independently administered composition comprising one or more effector partners as described herein, wherein each effector partner independently imparts some function or activity. In some embodiments, compositions described herein comprise effector partner combinations wherein each effector partner independently imparts a function or activity.

In some embodiments, the effector partner imparts some function or activity that is not provided by an effector protein. In some embodiments, the effector partner imparts some function or activity that is synergistic, complementary, and/or additive to the function or activity provided by an effector protein. In some embodiments, the effector partner is capable of cleaving or modifying the target nucleic acid, a non-target nucleic acid, or both. In some embodiments, the effector partner provides cleavage activity, such as cis cleavage activity, trans cleavage activity, nickase activity, nuclease activity, other activity, or a combination thereof. In some embodiments, the effector partner comprises a RuvC domain capable of cleavage activity. In some embodiments, the effector partner cleaves nucleic acids, including single stranded RNA (ssRNA), double stranded DNA (dsDNA), and single-stranded DNA (ssDNA). In some embodiments, the effector partner cleaves the target nucleic acid at the target sequence or adjacent to the target sequence. In some embodiments, the effector partner cleaves the non-target nucleic acid.

In some embodiments, the effector partner complexes with a guide nucleic acid and the complex interacts with the target nucleic acid, a non-target nucleic acid, or both. In some embodiments, the interaction comprises one or more of: recognition of a protospacer adjacent motif (PAM) sequence within the target nucleic acid by the effector partner, hybridization of the guide nucleic acid to the target nucleic acid, modification of the target nucleic acid, and/or the non-target nucleic acid by the effector partner cleaves, or combinations thereof. In some embodiments, recognition of a PAM sequence within a target nucleic acid directs the modification activity.

In some embodiments, modification activity of an effector partner described herein comprises cleavage activity, binding activity, insertion activity, and substitution activity. In some embodiments, modification activity of an effector partner results in: cleavage of at least one strand of a target nucleic acid, deletion of one or more nucleotides of a target nucleic acid, insertion of one or more nucleotides into a target nucleic acid, substitution of one or more nucleotides of a target nucleic acid with an alternative nucleotide, more than one of the foregoing, or any combination thereof. In some embodiments, an ability of an effector partner to edit a target nucleic acid depends upon the effector partner being complexed with a guide nucleic acid, the guide nucleic acid being hybridized to a target sequence of the target nucleic acid, the distance between the target sequence and a PAM sequence, or combinations thereof. In some embodiments, an effector partner edits a target nucleic acid, wherein the target nucleic acid comprises a target strand and/or a non-target strand. In some embodiments, an effector partner is fused to one or more heterologous polypeptide, optionally wherein the heterologous polypeptide is a NLS.

An effector partner may function as a single protein. Alternatively, an effector partner may function as part of a multiprotein complex, including, for example, a complex having two or more effector partners, including two or more of the same effector partners (e.g., a dimer or multimer). An effector partner, when functioning in a multiprotein complex, may have only one functional activity, while other effector partners present in the multiprotein complex are capable of a complementary or differing functional activity. An effector partner may be a modified effector partner having increased modification activity and/or increased substrate binding activity (e.g., substrate selectivity, specificity, and/or affinity). Alternatively, or in addition, an effector partner may be a catalytically inactive effector partner having reduced modification activity or no modification activity.

TABLE 1.1 provides illustrative amino acid sequences of effector partners that are useful in the compositions, systems and methods described herein.

In some embodiments, compositions, systems, and methods described herein provided herein comprise an effector partner, wherein the amino acid sequence of the effector partner comprises at least about 200 contiguous amino acids or more of any one of the sequences recited in TABLE 1.1. In some embodiments, the amino acid sequence of an effector partner provided herein comprises at least about 200, at least about 220, at least about 240, at least about 260, at least about 280, at least about 300, at least about 320, at least about 340, at least about 360, at least about 380, at least about 400 contiguous amino acids, at least about 420 contiguous amino acids, at least about 440 contiguous amino acids, at least about 460 contiguous amino acids, at least about 480 contiguous amino acids, at least about 500 contiguous amino acids, at least about 520 contiguous amino acids, at least about 540 contiguous amino acids, at least about 560 contiguous amino acids, at least about 580 contiguous amino acids, at least about 600 contiguous amino acids, or more of any one of the sequences of TABLE 1.1.

In some embodiments, other than a truncation of the first 10 amino acids, 20 amino acids, 30 amino acids, 40 amino acids, 50 amino acids, 60 amino acids, 70 amino acids, 80 amino acids, 90 amino acids, or 100 amino acids, and/or a truncation of the last 10 amino acids, 20 amino acids, 30 amino acids, 40 amino acids, 50 amino acids, 60 amino acids, 70 amino acids, 80 amino acids, 90 amino acids, or 100 amino acids, the amino acid sequence of an effector partner provided herein comprises any one of the sequences of TABLE 1.1.

In some embodiments, compositions, systems, and methods provided herein comprise an effector partner, wherein the effector partner comprises an amino acid sequence that is at least 65% identical to any one of the sequences as set forth in TABLE 1.1. In some embodiments, an effector partner provided herein is at least 65%, at least 70%, at least 75%, at least 80%, at least 85%, at least 90%, at least 95%, at least 97%, or at least 98%, at least 99%, or 100% identical to any one of the sequences as set forth in TABLE 1.1. In some embodiments, compositions, systems, and methods provided herein comprise an effector partner and an engineered guide nucleic acid, wherein the effector partner comprises an amino acid sequence that is at least 65%, at least 70%, at least 75%, at least 80%, at least 85%, at least 90%, at least 95%, at least 98%, at least 99%, or 100% identical to any one of the sequences as set forth in TABLE 1.1.

In some embodiments, an effector partner provided herein comprises an amino acid sequence that is at least 65% identical to any one of the sequences as set forth in TABLE 1.1. In some embodiments, an effector partner provided herein comprises an amino acid sequence that is at least 70% identical to any one of the sequences as set forth in TABLE 1.1. In some embodiments, an effector partner provided herein comprises an amino acid sequence that is at least 75% identical to any one of the sequences as set forth in TABLE 1.1. In some embodiments, an effector partner provided herein comprises an amino acid sequence that is at least 80% identical to any one of the sequences as set forth in TABLE 1.1. In some embodiments, an effector partner provided herein comprises an amino acid sequence that is at least 85% identical to any one of the sequences as set forth in TABLE 1.1. In some embodiments, an effector partner provided herein comprises an amino acid sequence that is at least 90% identical to any one of the sequences as set forth in TABLE 1.1. In some embodiments, an effector partner provided herein comprises an amino acid sequence that is at least 95% identical to any one of the sequences as set forth in TABLE 1.1. In some embodiments, an effector partner provided herein comprises an amino acid sequence that is at least 98% identical to any one of the sequences as set forth in TABLE 1.1. In some embodiments, an effector partner provided herein comprises an amino acid sequence that is at least 99% identical to any one of the sequences as set forth in TABLE 1.1. In some embodiments, an effector partner provided herein comprises an amino acid sequence that is identical to any one of the sequences as set forth in TABLE 1.1.

In some embodiments, compositions, systems, and methods provided herein comprise an effector partner, wherein the effector partner comprises one or more amino acid alteration relative to any one of the sequences of TABLE 1.1 wherein the effector partner comprising one or more amino acid alterations is a variant of an effector partner described herein. It is understood that any reference to an effector partner herein also refers to an effector partner variant as described herein. In some embodiments, an effector partner provided herein comprises: 1 amino acid alteration, 2 amino acid alterations, 3 amino acid alterations, 4 amino acid alterations, 5 amino acid alterations, 6 amino acid alterations, 7 amino acid alterations, 8 amino acid alterations, 9 amino acid alterations, 10 amino acid alterations or more relative to any one of the sequences of TABLE 1.1.

In some embodiments, the one or more amino acid alteration is a conservative or non-conservative substitution. In some embodiments, an effector partner provided herein comprises: 1 conservative amino acid substitution, 2 conservative amino acid substitutions, 3 conservative amino acid substitutions, 4 conservative amino acid substitutions, 5 conservative amino acid substitutions, 6 conservative amino acid substitutions, 7 conservative amino acid substitutions, 8 conservative amino acid substitutions, 9 conservative amino acid substitutions, 10 conservative amino acid substitutions or more relative to any one of the sequences of TABLE 1.1. In some embodiments, an effector partner provided herein comprises: 1 non-conservative amino acid substitution, 2 non-conservative amino acid substitutions, 3 non-conservative amino acid substitutions, 4 non-conservative amino acid substitutions, 5 non-conservative amino acid substitutions, 6 non-conservative amino acid substitutions, 7 non-conservative amino acid substitutions, 8 non-conservative amino acid substitutions, 9 non-conservative amino acid substitutions, 10 non-conservative amino acid substitutions or more relative to any one of the sequences of TABLE 1.1.

In some embodiments, the one or more amino acid alterations may result in a change in activity of the effector partner relative to a naturally-occurring counterpart. For example, and as described in further detail herein, the one or more amino acid alteration increases or decreases catalytic activity of the effector partner relative to a naturally-occurring counterpart. In some embodiments, the one or more amino acid alterations results in a catalytically inactive effector partner variant.

Effector Protein and Effector Partner Complexes

Compositions, systems, and methods of the present disclosure may comprise a complex or uses thereof, wherein the complex comprises one or more effector proteins and/or one or more effector partners, or combinations thereof. In some embodiments, the complex comprises at least one effector protein, and one or more effector partner. In some embodiments, the complex comprises at least one effector protein, and two effector partners. In some embodiments, the complex comprises at least one effector protein, and two, three, four or more effector partners. In some embodiments, the complex comprises two, three, four or more effector proteins, and two, three, four or more effector partners. In some embodiments, each effector protein of the complex independently comprises an amino acid sequence with at least 65%, at least 70%, at least 75%, at least 80%, at least 85%, at least 90%, at least 95% or 100% identity to any one of the sequences of TABLE 1 and each effector partner of the complex independently comprises an amino acid sequence with at least 65%, at least 70%, at least 75%, at least 80%, at least 85%, at least 90%, at least 95% or 100% identity to any one of the sequences of TABLE 1.1. The complex may comprise enhanced activity relative to the activity of any one of its effector proteins and/or effector partners alone. For example, the complex comprising one or more of a effector protein and/or effector partner (e.g., in dimeric form) may comprise greater nucleic acid binding affinity, nuclease activity, integrase activity, cointegrase activity, and the like than that of any of the proteins provided in monomeric form. It is understood that when discussing the use of an effector protein and an effector partner in compositions, systems, and methods provided herein, the complex form is also described. It is also understood that when discussing the use of more than one effector partner in compositions, systems, and methods provided herein, the complex form is also described.

Engineered Proteins

In some embodiments, effector proteins and/or effector partners described herein are modified (also referred to as an engineered protein or an engineered partner, respectively). In some embodiments, effector proteins disclosed herein are engineered proteins. In some embodiments, effector partners disclosed herein are engineered partners. Unless otherwise indicated, reference to effector proteins throughout the present disclosure include engineered proteins thereof. Similarly, unless otherwise indicated, reference to effector partners throughout the present disclosure include engineered partners thereof. Engineered proteins are not identical to a naturally-occurring protein. In some embodiments, an engineered protein may comprise a modified form of a naturally-occurring protein. Engineered partners are not identical to a naturally-occurring protein. In some embodiments, an engineered partner may comprise a modified form of a naturally-occurring protein.

For example, effector proteins and/or effector partners described herein may be modified with the addition of one or more heterologous peptides or heterologous polypeptides (referred to collectively herein as a heterologous polypeptide). In certain embodiments, an effector protein and/or effector partners modified with the addition of one or more heterologous peptides or heterologous polypeptides may be referred to herein as a fusion protein. Such fusion proteins are described herein and throughout.

In some embodiments, a heterologous peptide or heterologous polypeptide comprises a subcellular localization signal. In some embodiments, a subcellular localization signal may be a nuclear localization signal (NLS) for targeting the effector protein and/or effector partner to the nucleus. Non-limiting examples of NLS sequences are set forth in TABLE 2. In some embodiments, the subcellular localization signal is a nuclear export signal (NES), a sequence to keep an effector protein and/or effector partner retained in the cytoplasm, a mitochondrial localization signal for targeting to the mitochondria, a chloroplast localization signal for targeting to a chloroplast, an ER retention signal, and the like. In some embodiments, an effector protein and/or effector partner described herein is not modified with a subcellular localization signal so that the polypeptide is not targeted to the nucleus, which may be advantageous depending on the circumstance (e.g., when the target nucleic acid is an RNA that is present in the cytosol).

In some embodiments, a heterologous peptide or heterologous polypeptide comprises a chloroplast transit peptide (CTP), also referred to as a chloroplast localization signal or a plastid transit peptide, which targets the effector protein and/or effector partner to a chloroplast. Chromosomal transgenes from bacterial sources may require a sequence encoding a CTP sequence fused to a sequence encoding an expressed protein (e.g., the effector protein and/or effector partner) if the expressed protein is to be compartmentalized in the plant plastid (e.g., chloroplast). The CTP may be removed in a processing step during translocation into the plastid. Accordingly, localization of an effector protein and/or effector partner to a chloroplast is often accomplished by means of operably linking a polynucleotide sequence encoding a CTP sequence to the 5′ region of a polynucleotide encoding the exogenous protein.

In some embodiments, the heterologous polypeptide is an endosomal escape peptide (EEP). An EEP is an agent that quickly disrupts the endosome in order to minimize the amount of time that a delivered molecule, such an effector protein and/or effector partner, spends in the endosome-like environment, and to avoid getting trapped in the endosomal vesicles and degraded in the lysosomal compartment.

In some embodiments, the heterologous polypeptide is a cell penetrating peptide (CPP), also known as a Protein Transduction Domain (PTD). A CPP or PTD is a polypeptide, polynucleotide, carbohydrate, or organic or inorganic compound that facilitates traversing a lipid bilayer, micelle, cell membrane, organelle membrane, or vesicle membrane.

Further suitable heterologous polypeptides include, but are not limited to, proteins (or fragments/domains thereof) that are boundary elements (e.g., CTCF), proteins and fragments thereof that provide periphery recruitment (e.g., Lamin A, Lamin B, etc.), and protein docking elements (e.g., FKBP/FRB, Pill/Abyl, etc.).

In some embodiments, a heterologous peptide or heterologous polypeptide comprises a protein tag. In some instances, the protein tag is referred to as purification tag or a fluorescent protein. The protein tag may be detectable for use in detection of the effector protein and/or effector partner and/or purification of the effector protein and/or effector partner. Accordingly, in some embodiments, compositions, systems and methods comprise a protein tag or use thereof. Any suitable protein tag may be used depending on the purpose of its use. Non-limiting examples of protein tags include a fluorescent protein, a histidine tag, e.g., a 6×His tag (SEQ ID NO: 1009); a hemagglutinin (HA) tag; a FLAG tag; a Myc tag; and maltose binding protein (MBP). In some instances, the protein tag is a portion of MBP that can be detected and/or purified. Non-limiting examples of fluorescent proteins include green fluorescent protein (GFP), yellow fluorescent protein (YFP), red fluorescent protein (RFP), cyan fluorescent protein (CFP), mCherry, and tdTomato.

A heterologous polypeptide may be located at or near the amino terminus (N-terminus) of the effector protein and/or effector partner disclosed herein. A heterologous polypeptide may be located at or near the carboxy terminus (C-terminus) of the effector proteins and/or effector partners disclosed herein. In some embodiments, a heterologous polypeptide is located internally in an effector protein and/or effector partner described herein (i.e., is not at the N- or C-terminus of an effector protein and/or effector partner described herein) at a suitable insertion site.

In some embodiments, a vector encodes the effector proteins and/or effector partners described herein, wherein the vector or vector systems disclosed herein comprises one or more heterologous polypeptides, such as about or more than about 1, 2, 3, 4, 5, 6, 7, 8, 9, 10, or more heterologous polypeptides. In some embodiments, an effector protein and/or effector partner described herein comprises 1, 2, 3, 4, 5, 6, 7, 8, 9, 10, or more heterologous polypeptides at or near the N-terminus, 1, 2, 3, 4, 5, 6, 7, 8, 9, 10, or more heterologous polypeptides at or near the C-terminus, or a combination of these (e.g. one or more heterologous polypeptides at the amino-terminus and one or more heterologous polypeptides at the carboxy terminus). When more than one heterologous polypeptide is present, each may be selected independently of the others, such that a single heterologous polypeptide may be present in more than one copy and/or in combination with one or more other heterologous polypeptides present in one or more copies. In some embodiments, a heterologous polypeptide is considered near the N- or C-terminus when the nearest amino acid of the heterologous polypeptide is within about 1, 2, 3, 4, 5, 10, 15, 20, 25, 30, 40, 50, or more amino acids along the polypeptide chain from the N- or C-terminus.

In some embodiments, effector proteins and/or effector partners described herein are encoded by a codon optimized nucleic acid. In some embodiments, a nucleic acid sequence encoding an effector protein and/or effector partner described herein, is codon optimized. In some embodiments, effector proteins and/or effector partners described herein may be codon optimized for expression in a specific cell, for example, a bacterial cell, a plant cell, a eukaryotic cell, an animal cell, a mammalian cell, or a human cell. In some embodiments, the effector protein and/or effector partner is codon optimized for a human cell. It is understood that when describing coding sequences of polypeptides described herein, said coding sequences do not necessarily require a codon encoding a N-terminal Methionine (M) or a Valine (V) as described for the effector proteins described herein. One skilled in the art would understand that a start codon could be replaced or substituted with a start codon that encodes for an amino acid residue sufficient for initiating translation in a host cell. In some embodiments, when a modifying heterologous peptide, such as a fusion partner protein, protein tag or NLS, is located at the N terminus of the effector protein, a start codon for the heterologous peptide serves as a start codon for the effector protein as well. Thus, the natural start codon encoding an amino acid residue sufficient for initiating translation (e.g., Methionine (M) or a Valine (V)) of the effector protein may be removed or absent.

In another example, engineered proteins and/or engineered partners may comprise one or more modifications that may provide altered activity as compared to a naturally-occurring counterpart (e.g., a naturally-occurring nuclease, nickase, transposase, cointegrase, ATPase, etc. which may be a naturally-occurring effector protein and/or effector partner). In some embodiments, activity (e.g., nickase, nuclease, binding, transposase, cointegrase, ATPase, etc., activity) of engineered proteins and/or engineered partners described herein may be measured relative to a WT effector protein and/or effector partner or compositions containing the same in a cleavage assay.

For example, engineered proteins and/or engineered partners may comprise one or more modifications that may provide increased activity as compared to a naturally-occurring counterpart. For example, engineered proteins and/or engineered partners may provide increased catalytic activity (e.g., nickase, nuclease, binding, transposase or transposase-like, cointegrase or cointegrase-like, or ATPase activity) as compared to a naturally-occurring counterpart. Engineered proteins and/or engineered partners may provide enhanced nucleic acid binding activity (e.g., enhanced binding of a guide nucleic acid, donor nucleic acid, and/or target nucleic acid) as compared to a naturally-occurring counterpart. An engineered protein may have a 1%, 5%, 10%, 20%, 30%, 40%, 50%, 60%, 70%, 80%, 90%, 100%, 120%, 140%, 160%, 180%, 200%, or more, increase of the activity of a naturally-occurring counterpart. An engineered partner may have a 1%, 5%, 10%, 20%, 30%, 40%, 50%, 60%, 70%, 80%, 90%, 100%, 120%, 140%, 160%, 180%, 200%, or more, increase of the activity of a naturally-occurring counterpart.

Alternatively, engineered proteins and/or engineered partners may comprise modifications, that reduces the activity of the engineered protein relative to a naturally occurring nuclease, nickase, transposase, cointegrase, and/or ATPase. An engineered protein may have a 100%, 90%, 80%, 70%, 60%, 50%, 40%, 30%, 20%, 10%, 5%, 1%, or less, decrease of the activity of a naturally occurring counterpart. An engineered partner may have a 100%, 90%, 80%, 70%, 60%, 50%, 40%, 30%, 20%, 10%, 5%, 1%, or less, decrease of the activity of a naturally occurring counterpart. Decreased activity may be decreased catalytic activity (e.g., nickase, nuclease, binding, transposase or transposase-like, cointegrase or cointegrase-like, or ATPase activity) as compared to a naturally-occurring counterpart.

An engineered protein that has decreased catalytic activity may be referred to as catalytically or enzymatically inactive, catalytically or enzymatically dead, as a dead protein or a dCas protein. In some embodiments, such a protein may comprise an enzymatically inactive domain (e.g. inactive nuclease domain). For example, a nuclease domain (e.g., RuvC domain) of an effector protein may be deleted or mutated relative to a wild type counterpart so that it is no longer functional or comprises reduced nuclease activity. In some embodiments, a catalytically inactive effector protein may bind to a guide nucleic acid and/or a target nucleic acid but does not cleave the target nucleic acid. In some embodiments, a catalytically inactive effector protein may associate with a guide nucleic acid to activate or repress transcription of a target nucleic acid. In some embodiments, a catalytically inactive effector protein is fused to a fusion partner protein that confers an alternative activity to an effector protein activity. Such fusion proteins are described herein and throughout.

Fusion Partners

In some embodiments, compositions, systems, and methods comprise a fusion partner, a fusion protein, or uses thereof. In some embodiments, a fusion partner comprises a polypeptide or peptide that is fused or linked to an effector protein or an effector partner. In some embodiments, an effector protein is a fusion protein, wherein the fusion protein comprises an effector protein described herein (e.g., wherein the effector protein comprises an amino acid sequence that is at least 65% identical to any one of the sequences set forth in TABLE 1) and a fusion partner. In some embodiments, an effector partner is a fusion protein, wherein the fusion protein comprises an effector partner described herein (e.g., wherein the effector partner comprises an amino acid sequence that is at least 65% identical to any one of the sequences set forth in TABLE 1.1) and a fusion partner. Unless otherwise indicated, reference to effector proteins and/or effector partners throughout the present disclosure include fusion proteins thereof.

The fusion partner generally imparts some function or activity to the fusion protein that is not provided by the effector protein and/or effector partner. Such activities may include nuclease activity, methyltransferase activity, demethylase activity, DNA repair activity, DNA damage activity, deamination activity, dismutase activity, alkylation activity, depurination activity, oxidation activity, dimer forming activity (e.g., pyrimidine dimer forming activity), integrase activity, transposase activity, recombinase activity, polymerase activity, ligase activity, helicase activity, photolyase activity, glycosylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity or demyristoylation activity) that modifies a polypeptide associated with target nucleic acid (e.g., a histone), and/or signaling activity.

In some embodiments, a fusion partner may provide signaling activity. In some embodiments, a fusion partner may inhibit or promote the formation of multimeric complex of an effector protein and/or effector partner. In an additional example, the fusion partner may directly or indirectly modify a target nucleic acid. Modifications may be of a nucleobase, nucleotide, or nucleotide sequence of a target nucleic acid. In some embodiments, the fusion partner may interact with additional proteins, or functional fragments thereof, to make modifications to a target nucleic acid. In other embodiments, the fusion partner may modify proteins associated with a target nucleic acid. In some embodiments, a fusion partner may modulate transcription (e.g., inhibits transcription, increases transcription) of a target nucleic acid. In yet another example, a fusion partner may directly or indirectly inhibit, reduce, activate or increase expression of a target nucleic acid.

Linkers

In some embodiments, fusion proteins comprise an effector protein, or an effector partner, or both, and a fusion partner. The effector protein, or an effector partner, or both, may be fused or linked to the fusion partner. The terms “fused” and “linked” may be used interchangeably. In some instances, the effector protein, or an effector partner, or both, and the fusion partner are directly linked via a covalent bond. In some instances, effector proteins, or effector partners, or both, and fusion partners are connected via a linker.

The linker may comprise or consist of a covalent bond. The linker may comprise or consist of a chemical group. In some embodiments, the linker comprises an amino acid. In some embodiments, a linker comprises a bond or molecule that links a first polypeptide to a second polypeptide. In some embodiments, a peptide linker comprises at least two amino acids linked by an amide bond. In general, the linker connects a terminus of the effector protein or effector partner to a terminus of the fusion partner. In some embodiments, the carboxy terminus of the effector protein or effector partner is linked to the amino terminus of the fusion partner. In some embodiments, the carboxy terminus of the fusion partner is linked to the amino terminus of the effector protein or effector partner.

In some embodiments, a terminus of the effector protein or effector partner is linked to a terminus of the fusion partner through an amide bond. In some embodiments, a terminus of the effector protein or effector partner is linked to a terminus of the fusion partner through a peptide bond. In some instances, linkers comprise an amino acid. In some embodiments, linkers comprise a peptide. In some embodiments, an effector protein or effector partner is coupled to a fusion partner via a linker protein. The linker protein may have any of a variety of amino acid sequences. A linker protein may comprise a region of rigidity (e.g., beta sheet, alpha helix), a region of flexibility, or any combination thereof. In some embodiments, the linker comprises small amino acids, such as glycine and alanine, that impart high degrees of flexibility. The ordinarily skilled artisan will recognize that design of a peptide conjugated to any desired element may include linkers that are all or partially flexible, such that the linker may include a flexible linker as well as one or more portions that confer less flexible structure. Suitable linkers include proteins of 4 linked amino acids to 40 linked amino acids in length, or between 4 linked amino acids and 25 linked amino acids in length. In some embodiments, when a linked amino acids is described herein, it may refer to at least two amino acids linked by an amide bond.

These linkers may be produced by using synthetic, linker-encoding oligonucleotides to couple the proteins, or may be encoded by a nucleic acid sequence encoding a fusion protein (e.g., an effector protein coupled to a fusion partner). Examples of linker proteins include glycine polymers (G)n, glycine-serine polymers (including, for example, (GS)n, GSGGSn (SEQ ID NO: 1010), GGSGGSn (SEQ ID NO: 1011), and GGGSn (SEQ ID NO: 1012), where n is an integer of at least one), glycine-alanine polymers, and alanine-serine polymers. Exemplary linkers may comprise amino acid sequences including, but not limited to, GGSG (SEQ ID NO: 1013), GGSGG (SEQ ID NO: 1014), GSGSG (SEQ ID NO: 1015), GSGGG (SEQ ID NO: 1016), GGGSG (SEQ ID NO: 1017), and GSSSG (SEQ ID NO: 1018).

In some instances, linkers do not comprise an amino acid. In some instances, linkers do not comprise a peptide. In some instances, linkers comprise a nucleotide, a polynucleotide, a polymer, or a lipid.

Synthesis, Isolation and Assaying

Effector proteins and effector partners of the present disclosure of the present disclosure may be synthesized, using any suitable method. Effector proteins and effector partners of the present disclosure may be produced in vitro or by eukaryotic cells or by prokaryotic cells. Effector proteins and effector partners may be further processed by unfolding, e.g. heat denaturation, dithiothreitol reduction, etc. and may be further refolded, using any suitable method.

Any suitable method of generating and assaying the effector proteins and/or effector partners described herein may have used in the present disclosure. Such methods include, but are not limited to, site-directed mutagenesis, random mutagenesis, combinatorial libraries, and other mutagenesis methods described herein (see, e.g., Sambrook et al., Molecular Cloning. A Laboratory Manual, Third Ed., Cold Spring Harbor Laboratory, New York (2001); Ausubel et al., Current Protocols in Molecular Biology, John Wiley and Sons, Baltimore, M D (1999); Gillman et al., Directed Evolution Library Creation: Methods and Protocols (Methods in Molecular Biology) Springer, 2nd ed (2014)). One non-limiting example of a method for preparing an effector protein is to express recombinant nucleic acids encoding the effector protein and/or effector partners in a suitable microbial organism, such as a bacterial cell, a yeast cell, or other suitable cell, using methods well known in the art. Exemplary methods are also described in the Examples provided herein.

In some embodiments, an effector protein provided herein is an isolated effector protein. In some embodiments, effector proteins described herein may be isolated and purified for use in compositions, systems, and/or methods described herein. Similarly, an effector partner provided herein is an isolated effector partner. In some embodiments, effector partners described herein may be isolated and purified for use in compositions, systems, and/or methods described herein. Methods described here may include the step of isolating effector proteins and/or effector partners described herein. Any suitable method to provide isolated effector proteins and/or effector partners described herein may be used in the present disclosure, for example, recombinant expression systems, precipitation, gel filtration, ion-exchange, reverse-phase and affinity chromatography, and the like. Other well-known methods are described in Deutscher et al., Guide to Protein Purification: Methods in Enzymology, Vol. 182, (Academic Press, (1990)). Alternatively, the isolated polypeptides of the present disclosure may be obtained using well-known recombinant methods (see, e.g., Sambrook et al., Molecular Cloning: A Laboratory Manual, Third Ed., Cold Spring Harbor Laboratory, New York (2001); and Ausubel et al., Current Protocols in Molecular Biology, John Wiley and Sons, Baltimore, MD (1999)). The methods and conditions for biochemical purification of a polypeptide described herein may be chosen by those skilled in the art, and purification monitored, for example, by a functional assay.

For example, compositions and/or systems described herein may further comprise a purification tag that may be attached to an effector protein, or a nucleic acid encoding for a purification tag that may be attached to a nucleic acid encoding for an effector protein as described herein. In another example, compositions and/or systems described herein may further comprise a purification tag that may be attached to an effector partner, or a nucleic acid encoding for a purification tag that may be attached to a nucleic acid encoding for an effector partner as described herein. A purification tag, as used herein, may be an amino acid sequence which may attach or bind with high affinity to a separation substrate and assist in isolating the protein of interest from its environment, which may be its biological source, such as a cell lysate. Attachment of the purification tag may be at the N or C terminus of the effector protein and/or effector partner. Furthermore, an amino acid sequence recognized by a protease or a nucleic acid encoding for an amino acid sequence recognized by a protease, such as TEV protease or the HRV3C protease may be inserted between the purification tag and the effector protein and/or effector partner, such that biochemical cleavage of the sequence with the protease after initial purification liberates the purification tag. Purification and/or isolation may be through high performance liquid chromatography (HPLC), exclusion chromatography, gel electrophoresis, affinity chromatography, or other purification technique. Examples of purification tags are as described herein.

In some embodiments, effector proteins and/or effector partners described herein are isolated from cell lysate. In some embodiments, the compositions described herein may comprise 20% or more by weight, 75% or more by weight, 95% or more by weight, or 99.5% or more by weight of an effector protein and/or effector partner, related to the method of preparation of compositions described herein and its purification thereof, wherein percentages may be upon total protein content in relation to contaminants. Thus, in some embodiments, an effector protein and/or effector partner described herein is at least 80% pure, at least 85% pure, at least 90% pure, at least 95% pure, at least 98% pure, or at least 99% pure (e.g., free of contaminants, non-engineered polypeptide proteins or other macromolecules, etc.).

Protospacer Adjacent Motif (PAM)

Effector proteins and/or effector partners of the present disclosure may cleave or nick a target nucleic acid within or near a protospacer adjacent motif (PAM) sequence of the target nucleic acid. In some embodiments, cleavage occurs within 1, 2, 3, 4, 5, 6, 7, 8, 9, 10, 15, 20, 25, 30, 35, 40, 45, or 50 nucleotides of a 5′ or 3′ terminus of a PAM sequence. In some embodiments, effector proteins and/or effector partners described herein recognize a PAM sequence, wherein the effector proteins and/or effector partner binds to a sequence adjacent to the PAM. In some embodiments, recognizing a PAM sequence comprises binding to a sequence adjacent to the PAM. In some embodiments, a target nucleic acid comprises a target sequence that is adjacent to a PAM sequence. In some instances, effector protein and/or effector partners do not require a PAM to bind and/or cleave a target nucleic acid.

### V. NUCLEIC ACID SYSTEMS

Guide Nucleic Acids

The compositions, systems, and methods of the present disclosure may comprise a guide nucleic acid or a use thereof. Unless otherwise indicated, compositions, systems and methods comprising guide nucleic acids or uses thereof, as described herein and throughout, include DNA molecules, such as expression vectors, that encode a guide nucleic acid.

A guide nucleic acid may comprise a naturally occurring nucleotide sequence. A guide nucleic acid may comprise a non-naturally nucleotide occurring sequence, wherein the nucleotide sequence of the guide nucleic acid, or any portion thereof, may be different from a nucleotide sequence of a naturally occurring guide nucleic acid. A guide nucleic acid of the present disclosure comprises one or more of the following: a) a single nucleic acid molecule; b) a DNA base; c) an RNA base; d) a modified base; e) a modified sugar; f) a modified backbone; and the like. Modifications are described herein and throughout the present disclosure (e.g., in the section entitled “Engineered Modifications”). A guide nucleic acid may be chemically synthesized or recombinantly produced by any suitable methods. Guide nucleic acids and portions thereof may be found in or identified from a CRISPR array present in the genome of a host organism or cell.

The guide nucleic acid may also form complexes as described through herein. For example, a guide nucleic acid may hybridize to another nucleic acid, such target nucleic acid, or a portion thereof. In some embodiments, a complex of two nucleic acids may be a nucleic acid duplex. In another example, a guide nucleic acid may complex with an effector protein. In such embodiments, a guide nucleic acid-effector protein complex may be described herein as an RNP. In some embodiments, when in a complex, at least a portion of the complex may bind, recognize, and/or hybridize to a target nucleic acid. For example, when a guide nucleic acid and an effector protein are complexed to form an RNP, at least a portion of the RNP may bind, recognize, and/or hybridize to a target nucleic acid. Those skilled in the art in reading the below specific examples of guide nucleic acids as used in RNPs described herein, will understand that in some embodiments, a RNP may hybridize to one or more target sequences in a target nucleic acid, thereby allowing the RNP to modify and/or recognize a target nucleic acid or sequence contained therein or to modify and/or recognize non-target sequences depending on the guide nucleic acid, and in some embodiments, the effector protein, used.

In some embodiments, the compositions, systems, and methods of the present disclosure comprise two or more guide nucleic acids (e.g., 2, 3, 4, 5, 6, 7, 9, 10 or more guide nucleic acids), and/or uses thereof. Multiple guide nucleic acids may target an effector protein to different locations in the target nucleic acid by hybridizing to different locations within the target nucleic acid. A first guide nucleic acid may bind a first loci of a target nucleic acid and a second guide nucleic acid may bind a second loci of the target nucleic acid. The first loci and the second loci of the target nucleic acid may be located at least 1, at least 10, at least 20, at least 30, at least 40, at least 50, at least 60, at least 70, at least 80, at least 90 or at least 100 nucleotides apart. The first loci and the second loci of the target nucleic acid may be located between 100 and 200, 200 and 300, 300 and 400, 400 and 500, 500 and 600, 600 and 700, 700 and 800, 800 and 900 or 900 and 1000 nucleotides apart.

In some instances, the first loci and/or the second loci of the target nucleic acid are located in an intron of a gene, an exon of a gene, or combinations thereof. In some instances, the first loci and/or the second loci of the target nucleic acid are located in an intron of a gene. In some embodiments, the first loci and/or the second loci of the target nucleic acid are located in an exon of a gene. In some instances, the first loci and/or the second loci of the target nucleic acid span an exon-intron junction of a gene. In some instances, the first loci and/or the second loci of the target nucleic acid are located on either side of an exon and cutting at both sites results in deletion of the exon. In some embodiments, composition, systems and methods comprise a donor nucleic acid that may be inserted in replacement of a deleted or cleaved sequence of the target nucleic acid. In some embodiments, compositions, systems and methods comprising multiple guide nucleic acids or uses thereof comprise one or more effector proteins and/or one or more effector partners, or combinations thereof, wherein the effector proteins may be identical, non-identical, or combinations thereof.

In some embodiments, a guide nucleic acid comprises about: 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 linked nucleotides. In general, a guide nucleic acid comprises at least: 10, 15, 20, 25, 30, 35, 40, 45, 50, 55, 60 linked nucleotides. In some embodiments, the guide nucleic acid has about 10 to about 60, about 20 to about 50, or about 30 to about 40 linked nucleotides.

A guide nucleic acid may comprise: a first region (FR) that is not complementary to a target nucleic acid and a second region (SR) is, at least partially, complementary to a portion of a target nucleic acid. An FR may be located 5′ to a SR (FR-SR). Alternatively, a SR is located 5′ to FR (SR-FR). An FR and a SR may be coupled or linked. In some embodiments, a FR may interact with an effector protein as described herein (e.g., TABLE 1). In some embodiments, interaction between a FR and an effector protein may be a binding interaction. In some embodiments, a binding interaction may be non-covalent binding. In some embodiments, a FR comprises one or more of a handle sequence, an intermediary RNA sequence, a repeat sequence, a linker or combinations thereof.

In some embodiments, a SR is at least partially, complementary to an equal length of a portion of a target nucleic acid (e.g., a target sequence). In some embodiments, a SR may, at least partially, hybridize to an equal length of a portion of a target nucleic acid (e.g., a target sequence). In some embodiments, a SR is complementary with and hybridizes to an equal length portion of a target sequence of a target nucleic acid.

The conditions appropriate for hybridization between two nucleotide sequences depend on the length of the sequence and the degree of complementarity, variables which are well known in the art. For hybridizations between nucleic acids with short stretches of complementarity (e.g. complementarity over 35 or less, 30 or less, 25 or less, 22 or less, 20 or less, or 18 or less nucleotides) the position of mismatches may become important (see Sambrook et al., supra, 11.7-11.8). Typically, the length for a hybridizable nucleic acid is 8 nucleotides or more (e.g., 10 nucleotides or more, 12 nucleotides or more, 15 nucleotides or more, 20 nucleotides or more, 22 nucleotides or more, 25 nucleotides or more, or 30 nucleotides or more). Any suitable in vitro assay may be utilized to assess whether two sequence “hybridize”. One such assay is a melting point analysis where the greater the degree of complementarity between two nucleotide sequences, the greater the value of the melting temperature (Tm) for hybrids of nucleic acids having those sequences. The conditions of temperature and ionic strength determine the “stringency” of the hybridization. Temperature, wash solution salt concentration, and other conditions may be adjusted as necessary according to factors such as length of the region of complementation and the degree of complementation. Hybridization and washing conditions are well known and exemplified in Sambrook, J., Fritsch, E. F. and Maniatis, T. Molecular Cloning: A Laboratory Manual, Second Edition, Cold Spring Harbor Laboratory Press, Cold Spring Harbor (1989), particularly Chapter 11 and Table 11.1 therein; and Sambrook, J. and Russell, W., Molecular Cloning: A Laboratory Manual, Third Edition, Cold Spring Harbor Laboratory Press, Cold Spring Harbor (2001). In some embodiments, a SR comprises a spacer sequence.

In some embodiments, a guide nucleic acid comprises at least 10, 11, 12, 13, 14, 15, 16, 17, 18, 19, 20, 21, 22, 23, 24, 25, 26, 27, 28, 29, or 30 contiguous nucleotides that are complementary to a target sequence of a target nucleic acid. In some embodiments, the target nucleic acid comprises a eukaryotic sequence. Such a eukaryotic sequence is a sequence of nucleotides that is present in a host eukaryotic cell. Such a sequence of nucleotides is distinguished from nucleotide sequences present in other host cells, such as prokaryotic cells, or viruses. Said sequences present in a eukaryotic cell may be located in a gene, an exon, an intron, a non-coding (e.g., promoter or enhancer) region, a selectable marker, tag, signal, and the like. In some embodiments, a target sequence is a eukaryotic sequence.

In some embodiments, guide nucleic acids comprise additional elements that contribute additional functionality (e.g., stability, heat resistance, etc.) to the guide nucleic acid. Such elements may be one or more nucleotide alterations, nucleotide sequences, intermolecular secondary structures, or intramolecular secondary structures (e.g., one or more hair pin regions, one or more bulges, etc.). In some embodiments, guide nucleic acids comprise one or more linkers. A linker may comprise 1, 2, 3, 4, 5, 6, 7, 8, 9, 10, or more nucleotides. A linker may be any suitable linker, examples of which are described herein.

In some embodiments, guide nucleic acids comprise one or more nucleotide sequences as described herein (e.g., TABLE 3). Such nucleotide sequences described herein (e.g., TABLE 3) may be described as a nucleotide sequence of either DNA or RNA, however, no matter the form the sequence is described, it is readily understood that such nucleotide sequences may be revised to be RNA or DNA, as needed, for describing a sequence within a guide nucleic acid itself or the sequence that encodes a guide nucleic acid, such as a nucleotide sequence described herein for a vector. Similarly, disclosure of the nucleotide sequences described herein (e.g., TABLE 3) also discloses the complementary nucleotide sequence, the reverse nucleotide sequence, and the reverse complement nucleotide sequence, any one of which may be a nucleotide sequence for use in a guide nucleic acid as described herein. In some embodiments, guide nucleic acid sequence(s) comprises one or more nucleotide alterations at one or more positions in any one of the sequences described herein. Alternative nucleotides may be any one or more of A, C, G, T or U, or a deletion, or an insertion.

TABLE 3 provides illustrative nucleotide sequences for use with the compositions, systems and methods of the disclosure. A guide nucleic acid may comprise one or more of: a nucleotide sequence described herein, a portion thereof, a variant thereof, or combinations thereof. In some embodiments, a guide nucleic acid comprises one or more nucleotide sequences, wherein each of the one or more nucleotide sequences is at least 65%, at least 70%, at least 80%, at least 90%, at least 92%, at least 95%, at least 97%, or at least 99%, or 100% identical to an equal length portion of any one of the sequences recited in TABLE 3. In some embodiments, a guide nucleic acid comprises one or more, two or more, three or more, four or more nucleotide sequences, wherein each nucleotide sequence is at least 65%, at least 70%, at least 80%, at least 90%, at least 92%, at least 95%, at least 97%, or at least 99%, or 100% identical to any one of the sequences recited in TABLE 3.

Repeat Sequence

Guide nucleic acids described herein may comprise one or more repeat sequences. In some embodiments, a repeat sequence comprises a nucleotide sequence that is not complementary to a target sequence of a target nucleic acid. In some embodiments, a repeat sequence comprises a nucleotide sequence that may interact with an effector protein. In some embodiments, a repeat sequence is connected to another sequence of a guide nucleic acid, such as an intermediary RNA sequence, that is capable of being non-covalently bound by an effector protein. In some embodiments, a repeat sequence may be capable of forming a guide nucleic acid-effector protein complex (e.g., a RNP complex).

In some embodiments, the repeat sequence is between 10 and 50, 12 and 48, 14 and 46, 16 and 44, and 18 and 42 nucleotides in length. In some embodiments, the repeat sequence is between 19 and 37 nucleotides in length.

In some embodiments, a repeat sequence is adjacent to a spacer sequence. In some embodiments, a repeat sequence is followed by a spacer sequence in the 5′ to 3′ direction. In some embodiments, a repeat sequence is adjacent to an intermediary RNA sequence. In some embodiments, a repeat sequence is 3′ to an intermediary RNA sequence. In some embodiments, an intermediary RNA sequence is followed by a repeat sequence, which is followed by a spacer sequence in the 5′ to 3′ direction. In some embodiments, a repeat sequence is linked to a spacer sequence and/or an intermediary RNA sequence. In some embodiments, a guide nucleic acid comprises a repeat sequence linked to a spacer sequence and/or to an intermediary RNA sequence, which may be a direct link or by any suitable linker, examples of which are described herein.

In some embodiments, guide nucleic acids comprise more than one repeat sequence (e.g., two or more, three or more, or four or more repeat sequences). In some embodiments, a guide nucleic acid comprises more than one repeat sequence separated by another sequence of the guide nucleic acid. For example, in some embodiments, a guide nucleic acid comprises two repeat sequences, wherein the first repeat sequence is followed by a spacer sequence, and the spacer sequence is followed by a second repeat sequence in the 5′ to 3′ direction. In some embodiments, the more than one repeat sequences are identical. In some embodiments, the more than one repeat sequences are not identical.

In some embodiments, the repeat sequence comprises two sequences that are complementary to each other and hybridize to form a double stranded RNA duplex (dsRNA duplex). In some instances, the two sequences are not directly linked and hybridize to form a stem loop structure. In some embodiments, the dsRNA duplex comprises 5, 10, 15, 20 or 25 base pairs (bp). In some embodiments, not all nucleotides of the dsRNA duplex are paired, and therefore the duplex forming sequence may include a bulge. In some embodiments, the repeat sequence comprises a hairpin or stem-loop structure, optionally at the 5′ portion of the repeat region. In some embodiments, a strand of the stem portion comprises a sequence and the other strand of the stem portion comprises a sequence that is, at least partially, complementary. In some embodiments, such sequences may have 65% to 100% complementarity (e.g., 65%, 70%, 75%, 80%, 85%, 90%, 95%, or 100% complementarity). In some embodiments, a guide nucleic acid comprises nucleotide sequence that when involved in hybridization events may hybridize over one or more segments such that intervening or adjacent segments are not involved in the hybridization event (e.g., a bulge, a loop structure or hairpin structure, etc.).

In some embodiments, a repeat sequence comprises a nucleotide sequence that is at least 65%, at least 70%, at least 75%, at least 80%, at least 85%, at least 90%, at least 95%, at least 97%, or at least 98%, at least 99%, or 100% identical to any one of an equal length portion of the repeat sequences in TABLE 3. In some embodiments, a repeat sequence comprises a nucleotide sequence, wherein the nucleotide sequence comprises one or more nucleotide alterations at one or more positions in any one of the sequences of TABLE 3. Alternative nucleotides may be any one or more of A, C, G, T or U, or a deletion, or an insertion.

Spacer Sequence

Guide nucleic acids described herein may comprise one or more spacer sequences. In some embodiments, a spacer sequence may bind or hybridize a guide nucleic acid, or a complex thereof, or portions thereof, to a target sequence of a target nucleic acid. For example, a spacer sequence may bind or hybridize at least a portion of an RNP complex to a target sequence of a target nucleic acid. In some embodiments, a spacer sequence comprises a nucleotide sequence that is, at least partially, hybridizable to an equal length of a sequence (e.g., a target sequence) of a target nucleic acid. Exemplary hybridization conditions are described herein.

In some embodiments, a spacer sequence comprises a nucleotide sequence that is, at least partially, complementary to an equal length of a sequence (e.g., a target sequence) of a target nucleic acid. In some embodiments, a spacer sequence comprises at least 5 to about 50 contiguous nucleotides that are complementary to a target sequence in a target nucleic acid. A spacer sequence may comprise complementary to a target sequence that is adjacent to a PAM which is recognizable by an effector protein described herein.

In some embodiments, a spacer sequence comprises at least 5 to about 50 linked nucleotides. In some embodiments, a spacer sequence comprises at least 5 to about 50, at least 5 to about 25, at least about 10 to at least about 25, or at least about 15 to about 25 linked nucleotides. In some embodiments, the spacer sequence comprises 15-28 linked nucleotides in length. In some embodiments, a spacer sequence comprises 15-26, 15-24, 15-22, 15-20, 15-18, 16-28, 16-26, 16-24, 16-22, 16-20, 16-18, 17-26, 17-24, 17-22, 17-20, 17-18, 18-26, 18-24, or 18-22 linked nucleotides in length. In some embodiments, the spacer sequence comprises 10, 11, 12, 13, 14, 15, 16, 17, 18, 19, 20, 21, 22, 23, 24, 25, 26, 27, 28, 29, 30, or more nucleotides.

In some embodiments, a spacer sequence is adjacent to a repeat sequence. In some embodiments, a spacer sequence follows a repeat sequence in a 5′ to 3′ direction. In some embodiments, the spacer sequence(s) and the repeat sequence(s) of the guide nucleic acid are present within the same molecule. In some embodiments, the spacer(s) and repeat sequence(s) are linked directly to one another. In some embodiments, a linker is present between the spacer(s) and repeat sequences. Linkers may be any suitable linker, such as exemplary linkers described herein. In some embodiments, the spacer sequence(s) and the repeat sequence(s) of the guide nucleic acid are present in separate molecules, which are joined to one another by base pairing interactions.

In some embodiments, a spacer sequence comprises a nucleotide sequence that is at least 65%, at least 70%, at least 75%, at least 80%, at least 85%, at least 90%, at least 95% or 100% complementary to a target sequence of a target nucleic acid. In some embodiments, a target nucleic acid, such as DNA or RNA, may be a cancer gene or gene associated with a genetic disorder, or an amplicon thereof, as described herein. In some embodiments, a target nucleic acid is a gene selected from TABLE 4. In some embodiments, a spacer sequence comprises a sequence that is at least 65%, at least 70%, at least 75%, at least 80%, at least 85%, at least 90%, at least 95% or 100% complementary to a target sequence of a target nucleic acid selected from TABLE 4.

It is understood that the spacer sequence of a spacer sequence need not be 100% complementary to that of a target sequence of a target nucleic acid to hybridize or hybridize specifically to the target sequence. For example, the spacer sequence may comprise at least one modification, such as substituted or modified nucleotide, that is not complementary to the corresponding nucleotide of the target sequence. Spacer sequences are further described throughout herein.

In some embodiments, a spacer sequence comprises a nucleotide sequence that is at least 65%, at least 70%, at least 75%, at least 80%, at least 85%, at least 90%, at least 95% or 100% identical to a sequence set forth in EXAMPLE 2 herein.

Linker

In some embodiments, a guide nucleic acid for use with compositions, systems, and methods described herein comprises one or more linkers, or a nucleic acid encoding one or more linkers. In some embodiments, the guide nucleic acid comprises at least one, at least two, at least three, at least four, at least five, at least six, at least seven, at least eight, at least nine, or at least ten linkers. In some embodiments, the guide nucleic acid comprises one, two, three, four, five, six, seven, eight, nine, or ten linkers. In some embodiments, the guide nucleic acid comprises more than one linkers. In some embodiments, at least two of the more than one linkers are same. In some embodiments, at least two of the more than one linkers are not same.

In some embodiments, a linker comprises one to ten, one to seven, one to five, one to three, two to ten, two to eight, two to six, two to four, three to ten, three to seven, three to five, four to ten, four to eight, four to six, five to ten, five to seven, six to ten, six to eight, seven to ten, or eight to ten linked nucleotides. In some embodiments, the linker comprises one, two, three, four, five, six, seven, eight, nine, or ten linked nucleotides. In some embodiments, a linker comprises a nucleotide sequence of 5′-GAAA-3′.

In some embodiments, a guide nucleic acid comprises one or more linkers connecting one or more of a repeat sequence, a spacer sequence, a handle sequence, and an intermediary RNA sequence. In some embodiments, the guide nucleic acid comprises one or more linkers connecting one or more of: a repeat sequence and a spacer sequence; a handle sequence and a spacer sequence; an intermediary RNA sequence and a repeat sequence; and an intermediary RNA sequence and a spacer sequence. In some embodiments, the guide nucleic acid comprises at least two repeat sequences connected by a linker.

Intermediary Sequence

Guide nucleic acids described herein may comprise one or more intermediary sequences. In general, an intermediary sequence is not transactivated or transactivating. An intermediary sequence may also be referred to as an intermediary RNA, although it may comprise deoxyribonucleotides instead of or in addition to ribonucleotides. In general, the intermediary sequence non-covalently binds to an effector protein. In some embodiments, the intermediary sequence forms a secondary structure, for example in a cell, and an effector protein binds the secondary structure.

In such embodiments, an intermediary RNA is useful as part of a single nucleic acid system for compositions, methods, and systems described herein. In such embodiments, a single nucleic acid system comprises a guide nucleic acid comprising an intermediary RNA and a crRNA wherein, the intermediary RNA is linked to the crRNA. In some embodiments, a guide nucleic acid comprises an intermediary RNA and a crRNA wherein a repeat sequence of a crRNA is linked to the intermediary RNA.

In some instances, the length of an intermediary RNA sequence is not greater than 50, 56, 68, 71, 73, 95, or 105 linked nucleotides. In some embodiments, the length of an intermediary RNA sequence is about 30 to about 120 linked nucleotides. In some embodiments, the length of an intermediary RNA sequence is about 50 to about 105, about 50 to about 95, about 50 to about 73, about 50 to about 71, about 50 to about 68, or about 50 to about 56 linked nucleotides. In some embodiments, the length of an intermediary RNA sequence is 56 to 105 linked nucleotides, from 56 to 105 linked nucleotides, 68 to 105 linked nucleotides, 71 to 105 linked nucleotides, 73 to 105 linked nucleotides, or 95 to 105 linked nucleotides. In some embodiments, the length of an intermediary RNA sequence is 40 to 60 nucleotides. In some embodiments, the length of the intermediary RNA sequence is 50, 56, 68, 71, 73, 95, or 105 linked nucleotides. In some embodiments, the length of the intermediary RNA sequence is 50 nucleotides.

An intermediary RNA sequence may also comprise or form a secondary structure (e.g., one or more hairpin loops) that facilitates the binding of an effector protein to a guide nucleic acid and/or modification activity of an effector protein on a target nucleic acid (e.g., a hairpin region). An intermediary RNA sequence may comprise from 5′ to 3′, a 5′ region, a hairpin region, and a 3′ region. In some embodiments, the 5′ region may hybridize to the 3′ region. In some embodiments, the 5′ region of the intermediary RNA sequence does not hybridize to the 3′ region.

In some embodiments, the hairpin region may comprise a first sequence, a second sequence that is reverse complementary to the first sequence, and a stem-loop linking the first sequence and the second sequence. In some embodiments, an intermediary RNA sequence comprises a stem-loop structure comprising a stem region and a loop region. In some embodiments, the stem region is 4 to 8 linked nucleotides in length. In some embodiments, the stem region is 5 to 6 linked nucleotides in length. In some embodiments, the stem region is 4 to 5 linked nucleotides in length. In some embodiments, an intermediary RNA sequence comprises a pseudoknot (e.g., a secondary structure comprising a stem at least partially hybridized to a second stem or half-stem secondary structure). An effector protein may interact with an intermediary RNA sequence comprising a single stem region or multiple stem regions. In some embodiments, the nucleotide sequences of the multiple stem regions are identical to one another. In some embodiments, the nucleotide sequences of at least one of the multiple stem regions is not identical to those of the others. In some embodiments, an intermediary RNA sequence comprises 1, 2, 3, 4, 5 or more stem regions.

In some embodiments, the 3′ region of the intermediary RNA sequence is linked to a repeat sequence. In some embodiments, the 3′ region of the intermediary RNA sequence is linked to a crRNA. In some embodiments, a crRNA or a repeat sequence is linked to intermediary RNA sequence directly (e.g, covalently linked, such as through a phosphodiester bond). In some embodiments, a crRNA or a repeat sequence is linked to intermediary RNA sequence by any suitable linker, examples of which are provided herein. In some embodiments, an intermediary RNA sequence may comprise an unhybridized sequence at the 3′ end. The unhybridized sequence may have a length of about 1, about 2, about 3, about 4, about 5, about 6, about 7, about 8, about 9, about 10, about 12, about 14, about 16, about 18, or about 20 linked nucleotides. In some embodiments, the length of the un-hybridized sequence is 0 to 20 linked nucleotides.

Handle Sequence

Guide nucleic acids described herein may comprise one or more handle sequences.

In some embodiments, handle sequence comprises one or more of an intermediary RNA sequence, a repeat sequence, a linker, or combinations thereof. A handle sequence may comprise all or a portion of an intermediary RNA sequence. In such instances, at least a portion of an intermediary RNA non-covalently interacts with an effector protein. Additionally, or alternatively, the nucleotide sequence of a handle sequence may contain all or a portion of a repeat sequence. In such instances, at least a portion of an intermediary RNA or both, at least a portion of the intermediary RNA and at least a portion of repeat sequence, non-covalently interacts with an effector protein. In general, a single guide nucleic acid, also referred to as a single guide RNA (sgRNA), comprises a handle sequence.

In some embodiments, a handle sequence comprises an intermediary RNA sequence that is 5′ to a repeat sequence. In some embodiments, handle sequence comprises an intermediary RNA sequence, wherein the intermediary RNA sequence interacts, at least partially, with an effector protein in a sequence-specific manner. In some embodiments, handle sequence comprises an intermediary RNA sequence and a repeat sequence, wherein the repeat sequence is 3′ to the intermediary RNA sequence. In some embodiments, handle sequence comprises a linked intermediary RNA sequence and repeat sequence. In some embodiments, an intermediary RNA sequence and repeat sequence are directly linked (e.g., covalently linked, such as through a phosphodiester bond). In some embodiments, the intermediary RNA sequence and repeat sequence are linked by a suitable linker, examples of which are provided herein.

In some embodiments, a handle sequence is not greater than about 50, 56, 66, 67, 68, 69, 70, 71, 72, 73, 95, or 105 linked nucleotides. In some embodiments, a handle sequence is about 30 to about 120 linked nucleotides. In some embodiments, a handle sequence is about 50 to about 105, about 50 to about 95, about 50 to about 73, about 50 to about 71, about 50 to about 70, or about 50 to about 69 linked nucleotides. In some embodiments, a handle sequence is about 56 to 105 linked nucleotides, about 56 to 105 linked nucleotides, about 66 to 105 linked nucleotides, about 67 to 105 linked nucleotides, about 68 to 105 linked nucleotides, about 69 to 105 linked nucleotides, about 70 to 105 linked nucleotides, about 71 to 105 linked nucleotides, about 72 to 105 linked nucleotides, about 73 to 105 linked nucleotides, or about 95 to 105 linked nucleotides.

crRNA

In some embodiments, a guide nucleic acid comprises a crRNA. In some embodiments, a crRNA directs and/or binds the guide nucleic acid, or a complex thereof, to a target sequence of a target nucleic acid. For example, a crRNA may direct and bind an RNP complex to a target nucleic acid. In some embodiments, a crRNA comprises a spacer sequence as described herein. In some embodiments, a crRNA comprises a repeat sequence. In some embodiments, a crRNA comprises a repeat sequence which interacts with an effector protein described herein. In some embodiments, a crRNA comprises a repeat sequence and a spacer sequence. In some embodiments, a crRNA comprises a linked repeat sequence and a spacer sequence, which may be directly linked or linked by a suitable linker.

In such embodiments, a crRNA is useful as a single nucleic acid system for compositions, methods, and systems described herein or as part of a single nucleic acid system for compositions, methods, and systems described herein. In some embodiments, a crRNA is useful as part of a single nucleic acid system for compositions, methods, and systems described herein. In such embodiments, a single nucleic acid system comprises a guide nucleic acid comprising a crRNA wherein, a repeat sequence of a crRNA is capable of connecting a crRNA to an effector protein by being linked to another nucleotide sequence of a guide nucleic acid that is capable of non-covalently bonding with an effector protein. In such embodiments, a repeat sequence of a crRNA is linked to an intermediary RNA. In some embodiments, a single nucleic acid system comprises a guide nucleic acid comprising a crRNA and an intermediary RNA.

In some embodiments, a crRNA may be used as part of a single nucleic acid system in compositions, methods, and systems described herein. In such embodiments, a single nucleic acid system comprises a guide nucleic acid comprising a crRNA wherein a repeat sequence of a crRNA is capable of connecting a crRNA to an effector protein by interacting, at least partially, to an effector protein.

In some embodiments, a crRNA is useful as a part of a dual nucleic acid system for compositions, methods, and systems described herein. In such embodiments, a dual nucleic acid system comprises a tracrRNA and guide nucleic acid comprising a crRNA described herein. In some embodiments, a tracrRNA comprises a sequence that is capable of non-covalently bonding with an effector protein and a repeat hybridization sequence. In some embodiments, a dual nucleic acid system comprises a tracrRNA and guide nucleic acid comprising a crRNA wherein a repeat sequence of a crRNA is capable of connecting a crRNA to an effector protein by hybridizing, at least partially, to a repeat hybridization sequence of a tracrRNA. Exemplary hybridization conditions are described herein.

In some embodiments, the length of the crRNA is not greater than about 40, about 45, about 50, about 55, about 60, about 65, about 70 or about 75 linked nucleotides. In some embodiments, the length of the crRNA is about 30 to about 120 linked nucleotides. In some embodiments, the length of a crRNA is about 40 to about 100, about 40 to about 90, about 40 to about 80, about 40 to about 70, about 40 to about 60, about 40 to about 50, about 50 to about 90, about 50 to about 80, about 50 to about 70, or about 50 to about 60 linked nucleotides. In some embodiments, the length of a crRNA is about 40, about 45, about 50, about 55, about 60, about 65, about 70 or about 75 linked nucleotides.

sgRNA

Guide nucleic acids described herein may be single guide nucleic acid, as referred to herein as a single guide RNA (sgRNA). In general, an sgRNA may be used as part of a single nucleic acid system in compositions, methods, and systems described herein. In some embodiments, a sgRNA comprises one or more of one or more of a handle sequence, an intermediary RNA sequence, a crRNA, a repeat sequence, a spacer sequence, a linker, or combinations thereof. For example, a sgRNA comprises a handle sequence and a spacer sequence; an intermediary RNA sequence and an crRNA; an intermediary RNA sequence, a repeat sequence and a spacer sequence; and the like.

In some embodiments, a sgRNA comprises an intermediary RNA sequence and an crRNA. In some embodiments, an intermediary RNA sequence is 5′ to a crRNA in an sgRNA. In some embodiments, a sgRNA comprises a linked intermediary RNA sequence and crRNA. In some embodiments, an intermediary RNA sequence and a crRNA are linked in an sgRNA directly (e.g, covalently linked, such as through a phosphodiester bond) In some embodiments, an intermediary RNA sequence and a crRNA are linked in an sgRNA by any suitable linker, examples of which are provided herein.

In some embodiments, a sgRNA comprises a handle sequence and a spacer sequence. In some embodiments, a handle sequence is 5′ to a spacer sequence in an sgRNA. In some embodiments, a sgRNA comprises a linked handle sequence and spacer sequence. In some embodiments, a handle sequence and a spacer sequence are linked in an sgRNA directly (e.g., covalently linked, such as through a phosphodiester bond) In some embodiments, a handle sequence and a spacer sequence are linked in an sgRNA by any suitable linker, examples of which are provided herein.

In some embodiments, a sgRNA comprises an intermediary RNA sequence, a repeat sequence, and a spacer sequence. In some embodiments, an intermediary RNA sequence is 5′ to a repeat sequence in an sgRNA. In some embodiments, a sgRNA comprises a linked intermediary RNA sequence and repeat sequence. In some embodiments, an intermediary RNA sequence and a repeat sequence are linked in an sgRNA directly (e.g., covalently linked, such as through a phosphodiester bond) In some embodiments, an intermediary RNA sequence and a repeat sequence are linked in an sgRNA by any suitable linker, examples of which are provided herein. In some embodiments, a repeat sequence is 5′ to a spacer sequence in an sgRNA. In some embodiments, a sgRNA comprises a linked repeat sequence and spacer sequence. In some embodiments, a repeat sequence and a spacer sequence are linked in an sgRNA directly (e.g, covalently linked, such as through a phosphodiester bond) In some embodiments, a repeat sequence and a spacer sequence are linked in an sgRNA by any suitable linker, examples of which are provided herein.

### VI. ENGINEERED MODIFICATIONS

Polypeptides (e.g., effector proteins or effector partners) and nucleic acids (e.g., engineered guide nucleic acids) described herein may be further modified as described throughout and as further described herein. Examples are modifications of interest that do not alter primary sequence, including chemical derivatization of polypeptides, e.g., acylation, acetylation, carboxylation, amidation, etc. Also included are modifications of glycosylation, e.g. those made by modifying the glycosylation patterns of a polypeptide during its synthesis and processing or in further processing steps; e.g. by exposing the polypeptide to enzymes which affect glycosylation, such as mammalian glycosylating or deglycosylating enzymes. Also embraced are sequences that have phosphorylated amino acid residues, e.g. phosphotyrosine, phosphoserine, or phosphothreonine.

Modifications disclosed herein may also include modification of described polypeptides and/or engineered guide nucleic acids through any suitable method, such as molecular biological techniques and/or synthetic chemistry, to improve their resistance to proteolytic degradation, to change the target sequence specificity, to optimize solubility properties, to alter protein activity (e.g., transcription modulatory activity, enzymatic activity, etc.) or to render them more suitable. Analogs of such polypeptides include those containing residues other than naturally occurring L-amino acids, e.g. D-amino acids or non-naturally occurring synthetic amino acids. D-amino acids may be substituted for some or all of the amino acid residues. Modifications may also include modifications with non-naturally occurring unnatural amino acids. The particular sequence and the manner of preparation will be determined by convenience, economics, purity required, and the like.

Modifications may further include the introduction of various groups to polypeptides and/or engineered guide nucleic acids described herein. For example, groups may be introduced during synthesis or during expression of a polypeptide (e.g., an effector protein and/or an effector partner), which allow for linking to other molecules or to a surface. Thus, e.g., cysteines may be used to make thioethers, histidines for linking to a metal ion complex, carboxyl groups for forming amides or esters, amino groups for forming amides, and the like.

Modifications may further include modification of nucleic acids described herein (e.g., engineered guide nucleic acids) to provide the nucleic acid with a new or enhanced feature, such as improved stability. Such modifications of a nucleic acid include a base modification, a backbone modification, a sugar modification, or combinations thereof, of one or more nucleotides, nucleosides, or nucleobases in a nucleic acid.

In some embodiments, nucleic acids (e.g., nucleic acids encoding effector proteins, nucleic acids encoding effector partners, engineered guide nucleic acids, or nucleic acids encoding engineered guide nucleic acids) described herein comprise one or more modifications comprising: 2′O-methyl modified nucleotides, 2′ Fluoro modified nucleotides; locked nucleic acid (LNA) modified nucleotides; peptide nucleic acid (PNA) modified nucleotides; nucleotides with phosphorothioate linkages; a 5′ cap (e.g., a 7-methylguanylate cap (m7G)), phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkyl phosphoramidates, phosphorodiamidates, thionophosphor amidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage; phosphorothioate and/or heteroatom internucleoside linkages, such as —CH2-NH—O—CH2-, —CH2-N(CH3)-O—CH2- (known as a methylene (methylimino) or MMI backbone), —CH2-O—N(CH3)-CH2-, —CH2-N(CH3)-N(CH3)-CH2- and —O—N(CH3)-CH2-CH2- (wherein the native phosphodiester internucleotide linkage is represented as —O—P(═O)(OH)—O—CH2-); morpholino linkages (formed in part from the sugar portion of a nucleoside); morpholino backbones; phosphorodiamidate or other non-phosphodiester internucleoside linkages; siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; other backbone modifications having mixed N, O, S and CH₂ component parts; and combinations thereof.

### VII. VECTORS AND MULTIPLEXED EXPRESSION VECTORS

Compositions, systems, and methods described herein comprise a vector or a use thereof. A vector may encode one component of a composition or system described herein, or may encode multiple components (e.g., effector proteins, effector partners, guide nucleic acids, donor nucleic acids, target nucleic acids, etc., as described herein). The vector may be part of a vector system, wherein a vector system comprises a library of vectors each encoding one or more component of a composition or system described herein. In some embodiments, the vector system may be a multi-vector system, wherein a multi-vector system comprises a library of vectors wherein at least two vectors encode different components of a composition or system described herein. In some embodiments, components described herein (e.g., an effector protein, a guide nucleic acid, an effector partner, and/or a target nucleic acid) are encoded by the same vector. In some embodiments, components described herein (e.g., an effector protein, a guide nucleic acid, an effector partner, and/or a target nucleic acid) are each encoded by different vectors of the system.

In some embodiments, a vector may encode one or more of any system component, including but not limited to effector proteins, effector partners, guide nucleic acids, donor nucleic acids, and target nucleic acids as described herein. In some embodiments, a system component encoding sequence is operably linked to a promoter that is operable in a target cell, such as a eukaryotic cell. In some embodiments, a vector may encode 1, 2, 3, 4 or more of any system component. For example, a vector may encode two or more guide nucleic acids, wherein each guide nucleic acid comprises a different sequence. Also by way of non-limiting example, a single vector may encode an effector protein and an effector partner. A vector may encode an effector protein, an effector partner, and a guide nucleic acid. A vector may encode an effector protein, an effector partner, a guide nucleic acid, and a donor nucleic acid.

In some embodiments, a vector may comprise or encode one or more regulatory elements. Regulatory elements may refer to transcriptional and translational control sequences, such as promoters, enhancers, polyadenylation signals, terminators, protein degradation signals, and the like, that provide for and/or regulate transcription of a non-coding sequence or a coding sequence and/or regulate translation of an encoded polypeptide. In some embodiments, a vector may comprise or encode for one or more additional elements, such as, for example, replication origins, antibiotic resistance (or a nucleic acid encoding the same), a tag (or a nucleic acid encoding the same), selectable markers, and the like.

Vectors described herein may encode a promoter—a regulatory region on a nucleic acid, such as a DNA sequence, capable of initiating transcription of a downstream (3′ direction) coding or non-coding sequence. As used herein, a promoter may be bound at its 3′ terminus to a nucleic acid the expression or transcription of which is desired, and extends upstream (5′ direction) to include bases or elements necessary to initiate transcription or induce expression, which could be measured at a detectable level. A promoter may comprise a nucleotide sequence, referred to herein as a “promoter sequence”. A promoter sequence may include a transcription initiation site, and one or more protein binding domains responsible for the binding of transcription machinery, such as RNA polymerase. When eukaryotic promoters are used, such promoters may contain “TATA” boxes and “CAT” boxes. Various promoters, including inducible promoters, may be used to drive expression, i.e., transcriptional activation, of the nucleic acid of interest. Accordingly, in some embodiments, the nucleic acid of interest may be operably linked to a promoter.

Promotors may be any suitable type of promoter envisioned for the compositions, systems, and methods described herein. Examples include constitutively active promoters (e.g., CMV promoter), inducible promoters (e.g., heat shock promoter, tetracycline-regulated promoter, steroid-regulated promoter, metal-regulated promoter, estrogen receptor-regulated promoter, etc.), spatially restricted and/or temporally restricted promoters (e.g., a tissue specific promoter, a cell type specific promoter, etc.), etc. Suitable promoters include, but are not limited to: SV40 early promoter, mouse mammary tumor virus long terminal repeat (LTR) promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, a human U6 small nuclear promoter (U6), an enhanced U6 promoter, and a human Hl promoter (H1). By transcriptional activation, it is intended that transcription will be increased above basal levels in the target cell by 10 fold, by 100 fold, or by 1000 fold, or more. In addition, vectors used for providing a nucleic acid that, when transcribed, produces an engineered guide nucleic acid and/or a nucleic acid that encodes an effector protein to a cell may include nucleic acid sequences that encode for selectable markers in the target cells, so as to identify cells that have taken up the engineered guide nucleic acid and/or an effector protein.

In general, vectors provided herein comprise at least one promotor or a combination of promoters driving expression or transcription of one or more genome editing tools described herein. In some embodiments, the viral vector comprises a nucleotide sequence of a promoter. In some embodiments, the viral vector comprises two promoters. In some embodiments, the viral vector comprises three promoters. In some embodiments, the length of the promoter is less than about 500, less than about 400, or less than about 300 linked nucleotides. In some embodiments, the length of the promoter is at least 100 linked nucleotides. Non-limiting examples of promoters include CMV, 7SK, EF1a, RPBSA, hPGK, EFS, SV40, PGK1, Ubc, human beta actin promoter, CAG, TRE, UAS, Ac5, Polyhedrin, CaMKIIa, GAL1, H1, TEF1, GDS, ADH1, CaMV35S, Ubi, U6, MNDU3, MSCV, Ck8e, SPC5-12, Desmin, MND and CAG.

In some embodiments, the promoter is an inducible promoter that only drives expression of its corresponding gene when a signal is present, e.g., a hormone, a small molecule, a peptide. Non-limiting examples of inducible promoters are the T7 RNA polymerase promoter, the T3 RNA polymerase promoter, the Isopropyl-beta-D-thiogalactopyranoside (IPTG)-regulated promoter, a lactose induced promoter, a heat shock promoter, a tetracycline-regulated promoter (tetracycline-inducible or tetracycline-repressible), a steroid regulated promoter, a metal-regulated promoter, and an estrogen receptor-regulated promoter. In some embodiments, the promoter is an activation-inducible promoter, such as a CD69 promoter, as described further in Kulemzin et al., (2019), BMC Med Genomics, 12:44. In some embodiments, the promoter for expressing effector protein is a muscle-specific promoter. In some embodiments, the muscle-specific promoter comprises Ck8e, SPC5-12, or Desmin promoter sequence. In some embodiments, the promoter for expressing effector protein is a ubiquitous promoter. In some embodiments, the ubiquitous promoter comprises MND or CAG promoter sequence.

In some embodiments, a vector used herein is an nucleic acid expression vector. In some embodiments, a vector used herein is a recombinant expression vector. In some embodiments, a vector used herein is a messenger RNA.

In some embodiments, one or more components of a composition or system described herein (e.g., effector proteins, effector partners, guide nucleic acids, donor nucleic acids, target nucleic acids, etc., as described herein) are independently administered. In some embodiments, an effector protein (or a nucleic acid encoding same) and an effector partner (or a nucleic acid encoding same) are independently administered. In some embodiments, an effector protein (or a nucleic acid encoding same), an effector partner (or a nucleic acid encoding same), an engineered guide nucleic acid (or a nucleic acid that, when transcribed, produces same), and/or a donor nucleic acid are each independently administered. Independent administration may be contact with a target nucleic acid, administered to a cell, such as a host cell, or administered as method of nucleic acid detection, editing, and/or treatment as described herein, in a single vehicle, such as a single expression vector. In some embodiments, each components of a composition or system described herein are each independently administered in a single vehicle or expression vector.

In some embodiments, one or more components of a composition or system described herein are co-administered. In some embodiments, an effector protein (or a nucleic acid encoding same) and/or an engineered guide nucleic acid (or a nucleic acid that, when transcribed, produces same) are co-administered with a donor nucleic acid. Co-administration may be contact with a target nucleic acid, administered to a cell, such as a host cell, or administered as method of nucleic acid detection, editing, and/or treatment as described herein, in a single vehicle, such as a single expression vector. In some embodiments, an effector protein (or a nucleic acid encoding same) and/or an engineered guide nucleic acid (or a nucleic acid that, when transcribed, produces same) are not co-administered with donor nucleic acid in a single vehicle. In some embodiments, an effector protein (or a nucleic acid encoding same), an engineered guide nucleic acid (or a nucleic acid that, when transcribed, produces same), and/or donor nucleic acid are administered in one or more or two or more vehicles, such as one or more, or two or more expression vectors.

In some embodiments, a cell comprises a vector, a nucleic acid expression vector, or a library of nucleic acid expression vectors as described herein. In some embodiments, a cell comprises a target nucleic acid modified by any one of the compositions described herein, by any one of the nucleic acid expression vectors described herein, or by any one library of nucleic acid expression vectors as described herein. In some embodiments, the cell is a eukaryotic cell. In some embodiments, the cell is a mammalian cell. In some embodiments, the cell is a human cell. In some embodiments, a population of cells comprises at least one cell comprising a vector, a nucleic acid expression vector, or a library of nucleic acid expression vectors as described herein.

Viral Vectors

An expression vector may be a viral vector. In some embodiments, a viral vector comprises a nucleic acid to be delivered into a host cell via a recombinantly produced virus or viral particle. The nucleic acid may be single-stranded or double stranded, linear or circular, segmented or non-segmented. The nucleic acid may comprise DNA, RNA, or a combination thereof. In some embodiments, the expression vector is an adeno-associated viral vector. There are a variety of viral vectors that are associated with various types of viruses, including but not limited to retroviruses (e.g., lentiviruses and γ-retroviruses), adenoviruses, arenaviruses, alphaviruses, adeno-associated viruses (AAVs), baculoviruses, vaccinia viruses, herpes simplex viruses and poxviruses. A viral vector provided herein may be derived from or based on any such virus. Often the viral vectors provided herein are an adeno-associated viral vector (AAV vector). Generally, an AAV vector has two inverted terminal repeats (ITRs). According, in some embodiments, the viral vector provided herein comprises two inverted terminal repeats of AAV. The DNA sequence in between the ITRs of an AAV vector provided herein may be referred to herein as the sequence encoding the genome editing tools. These genome editing tools may include, but are not limited to, an effector protein, effector protein modifications (e.g., nuclear localization signal (NLS), polyA tail), guide nucleic acid(s), respective promoter(s), and a donor nucleic acid, or combinations thereof. In some embodiments, a nuclear localization signal comprises an entity (e.g., peptide) that facilitates localization of a nucleic acid, protein, or small molecule to the nucleus, when present in a cell that contains a nuclear compartment.

In general, viral vectors provided herein comprise at least one promotor or a combination of promoters driving expression or transcription of one or more genome editing tools described herein. In some embodiments, the length of the promoter is less than about 500, less than about 400, or less than about 300 linked nucleotides. In some embodiments, the length of the promoter is at least 100 linked nucleotides. Non-limiting examples of promoters include CMV, EF1a, RPBSA, hPGK, EFS, SV40, PGK1, Ubc, human beta actin promoter, CAG, TRE, UAS, Ac5, Polyhedrin, CaMKIIa, GAL1, H1, TEF1, GDS, ADH1, CaMV35S, Ubi, U6, MNDU3, and MSCV. In some embodiments, the promoter is an inducible promoter that only drives expression of its corresponding gene when a signal is present, e.g., a hormone, a small molecule, a peptide. Non-limiting examples of inducible promoters are the T7 RNA polymerase promoter, the T3 RNA polymerase promoter, the Isopropyl-beta-D-thiogalactopyranoside (IPTG)-regulated promoter, a lactose induced promoter, a heat shock promoter, a tetracycline-regulated promoter (tetracycline-inducible or tetracycline-repressible), a steroid regulated promoter, a metal-regulated promoter, and an estrogen receptor-regulated promoter. In some embodiments, the promoter is an activation-inducible promoter, such as a CD69 promoter, as described further in Kulemzin et al., (2019), BMC Med Genomics, 12:44.

In some embodiments, the coding region of the AAV vector forms an intramolecular double-stranded DNA template thereby generating an AAV vector that is a self-complementary AAV (scAAV) vector. In general, the sequence encoding the genome editing tools of an scAAV vector has a length of about 2 kb to about 3 kb. The scAAV vector may comprise nucleotide sequences encoding an effector protein, providing guide nucleic acids described herein, and a donor nucleic acid described herein. In some embodiments, the AAV vector provided herein is a self-inactivating AAV vector.

In some embodiments, an AAV vector provided herein comprises a modification, such as an insertion, deletion, chemical alteration, or synthetic modification, relative to a wild-type AAV vector.

In some embodiments, the viral particle that delivers the viral vector described herein is an AAV. AAVs are characterized by their serotype. Non-limiting examples of AAV serotypes are AAV1, AAV2, AAV3, AAV4, AAV5, AAV6, AAV7, AAV8, AAV9, AAV10, AAV11, AAV12, scAAV, AAV-rh10, chimeric or hybrid AAV, or any combination, derivative, or variant thereof.

Producing AAV Particles

The AAV particles described herein may be referred to as recombinant AAV (rAAV). Often, rAAV particles are generated by transfecting AAV producing cells with an AAV-containing plasmid carrying the sequence encoding the genome editing tools, a plasmid that carries viral encoding regions, i.e., Rep and Cap gene regions; and a plasmid that provides the helper genes such as EIA, EIB, E2A, E4ORF6 and VA. In some embodiments, the AAV producing cells are mammalian cells. In some embodiments, host cells for rAAV viral particle production are mammalian cells. In some embodiments, a mammalian cell for rAAV viral particle production is a COS cell, a HEK293T cell, a HeLa cell, a KB cell, a derivative thereof, or a combination thereof. In some embodiments, rAAV virus particles may be produced in the mammalian cell culture system by providing the rAAV plasmid to the mammalian cell. In some embodiments, producing rAAV virus particles in a mammalian cell may comprise transfecting vectors that express the rep protein, the capsid protein, and the gene-of-interest expression construct flanked by the ITR sequence on the 5′ and 3′ ends. Methods of such processes are provided in, for example, Naso et al., BioDrugs, 2017 August; 31(4):317-334 and Benskey et al., (2019), Methods Mol Biol., 1937:3-26, each of which is incorporated by reference in their entireties.

In some embodiments, rAAV is produced in a non-mammalian cell. In some embodiments, rAAV is produced in an insect cell. In some embodiments, an insect cell for producing rAAV viral particles comprises a Sf9 cell. In some embodiments, production of rAAV virus particles in insect cells may comprise baculovirus. In some embodiments, production of rAAV virus particles in insect cells may comprise infecting the insect cells with three recombinant baculoviruses, one carrying the cap gene, one carrying the rep gene, and one carrying the gene-of-interest expression construct enclosed by an ITR on both the 5′ and 3′ end. In some embodiments, rAAV virus particles are produced by the One Bac system. In some embodiments, rAAV virus particles may be produced by the Two Bac system. In some embodiments, in the Two Bac system, the rep gene and the cap gene of the AAV is integrated into one baculovirus virus genome, and the ITR sequence and the gene-of-interest expression construct is integrated into another baculovirus virus genome. In some embodiments, in the One Bac system, an insect cell line that expresses both the rep protein and the capsid protein is established and infected with a baculovirus virus integrated with the ITR sequence and the gene-of-interest expression construct. Details of such processes are provided in, for example, Smith et. al., (1983), Mol. Cell. Biol., 3(12):2156-65; Urabe et al., (2002), Hum. Gene. Ther., 1; 13(16):1935-43; and Benskey et al., (2019), Methods Mol Biol., 1937:3-26, each of which is incorporated by reference in its entirety.

Lipid Particles

In some embodiments, compositions and systems provided herein comprise a lipid particle. In some embodiments, a lipid particle is a lipid nanoparticle (LNP). In some embodiments, a lipid or a lipid nanoparticle may encapsulate an expression vector. In some embodiments, the expression vector incorporates the effector protein, the guide nucleic acid, the nucleic acid encoding the effector protein and/or the DNA molecule encoding the guide nucleic acid. LNPs are a non-viral delivery system for gene therapy. LNPs are effective for delivery of nucleic acids. Beneficial properties of LNP include ease of manufacture, low cytotoxicity and immunogenicity, high efficiency of nucleic acid encapsulation and cell transfection, multi-dosing capabilities and flexibility of design (Kulkarni et al., (2018) Nucleic Acid Therapeutics, 28(3):146-157). In some embodiments, a method may comprise contacting a cell with an expression vector. In some embodiments, contacting may comprise electroporation, lipofection, or lipid nanoparticle (LNP) delivery of an expression vector. In some embodiments, a nucleic acid expression vector is a non-viral vector. In some embodiments, compositions and methods comprise a lipid, polymer, nanoparticle, or a combination thereof, or use thereof, to introduce a Cas protein, guide nucleic acid, donor template or any combination thereof to a cell. Non-limiting examples of lipids and polymers are cationic polymers, cationic lipids, ionizable lipids, or bio-responsive polymers. In some embodiments, the ionizable lipids exploits chemical-physical properties of the endosomal environment (e.g., pH) offering improved delivery of nucleic acids. In some embodiments, the ionizable lipids are neutral at physiological pH. In some embodiments, the ionizable lipids are protonated under acidi pH. In some embodiments, the bio-responsive polymer exploits chemical-physical properties of the endosomal environment (e.g., pH) to preferentially release the genetic material in the intracellular space.

In some embodiments, a LNP comprises an outer shell and an inner core. In some embodiments, the outer shell comprises lipids. In some embodiments, the lipids comprise modified lipids. In some embodiments, the modified lipids comprise pegylated lipids. In some embodiments, the lipids comprise one or more of cationic lipids, anionic lipids, ionizable lipids, and non-ionic lipids. In some embodiments, the LNP comprises one or more of N¹,N³,N⁵-tris(3-(didodecylamino)propyl)benzene-1,3,5-tricarboxamide (TT3), 2-dioleoyl-sn-glycero-3-phosphoethanolamine (DOPE), 1-palmitoyl-2-oleoylsn-glycero-3-phosphoethanolamine (POPE), 1,2-distearoyl-sn-glycero-3-phosphocholine (DSPC), cholesterol (Choi), 1,2-dimyristoyl-sn-glycerol, and methoxypolyethylene glycol (DMG-PECh000), derivatives, analogs, or variants thereof. In some embodiments, the LNP has a negative net overall charge prior to complexation with one or more of a guide RNA, a nucleic acid encoding the guide RNA, an effector protein, and a nucleic acid encoding the effector protein. In some embodiments, the inner core is a hydrophobic core. In some embodiments, the guide RNA or the nucleic acid encoding the guide RNA forms a complex with one or more of the cationic lipids and the ionizable lipids. In some embodiments, the nucleic acid encoding the effector protein or the nucleic acid encoding the guide RNA is self-replicating.

In some embodiments, a LNP comprises one or more of a cationic lipid, an ionizable lipid and a modified version thereof. In some embodiments, the ionizable lipid comprises TT3 or a derivative thereof. Accordingly, in some embodiments, the LNP comprises one or more of TT3 and pegylated TT3. The publication WO2016187531 is hereby incorporated by reference in its entirety, which describes representative LNP formulations in Table 2, and Table 3, and representative methods of delivering LNP formulations in Example 7.

### VIII. TARGET NUCLEIC ACIDS AND SAMPLES

Described herein are compositions, systems and methods for modifying or detecting a target nucleic acid, wherein the target nucleic acid is a gene, a portion thereof, a transcript thereof. In some embodiments, the target nucleic acid is a reverse transcript (e.g. a cDNA) of an mRNA transcribed from the gene, or an amplicon thereof acid. In some embodiments, the target nucleic acid is a single stranded nucleic acid. Alternatively, or in combination, the target nucleic acid is a double stranded nucleic acid and is prepared into single stranded nucleic acids before or upon contacting the reagents. In some embodiments, the target nucleic acid is a double stranded nucleic acid. In some embodiments, the double stranded nucleic acid is DNA. The target nucleic acid may be a RNA. The target nucleic acids include but are not limited to mRNA, rRNA, tRNA, non-coding RNA, long non-coding RNA, and microRNA (miRNA). In some embodiments, the target nucleic acid is complementary DNA (cDNA) synthesized from a single-stranded RNA template in a reaction catalyzed by a reverse transcriptase. In some embodiments, the target nucleic acid is single-stranded RNA (ssRNA) or mRNA. In some embodiments, the target nucleic acid is from a virus, a parasite, or a bacterium described herein. As another non-limiting example, the target nucleic acid may be responsible for a disease, contain a mutation (e.g., single strand polymorphism, point mutation, insertion, or deletion), be contained in an amplicon, or be uniquely identifiable from the surrounding nucleic acids (e.g., contain a unique sequence of nucleotides).

In some embodiments, the target nucleic acid is a double stranded nucleic acid comprising a target strand and a non-target strand, wherein the target strand comprises a target sequence. In some embodiments, where a target strand comprises a target sequence, at least a portion of the engineered guide nucleic acid is complementary to the target sequence on the target strand. In some embodiments, where the target nucleic acid is a double stranded nucleic acid comprising a target strand and a non-target strand, and wherein the target strand comprises a target sequence, at least a portion of the engineered guide nucleic acid is complementary to the target sequence on the target strand. In some embodiments, a target nucleic acid comprises a PAM as described herein that is located on the non-target strand. Such a PAM described herein, in some embodiments, is adjacent (e.g., within 1, 2, 3, 4 or 5 nucleotides) to the 5′ end of the target sequence on the non-target strand of the double stranded DNA molecule. In some embodiments, such a PAM described herein is directly adjacent to the 5′ end of a target sequence on the non-target strand of the double stranded DNA molecule.

In some embodiments, an effector protein and/or effector partner described herein or a multimeric complex thereof recognizes a PAM on a target nucleic acid. In some embodiments, one or more effector proteins and/or one or more effector partners, or combinations thereof, of the multimeric complex recognize a PAM on a target nucleic acid. In some embodiments, only one effector protein of the multimeric complex recognizes a PAM on a target nucleic acid. In some embodiments, the PAM is 3′ to the spacer region of the crRNA. In some embodiments, the PAM is directly 3′ to the spacer region of the crRNA.

An effector protein of the present disclosure, a dimer thereof, or a multimeric complex thereof may cleave or nick a target nucleic acid within or near a protospacer adjacent motif (PAM) sequence of the target nucleic acid. In some embodiments, cleavage occurs within 1, 2, 3, 4, 5, 6, 7, 8, 9 or 10 nucleotides of a 5′ or 3′ terminus of a PAM sequence. A target nucleic acid may comprise a PAM sequence adjacent to a sequence that is complementary to a guide nucleic acid spacer region.

In some embodiments, the target nucleic acid comprises 5 to 100, 5 to 90, 5 to 80, 5 to 70, 5 to 60, 5 to 50, 5 to 40, 5 to 30, 5 to 25, 5 to 20, 5 to 15, or 5 to 10 linked nucleotides. In some embodiments, the target nucleic acid comprises 10 to 90, 20 to 80, 30 to 70, or 40 to 60 linked nucleotides. In some embodiments, the target nucleic acid comprises 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 60, 70, 80, 90, or 100 linked nucleotides. In some embodiments, the target nucleic acid comprises at least 10, at least 20, at least 30, at least 40, at least 50, at least 60, at least 70, at least 80, at least 90, or at least 100 linked nucleotides.

In some embodiments, the target nucleic acid comprises a portion or a specific region of a nucleic acid from a genomic locus, any DNA amplicon of, a reverse transcribed mRNA, or a cDNA from a gene described herein. In some embodiments, the target nucleic acid is an amplicon of at least a portion of a gene. Non-limiting examples of genes are set forth in TABLE 4. Nucleic acid sequences of target nucleic acids and/or corresponding genes are readily available in public databases as known and used in the art. In some embodiments, the target nucleic acid is selected from TABLE 4.

In some embodiments, the target nucleic acid comprises a target locus. In some embodiments, the target nucleic acid comprises more than one target loci. In some embodiments, the target nucleic acid comprises two target loci. Accordingly, in some embodiments, the target nucleic acid may comprise one or more target sequences.

In some embodiments, the one or more target sequence is within any one of the genes set forth in TABLE 4. In some embodiments, the target sequence is within an exon of any one of the genes set forth in TABLE 4. In some embodiments, then target sequence covers the junction of two exons. In some embodiments, the target sequence is located within about 1 to about 300 nucleotides, about 10 to about 250, about 20 to about 200, about 30 to about 150, about 40 to about 100, or about 50 nucleotides of the 5′ untranslated region (UTR). In some embodiments, the target sequence is located within about 1 to about 300 nucleotides, about 10 to about 250, about 20 to about 200, about 30 to about 150, about 40 to about 100, or about 50 nucleotides of the 3′ UTR.

In some embodiments, the target sequence is at least partially within a targeted exon within any one of the genes set forth in TABLE 4. A targeted exon may mean any portion within, contiguous with, or adjacent to a specified exon of interest may be targeted by the compositions, systems, and methods described herein. In some embodiments, one or more of the exons are targeted. In some embodiments, one or more of exons of any one the genes set forth in TABLE 4 are targeted.

In some embodiments, the start of an exon is referred to interchangeably herein as the 5′ end of an exon. In some embodiments, the 5′ region of an exon comprises a sequence about 1 to about 300 nucleotides adjacent to the 5′ end of an exon when moving upstream in the 5′ direction, or a sequence about 1 to about 300 nucleotides adjacent to the 5′ end of an exon when moving downstream in the 3′ direction, or both.

In some embodiments, the end of an exon is referred to interchangeably herein as the 3′ end of an exon. In some embodiments, the 3′ region of an exon comprises a sequence about 1 to about 300 nucleotides adjacent to the 3′ end of an exon when moving upstream in the 5′ direction, or a sequence about 1 to about 300 nucleotides adjacent to the 3′ end of an exon when moving downstream in the 3′ direction, or both.

Nucleic acids, such as DNA and pre-mRNA, may contain at least one intron and at least one exon, wherein as read in the 5′ to the 3′ direction of a nucleic acid strand, the 3′ end of an intron may be adjacent to the 5′ end of an exon, and wherein said intron and exon correspond for transcription purposes. If a nucleic acid strand contains more than one intron and exon, the 5′ end of the second intron is adjacent to the 3′ end of the first exon, and 5′ end of the second exon is adjacent to the 3′ end of the second intron. The junction between an intron and an exon may be referred to herein as a splice junction, wherein a 5′ splice site (SS) may refer to the +1/+2 position at the 5′ end of intron and a 3′ SS may refer to the last two positions at the 3′ end of an intron. Alternatively, a 5′ SS may refer to the 5′ end of an exon and a 3′ SS may refer to the 3′ end of an exon. In some embodiments, nucleic acids may contain one or more elements that act as a signal during transcription, splicing, and/or translation. In some embodiments, signaling elements include a 5′ SS, a 3′SS, a premature stop codon, U1 and/or U2 binding sequences, and cis acting elements such as branch site (BS), polypyridine tract (PYT), exonic and intronic splicing enhancers (ESEs and ISEs) or silencers (ESSs and ISSs).

In some embodiments, a target sequence that a guide nucleic acid binds is at least partially within a targeted exon within any one of the genes set forth in TABLE 4, and wherein at least a portion of the target nucleic acid is within a sequence about 1 to about 300 nucleotides adjacent to: the start of a targeted exon, the end of a targeted exon, or both. In some embodiments, at least a portion of the target sequence that a guide nucleic acid binds may comprise a sequence about 1 to about 300 nucleotides, about 10 to about 250, about 20 to about 200, about 30 to about 150, about 40 to about 100, or about 50 nucleotides adjacent to: the start of a targeted exon, the end of a targeted exon, or both.

In some embodiments, at least a portion of the target nucleic acid that a guide nucleic acid binds is within a sequence about 5 or more, about 10 or more, about 15 or more, about 20 or more, about 25 or more, about 30 or more, about 35 or more, about 40 or more, about 45 or more, about 50 or more, about 55 or more, about 60 or more, about 65 or more, about 70 or more, about 75 or more, about 80 or more, about 85 or more, about 90 or more, about 95 or more, about 100 or more, about 105 or more, about 110 or more, about 115 or more, about 120 or more, about 125 or more, about 130 or more, about 135 or more, about 140 or more, about 145 or more, or about 150 or more nucleotides adjacent to: the start of a targeted exon, the end of a targeted exon, or both.

In some embodiments, a target sequence that a guide nucleic acid binds is at least partially within a targeted exon within any one of the genes set forth in TABLE 4, and wherein at least a portion of the target nucleic acid is within a sequence about 1 to about 300 nucleotides adjacent to: the start of a targeted exon, the end of a targeted exon, or both. In some embodiments, at least a portion of the target sequence that a guide nucleic acid binds may comprise a sequence about 1 to about 300 nucleotides, about 10 to about 250, about 20 to about 200, about 30 to about 150, about 40 to about 100, or about 50 nucleotides adjacent to: one or more signaling element comprising a 5'S S, a 3′ SS, a premature stop codon, U1 binding sequence, U2 binding sequence, a BS, a PYT, ESE, an ISE, an ESS, an ISS, more than one of the foregoing, or any combination thereof.

In some embodiments, at least a portion of the target nucleic acid that a guide nucleic acid binds is within a sequence about 5 or more, about 10 or more, about 15 or more, about 20 or more, about 25 or more, about 30 or more, about 35 or more, about 40 or more, about 45 or more, about 50 or more, about 55 or more, about 60 or more, about 65 or more, about 70 or more, about 75 or more, about 80 or more, about 85 or more, about 90 or more, about 95 or more, about 100 or more, about 105 or more, about 110 or more, about 115 or more, about 120 or more, about 125 or more, about 130 or more, about 135 or more, about 140 or more, about 145 or more, or about 150 or more nucleotides adjacent to: one or more signaling element comprising a 5′ SS, a 3′ SS, a premature stop codon, U1 binding sequence, U2 binding sequence, a BS, a PYT, ESE, an ISE, an ESS, an ISS, more than one of the foregoing, or any combination thereof.

Further description of editing or detecting a target nucleic acid in the foregoing genes may be found in more detail in Kim et al., “Enhancement of target specificity of CRISPR-Cas12a by using a chimeric DNA-RNA guide”, Nucleic Acids Res. 2020 Sep. 4; 48(15):8601-8616; Wang et al., “Specificity profiling of CRISPR system reveals greatly enhanced off-target gene editing”, Scientific Reports volume 10, Article number: 2269 (2020); Tuladhar et al., “CRISPR-Cas9-based mutagenesis frequently provokes on-target mRNA misregulation”, Nature Communications volume 10, Article number: 4056 (2019); Dong et al., “Genome-Wide Off-Target Analysis in CRISPR-Cas9 Modified Mice and Their Offspring”, G3, Volume 9, Issue 11, 1 Nov. 2019, Pages 3645-3651; Winter et al., “Genome-wide CRISPR screen reveals novel host factors required for Staphylococcus aureus α-hemolysin-mediated toxicity”, Scientific Reports volume 6, Article number: 24242 (2016); and Ma et al., “A CRISPR-Based Screen Identifies Genes Essential for West-Nile-Virus-Induced Cell Death”, Cell Rep. 2015 Jul. 28; 12(4):673-83, which are hereby incorporated by reference in their entirety.

In some embodiments, the target nucleic acid is in a cell described herein. In some embodiments, a cell described herein comprises a composition described herein, or a nucleic acid expression vector or library described herein. In some embodiments, a cell described herein comprises a target nucleic acid modified by a composition described herein, or a nucleic acid expression vector or library described herein. In some embodiments, a cell is a eukaryotic cell. In some embodiments, a cell is a mammalian cell. In some embodiments, a cell is a human cell. In some embodiments, the human cell is a: muscle cell, cardiac cell, visceral cell, cardiac muscle cell, smooth muscle cell, cardiomyocyte, nodal cardiac muscle cell, smooth muscle cell, visceral muscle cell, skeletal muscle cell, myocyte, red (or slow) skeletal muscle cell, white (fast) skeletal muscle cell, intermediate skeletal muscle, muscle satellite cell, muscle stem cell, myoblast, muscle progenitor cell, induced pluripotent stem cell (iPS), or a cell derived from an iPS cell, modified to have its gene edited and differentiated into myoblasts, muscle progenitor cells, muscle satellite cells, muscle stem cells, skeletal muscle cells, cardiac muscle cells or smooth muscle cells. In some embodiments, a population of cells comprises at least one cell, wherein the at least one cell is a cell described herein.

An RNP complex may comprise high selectivity for a target sequence. In some embodiments, an RNP complex may comprise a selectivity of at least 200:1, 100:1, 50:1, 20:1, 10:1, or 5:1 fora target nucleic acid over a single nucleotide variant of the target nucleic acid. In some embodiments, a ribonucleoprotein may comprise a selectivity of at least 5:1 for a target nucleic acid over a single nucleotide variant of the target nucleic acid. Leveraging effector protein selectivity, some methods described herein may detect a target nucleic acid present in the sample in various concentrations or amounts as a target nucleic acid population. In some embodiments, the sample has at least 2 target nucleic acids. In some embodiments, the sample has at least 3, 5, 10, 20, 30, 40, 50, 100, 200, 300, 400, 500, 600, 700, 800, 900, 1000, 2000, 3000, 4000, 5000, 6000, 7000, 8000, 9000, or 10000 target nucleic acids. In some embodiments, the sample comprises 1 to 10,000, 100 to 8000, 400 to 6000, 500 to 5000, 1000 to 4000, or 2000 to 3000 target nucleic acids. In some embodiments, the method detects target nucleic acid present at least at one copy per 10 non-target nucleic acids, 10² non-target nucleic acids, 10³ non-target nucleic acids, 10⁴ non-target nucleic acids, 10⁵ non-target nucleic acids, 10⁶ non-target nucleic acids, 10⁷ non-target nucleic acids, 10⁸ non-target nucleic acids, 10⁹ non-target nucleic acids, or 10¹⁰ non-target nucleic acids.

Often, the target nucleic acid may be from 0.05% to 20% of total nucleic acids in the sample. Sometimes, the target nucleic acid is 0.1% to 10% of the total nucleic acids in the sample. The target nucleic acid, in some embodiments, is 0.1% to 5% of the total nucleic acids in the sample. The target nucleic acid may also be 0.1% to 1% of the total nucleic acids in the sample. The target nucleic acid may be DNA or RNA. The target nucleic acid may be any amount less than 100% of the total nucleic acids in the sample. The target nucleic acid may be 100% of the total nucleic acids in the sample.

The target nucleic acid may be 0.05% to 20% of total nucleic acids in the sample. Sometimes, the target nucleic acid is 0.1% to 10% of the total nucleic acids in the sample. The target nucleic acid, in some embodiments, is 0.1% to 5% of the total nucleic acids in the sample. Often, a sample comprises the segment of the target nucleic acid and at least one nucleic acid comprising less than 100% sequence identity to the segment of the target nucleic acid but no less than 50% sequence identity to the segment of the target nucleic acid. For example, the segment of the target nucleic acid comprises a mutation as compared to at least one nucleic acid comprising less than 100% sequence identity to the segment of the target nucleic acid but no less than 50% sequence identity to the segment of the target nucleic acid. Often, the segment of the target nucleic acid comprises a single nucleotide mutation as compared to at least one nucleic acid comprising less than 100% sequence identity to the segment of the target nucleic acid but no less than 50% sequence identity to the segment of the target nucleic acid.

A target nucleic acid may be an amplified nucleic acid of interest. The nucleic acid of interest may be any nucleic acid disclosed herein or from any sample as disclosed herein. The nucleic acid of interest may be an RNA that is reverse transcribed before amplification. The nucleic acid of interest may be amplified then the amplicons may be transcribed into RNA.

In some embodiments, compositions described herein exhibit indiscriminate trans-cleavage of ssRNA, enabling their use for detection of RNA in samples. In some embodiments, target ssRNA are generated from many nucleic acid templates (RNA) in order to achieve cleavage of the FQ reporter in the DETECTR platform. Certain effector proteins may be activated by ssRNA, upon which they may exhibit trans-cleavage of ssRNA and may, thereby, be used to cleave ssRNA FQ reporter molecules in the DETECTR system. These effector proteins may target ssRNA present in the sample or ssRNA generated and/or amplified from any number of nucleic acid templates (RNA). Described herein are reagents comprising a single stranded reporter nucleic acid comprising a detection moiety, wherein the reporter nucleic acid (e.g., the ssDNA-FQ reporter described above) is capable of being cleaved by the Effector protein, upon generation and amplification of ssRNA from a nucleic acid template using the methods disclosed herein, thereby generating a first detectable signal.

In some embodiments, target nucleic acids comprise at least one nucleic acid comprising at least 50% sequence identity to the target nucleic acid or a portion thereof. Sometimes, the at least one nucleic acid comprises an amino acid sequence that is at least 60%, 70%, 75%, 80%, 85%, 90%, 91%, 92%, 93%, 94%, 95%, 96%, 97%, 98%, or 99% identical to an equal length portion of the target nucleic acid. Sometimes, the at least one nucleic acid comprises an amino acid sequence that is 100% identical to an equal length portion of the target nucleic acid. Sometimes, the amino acid sequence of the at least one nucleic acid is at least 60%, 70%, 75%, 80%, 85%, 90%, 91%, 92%, 93%, 94%, 95%, 96%, 97%, 98%, or 99% identical to the target nucleic acid. Sometimes, the target nucleic acid comprises an amino acid sequence that is less than 60%, 70%, 75%, 80%, 85%, 90%, 91%, 92%, 93%, 94%, 95%, 96%, 97%, 98%, or 99% identical to an equal length portion of the at least one nucleic acid.

In some embodiments, samples comprise a target nucleic acid at a concentration of less than 1 nM, less than 2 nM, less than 3 nM, less than 4 nM, less than 5 nM, less than 6 nM, less than 7 nM, less than 8 nM, less than 9 nM, less than 10 nM, less than 20 nM, less than 30 nM, less than 40 nM, less than 50 nM, less than 60 nM, less than 70 nM, less than 80 nM, less than 90 nM, less than 100 nM, less than 200 nM, less than 300 nM, less than 400 nM, less than 500 nM, less than 600 nM, less than 700 nM, less than 800 nM, less than 900 nM, less than 1 μM, less than 2 μM, less than 3 μM, less than 4 μM, less than 5 μM, less than 6 μM, less than 7 μM, less than 8 μM, less than 9 μM, less than 10 μM, less than 100 μM, or less than 1 mM. In some embodiments, the sample comprises a target nucleic acid at a concentration of 1 nM to 2 nM, 2 nM to 3 nM, 3 nM to 4 nM, 4 nM to 5 nM, 5 nM to 6 nM, 6 nM to 7 nM, 7 nM to 8 nM, 8 nM to 9 nM, 9 nM to 10 nM, 10 nM to 20 nM, 20 nM to 30 nM, 30 nM to 40 nM, 40 nM to 50 nM, 50 nM to 60 nM, 60 nM to 70 nM, 70 nM to 80 nM, 80 nM to 90 nM, 90 nM to 100 nM, 100 nM to 200 nM, 200 nM to 300 nM, 300 nM to 400 nM, 400 nM to 500 nM, 500 nM to 600 nM, 600 nM to 700 nM, 700 nM to 800 nM, 800 nM to 900 nM, 900 nM to 1 μM, 1 μM to 2 μM, 2 μM to 3 μM, 3 μM to 4 μM, 4 μM to 5 μM, 5 μM to 6 μM, 6 μM to 7 μM, 7 μM to 8 μM, 8 μM to 9 μM, 9 μM to 10 μM, 10 μM to 100 μM, 100 μM to 1 mM, 1 nM to 10 nM, 1 nM to 100 nM, 1 nM to 1 μM, 1 nM to 10 μM, 1 nM to 100 μM, 1 nM to 1 mM, 10 nM to 100 nM, 10 nM to 1 μM, 10 nM to 10 μM, 10 nM to 100 μM, 10 nM to 1 mM, 100 nM to 1 μM, 100 nM to 10 μM, 100 nM to 100 μM, 100 nM to 1 mM, 1 μM to 10 μM, 1 μM to 100 μM, 1 μM to 1 mM, 10 μM to 100 μM, 10 μM to 1 mM, or 100 μM to 1 mM. In some embodiments, the sample comprises a target nucleic acid at a concentration of 20 nM to 200 μM, 50 nM to 100 μM, 200 nM to 50 μM, 500 nM to 20 μM, or 2 μM to 10 μM. In some embodiments, the target nucleic acid is not present in the sample.

In some embodiments, samples comprise fewer than 10 copies, fewer than 100 copies, fewer than 1000 copies, fewer than 10,000 copies, fewer than 100,000 copies, or fewer than 1,000,000 copies of a target nucleic acid. In some embodiments, the sample comprises 10 copies to 100 copies, 100 copies to 1000 copies, 1000 copies to 10,000 copies, 10,000 copies to 100,000 copies, 100,000 copies to 1,000,000 copies, 10 copies to 1000 copies, 10 copies to 10,000 copies, 10 copies to 100,000 copies, 10 copies to 1,000,000 copies, 100 copies to 10,000 copies, 100 copies to 100,000 copies, 100 copies to 1,000,000 copies, 1,000 copies to 100,000 copies, or 1,000 copies to 1,000,000 copies of a target nucleic acid. In some embodiments, the sample comprises 10 copies to 500,000 copies, 200 copies to 200,000 copies, 500 copies to 100,000 copies, 1000 copies to 50,000 copies, 2000 copies to 20,000 copies, 3000 copies to 10,000 copies, or 4000 copies to 8000 copies. In some embodiments, the target nucleic acid is not present in the sample.

A number of target nucleic acid populations are consistent with the methods and compositions disclosed herein. Some methods described herein may detect two or more target nucleic acid populations present in the sample in various concentrations or amounts. In some embodiments, the sample has at least 2 target nucleic acid populations. In some embodiments, the sample has at least 3, 4, 5, 6, 7, 8, 9, 10, 20, 30, 40, or 50 target nucleic acid populations. In some embodiments, the sample has 3 to 50, 5 to 40, or 10 to 25 target nucleic acid populations. In some embodiments, the method detects target nucleic acid populations that are present at least at one copy per 10¹ non-target nucleic acids, 10² non-target nucleic acids, 10³ non-target nucleic acids, 10⁴ non-target nucleic acids, 10⁵ non-target nucleic acids, 10⁶ non-target nucleic acids, 10⁷ non-target nucleic acids, 10⁸ non-target nucleic acids, 10⁹ non-target nucleic acids, or 10¹⁰ non-target nucleic acids. The target nucleic acid populations may be present at different concentrations or amounts in the sample.

In some embodiments, target nucleic acids may activate an effector protein to initiate sequence-independent cleavage of a nucleic acid-based reporter (e.g., a reporter comprising an RNA sequence, or a reporter comprising DNA and RNA). For example, an effector protein of the present disclosure is activated by a target nucleic acid to cleave reporters having an RNA (also referred to herein as an “RNA reporter”). Alternatively, an effector protein of the present disclosure is activated by a target nucleic acid to cleave reporters having an RNA. Alternatively, an effector protein of the present disclosure is activated by a target RNA to cleave reporters having an RNA (also referred to herein as a “RNA reporter”). The RNA reporter may comprise a single-stranded RNA labelled with a detection moiety or may be any RNA reporter as disclosed herein.

In some embodiments, the target nucleic acid as described in the methods herein does not initially comprise a PAM sequence. However, any target nucleic acid of interest may be generated using the methods described herein to comprise a PAM sequence, and thus be a PAM target nucleic acid. A PAM target nucleic acid, as used herein, refers to a target nucleic acid that has been amplified to insert a PAM sequence that is recognized by an effector protein system.

In some embodiments, the target nucleic acid is in a cell. In some embodiments, the cell is a single-cell eukaryotic organism; a plant cell an algal cell; a fungal cell; an animal cell; a cell an invertebrate animal; a cell a vertebrate animal such as fish, amphibian, reptile, bird, and mammal; or a cell a mammal such as a human, a non-human primate, an ungulate, a feline, a bovine, an ovine, and a caprine. In preferred embodiments, the cell is a eukaryotic cell. In preferred embodiments, the cell is a mammalian cell, a human cell, or a plant cell.

In some embodiments, the target nucleic acid comprises a nucleic acid sequence from a pathogen responsible for a disease. Non-limiting examples of pathogens are bacteria, a virus and a fungus. The target nucleic acid, in some embodiments, is a portion of a nucleic acid from a sexually transmitted infection or a contagious disease. In some embodiments, the target nucleic acid is a portion of a nucleic acid from a genomic locus, or any DNA amplicon, such as a reverse transcribed mRNA or a cDNA from a gene locus, a transcribed mRNA, or a reverse transcribed cDNA from a gene locus in at least one of: human immunodeficiency virus (HIV), human papillomavirus (HPV), chlamydia, gonorrhea, syphilis, trichomoniasis, sexually transmitted infection, malaria, Dengue fever, Ebola, chikungunya, and leishmaniasis. Pathogens include viruses, fungi, helminths, protozoa, malarial parasites, Plasmodium parasites, Toxoplasma parasites, and Schistosoma parasites. Helminths include roundworms, heartworms, and phytophagous nematodes, flukes, Acanthocephala, and tapeworms. Protozoan infections include infections from Giardia spp., Trichomonas spp., African trypanosomiasis, amoebic dysentery, babesiosis, balantidial dysentery, Chaga's disease, coccidiosis, malaria and toxoplasmosis. Examples of pathogens such as parasitic/protozoan pathogens include, but are not limited to: Plasmodium falciparum, P. vivax, Trypanosoma cruzi and Toxoplasma gondii. Fungal pathogens include, but are not limited to Cryptococcus neoformans, Histoplasma capsulatum, Coccidioides immitis, Blastomyces dermatitides, Chlamydia trachomatis, and Candida albicans. Pathogenic viruses include but are not limited to coronavirus (e.g., SARS-CoV-2); immunodeficiency virus (e.g., HIV); influenza virus; dengue; West Nile virus; herpes virus; yellow fever virus; Hepatitis Virus C; Hepatitis Virus A; Hepatitis Virus B; papillomavirus; and the like. Pathogens include, e.g., HIV virus, Mycobacterium tuberculosis, Streptococcus agalactiae, methicillin-resistant Staphylococcus aureus, Legionella pneumophila, Streptococcus pyogenes, Escherichia coli, Neisseria gonorrhoeae, Neisseria meningitidis, Pneumococcus, Cryptococcus neoformans, Histoplasma capsulatum, Hemophilus influenzae B, Treponema pallidum, Lyme disease spirochetes, Pseudomonas aeruginosa, Mycobacterium leprae, Brucella abortus, rabies virus, influenza virus, cytomegalovirus, herpes simplex virus I, herpes simplex virus II, human serum parvo-like virus, respiratory syncytial virus (RSV), M. genitalium, T. vaginalis, varicella-zoster virus, hepatitis B virus, hepatitis C virus, measles virus, adenovirus, human T-cell leukemia viruses, Epstein-Barr virus, murine leukemia virus, mumps virus, vesicular stomatitis virus, Sindbis virus, lymphocytic choriomeningitis virus, wart virus, blue tongue virus, Sendai virus, feline leukemia virus, Reovirus, polio virus, simian virus 40, mouse mammary tumor virus, dengue virus, rubella virus, West Nile virus, Plasmodium falciparum, Plasmodium vivax, Toxoplasma gondii, Trypanosoma rangeli, Trypanosoma cruzi, Trypanosoma rhodesiense, Trypanosoma brucei, Schistosoma mansoni, Schistosoma japonicum, Babesia bovis, Eimeria tenella, Onchocerca volvulus, Leishmania tropica, Mycobacterium tuberculosis, Trichinella spiralis, Theileria parva, Taenia hydatigena, Taenia ovis, Taenia saginata, Echinococcus granulosus, Mesocestoides corti, Mycoplasma arthritidis, M. hyorhinis, M. orale, M. arginini, Acholeplasma laidlawii, M. salivarium and M. pneumoniae. In some embodiments, the target sequence is a portion of a nucleic acid from a genomic locus, a transcribed mRNA, or a reverse transcribed cDNA from a gene locus of bacterium or other agents responsible for a disease in the sample comprising a mutation that confers resistance to a treatment, such as a single nucleotide mutation that confers resistance to antibiotic treatment.

In some embodiments, the target nucleic acid comprises a nucleic acid sequence of a virus, a bacterium, or other pathogen responsible for a disease in a plant (e.g., a crop). Methods and compositions of the disclosure may be used to treat or detect a disease in a plant. For example, the methods of the disclosure may be used to target a viral nucleic acid sequence in a plant. An effector protein of the disclosure may cleave the viral nucleic acid. In some embodiments, the target nucleic acid comprises a nucleic acid sequence of a virus or a bacterium or other agents (e.g., any pathogen) responsible for a disease in the plant (e.g., a crop). In some embodiments, the target nucleic acid comprises RNA. The target nucleic acid, in some embodiments, is a portion of a nucleic acid from a virus or a bacterium or other agents responsible for a disease in the plant (e.g., a crop). In some embodiments, the target nucleic acid is a portion of a nucleic acid from a genomic locus, or any NA amplicon, such as a reverse transcribed mRNA or a cDNA from a gene locus, a transcribed mRNA, or a reverse transcribed cDNA from a gene locus in at a virus or a bacterium or other agents (e.g., any pathogen) responsible for a disease in the plant (e.g., a crop). A virus infecting the plant may be an RNA virus. A virus infecting the plant may be a DNA virus. Non-limiting examples of viruses that may be targeted with the disclosure include Tobacco mosaic virus (TMV), Tomato spotted wilt virus (TSWV), Cucumber mosaic virus (CMV), Potato virus Y (PVY), Cauliflower mosaic virus (CaMV) (RT virus), Plum pox virus (PPV), Brome mosaic virus (BMV) and Potato virus X (PVX).

In some embodiments, compositions, systems, and methods described herein comprise a modified target nucleic acid. In some embodiments, compositions, systems, and methods described herein comprise a modified target nucleic acid which may describe a target nucleic acid wherein the target nucleic acid has undergone a modification, for example, after contact with an effector protein. In some embodiments, the modified target nucleic acid comprises a modification. In some embodiments, the modification is an alteration in the sequence of the target nucleic acid. In some embodiments, the modified target nucleic acid comprises an insertion, deletion, or replacement of one or more nucleotides compared to the unmodified target nucleic acid. In some embodiments, the modification is a mutation. In some embodiments, the modification of the target nucleic acid comprises insertion of a donor nucleic acid, deletion of a target nucleic acid, insertion of a donor nucleic acid fragment, deletion of a target nucleic acid fragment, or combinations thereof. In some embodiments, the modification of the target nucleic acid comprises insertion of a donor nucleic acid or donor nucleic acid fragment into the target nucleic acid. In some embodiments, the modification of the target nucleic acid comprises deletion of a target nucleic acid or a target nucleic acid fragment from the target nucleic acid.

Mutations

In some embodiments, target nucleic acids comprise a mutation. In some embodiments, a composition, system or method described herein may be used to modify a target nucleic acid comprising a mutation such that the mutation is modified to be a wild-type nucleotide or nucleotide sequence. In some embodiments, a composition, system or method described herein may be used to detect a target nucleic acid comprising a mutation. In some embodiments, a sequence comprising a mutation may be modified to a wildtype sequence with a composition, system or method described herein. In some embodiments, a sequence comprising a mutation may be detected with a composition, system or method described herein. The mutation may be a mutation of 1, 2, 3, 4, 5, 6, 7, 8, 9, 10, 11, 12, 13, 14, 15, 16, 17, 18, 19, 20 or more nucleotides. The mutation may comprise a deletion of about 5, about 10, about 15, about 20, about 25, about 30, about 35, about 40, about 45, about 50, about 55, about 60, about 65, about 70, about 75, about 80, about 85, about 90, about 95, about 100, about 200, about 300, about 400, about 500, about 600, about 700, about 800, about 900, or about 1000 nucleotides. The mutation may comprise a deletion of 1 to 5, 5 to 10, 10 to 15, 15 to 20, 20 to 25, 25 to 30, 30 to 35, 35 to 40, 40 to 45, 45 to 50, 50 to 55, 55 to 60, 60 to 65, 65 to 70, 70 to 75, 75 to 80, 80 to 85, 85 to 90, 90 to 95, 95 to 100, 100 to 200, 200 to 300, 300 to 400, 400 to 500, 500 to 600, 600 to 700, 700 to 800, 800 to 900, 900 to 1000, 1 to 50, 1 to 100, 25 to 50, 25 to 100, 50 to 100, 100 to 500, 100 to 1000, or 500 to 1000 nucleotides. Non-limiting examples of mutations are insertion-deletion (indel), single nucleotide polymorphism (SNP), and frameshift mutations. In some embodiments, guide nucleic acids described herein hybridize to a region of the target nucleic acid comprising the mutation. The mutation may be located in a non-coding region or a coding region of a gene.

A mutation may be in an open reading frame of a target nucleic acid. A mutation may result in the insertion of at least one amino acid in a protein encoded by the target nucleic acid. A mutation may result in the deletion of at least one amino acid in a protein encoded by the target nucleic acid. A mutation may result in the substitution of at least one amino acid in a protein encoded by the target nucleic acid. A mutation that results in the deletion, insertion, or substitution of one or more amino acids of a protein encoded by the target nucleic acid may result in misfolding of a protein encoded by the target nucleic acid. A mutation may result in a premature stop codon, thereby resulting in a truncation of the encoded protein.

In some embodiments, a mutation comprises a point mutation or single nucleotide polymorphism (SNP), a chromosomal mutation, a copy number mutation, or any combination thereof. A point mutation optionally comprises a substitution, insertion, or deletion. In some embodiments, a mutation comprises a chromosomal mutation. A chromosomal mutations may comprise an inversion, a deletion, a duplication, or a translocation of one or more nucleotides. In some embodiments, a mutation comprises a copy number variation. A copy number variation may comprise a gene amplification or an expanding trinucleotide repeat. In some embodiments, guide nucleic acids described herein hybridize to a target sequence of a target nucleic acid comprising the mutation. In some embodiments, mutations are located in a non-coding region of a gene.

In some embodiments, target nucleic acids comprise a mutation, wherein the mutation is a SNP. The single nucleotide mutation or SNP may be associated with a phenotype of the sample or a phenotype of the organism from which the sample was taken. The SNP, in some embodiments, is associated with altered phenotype from wild type phenotype. In some embodiments, a single nucleotide mutation, SNP, or deletion described herein is associated with a disease, such as a genetic disease. The SNP may be a synonymous substitution or a nonsynonymous substitution. The nonsynonymous substitution may be a missense substitution or a nonsense point mutation. The synonymous substitution may be a silent substitution. The mutation may be a deletion of one or more nucleotides. Often, the single nucleotide mutation, SNP, or deletion is associated with a disease such as cancer or a genetic disorder. The mutation, such as a single nucleotide mutation, a SNP, or a deletion, may be encoded in the sequence of a target nucleic acid from the germline of an organism or may be encoded in a target nucleic acid from a diseased cell, such as a cancer cell.

In some embodiments, the target nucleic acid comprises a mutation associated with a disease. In some examples, a mutation associated with a disease refers to a mutation whose presence in a subject indicates that the subject is susceptible to or suffers from, a disease, disorder, condition, or syndrome. In some examples, a mutation associated with a disease refers to a mutation which causes, contributes to the development of, or indicates the existence of the disease, disorder, condition, or syndrome. A mutation associated with a disease may also refer to any mutation which generates transcription or translation products at an abnormal level, or in an abnormal form, in cells affected by a disease relative to a control without the disease. In some examples, a mutation associated with a disease refers to a mutation whose presence in a subject indicates that the subject is susceptible to, or suffers from, a disease, disorder, or pathological state. In some embodiments, a mutation associated with a disease, comprises the co-occurrence of a mutation and the phenotype of a disease. The mutation may occur in a gene, wherein transcription or translation products from the gene occur at a significantly abnormal level or in an abnormal form in a cell or subject harboring the mutation as compared to a non-disease control subject not having the mutation.

In some embodiments, target nucleic acids comprise a mutation, wherein the mutation is a deletion of 1, 2, 3, 4, 5, 6, 7, 8, 9, 10, 11, 12, 13, 14, 15, 16, 17, 18, 19, 20 or more nucleotides. In some embodiments, a target nucleic acid comprises a mutation of 1, 2, 3, 4, 5, 6, 7, 8, 9, 10, 11, 12, 13, 14, 15, 16, 17, 18, 19, 20 or more nucleotides. The mutation may be a deletion of about 5, about 10, about 15, about 20, about 25, about 30, about 35, about 40, about 45, about 50, about 55, about 60, about 65, about 70, about 75, about 80, about 85, about 90, about 95, about 100, about 200, about 300, about 400, about 500, about 600, about 700, about 800, about 900, or about 1000 nucleotides. The mutation may be a deletion of 1 to 5, 5 to 10, 10 to 15, 15 to 20, 20 to 25, 25 to 30, 30 to 35, 35 to 40, 40 to 45, 45 to 50, 50 to 55, 55 to 60, 60 to 65, 65 to 70, 70 to 75, 75 to 80, 80 to 85, 85 to 90, 90 to 95, 95 to 100, 100 to 200, 200 to 300, 300 to 400, 400 to 500, 500 to 600, 600 to 700, 700 to 800, 800 to 900, 900 to 1000, 1 to 50, 1 to 100, 25 to 50, 25 to 100, 50 to 100, 100 to 500, 100 to 1000, or 500 to 1000 nucleotides.

Certain Samples

Various sample types comprising a target nucleic acid of interest are consistent with the present disclosure. These samples may comprise a target nucleic acid for detection. In some embodiments, the detection of the target nucleic indicates an ailment, such as a disease, cancer, or genetic disorder, or genetic information, such as for phenotyping, genotyping, or determining ancestry and are compatible with the reagents and support mediums as described herein. Generally, a sample from an individual or an animal or an environmental sample may be obtained to test for presence of a disease, cancer, genetic disorder, or any mutation of interest.

In some embodiments, the sample is a biological sample, an environmental sample, or a combination thereof. Non-limiting examples of biological samples are blood, serum, plasma, saliva, urine, mucosal sample, peritoneal sample, cerebrospinal fluid, gastric secretions, nasal secretions, sputum, pharyngeal exudates, urethral or vaginal secretions, an exudate, an effusion, and a tissue sample (e.g., a biopsy sample). A tissue sample from a subject may be dissociated or liquified prior to application to detection system of the present disclosure. Non-limiting examples of environmental samples are soil, air, or water. In some embodiments, an environmental sample is taken as a swab from a surface of interest or taken directly from the surface of interest.

In some embodiments, the sample is a raw (unprocessed, unmodified) sample. Raw samples may be applied to a system for detecting or modifying a target nucleic acid, such as those described herein. In some embodiments, the sample is diluted with a buffer or a fluid or concentrated prior to its application to the system or be applied neat to the detection system. Sometimes, the sample contains no more 20 μl of buffer or fluid. The sample, in some embodiments, is contained in no more than 1, 5, 10, 15, 20, 25, 30, 35 40, 45, 50, 55, 60, 65, 70, 75, 80, 90, 100, 200, 300, 400, 500 μl, or any of value 1 μl to 500 μl, preferably 10 μL to 200 μL, or more preferably 50 μL to 100 μL of buffer or fluid. Sometimes, the sample is contained in more than 500 μl.

In some embodiments, the sample is taken from a single-cell eukaryotic organism; a plant or a plant cell; an algal cell; a fungal cell; an animal cell, tissue, or organ; a cell, tissue, or organ from an invertebrate animal; a cell, tissue, fluid, or organ from a vertebrate animal such as fish, amphibian, reptile, bird, and mammal; a cell, tissue, fluid, or organ from a mammal such as a human, a non-human primate, an ungulate, a feline, a bovine, an ovine, and a caprine. In some embodiments, the sample is taken from nematodes, protozoans, helminths, or malarial parasites. In some embodiments, the sample comprises nucleic acids from a cell lysate from a eukaryotic cell, a mammalian cell, a human cell, a prokaryotic cell, or a plant cell. In some embodiments, the sample comprises nucleic acids expressed from a cell.

In some embodiments, samples are used for diagnosing a disease. In some embodiments the disease is cancer. The sample used for cancer testing may comprise at least one target nucleic acid that may bind to a guide nucleic acid of the reagents described herein. The target nucleic acid, in some embodiments, comprises a portion of a gene comprising a mutation associated with a disease, such as cancer, a gene whose overexpression is associated with cancer, a tumor suppressor gene, an oncogene, a checkpoint inhibitor gene, a gene associated with cellular growth, a gene associated with cellular metabolism, or a gene associated with cell cycle. Sometimes, the target nucleic acid encodes a cancer biomarker, such as a prostate cancer biomarker or non-small cell lung cancer. In some embodiments, the assay may be used to detect “hotspots” in target nucleic acids that may be predictive of lung cancer. In some embodiments, the target nucleic acid comprises a portion of a nucleic acid that is associated with a blood fever. In some embodiments, the target nucleic acid is a portion of a nucleic acid from a genomic locus, any DNA amplicon of, a reverse transcribed mRNA, or a cDNA from a locus of at least one of: ALK, APC, ATM, AXIN2, BAP1, BARD1, BLM, BMPR1A, BRCA1, BRCA2, BRIP1, CASR, CDC73, CDH1, CDK4, CDKN1B, CDKN1C, CDKN2A, CEBPA, CHEK2, CTNNA1, DICER1, DIS3L2, EGFR, EPCAM, FH, FLCN, GATA2, GPC3, GREM1, HOXB13, HRAS, MAX, MEN1, MET, MITF, MLH1, MSH2, MSH3, MSH6, MUTYH, NBN, NF1, NF2, NTHL1, PALB2, PDGFRA, PHOX2B, PMS2, POLD1, POLE, POT1, PRKAR1A, PTCH1, PTEN, RAD50, RAD51C, RAD51D, RB1, RECQL4, RET, RUNX1, SDHA, SDHAF2, SDHB, SDHC, SDHD, SMAD4, SMARCA4, SMARCB1, SMARCE1, STK11, SUFU, TERC, TERT, TMEM127, TP53, TSC1, TSC2, VHL, WRN, and WT1. Any region of the aforementioned gene loci may be probed for a mutation or deletion using the compositions and methods disclosed herein. For example, in the EGFR gene locus, the compositions and methods for detection disclosed herein may be used to detect a single nucleotide polymorphism or a deletion.

In some embodiments, samples are used to diagnose a genetic disorder, also referred to as genetic disorder testing. The sample used for genetic disorder testing may comprise at least one target nucleic acid that may bind to a guide nucleic acid of the reagents described herein. In some embodiments, the genetic disorder is hemophilia, sickle cell anemia, 0-thalassemia, Duchene muscular dystrophy, severe combined immunodeficiency, Huntington's disease, or cystic fibrosis. The target nucleic acid, in some embodiments, is from a gene with a mutation associated with a genetic disorder, from a gene whose overexpression is associated with a genetic disorder, from a gene associated with abnormal cellular growth resulting in a genetic disorder, or from a gene associated with abnormal cellular metabolism resulting in a genetic disorder. In some embodiments, the target nucleic acid is a nucleic acid from a genomic locus, a transcribed mRNA, or a reverse transcribed mRNA, a DNA amplicon of or a cDNA from a locus of at least one of: CFTR, FMR1, SMN1, ABCB11, ABCC8, ABCD1, ACAD9, ACADM, ACADVL, ACAT1, ACOX1, ACSF3, ADA, ADAMTS2, ADGRG1, AGA, AGL, AGPS, AGXT, AIRE, ALDH3A2, ALDOB, ALG6, ALMS1, ALPL, ANGPTL3, AMT, Apo(a), ApoC111, APOEE4, APP, AQP2, ARG1, ARSA, ARSB, ASL, ASNS, ASPA, ASS1, ATM, ATP6V1B1, ATP7A, ATP7B, ATRX, ATXN2, BACE-1, BBS1, BBS10, BBS12, BBS2, BCKDHA, BCKDHB, BCS1L, BLM, BSND, C9ORF72, CAH1, CAPN3, CBS, CDH23, CEP290, CERKL, CHCHD10, CHM, CHRNE, CIITA, CLN3, CLN5, CLN6, CLN8, CLRN1, CMT1A, CNGB3, COL27A1, COL4A3, COL4A4, COL4A5, COL7A1, CPS1, CPT1A, CPT2, CRB1, CTNS, CTSK, CYBA, CYBB, CYP11B1, CYP11B2, CYP17A1, CYP19A1, CYP27A1, DBT, DCLRE1C, DHCR7, DHDDS, DLD, DMD, DNAH5, DNAI1, DNAI2, DYSF, EDA, EIF2B5, EMD, ERCC6, ERCC8, ESCO2, ETFA, ETFDH, ETHE1, EVC, EVC2, EYS, F9, FAH, FAM161A, FANCA, FANCC, FANCG, FH, FKRP, FKTN, FSHD1, FUS, FVIII, FXI, G6PC, GAA, GALC, GALK1, GALT, GAMT, GBA, GBE1, GCDH, GFM1, GJB1, GJB2, GLA, GLB1, GLDC, GLE1, GNE, GNPTAB, GNPTG, GNS, GRHPR, HADHA, HAX1, HBA1, HBA2, HBB, HEXA, HEXB, HGSNAT, HLCS, HMGCL, HOGA1, HPS1, HPS3, HSD17B4, HSD3B2, HTT, HYAL1, HYLS1, IDS, IDUA, IKBKAP, IL2RG, IVD, KCNJ11, LAMA2, LAMA3, LAMB3, LAMC2, LCA5, LDLR, LDLRAP1, LHX3, LIFR, LIPA, LOXHD1, LPL, LRPPRC, MAN2B1, MAPT, MCOLN1, MED17, MESP2, MFSD8, MKS1, MLC1, MMAA, MMAB, MMACHC, MMADHC, MPI, MPL, MPV17, MTHFR, MTM1, MTRR, MTTP, MUT, MYO7A, NAGLU, NAGS, NBN, NDRG1, NDUFAF5, NDUFS6, NEB, NPC1, NPC2, NPHS1, NPHS2, NR2E3, NTRK1, OAT, OPA3, OTC, PAH, PC, PCCA, PCCB, PCDH15, PCSK9, PDHA1, PDHB, PEX1, PEX10, PEX12, PEX2, PEX6, PEX7, PFKM, PHGDH, PKHD1, PMP22, PMM2, POMGNT1, PPT1, PROP1, PRPS1, PSEN1, PSEN2, PSAP, PSD95, PTS, PUS1, PYGM, RAB23, RAG2, RAPSN, RARS2, RDH12, RMRP, RPE65, RPGRIP1L, RS1, RTEL1, SACS, SAMHD1, SEPSECS, SGCA, SGCB, SGCG, SGSH, SLC12A3, SLC12A6, SLC17A5, SLC22A5, SLC25A13, SLC25A15, SLC26A2, SLC26A4, SLC35A3, SLC37A4, SLC39A4, SLC4A11, SLC6A8, SLC7A7, SMARCAL1, SMPD1, SOD1, SERPINC1, SERPING1, STAR, SUMF1, TARDBP, TAT, TCIRG1, TECPR2, TFR2, TGM1, TH, TMEM216, TPP1, TRMU, TSFM, TTR, TTPA, TYMP, USH1C, USH2A, VPS13A, VPS13B, VPS45, VRK1, VSX2, WNT10A, XPA, XPC, and ZFYVE26.

The sample used for phenotyping testing may comprise at least one target nucleic acid that may bind to a guide nucleic acid of the reagents described herein. The target nucleic acid, in some embodiments, is a nucleic acid encoding a sequence associated with a phenotypic trait.

The sample used for genotyping testing may comprise at least one target nucleic acid that may bind to a guide nucleic acid of the reagents described herein. The target nucleic acid, in some embodiments, is a nucleic acid encoding a sequence associated with a genotype of interest.

The sample used for ancestral testing may comprise at least one target nucleic acid that may bind to a guide nucleic acid of the reagents described herein. The target nucleic acid, in some embodiments, is a nucleic acid encoding a sequence associated with a geographic region of origin or ethnic group.

The sample may be used for identifying a disease status. For example, a sample is any sample described herein, and is obtained from a subject for use in identifying a disease status of a subject. The disease may be a cancer or genetic disorder. Sometimes, a method comprises obtaining a serum sample from a subject; and identifying a disease status of the subject. Often, the disease status is prostate disease status, but the status of any disease may be assessed.

Any of the above disclosed samples are consistent with the methods, compositions, reagents, enzymes, and systems disclosed herein.

### IX. SYSTEMS

Disclosed herein are systems for detecting and/or modifying target nucleic acid. In some embodiments, systems comprise components comprising one or more: effector protein described herein; guide nucleic acid described herein; target nucleic acid described herein; donor nucleic acid described herein; a solution or buffer; a reagent; a support medium; other components or appurtenances as described herein; or combinations thereof. In some embodiments, a system comprising one or more components (e.g., effector proteins, effector partners, guide nucleic acids, donor nucleic acids, target nucleic acids, etc., as described herein), wherein one or more compositions comprise the one or more components. In some embodiments, a composition comprises one system component as described herein. In some embodiments, a composition comprising a system component is individually administered. In some embodiments, one or more components are individually administered. In some embodiments, each of the one or more components individually administered can interact with other components following independent administration. In some embodiments, each component of a composition or system described herein are each independently administered in a single composition. In some embodiments, independent administration comprises contact with a target nucleic acid, a target cell or host cell, or administration as a method of nucleic acid detection, editing, and/or treatment as described herein.

In some embodiments, one or more components are administered in multiple compositions, wherein the one or more components can interact with each other following administration. In some embodiments, the interaction between the one or more components occurs within a target cell or a host cell.

In some embodiments, one or more components of a composition or system described herein are co-administered. In some embodiments, an effector protein (or a nucleic acid encoding same) and/or an engineered guide nucleic acid (or a nucleic acid that, when transcribed, produces same) are co-administered with a donor nucleic acid. Co-administration may be contact with a target nucleic acid, administered to a cell, such as a host cell, or administered as method of nucleic acid detection, editing, and/or treatment as described herein, in a single composition. In some embodiments, an effector protein (or a nucleic acid encoding same) and/or an engineered guide nucleic acid (or a nucleic acid that, when transcribed, produces same) are not co-administered with donor nucleic acid in a single composition. In some embodiments, an effector protein (or a nucleic acid encoding same), an engineered guide nucleic acid (or a nucleic acid that, when transcribed, produces same), and/or donor nucleic acid are administered in one or more compositions.

Systems may be used to modify the activity or expression of a target nucleic acid. In some embodiments, systems comprise an effector protein described herein, a reagent, support medium, or a combination thereof.

In some embodiments, systems comprise an effector protein described herein, a guide nucleic acid described herein, a reagent, support medium, or a combination thereof. In some embodiments, the effector protein comprises an effector protein, or a fusion protein thereof, described herein. In some embodiments, effector protein comprises an amino acid sequence that is at least 65%, at least 70%, at least 75%, at least 80%, at least 85%, at least 90%, at least 95%, or 100% identical to any one of the sequences of TABLE 1.

Systems may be used for detecting the presence or the absence of a target nucleic acid as described herein. Systems may be used for detecting the presence or the absence of a target nucleic acid associated with or causative of a disease or disorder, such as a genetic disorder. Systems may be used for detecting the presence or the absence of a target nucleic acid associated with or causative of a disease or disorder as described herein. In some embodiments, systems are useful for phenotyping, genotyping, or determining ancestry. Unless specified otherwise, systems include kits and may be referred to as kits. Unless specified otherwise, systems include devices and may also be referred to as devices. Systems described herein may be provided in the form of a companion diagnostic assay or device, a point-of-care assay or device, or an over-the-counter diagnostic assay/device.

Reagents and effector proteins and/or effector partners of various systems may be provided in a reagent chamber or on a support medium. Alternatively, the reagent and/or effector protein and/or effector partners may be contacted with the reagent chamber or the support medium by the individual using the system. An exemplary reagent chamber is a test well or container. The opening of the reagent chamber may be large enough to accommodate the support medium. Optionally, the system comprises a buffer and a dropper. The buffer may be provided in a dropper bottle for ease of dispensing. The dropper may be disposable and transfer a fixed volume. The dropper may be used to place a sample into the reagent chamber or on the support medium.

System Solutions

In general, system components comprise a solution in which the activity of an effector protein occurs. Often, the solution comprises or consists essentially of a buffer. The solution or buffer may comprise a buffering agent, a salt, a crowding agent, a detergent, a reducing agent, a competitor, or a combination thereof. Often the buffer is the primary component or the basis for the solution in which the activity occurs. Thus, concentrations for components of buffers described herein (e.g., buffering agents, salts, crowding agents, detergents, reducing agents, and competitors) are the same or essentially the same as the concentration of these components in the solution in which the activity occurs. In some embodiments, a buffer is required for cell lysis activity or viral lysis activity.

In some embodiments, systems comprise a buffer, wherein the buffer comprise at least one buffering agent. Exemplary buffering agents include HEPES, TRIS, MES, ADA, PIPES, ACES, MOPSO, BIS-TRIS propane, BES, MOPS, TES, DISO, Trizma, TRICINE, GLY-GLY, HEPPS, BICINE, TAPS, A MPD, A MPSO, CHES, CAPSO, AMP, CAPS, phosphate, citrate, acetate, imidazole, or any combination thereof. In some embodiments, the concentration of the buffering agent in the buffer is 1 mM to 200 mM. A buffer compatible with an effector protein may comprise a buffering agent at a concentration of 10 mM to 30 mM. A buffer compatible with an effector protein may comprise a buffering agent at a concentration of about 20 mM. A buffering agent may provide a pH for the buffer or the solution in which the activity of the effector protein occurs. The pH may be 3 to 4, 3.5 to 4.5, 4 to 5, 4.5 to 5.5, 5 to 6, 5.5 to 6.5, 6 to 7, 6.5 to 7.5, 7 to 8, 7.5 to 8.5, 8 to 9, 8.5 to 9.5, 9 to 10, or 9.5 to 10.5.

In some embodiments, systems comprise a solution, wherein the solution comprises at least one salt. In some embodiments, the at least one salt is selected from potassium acetate, magnesium acetate, sodium chloride, potassium chloride, magnesium chloride, calcium chloride, and any combination thereof. In some embodiments, the concentration of the at least one salt in the solution is 5 mM to 100 mM, 5 mM to 10 mM, 1 mM to 60 mM, or 1 mM to 10 mM. In some embodiments, the concentration of the at least one salt is about 105 mM. In some embodiments, the concentration of the at least one salt is about 55 mM. In some embodiments, the concentration of the at least one salt is about 7 mM. In some embodiments, the solution comprises potassium acetate and magnesium acetate. In some embodiments, the solution comprises sodium chloride and magnesium chloride. In some embodiments, the solution comprises potassium chloride and magnesium chloride. In some embodiments, the salt is a magnesium salt and the concentration of magnesium in the solution is at least 5 mM, 7 mM, at least 9 mM, at least 11 mM, at least 13 mM, or at least 15 mM. In some embodiments, the concentration of magnesium is less than 20 mM, less than 18 mM, or less than 16 mM.

In some embodiments, systems comprise a solution, wherein the solution comprises at least one crowding agent. A crowding agent may reduce the volume of solvent available for other molecules in the solution, thereby increasing the effective concentrations of said molecules. Exemplary crowding agents include glycerol and bovine serum albumin. In some embodiments, the crowding agent is glycerol. In some embodiments, the concentration of the crowding agent in the solution is 0.01% (v/v) to 10% (v/v). In some embodiments, the concentration of the crowding agent in the solution is 0.5% (v/v) to 10% (v/v).

In some embodiments, systems comprise a solution, wherein the solution comprises at least one detergent. Exemplary detergents include Tween, Triton-X, and IGEPAL. A solution may comprise Tween, Triton-X, or any combination thereof. A solution may comprise Triton-X. A solution may comprise IGEPAL CA-630. In some embodiments, the concentration of the detergent in the solution is 2% (v/v) or less. In some embodiments, the concentration of the detergent in the solution is 1% (v/v) or less. In some embodiments, the concentration of the detergent in the solution is 0.00001% (v/v) to 0.01% (v/v). In some embodiments, the concentration of the detergent in the solution is about 0.01% (v/v).

In some embodiments, systems comprise a solution, wherein the solution comprises at least one reducing agent. Exemplary reducing agents comprise dithiothreitol (DTT), ß-mercaptoethanol (BME), or tris(2-carboxyethyl) phosphine (TCEP). In some embodiments, the reducing agent is DTT. In some embodiments, the concentration of the reducing agent in the solution is 0.01 mM to 100 mM. In some embodiments, the concentration of the reducing agent in the solution is 0.1 mM to 10 mM. In some embodiments, the concentration of the reducing agent in the solution is 0.5 mM to 2 mM. In some embodiments, the concentration of the reducing agent in the solution is 0.01 mM to 100 mM. In some embodiments, the concentration of the reducing agent in the solution is 0.1 mM to 10 mM. In some embodiments, the concentration of the reducing agent in the solution is about 1 mM.

In some embodiments, systems comprise a solution, wherein the solution comprises a competitor. In general, competitors compete with the target nucleic acid or the reporter nucleic acid for cleavage by the effector protein or a dimer thereof. Exemplary competitors include heparin, and imidazole, and salmon sperm DNA. In some embodiments, the concentration of the competitor in the solution is 1 μg/mL to 100 μg/mL. In some embodiments, the concentration of the competitor in the solution is 40 μg/mL to 60 μg/mL.

In some embodiments, systems comprise a solution, wherein the solution comprises a co-factor. In some embodiments, the co-factor allows an effector protein or a multimeric complex thereof to perform a function, including pre-crRNA processing and/or target nucleic acid cleavage. The suitability of a cofactor for an effector protein or a multimeric complex thereof may be assessed, such as by methods based on those described by Sundaresan et al. (Cell Rep. 2017 Dec. 26; 21(13): 3728-3739). In some embodiments, an effector or a multimeric complex thereof forms a complex with a co-factor. In some embodiments, the co-factor is a divalent metal ion. In some embodiments, the divalent metal ion is selected from Mg²⁺, Mn²⁺, Zn²⁺, Ca²⁺, Cu²⁺. In some embodiments, the divalent metal ion is Mg′. In some embodiments, the co-factor is Mg²⁺.

Reporters

In some embodiments, systems disclosed herein comprise a reporter. By way of non-limiting and illustrative example, a reporter may comprise a single stranded nucleic acid and a detection moiety (e.g., a labeled single stranded RNA reporter), wherein the nucleic acid is capable of being cleaved by an effector protein (e.g., a CRISPR/Cas protein as disclosed herein) or a multimeric complex thereof, releasing the detection moiety, and generating a detectable signal. As used herein, “reporter” is used interchangeably with “reporter nucleic acid” or “reporter molecule”. The effector proteins disclosed herein, activated upon hybridization of a guide nucleic acid to a target nucleic acid, may cleave the reporter. Cleaving the “reporter” may be referred to herein as cleaving the “reporter nucleic acid,” the “reporter molecule,” or the “nucleic acid of the reporter.” Reporters may comprise RNA. Reporters may comprise DNA. Reporters may be double-stranded. Reporters may be single-stranded.

In some embodiments, reporters comprise a protein capable of generating a signal. A signal may be a calorimetric, potentiometric, amperometric, optical (e.g., fluorescent, colorimetric, etc.), or piezo-electric signal. In some embodiments, the reporter comprises a detection moiety. Suitable detectable labels and/or moieties that may provide a signal include, but are not limited to, an enzyme, a radioisotope, a member of a specific binding pair; a fluorophore; a fluorescent protein; a quantum dot; and the like.

In some embodiments, the reporter comprises a detection moiety and a quenching moiety. In some embodiments, the reporter comprises a cleavage site, wherein the detection moiety is located at a first site on the reporter and the quenching moiety is located at a second site on the reporter, wherein the first site and the second site are separated by the cleavage site. Sometimes the quenching moiety is a fluorescence quenching moiety. In some embodiments, the quenching moiety is 5′ to the cleavage site and the detection moiety is 3′ to the cleavage site. In some embodiments, the detection moiety is 5′ to the cleavage site and the quenching moiety is 3′ to the cleavage site. Sometimes the quenching moiety is at the 5′ terminus of the nucleic acid of a reporter. Sometimes the detection moiety is at the 3′ terminus of the nucleic acid of a reporter. In some embodiments, the detection moiety is at the 5′ terminus of the nucleic acid of a reporter. In some embodiments, the quenching moiety is at the 3′ terminus of the nucleic acid of a reporter.

Suitable fluorescent proteins include, but are not limited to, green fluorescent protein (GFP) or variants thereof, blue fluorescent variant of GFP (BFP), cyan fluorescent variant of GFP (CFP), yellow fluorescent variant of GFP (YFP), enhanced GFP (EGFP), enhanced CFP (ECFP), enhanced YFP (EYFP), GFPS65T, Emerald, Topaz (TYFP), Venus, Citrine, mCitrine, GFPuv, destabilised EGFP (dEGFP), destabilised ECFP (dECFP), destabilised EYFP (dEYFP), mCFPm, Cerulean, T-Sapphire, CyPet, YPet, mKO, HcRed, t-HcRed, DsRed, DsRed2, DsRed-monomer, J-Red, dimer2, t-dimer2(12), mRFP1, pocilloporin, Renilla GFP, Monster GFP, paGFP, Kaede protein and kindling protein, Phycobiliproteins and Phycobiliprotein conjugates including B-Phycoerythrin, R-Phycoerythrin and Allophycocyanin. Suitable enzymes include, but are not limited to, horseradish peroxidase (HRP), alkaline phosphatase (AP), beta-galactosidase (GAL), glucose-6-phosphate dehydrogenase, beta-N-acetylglucosaminidase, glucuronidase, invertase, Xanthine Oxidase, firefly luciferase, and glucose oxidase (GO).

In some embodiments, the detection moiety comprises an invertase. The substrate of the invertase may be sucrose. A DNS reagent may be included in the system to produce a colorimetric change when the invertase converts sucrose to glucose. In some embodiments, the reporter nucleic acid and invertase are conjugated using a heterobifunctional linker via sulfo-SMCC chemistry.

Suitable fluorophores may provide a detectable fluorescence signal in the same range as 6-Fluorescein (Integrated DNA Technologies), IRDye 700 (Integrated DNA Technologies), TYE 665 (Integrated DNA Technologies), Alex Fluor 594 (Integrated DNA Technologies), or ATTO TM 633 (NHS Ester) (Integrated DNA Technologies). Non-limiting examples of fluorophores are fluorescein amidite, 6-Fluorescein, IRDye 700, TYE 665, Alex Fluor 594, or ATTO TM 633 (NHS Ester). The fluorophore may be an infrared fluorophore. The fluorophore may emit fluorescence in the range of 500 nm and 720 nm. In some embodiments, the fluorophore emits fluorescence at a wavelength of 700 nm or higher. In other embodiments, the fluorophore emits fluorescence at about 665 nm. In some embodiments, the fluorophore emits fluorescence in the range of 500 nm to 520 nm, 500 nm to 540 nm, 500 nm to 590 nm, 590 nm to 600 nm, 600 nm to 610 nm, 610 nm to 620 nm, 620 nm to 630 nm, 630 nm to 640 nm, 640 nm to 650 nm, 650 nm to 660 nm, 660 nm to 670 nm, 670 nm to 680 nm, 690 nm to 690 nm, 690 nm to 700 nm, 700 nm to 710 nm, 710 nm to 720 nm, or 720 nm to 730 nm. In some embodiments, the fluorophore emits fluorescence in the range 450 nm to 750 nm, 500 nm to 650 nm, or 550 to 650 nm.

Systems may comprise a quenching moiety. A quenching moiety may be chosen based on its ability to quench the detection moiety. A quenching moiety may be a non-fluorescent fluorescence quencher. A quenching moiety may quench a detection moiety that emits fluorescence in the range of 500 nm and 720 nm. A quenching moiety may quench a detection moiety that emits fluorescence in the range of 500 nm and 720 nm. In some embodiments, the quenching moiety quenches a detection moiety that emits fluorescence at a wavelength of 700 nm or higher. In other embodiments, the quenching moiety quenches a detection moiety that emits fluorescence at about 660 nm or about 670 nm. In some embodiments, the quenching moiety quenches a detection moiety that emits fluorescence in the range of 500 to 520, 500 to 540, 500 to 590, 590 to 600, 600 to 610, 610 to 620, 620 to 630, 630 to 640, 640 to 650, 650 to 660, 660 to 670, 670 to 680, 690 to 690, 690 to 700, 700 to 710, 710 to 720, or 720 to 730 nm. In some embodiments, the quenching moiety quenches a detection moiety that emits fluorescence in the range 450 nm to 750 nm, 500 nm to 650 nm, or 550 to 650 nm. A quenching moiety may quench fluorescein amidite, 6-Fluorescein, IRDye 700, TYE 665, Alex Fluor 594, or ATTO TM 633 (NHS Ester). A quenching moiety may be Iowa Black RQ, Iowa Black FQ or IRDye QC-1 Quencher. A quenching moiety may quench fluorescein amidite, 6-Fluorescein (Integrated DNA Technologies), IRDye 700 (Integrated DNA Technologies), TYE 665 (Integrated DNA Technologies), Alex Fluor 594 (Integrated DNA Technologies), or ATTO TM 633 (NHS Ester) (Integrated DNA Technologies). A quenching moiety may be Iowa Black RQ (Integrated DNA Technologies), Iowa Black FQ (Integrated DNA Technologies) or IRDye QC-1 Quencher (LiCor). Any of the quenching moieties described herein may be from any commercially available source, may be an alternative with a similar function, a generic, or a non-tradename of the quenching moieties listed.

The generation of the detectable signal from the release of the detection moiety may indicate that cleavage by the effector protein has occurred and that the sample contains the target nucleic acid. In some embodiments, the detection moiety comprises a fluorescent dye. Sometimes the detection moiety comprises a fluorescence resonance energy transfer (FRET) pair. In some embodiments, the detection moiety comprises an infrared (IR) dye. In some embodiments, the detection moiety comprises an ultraviolet (UV) dye. Alternatively, or in combination, the detection moiety comprises a protein. Sometimes the detection moiety comprises a biotin. Sometimes the detection moiety comprises at least one of avidin or streptavidin. In some embodiments, the detection moiety comprises a polysaccharide, a polymer, or a nanoparticle. In some embodiments, the detection moiety comprises a gold nanoparticle or a latex nanoparticle.

A detection moiety may be any moiety capable of generating a calorimetric, potentiometric, amperometric, optical (e.g., fluorescent, colorimetric, etc.), or piezo-electric signal. A nucleic acid of a reporter, sometimes, is protein-nucleic acid that is capable of generating a calorimetric, potentiometric, amperometric, optical (e.g., fluorescent, colorimetric, etc.), or piezo-electric signal upon cleavage of the nucleic acid. Often a calorimetric signal is heat produced after cleavage of the nucleic acids of a reporter. Sometimes, a calorimetric signal is heat absorbed after cleavage of the nucleic acids of a reporter. A potentiometric signal, for example, is electrical potential produced after cleavage of the nucleic acids of a reporter. An amperometric signal may be movement of electrons produced after the cleavage of nucleic acid of a reporter. Often, the signal is an optical signal, such as a colorimetric signal or a fluorescence signal. An optical signal is, for example, a light output produced after the cleavage of the nucleic acids of a reporter. Sometimes, an optical signal is a change in light absorbance between before and after the cleavage of nucleic acids of a reporter. Often, a piezo-electric signal is a change in mass between before and after the cleavage of the nucleic acid of a reporter.

The detectable signal may be a colorimetric signal or a signal visible by eye. In some embodiments, the detectable signal may be fluorescent, electrical, chemical, electrochemical, or magnetic. In some embodiments, the first detection signal may be generated by binding of the detection moiety to the capture molecule in the detection region, where the first detection signal indicates that the sample contained the target nucleic acid. Sometimes systems are capable of detecting more than one type of target nucleic acid, wherein the system comprises more than one type of guide nucleic acid and more than one type of reporter nucleic acid. In some embodiments, the detectable signal may be generated directly by the cleavage event. Alternatively, or in combination, the detectable signal may be generated indirectly by the signal event. Sometimes the detectable signal is not a fluorescent signal. In some embodiments, the detectable signal may be a colorimetric or color-based signal. In some embodiments, the detected target nucleic acid may be identified based on its spatial location on the detection region of the support medium. In some embodiments, the second detectable signal may be generated in a spatially distinct location than the first generated signal.

In some embodiments, the reporter nucleic acid is a single-stranded nucleic acid sequence comprising ribonucleotides. The nucleic acid of a reporter may be a single-stranded nucleic acid sequence comprising at least one ribonucleotide. In some embodiments, the nucleic acid of a reporter is a single-stranded nucleic acid comprising at least one ribonucleotide residue at an internal position that functions as a cleavage site. In some embodiments, the nucleic acid of a reporter comprises at least 2, at least 3, at least 4, at least 5, at least 6, at least 7, at least 8, at least 9, or at least 10 ribonucleotide residues at an internal position. In some embodiments, the nucleic acid of a reporter comprises from 2 to 10, from 3 to 9, from 4 to 8, or from 5 to 7 ribonucleotide residues at an internal position. Sometimes the ribonucleotide residues are continuous. Alternatively, the ribonucleotide residues are interspersed in between non-ribonucleotide residues. In some embodiments, the nucleic acid of a reporter has only ribonucleotide residues. In some embodiments, the nucleic acid of a reporter has only DNA residues. In some embodiments, the nucleic acid comprises nucleotides resistant to cleavage by the effector protein described herein. In some embodiments, the nucleic acid of a reporter comprises synthetic nucleotides. In some embodiments, the nucleic acid of a reporter comprises at least one ribonucleotide residue and at least one non-ribonucleotide residue.

In some embodiments, the nucleic acid of a reporter comprises at least one uracil ribonucleotide. In some embodiments, the nucleic acid of a reporter comprises at least two uracil ribonucleotides. Sometimes the nucleic acid of a reporter has only uracil ribonucleotides. In some embodiments, the nucleic acid of a reporter comprises at least one adenine ribonucleotide. In some embodiments, the nucleic acid of a reporter comprises at least two adenine ribonucleotides. In some embodiments, the nucleic acid of a reporter has only adenine ribonucleotides. In some embodiments, the nucleic acid of a reporter comprises at least one cytosine ribonucleotide. In some embodiments, the nucleic acid of a reporter comprises at least two cytosine ribonucleotides. In some embodiments, the nucleic acid of a reporter comprises at least one guanine ribonucleotide. In some embodiments, the nucleic acid of a reporter comprises at least two guanine ribonucleotides. In some embodiments, a nucleic acid of a reporter comprises a single unmodified ribonucleotide. In some embodiments, a nucleic acid of a reporter comprises only unmodified DNAs.

In some embodiments, the nucleic acid of a reporter is 5 to 20, 5 to 15, 5 to 10, 7 to 20, 7 to 15, or 7 to 10 nucleotides in length. In some embodiments, the nucleic acid of a reporter is 3 to 20, 4 to 10, 5 to 10, or 5 to 8 nucleotides in length. In some embodiments, the nucleic acid of a reporter is 5 to 12 nucleotides in length. In some embodiments, the reporter nucleic acid is at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 nucleotides in length. In some embodiments, the reporter nucleic acid is 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides in length.

In some embodiments, systems comprise a plurality of reporters. The plurality of reporters may comprise a plurality of signals. In some embodiments, systems comprise at least 2, at least 3, at least 4, at least 5, at least 6, at least 7, at least 8, at least 9, at least 10, at least 11, at least 12, at least 13, at least 14, at least 15, at least 20, at least 30, at least 40, or at least 50 reporters. In some embodiments, there are 2 to 50, 3 to 40, 4 to 30, 5 to 20, or 6 to 10 different reporters.

In some embodiments, systems comprise an effector protein and a reporter nucleic acid configured to undergo trans cleavage by the effector protein. Trans cleavage of the reporter may generate a signal from the reporter or alter a signal from the reporter. In some embodiments, the signal is an optical signal, such as a fluorescence signal or absorbance band. Trans cleavage of the reporter may alter the wavelength, intensity, or polarization of the optical signal. For example, the reporter may comprise a fluorophore and a quencher, such that trans cleavage of the reporter separates the fluorophore and the quencher thereby increasing a fluorescence signal from the fluorophore. Herein, detection of reporter cleavage to determine the presence of a target nucleic acid may be referred to as ‘DETECTR’. In some embodiments described herein is a method of assaying for a target nucleic acid in a sample comprising contacting the target nucleic acid with an effector protein, a non-naturally occurring guide nucleic acid that hybridizes to a segment of the target nucleic acid, and a reporter nucleic acid, and assaying for a change in a signal, wherein the change in the signal is produced by cleavage of the reporter nucleic acid.

In the presence of a large amount of non-target nucleic acids, an activity of an effector protein (e.g., an effector protein as disclosed herein) may be inhibited. This is because the activated effector proteins collaterally cleave any nucleic acids. If total nucleic acids are present in large amounts, they may outcompete reporters for the effector proteins. In some embodiments, systems comprise an excess of reporter(s), such that when the system is operated and a solution of the system comprising the reporter is combined with a sample comprising a target nucleic acid, the concentration of the reporter in the combined solution-sample is greater than the concentration of the target nucleic acid. In some embodiments, the sample comprises amplified target nucleic acid. In some embodiments, the sample comprises an unamplified target nucleic acid. In some embodiments, the concentration of the reporter is greater than the concentration of target nucleic acids and non-target nucleic acids. The non-target nucleic acids may be from the original sample, either lysed or unlysed. The non-target nucleic acids may comprise byproducts of amplification. In some embodiments, systems comprise a reporter wherein the concentration of the reporter in a solution 1.5 fold, at least 2 fold, at least 3 fold, at least 4 fold, at least 5 fold, at least 6 fold, at least 7 fold, at least 8 fold, at least 9 fold, at least 10 fold, at least 11 fold, at least 12 fold, at least 13 fold, at least 14 fold, at least 15 fold, at least 16 fold, at least 17 fold, at least 18 fold, at least 19 fold, at least 20 fold, at least 30 fold, at least 40 fold, at least 50 fold, at least 60 fold, at least 70 fold, at least 80 fold, at least 90 fold, at least 100 fold excess of total nucleic acids.

Amplification Reagents/Components

In some embodiments, systems described herein comprise a reagent or component for amplifying a nucleic acid. Non-limiting examples of reagents for amplifying a nucleic acid include polymerases, primers, and nucleotides. In some embodiments, systems comprise reagents for nucleic acid amplification of a target nucleic acid in a sample. Nucleic acid amplification of the target nucleic acid may improve at least one of sensitivity, specificity, or accuracy of the assay in detecting the target nucleic acid. In some embodiments, nucleic acid amplification is isothermal nucleic acid amplification, providing for the use of the system or system in remote regions or low resource settings without specialized equipment for amplification. In some embodiments, amplification of the target nucleic acid increases the concentration of the target nucleic acid in the sample relative to the concentration of nucleic acids that do not correspond to the target nucleic acid.

The reagents for nucleic acid amplification may comprise a recombinase, an oligonucleotide primer, a single-stranded DNA binding (SSB) protein, a polymerase, or a combination thereof that is suitable for an amplification reaction. Non-limiting examples of amplification reactions are transcription mediated amplification (TMA), helicase dependent amplification (HDA), or circular helicase dependent amplification (cHDA), strand displacement amplification (SDA), recombinase polymerase amplification (RPA), loop mediated amplification (LAMP), exponential amplification reaction (EXPAR), rolling circle amplification (RCA), ligase chain reaction (LCR), simple method amplifying RNA targets (SMART), single primer isothermal amplification (SPIA), multiple displacement amplification (MDA), nucleic acid sequence based amplification (NASBA), hinge-initiated primer-dependent amplification of nucleic acids (HIP), nicking enzyme amplification reaction (NEAR), and improved multiple displacement amplification (IMDA).

In some embodiments, systems comprise a PCR tube, a PCR well or a PCR plate. The wells of the PCR plate may be pre-aliquoted with the reagent for amplifying a nucleic acid, as well as a guide nucleic acid, an effector protein, a multimeric complex, or any combination thereof. The wells of the PCR plate may be pre-aliquoted with a guide nucleic acid targeting a target sequence, an effector protein capable of being activated when complexed with the guide nucleic acid and the target sequence, and at least one population of a single stranded reporter nucleic acid comprising a detection moiety. A user may thus add the biological sample of interest to a well of the pre-aliquoted PCR plate and measure for the detectable signal with a fluorescent light reader or a visible light reader.

In some embodiments, systems comprise a PCR plate; a guide nucleic acid targeting a target sequence; an effector protein capable of being activated when complexed with the guide nucleic acid and the target sequence; and a single stranded reporter nucleic acid comprising a detection moiety, wherein the reporter nucleic acid is capable of being cleaved by the activated nuclease, thereby generating a detectable signal.

In some embodiments, systems comprise a support medium; a guide nucleic acid targeting a target sequence; and an effector protein capable of being activated when complexed with the guide nucleic acid and the target sequence. In some embodiments, nucleic acid amplification is performed in a nucleic acid amplification region on the support medium. Alternatively, or in combination, the nucleic acid amplification is performed in a reagent chamber, and the resulting sample is applied to the support medium.

In some embodiments, a system for modifying a target nucleic acid comprises a PCR plate; a guide nucleic acid targeting a target sequence; and an effector protein capable of being activated when complexed with the guide nucleic acid and the target sequence. The wells of the PCR plate may be pre-aliquoted with the guide nucleic acid targeting a target sequence, and an effector protein capable of being activated when complexed with the guide nucleic acid and the target sequence. A user may thus add the biological sample of interest to a well of the pre-aliquoted PCR plate.

Often, the nucleic acid amplification is performed for no greater than 1, 2, 3, 4, 5, 6, 7, 8, 9, 10, 11, 12, 13, 14, 15, 16, 17, 18, 19, 20, 25, 30, 40, 50, or 60 minutes, or any value 1 to 60 minutes. Sometimes, the nucleic acid amplification is performed for 1 to 60, 5 to 55, 10 to 50, 15 to 45, 20 to 40, or 25 to 35 minutes. Sometimes, the nucleic acid amplification reaction is performed at a temperature of around 20-45° C. In some embodiments, the nucleic acid amplification reaction is performed at a temperature no greater than 20° C., 25° C., 30° C., 35° C., 37° C., 40° C., 45° C., or any value 20° C. to 45° C. In some embodiments, the nucleic acid amplification reaction is performed at a temperature of at least 20° C., 25° C., 30° C., 35° C., 37° C., 40° C., or 45° C., or any value 20° C. to 45° C. In some embodiments, the nucleic acid amplification reaction is performed at a temperature of 20° C. to 45° C., 25° C. to 40° C., 30° C. to 40° C., or 35° C. to 40° C.

Often, systems comprise primers for amplifying a target nucleic acid to produce an amplification product comprising the target nucleic acid and a PAM. For embodiment, at least one of the primers may comprise the PAM that is incorporated into the amplification product during amplification. The compositions for amplification of target nucleic acids and methods of use thereof, as described herein, are compatible with any of the methods disclosed herein including methods of assaying for at least one base difference (e.g., assaying for a SNP or a base mutation) in a target nucleic acid, methods of assaying for a target nucleic acid that lacks a PAM by amplifying the target nucleic acid to introduce a PAM, and compositions used in introducing a PAM via amplification into the target nucleic acid.

Additional System Components

In some embodiments, systems include a package, carrier, or container that is compartmentalized to receive one or more containers such as vials, tubes, and the like, each of the container(s) comprising one of the separate elements to be used in a method described herein. Suitable containers include, for example, test wells, bottles, vials, and test tubes. In one embodiment, the containers are formed from a variety of materials such as glass, plastic, or polymers. The system or systems described herein contain packaging materials. Examples of packaging materials include, but are not limited to, pouches, blister packs, bottles, tubes, bags, containers, bottles, and any packaging material suitable for intended mode of use.

A system may include labels listing contents and/or instructions for use, or package inserts with instructions for use. A set of instructions will also typically be included. In one embodiment, a label is on or associated with the container. In some embodiments, a label is on a container when letters, numbers or other characters forming the label are attached, molded, or etched into the container itself; a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein. After packaging the formed product and wrapping or boxing to maintain a sterile barrier, the product may be terminally sterilized by heat sterilization, gas sterilization, gamma irradiation, or by electron beam sterilization. Alternatively, the product may be prepared and packaged by aseptic processing.

In some embodiments, systems comprise a solid support. An RNP or effector protein may be attached to a solid support. The solid support may be an electrode or a bead. The bead may be a magnetic bead. Upon cleavage, the RNP is liberated from the solid support and interacts with other mixtures. For example, upon cleavage of the nucleic acid of the RNP, the effector protein of the RNP flows through a chamber into a mixture comprising a substrate. When the effector protein meets the substrate, a reaction occurs, such as a colorimetric reaction, which is then detected. As another example, the protein is an enzyme substrate, and upon cleavage of the nucleic acid of the enzyme substrate-nucleic acid, the enzyme flows through a chamber into a mixture comprising the enzyme. When the enzyme substrate meets the enzyme, a reaction occurs, such as a calorimetric reaction, which is then detected.

Certain System Conditions

In some embodiments, systems and methods are employed under certain conditions that enhance an activity of the effector protein relative to alternative conditions, as measured by a detectable signal released from cleavage of a reporter in the presence of the target nucleic acid. The detectable signal may be generated at about the rate of trans cleavage of a reporter nucleic acid. In some embodiments, the reporter nucleic acid is a homopolymeric reporter nucleic acid comprising 5 to 20 consecutive adenines (SEQ ID NO: 1019), 5 to 20 consecutive thymines (SEQ ID NO: 1020), 5 to 20 consecutive cytosines (SEQ ID NO: 1021), or 5 to 20 consecutive guanines (SEQ ID NO: 1022). In some embodiments, the reporter is an RNA-FQ reporter.

In some embodiments, effector proteins disclosed herein recognize, bind, or are activated by, different target nucleic acids having different sequences, but are active toward the same reporter nucleic acid, allowing for facile multiplexing in a single assay having a single ssRNA-FQ reporter.

In some embodiments, systems are employed under certain conditions that enhance trans cleavage activity of an effector protein. In some embodiments, under certain conditions, transcolatteral cleavage occurs at a rate of at least 0.005 mmol/min, at least 0.01 mmol/min, at least 0.05 mmol/min, at least 0.1 mmol/min, at least 0.2 mmol/min, at least 0.5 mmol/min, or at least 1 mmol/min. In some embodiments, systems and methods are employed under certain conditions that enhance cis-cleavage activity of the effector protein.

Certain conditions that may enhance the activity of an effector protein include a certain salt presence or salt concentration of the solution in which the activity occurs. For example, cis-cleavage activity of an effector protein may be inhibited or halted by a high salt concentration. The salt may be a sodium salt, a potassium salt, or a magnesium salt. In some embodiments, the salt is NaCl. In some embodiments, the salt is KNO3. In some embodiments, the salt concentration is less than 150 mM, less than 125 mM, less than 100 mM, less than 75 mM, less than 50 mM, or less than 25 mM.

Certain conditions that may enhance the activity of an effector protein include the pH of a solution in which the activity. For example, increasing pH may enhance trans cleavage activity. For example, the rate of trans cleavage activity may increase with increase in pH up to pH 9. In some embodiments, the pH is about 7, about 7.1, about 7.2, about 7.3, about 7.4, about 7.5, about 7.6, about 7.7, about 7.8, about 7.9, about 8, about 8.1, about 8.2, about 8.3, about 8.4, about 8.5, about 8.6, about 8.7, about 8.8, about 8.9, or about 9. In some embodiments, the pH is 7 to 7.5, 7.5 to 8, 8 to 8.5, 8.5 to 9, or 7 to 8.5. In some embodiments, the pH is less than 7. In some embodiments, the pH is greater than 7.

Certain conditions that may enhance the activity of an effector protein includes the temperature at which the activity is performed. In some embodiments, the temperature is about 25° C. to about 50° C. In some embodiments, the temperature is about 20° C. to about 40° C., about 30° C. to about 50° C., or about 40° C. to about 60° C. In some embodiments, the temperature is about 25° C., about 30° C., about 35° C., about 40° C., about 45° C., or about 50° C.

### X. PHARMACEUTICAL COMPOSITIONS AND MODES OF ADMINISTRATION

Disclosed herein, in some aspects, are pharmaceutical compositions for modifying a target nucleic acid in a cell or a subject, comprising any one of the effector proteins, engineered effector proteins, fusion effector proteins, or guide nucleic acids as described herein and any combination thereof. Also disclosed herein, in some aspects, are pharmaceutical compositions comprising a nucleic acid encoding any one of the effector proteins, engineered effector proteins, fusion effector proteins, or guide nucleic acids as described herein and any combination thereof. In some embodiments, pharmaceutical compositions comprise a plurality of guide nucleic acids. Pharmaceutical compositions may be used to modify a target nucleic acid or the expression thereof in a cell in vitro, in vivo or ex vivo.

In some embodiments, pharmaceutical compositions comprise one or more nucleic acids encoding an effector protein, fusion effector protein, fusion partner, a guide nucleic acid, or a combination thereof; and a pharmaceutically acceptable carrier or diluent. The effector protein, fusion effector protein, fusion partner protein, or combination thereof may be any one of those described herein. The one or more nucleic acids may comprise a plasmid. The one or more nucleic acids may comprise a nucleic acid expression vector. The one or more nucleic acids may comprise a viral vector. In some embodiments, the viral vector is a lentiviral vector. In some embodiments, the vector is an adeno-associated viral (AAV) vector. In some embodiments, compositions, including pharmaceutical compositions, comprise a viral vector encoding a fusion effector protein and a guide nucleic acid, wherein at least a portion of the guide nucleic acid binds to the effector protein of the fusion effector protein.

In some embodiments, pharmaceutical compositions comprise a virus comprising a viral vector encoding a fusion effector protein, an effector protein, a fusion partner, a guide nucleic acid, or a combination thereof; and a pharmaceutically acceptable carrier or diluent. The virus may be a lentivirus. The virus may be an adenovirus. The virus may be a non-replicating virus. The virus may be an adeno-associated virus (AAV). The viral vector may be a retroviral vector. Retroviral vectors may include gamma-retroviral vectors such as vectors derived from the Moloney Murine Leukemia Virus (MoMLV, MMLV, MuLV, or MLV) or the Murine Stem cell Virus (MSCV) genome. Retroviral vectors may include lentiviral vectors such as those derived from the human immunodeficiency virus (HIV) genome. In some embodiments, the viral vector is a chimeric viral vector, comprising viral portions from two or more viruses. In some embodiments, the viral vector is a recombinant viral vector.

In some embodiments, the viral vector is an AAV. The AAV may be any AAV known in the art. In some embodiments, the viral vector corresponds to a virus of a specific serotype. In some examples, the serotype is selected from an AAV1 serotype, an AAV2 serotype, AAV3 serotype, an AAV4 serotype, AAV5 serotype, an AAV6 serotype, AAV7 serotype, an AAV8 serotype, an AAV9 serotype, an AAV10 serotype, an AAV11 serotype, and an AAV12 serotype. In some embodiments the AAV vector is a recombinant vector, a hybrid AAV vector, a chimeric AAV vector, a self-complementary AAV (scAAV) vector, a single-stranded AAV or any combination thereof scAAV genomes are generally known in the art and contain both DNA strands which may anneal together to form double-stranded DNA.

In some embodiments, methods of producing delivery vectors herein comprise packaging a nucleic acid encoding an effector protein and a nucleic acid that, when transcribed, produces a guide nucleic acid, or a combination thereof, into an AAV vector. In some embodiments, methods of producing the delivery vector comprises, (a) contacting a cell with at least one nucleic acid that, when transcribed, produces a guide nucleic acid; at least one nucleic acid that encodes: (i) a Replication (Rep) gene; and (ii) a Capsid (Cap) gene that encodes an AAV capsid protein; (b) expressing the AAV capsid protein in the cell; (c) assembling an AAV particle; and (d) packaging a Cas effector encoding nucleic acid into the AAV particle, thereby generating an AAV delivery vector. In some embodiments, promoters, stuffer sequences, and any combination thereof may be packaged in the AAV vector. In some embodiments, the AAV vector comprises a sequence encoding a guide nucleic acid. In some embodiments, the guide nucleic acid comprises a crRNA. In some embodiments, the guide nucleic acid is a crRNA. In some embodiments, the guide nucleic acid comprises a sgRNA. In some embodiments, the guide nucleic acid is a sgRNA. In some examples, the AAV vector may package 1, 2, 3, 4, or 5 nucleotide sequences encoding guide nucleic acids or copies thereof. In some examples, the AAV vector packages 1 or 2 nucleotide sequences encoding guide nucleic acids or copies thereof. In some embodiments, the AAV vector packages a nucleotide sequence encoding a first guide nucleic acid and a nucleotide sequence encoding a second guide nucleic acid, wherein the first guide nucleic acid and the second guide nucleic acid are the same. In some embodiments, the AAV vector packages a nucleotide sequence encoding a first guide nucleic acid and a nucleotide sequence encoding a second guide nucleic acid, wherein the first guide nucleic acid and the second guide nucleic acid are different. In some embodiments, the AAV vector comprises inverted terminal repeats, e.g., a 5′ inverted terminal repeat and a 3′ inverted terminal repeat. In some embodiments, the inverted terminal repeat comprises inverted terminal repeats from AAV. In some embodiments, the inverted terminal repeat comprises inverted terminal repeats of ssAAV vector or scAAV vector. In some embodiments, the AAV vector comprises a mutated inverted terminal repeat that lacks a terminal resolution site.

In some embodiments, a hybrid AAV vector is produced by transcapsidation, e.g., packaging an inverted terminal repeat (ITR) from a first serotype into a capsid of a second serotype, wherein the first and second serotypes may be not the same. In some examples, the Rep gene and ITR from a first AAV serotype (e.g., AAV2) may be used in a capsid from a second AAV serotype (e.g., AAV9), wherein the first and second AAV serotypes may be not the same. As a non-limiting example, a hybrid AAV serotype comprising the AAV2 ITRs and AAV9 capsid protein may be indicated AAV2/9. In some examples, the hybrid AAV delivery vector comprises an AAV2/1, AAV2/2, AAV 2/4, AAV2/5, AAV2/8, or AAV2/9 vector.

In some embodiments, the AAV vector may be a chimeric AAV vector. In some embodiments, the chimeric AAV vector comprises an exogenous amino acid or an amino acid substitution, or capsid proteins from two or more serotypes. In some examples, a chimeric AAV vector may be genetically engineered to increase transduction efficiency, selectivity, or a combination thereof.

In some examples, the delivery vector may be a eukaryotic vector, a prokaryotic vector (e.g., a bacterial vector) a viral vector, or any combination thereof. In some embodiments, the delivery vehicle may be a non-viral vector. In some embodiments, the delivery vehicle may be a plasmid. In some embodiments, the plasmid comprises DNA. In some embodiments, the plasmid comprises RNA. In some examples, the plasmid comprises circular double-stranded DNA. In some examples, the plasmid may be linear. In some examples, the plasmid comprises one or more genes of interest and one or more regulatory elements. In some examples, the plasmid comprises a bacterial backbone containing an origin of replication and an antibiotic resistance gene or other selectable marker for plasmid amplification in bacteria. In some examples, the plasmid may be a minicircle plasmid. In some examples, the plasmid contains one or more genes that provide a selective marker to induce a target cell to retain the plasmid. In some examples, the plasmid may be formulated for delivery through injection by a needle carrying syringe. In some examples, the plasmid may be formulated for delivery via electroporation. In some examples, the plasmids may be engineered through synthetic or other suitable means known in the art. For example, in some embodiments, the genetic elements may be assembled by restriction digest of the desired genetic sequence from a donor plasmid or organism to produce ends of the DNA which may then be readily ligated to another genetic sequence.

In some embodiments, the vector is a non-viral vector, and a physical method or a chemical method is employed for delivery into the somatic cell. Exemplary physical methods include electroporation, gene gun, sonoporation, magnetofection, or hydrodynamic delivery. Exemplary chemical methods include delivery of the recombinant polynucleotide via liposomes such as, cationic lipids or neutral lipids; dendrimers; nanoparticles; or cell-penetrating peptides.

In some embodiments, a fusion effector protein as described herein is inserted into a vector. In some embodiments, the vector comprises a nucleotide sequence of one or more promoters, enhancers, ribosome binding sites, RNA splice sites, polyadenylation sites, a replication origin, and/or transcriptional terminator sequences.

In some embodiments, the AAV vector comprises a self-processing array system for guide nucleic acid. Such a self-processing array system refers to a system for multiplexing, stringing together multiple guide nucleic acids under the control of a single promoter. In general, plasmids and vectors described herein comprise at least one promoter. In some embodiments, the promoters are constitutive promoters. In other embodiments, the promoters are inducible promoters. In additional embodiments, the promoters are prokaryotic promoters (e.g., drive expression of a gene in a prokaryotic cell). In some embodiments, the promoters are eukaryotic promoters, (e.g., drive expression of a gene in a eukaryotic cell). Exemplary promoters include, but are not limited to, CMV, EF1a, SV40, PGK1, Ubc, human beta actin, CAG, TRE, UAS, Ac5, polyhedron, CaMKIIa, GALL-10, TEF1, GDS, ADH1, CaMV35S, Ubi, H1, U6, CaMV35S, SV40, CMV, 7SK, and HSV TK promoter. In some embodiments, the promoter is CMV. In some embodiments, the promoter is EF1a. In some embodiments, the promoter is U6. In some embodiments, the promote is H1. In some embodiments, the promoter is 7SK. In some embodiments, the promoter is ubiquitin. In some embodiments, vectors are bicistronic or polycistronic vector (e.g., having or involving two or more loci responsible for generating a protein) having an internal ribosome entry site (IRES) is for translation initiation in a cap-independent manner.

In some embodiments, the AAV vector comprises a promoter for expressing effector proteins. In some embodiments, the promoter for expressing effector protein is a site-specific promoter. In some embodiments, the promoter for expressing effector protein is a muscle-specific promoter. In some embodiments, the muscle-specific promoter comprises Ck8e, SPC5-12, or Desmin promoter sequence. In some embodiments, the promoter for expressing effector protein is a ubiquitous promoter. In some embodiments, the ubiquitous promoter comprises MND or CAG promoter sequence.

In some embodiments, the AAV vector comprises a stuffer sequence. A stuffer sequence may refer to a non-coding sequence of nucleotides that adjusts the length of the viral genome when inserted into a vector to increase packaging efficiency, increase overall viral titer during production, increase transfection efficacy, increase transfection efficiency, and/or decrease vector toxicity. In some embodiments, the stuffer sequence comprises 5′ untranslated region, 3′ untranslated region or combination thereof. In some embodiments, a stuffer sequence serves no other functional purpose than to increase the length of the viral genome. In some embodiments, a stuffer sequence may increase the length of the viral genome as well as have other functional elements

In some embodiments, the 3′-untranslated region comprises a nucleotide sequence of an intron. In some embodiments, the 3′-untranslated region comprises one or more sequence elements, such as an intron sequence or an enhancer sequence. In some embodiments, the 3′-untranslated region comprises an enhancer. In some embodiments, vectors comprise an enhancer Enhancers are nucleotide sequences that have the effect of enhancing promoter activity. In some embodiments, enhancers augment transcription regardless of the orientation of their sequence. In some embodiments, enhancers activate transcription from a distance of several kilo basepairs. Furthermore, enhancers are located optionally upstream or downstream of a gene region to be transcribed, and/or located within the gene, to activate the transcription. Exemplary enhancers include, but are not limited to, WPRE; CMV enhancers; the R-U5′ segment in LTR of HTLV-I (Mol. Cell. Biol., Vol. 8(1), p. 466-472, 1988); SV40 enhancer; the intron sequence between exons 2 and 3 of rabbit 0-globin (Proc. Natl. Acad. Sci. USA., Vol. 78(3), p. 1527-31, 1981); and the genome region of human growth hormone (J Immunol., Vol. 155(3), p. 1286-95, 1995). In some embodiments, the enhancer is WPRE.

In some embodiments, the AAV vector comprises one or more polyadenylation (poly A) signal sequences. In some embodiments, the polyadenlyation signal sequence comprises hGH poly A signal sequence. In some embodiments, the polyadenlyation signal sequence comprises sv40 poly A signal sequence.

Pharmaceutical compositions described herein may comprise a salt. In some embodiments, the salt is a sodium salt. In some embodiments, the salt is a potassium salt. In some embodiments, the salt is a magnesium salt. In some embodiments, the salt is NaCl. In some embodiments, the salt is KNO3. In some embodiments, the salt is Mg²⁺SO₄²⁻.

Non-limiting examples of pharmaceutically acceptable carriers and diluents suitable for the pharmaceutical compositions disclosed herein include buffers (e.g., neutral buffered saline, phosphate buffered saline); carbohydrates (e.g., glucose, mannose, sucrose, dextran, mannitol); polypeptides or amino acids (e.g., glycine); antioxidants; chelating agents (e.g., EDTA, glutathione); adjuvants (e.g., aluminum hydroxide); surfactants (Polysorbate 80, Polysorbate 20, or Pluronic F68); glycerol; sorbitol; mannitol; polyethyleneglycol; and preservatives.

In some embodiments, pharmaceutical compositions are in the form of a solution (e.g., a liquid). The solution may be formulated for injection, e.g., intravenous or subcutaneous injection. In some embodiments, the pH of the solution is about 7, about 7.1, about 7.2, about 7.3, about 7.4, about 7.5, about 7.6, about 7.7, about 7.8, about 7.9, about 8, about 8.1, about 8.2, about 8.3, about 8.4, about 8.5, about 8.6, about 8.7, about 8.8, about 8.9, or about 9. In some embodiments, the pH is 7 to 7.5, 7.5 to 8, 8 to 8.5, 8.5 to 9, or 7 to 8.5. In some embodiments, the pH of the solution is less than 7. In some embodiments, the pH is greater than 7.

In some embodiments, pharmaceutical compositions comprise an: effector protein, fusion effector protein, fusion partner, a guide nucleic acid, or a combination thereof; and a pharmaceutically acceptable carrier or diluent. In some embodiments, pharmaceutical compositions comprise one or more nucleic acids encoding an: effector protein, fusion effector protein, fusion partner, a guide nucleic acid, or a combination thereof; and a pharmaceutically acceptable carrier or diluent. In some embodiments, guide nucleic acid may be a plurality of guide nucleic acids. In some embodiments, the effector protein comprises a sequence that is at least 65%, at least 70%, at least 75%, at least 80%, at least 85%, at least 90%, at least 95%, at least 97%, or at least 98%, at least 99%, or 100% identical to any one of the sequences of TABLE 1. In some embodiments, the guide nucleic acid may be a guide nucleic acid described herein.

### XI. METHODS AND FORMULATIONS FOR INTRODUCING SYSTEMS AND COMPOSITIONS INTO A TARGET CELL

A guide nucleic acid (or a nucleic acid comprising a nucleotide sequence encoding same) and/or an effector protein described herein may be introduced into a host cell by any of a variety of well-known methods. As a non-limiting example, a guide nucleic acid and/or effector protein may be combined with a lipid. As another non-limiting example, a guide nucleic acid and/or effector protein may be combined with a particle, or formulated into a particle.

Methods for Introducing Systems and Compositions to a Host

Described herein are methods of introducing various components described herein to a host. A host may be any suitable host, such as a host cell. When described herein, a host cell may be an in vivo or in vitro eukaryotic cell, a prokaryotic cell (e.g., bacterial or archaeal cell), or a cell from a multicellular organism (e.g., a cell line) cultured as a unicellular entity, which eukaryotic or prokaryotic cells may be, or have been, used as recipients for methods of introduction described herein, and include the progeny of the original cell which has been transformed by the methods of introduction described herein. It is understood that the progeny of a single cell may not necessarily be completely identical in morphology or in genomic or total DNA complement as the original parent, due to natural, accidental, or deliberate mutation. A host cell may be a recombinant host cell or a genetically modified host cell, if a heterologous nucleic acid, e.g., an expression vector, has been introduced into the cell.

Methods of introducing a nucleic acid and/or protein into a host cell are known in the art, and any convenient method may be used to introduce a subject nucleic acid (e.g., an expression construct/vector) into a target cell (e.g., a human cell, and the like). Suitable methods include, e.g., viral infection, transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct micro injection, nanoparticle-mediated nucleic acid delivery (see, e.g., Panyam et al. Adv Drug Deliv Rev. 2012 Sep. 13. pii: S0169-409X(12)00283-9. doi: 10.1016/j.addr.2012.09.023), and the like. In some embodiments, the nucleic acid and/or protein are introduced into a disease cell comprised in a pharmaceutical composition comprising the guide nucleic acid and/or effector protein and a pharmaceutically acceptable excipient.

In some embodiments, molecules of interest, such as nucleic acids of interest, are introduced to a host. In some embodiments, polypeptides, such as an effector protein are introduced to a host. In some embodiments, vectors, such as lipid particles and/or viral vectors may be introduced to a host. Introduction may be for contact with a host or for assimilation into the host, for example, introduction into a host cell.

In some embodiments, described herein are methods of introducing one or more nucleic acids, such as a nucleic acid encoding an effector protein, a nucleic acid that, when transcribed, produces an engineered guide nucleic acid, and/or a donor nucleic acid, or combinations thereof, into a host cell. Any suitable method may be used to introduce a nucleic acid into a cell. Suitable methods include, for example, viral infection, transfection, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct microinjection, nanoparticle-mediated nucleic acid delivery, and the like. Further methods are described throughout.

Introducing one or more nucleic acids into a host cell may occur in any culture media and under any culture conditions that promote the survival of the cells. Introducing one or more nucleic acids into a host cell may be carried out in vivo or ex vivo. Introducing one or more nucleic acids into a host cell may be carried out in vitro.

In some embodiments, an effector protein may be provided as RNA. The RNA may be provided by direct chemical synthesis or may be transcribed in vitro from a DNA (e.g., encoding the effector protein). Once synthesized, the RNA may be introduced into a cell by way of any suitable technique for introducing nucleic acids into cells (e.g., microinjection, electroporation, transfection, etc.). In some embodiments, introduction of one or more nucleic acid may be through the use of a vector and/or a vector system, accordingly, in some embodiments, compositions and system described herein comprise a vector and/or a vector system.

Vectors may be introduced directly to a host. In some embodiments, host cells may be contacted with one or more vectors as described herein, and In some embodiments, said vectors are taken up by the cells. Methods for contacting cells with vectors include but are not limited to electroporation, calcium chloride transfection, microinjection, lipofection, micro-injection, contact with the cell or particle that comprises a molecule of interest, or a package of cells or particles that comprise molecules of interest.

Components described herein may also be introduced directly to a host. For example, an engineered guide nucleic acid may be introduced to a host, specifically introduced into a host cell. Methods of introducing nucleic acids, such as RNA into cells include, but are not limited to direct injection, transfection, or any other method used for the introduction of nucleic acids.

Polypeptides (e.g., effector proteins) described herein may also be introduced directly to a host. In some embodiments, polypeptides described herein may be modified to promote introduction to a host. For example, polypeptides described herein may be modified to increase the solubility of the polypeptide. Such a polypeptide may optionally be fused to a polypeptide domain that increases solubility. The domain may be linked to the polypeptide through a defined protease cleavage site, such as TEV sequence which is cleaved by TEV protease. The linker may also include one or more flexible sequences, e.g. from 1 to 10 glycine residues. In some embodiments, the cleavage of the polypeptide is performed in a buffer that maintains solubility of the product, e.g. in the presence of from 0.5 to 2 M. urea, in the presence of polypeptides and/or polynucleotides that increase solubility, and the like. Domains of interest include endosomolytic domains, e.g. influenza HA domain; and other polypeptides that aid in production, e.g. IF2 domain, GST domain, GRPE domain, and the like. In another example, the polypeptide may be modified to improve stability. For example, the polypeptides may be PEGylated, where the polyethyleneoxy group provides for enhanced lifetime in the blood stream. Polypeptides may also be modified to promote uptake by a host, such as a host cell. For example, a polypeptide described herein may be fused to a polypeptide permeant domain to promote uptake by a host cell. Any suitable permeant domains may be used in the non-integrating polypeptides of the present disclosure, including peptides, peptidomimetics, and non-peptide carriers. Examples include penetratin, a permeant peptide may be derived from the third alpha helix of Drosophila melanogaster transcription factor Antennapaedia; the HIV-1 tat basic region amino acid sequence, e.g., amino acids 49-57 of a naturally-occurring tat protein; and poly-arginine motifs, for example, the region of amino acids 34-56 of HIV-1 rev protein, nonaarginine, octa-arginine, and the like. The site at which the fusion is made may be selected in order to optimize the biological activity, secretion or binding characteristics of the polypeptide. The optimal site may be determined by suitable methods.

Formulations for Introducing Systems and Compositions to a Host

Described herein are formulations of introducing systems and compositions described herein to a host. In some embodiments, such formulations, systems and compositions described herein comprise an effector protein and a carrier (e.g., excipient, diluent, vehicle, or filling agent). In some aspects of the present invention the effector protein is provided in a pharmaceutical composition comprising the effector protein and any pharmaceutically acceptable excipient, carrier, or diluent.

### XII. METHODS OF NUCLEIC ACID EDITING

Provided herein are compositions, methods, and systems for editing target nucleic acids. In general, editing refers to modifying the nucleotide sequence of a target nucleic acid. However, compositions, methods, and systems disclosed herein may also be capable of making epigenetic modifications of target nucleic acids. Effector proteins, compositions and systems described herein may be used for editing or modifying a target nucleic acid. Editing a target nucleic acid may comprise one or more of: cleaving the target nucleic acid, deleting one or more nucleotides of the target nucleic acid, inserting one or more nucleotides into the target nucleic acid, mutating one or more nucleotides of the target nucleic acid, or modifying (e.g., methylating, demethylating, deaminating, or oxidizing) of one or more nucleotides of the target nucleic acid.

The target nucleic acid may be a gene or a portion thereof. Methods and compositions may modify a coding portion of a gene, a non-coding portion of a gene, or a combination thereof. Modifying at least one gene using the compositions and methods described herein may reduce or increase expression of one or more genes. In some embodiments, compositions and methods reduce expression of one or more genes by at least 10%, at least 20%, at least 30%, at least 40%, at least 50%, at least 60%, at least 70%, at least 80%, at least 90%, or at least 95%. In some embodiments, compositions and methods remove all expression of a gene, also referred to as genetic knock out. In some embodiments, compositions and methods increase expression of one or more genes by at least 10%, at least 20%, at least 30%, at least 40%, at least 50%, at least 60%, at least 70%, at least 80%, at least 90%, or at least 100%.

In some embodiments, compositions and methods comprise a nucleic acid expression vector, or use thereof, to introduce an effector protein, guide nucleic acid, donor template or any combination thereof to a cell. In some embodiments, the nucleic acid expression vector is a viral vector. Viral vectors include, but are not limited to, retroviruses, adenoviruses, adeno-associated viruses, and herpes simplex viruses. In some embodiments, the viral vector is a replication-defective viral vector, comprising an insertion of a therapeutic gene inserted in genes essential to the lytic cycle, preventing the virus from replicating and exerting cytotoxic effects. In some embodiments, the viral vector is an adeno associated viral (AAV) vector. In some embodiments, the nucleic acid expression vector is a non-viral vector. In some embodiments, compositions and methods comprise a lipid, polymer, nanoparticle, or a combination thereof, or use thereof, to introduce a Cas protein, guide nucleic acid, donor template or any combination thereof to a cell. Non-limiting examples of lipids and polymers are cationic polymers, cationic lipids, or bio-responsive polymers. In some embodiments, the bio-responsive polymer exploits chemical-physical properties of the endosomal environment (e.g., pH) to preferentially release the genetic material in the intracellular space.

Methods of editing may comprise contacting a target nucleic acid with one or more components, compositions or systems described herein. In some embodiments, a method of editing comprises contacting a target nucleic acid with at least one of: a) one or more effector proteins, or one or more nucleic acids encoding one or more effector proteins; or b) one or more guide nucleic acids, or one or more nucleic acids encoding one or more guide nucleic acids. In some embodiments, a method of editing comprises contacting a target nucleic acid with a system described herein wherein the system comprises components comprising at least one of: a) one or more effector proteins, or one or more nucleic acids encoding one or more effector proteins; or b) one or more guide nucleic acids, or one or more nucleic acids encoding one or more guide nucleic acids. In some embodiments, a method of editing comprises contacting a target nucleic acid with a composition described herein comprising at least one of: a) one or more effector proteins, or one or more nucleic acids encoding one or more effector proteins; or b) one or more guide nucleic acids, or one or more nucleic acids encoding one or more guide nucleic acids; in a composition.

Editing may introduce a mutation (e.g., point mutations, deletions) in a target nucleic acid relative to a corresponding wildtype nucleotide sequence. Editing may remove or correct a disease-causing mutation in a nucleic acid sequence to produce a corresponding wildtype nucleotide sequence. Editing may remove/correct point mutations, deletions, null mutations, or tissue-specific mutations in a target nucleic acid. Editing may be used to generate gene knock-out, gene knock-in, gene editing, gene tagging, or a combination thereof. Methods of the disclosure may be targeted to any locus in a genome of a cell.

Editing may comprise single stranded cleavage, double stranded cleavage, donor nucleic acid insertion, epigenetic modification (e.g., methylation, demethylation, acetylation, or deacetylation), or a combination thereof. In some embodiments, cleavage (single-stranded or double-stranded) is site-specific, meaning cleavage occurs at a specific site in the target nucleic acid, often within the region of the target nucleic acid that hybridizes with the guide nucleic acid spacer region. In some embodiments, the effector proteins introduce a single-stranded break in a target nucleic acid to produce a cleaved nucleic acid. In some embodiments, the effector protein is capable of introducing a break in a single stranded RNA (ssRNA). The effector protein may be coupled to a guide nucleic acid that targets a particular region of interest in the ssRNA. In some embodiments, the target nucleic acid, and the resulting cleaved nucleic acid is contacted with a nucleic acid for homologous recombination (e.g., homology directed repair (HDR)) or non-homologous end joining (NHEJ). In some embodiments, a double-stranded break in the target nucleic acid may be repaired (e.g., by NHEJ or HDR) without insertion of a donor template, such that the repair results in an indel in the target nucleic acid at or near the site of the double-stranded break. In some embodiments, an indel, sometimes referred to as an insertion-deletion or indel mutation, is a type of genetic mutation that results from the insertion and/or deletion of one or more nucleotide in a target nucleic acid. An indel may vary in length (e.g., 1 to 1,000 nucleotides in length) and be detected using methods well known in the art, including sequencing. If the number of nucleotides in the insertion/deletion is not divisible by three, and it occurs in a protein coding region, it is also a frameshift mutation. Indel percentage is the percentage of sequencing reads that show at least one nucleotide has been mutation that results from the insertion and/or deletion of nucleotides regardless of the size of insertion or deletion, or number of nucleotides mutated. For example, if there is at least one nucleotide deletion detected in a given target nucleic acid, it counts towards the percent indel value. As another example, if one copy of the target nucleic acid has one nucleotide deleted, and another copy of the target nucleic acid has 10 nucleotides deleted, they are counted the same. This number reflects the percentage of target nucleic acids that are modified by a given effector protein.

In some embodiments, wherein the compositions, systems, and methods of the present disclosure comprise an additional guide nucleic acid or a use thereof, the dual-guided compositions, systems, and methods described herein may modify the target nucleic acid in two locations. In some embodiments, dual-guided editing may comprise cleavage of the target nucleic acid in the two locations targeted by the guide nucleic acids. In some embodiments, upon removal of the sequence between the guide nucleic acids, the wild-type reading frame is restored. A wild-type reading frame may be a reading frame that produces at least a partially, or fully, functional protein. A non-wild-type reading frame may be a reading frame that produces a non-functional or partially non-functional protein.

Accordingly, in some embodiments, compositions, systems, and methods described herein may edit 1 to 1,000 nucleotides or any integer in between, in a target nucleic acid. In some embodiments, 1 to 1,000, 2 to 900, 3 to 800, 4 to 700, 5 to 600, 6 to 500, 7 to 400, 8 to 300, 9 to 200, or 10 to 100 nucleotides, or any integer in between, may be edited by the compositions, systems, and methods described herein. In some embodiments, 1, 2, 3, 4, 5, 6, 7, 8, 9, 10, or more nucleotides may be edited by the compositions, systems, and methods described herein. In some embodiments, 10, 20, 30, 40, 50, 60, 70, 80 90, 100 or more nucleotides, or any integer in between, may be edited by the compositions, systems, and methods described herein. In some embodiments, 100, 200, 300, 400, 500, 600, 700, 800, 900 or more nucleotides, or any integer in between, may be edited by the compositions, systems, and methods described herein.

In some embodiments, the effector protein is fused to a chromatin-modifying enzyme. In some embodiments, the fusion protein chemically modifies the target nucleic acid, for example by methylating, demethylating, or acetylating the target nucleic acid in a sequence specific or non-specific manner.

Methods may comprise use of two or more effector proteins. An illustrative method for introducing a break in a target nucleic acid comprises contacting the target nucleic acid with: (a) a first engineered guide nucleic acid comprising a region that binds to a first effector protein described herein; and (b) a second engineered guide nucleic acid comprising a region that binds to a second effector protein described herein, wherein the first engineered guide nucleic acid comprises an additional region that binds to the target nucleic acid and wherein the second engineered guide nucleic acid comprises an additional region that binds to the target nucleic acid. The first and second effector protein may be identical or may be non-identical.

In some embodiments, editing a target nucleic acid comprises genome editing. Genome editing may comprise modifying a genome, chromosome, plasmid, or other genetic material of a cell or organism. In some embodiments, the genome, chromosome, plasmid, or other genetic material of the cell or organism is modified in vivo. In some embodiments, the genome, chromosome, plasmid, or other genetic material of the cell or organism is modified in a cell. In some embodiments, the genome, chromosome, plasmid, or other genetic material of the cell or organism is modified in vitro. For example, a plasmid may be modified in vitro using a composition described herein and introduced into a cell or organism. In some embodiments, modifying a target nucleic acid may comprise deleting a sequence from a target nucleic acid. For example, a mutated sequence or a sequence associated with a disease may be removed from a target nucleic acid. In some embodiments, modifying a target nucleic acid may comprise replacing a sequence in a target nucleic acid with a second sequence. For example, a mutated sequence or a sequence associated with a disease may be replaced with a second sequence lacking the mutation or that is not associated with the disease. In some embodiments, modifying a target nucleic acid may comprise introducing a sequence into a target nucleic acid. For example, a beneficial sequence or a sequence that may reduce or eliminate a disease may be inserted into the target nucleic acid.

In some embodiments, methods comprise inserting a donor nucleic acid into a cleaved target nucleic acid. The donor nucleic acid may be inserted at a specified (e.g., effector protein targeted) point within the target nucleic acid. In some embodiments, methods comprise contacting a target nucleic acid with an effector protein described herein, thereby introducing a single-stranded break in the target nucleic acid; contacting the target nucleic acid with a second effector protein described herein, to generate a second cleavage site in the target nucleic acid, ligating the regions flanking the first and second cleavage site, optionally through NHEJ or single-strand annealing, thereby resulting in the excision of a portion of the target nucleic acid between the first and second cleavage sites from the target nucleic acid; and contacting the target nucleic acid with a donor nucleic acid for homologous recombination, optionally via HDR or NHEJ, thereby introducing a new sequence into the target nucleic acid (e.g., at a cleavage site or in between two cleavage sites).

In some embodiments, methods comprise editing a target nucleic acid with two or more effector proteins. Editing a target nucleic acid may comprise introducing a two or more single-stranded breaks in a target nucleic acid. In some embodiments, a break may be introduced by contacting a target nucleic acid with an effector protein and a guide nucleic acid. The guide nucleic acid may bind to the effector protein and hybridize to a region of the target nucleic acid, thereby recruiting the effector protein to the region of the target nucleic acid. Binding of the effector protein to the guide nucleic acid and the region of the target nucleic acid may activate the effector protein, and the effector protein may introduce a break (e.g., a single stranded break) in the region of the target nucleic acid. In some embodiments, modifying a target nucleic acid may comprise introducing a first break in a first region of the target nucleic acid and a second break in a second region of the target nucleic acid. For example, modifying a target nucleic acid may comprise contacting a target nucleic acid with a first guide nucleic acid that binds to a first effector protein and hybridizes to a first region of the target nucleic acid and a second guide nucleic acid that binds to a second programmable nickase and hybridizes to a second region of the target nucleic acid. The first effector protein may introduce a first break in a first strand at the first region of the target nucleic acid, and the second effector protein may introduce a second break in a second strand at the second region of the target nucleic acid. In some embodiments, a segment of the target nucleic acid between the first break and the second break may be removed, thereby modifying the target nucleic acid. In some embodiments, a segment of the target nucleic acid between the first break and the second break may be replaced (e.g., with donor nucleic acid), thereby modifying the target nucleic acid.

In some embodiments, editing is achieved by fusing an effector protein to a heterologous sequence. The heterologous sequence may be a suitable fusion partner, e.g., a protein that provides recombinase activity by acting on the target nucleic acid. In some embodiments, the fusion protein comprises an effector protein fused to a heterologous sequence by a linker. The heterologous sequence or fusion partner may be a base editing domain. The base editing domain may be an ADAR1/2 or any functional variant thereof. The heterologous sequence or fusion partner may be fused to the C-terminus, N-terminus, or an internal portion (e.g., a portion other than the N- or C-terminus) of the effector protein. The heterologous sequence or fusion partner may be fused to the effector protein by a linker. A linker may be a peptide linker or a non-peptide linker. In some embodiments, the linker is an XTEN linker. In some embodiments, the linker comprises one or more repeats a tri-peptide GGS. In some embodiments, the linker is from 1 to 100 amino acids in length. In some embodiments, the linker is more 100 amino acids in length. In some embodiments, the linker is from 10 to 27 amino acids in length. A non-peptide linker may be a polyethylene glycol (PEG), polypropylene glycol (PPG), co-poly(ethylene/propylene) glycol, polyoxyethylene (POE), polyurethane, polyphosphazene, polysaccharides, dextran, polyvinyl alcohol, polyvinylpyrrolidones, polyvinyl ethyl ether, polyacrylamide, polyacrylate, polycyanoacrylates, lipid polymers, chitins, hyaluronic acid, heparin, or an alkyl linker.

Methods, systems and compositions described herein may edit or modify a target nucleic acid wherein such editing or modification may effect one or more indels. In some embodiments, where compositions, systems, and/or methods described herein effect one or more indels, then In some embodiments, the impact on the transcription and/or translation of the target nucleic acid may be predicted depending on: 1) the amount of indels generated; and 2) the location of the indel on the target nucleic acid. For example, as described herein, in some embodiments, if the amount of indels is not divisible by three, and the indels occur within or along a protein coding region, then the modification or mutation may be a frameshift mutation.

In some embodiments, if the amount of indels is divisible by three, then a frameshift mutation may not be effected, but a splicing disruption mutation and/or sequence skip mutation may be effected, such as an exon skip mutation. In some embodiments, if the amount of indels is not evenly divisible by three, then a frameshift mutation may be effected.

Methods, systems and compositions described herein may edit or modify a target nucleic acid wherein such editing or modification may be measured by indel activity. Indel activity measures the amount of change in a target nucleic acid (e.g., nucleotide deletion(s) and/or insertion(s)) compared to a target nucleic acid that has not been contacted by a polypeptide described in compositions, systems, and methods described herein. For example, indel activity may be detected by next generation sequencing of one or more target loci of a target nucleic acid where indel percentage is calculated as the fraction of sequencing reads containing insertions or deletions relative to an unedited reference sequence. In some embodiments, methods, systems, and compositions comprising an effector protein and guide nucleic acid described herein may exhibit about 0.0001% to about 65% or more indel activity upon contact to a target nucleic acid compared to a target nucleic acid non-contacted with compositions, systems, or by methods described herein. For example, methods, systems, and compositions comprising an effector protein and guide nucleic acid described herein may exhibit about 0.0001%, about 0.001%, about 0.01%, about 0.1%, about 1%, about 5%, about 10%, about 15%, about 20%, about 25%, about 30%, about 35%, about 40%, about 45%, about 50%, about 55%, about 60%, about 65% or more indel activity.

In some embodiments, editing or modifications of a target nucleic acid as described herein effects one or more mutations comprising splicing disruption mutations, frameshift mutations (e.g., 1+ or 2+frameshift mutation), sequence deletion, sequence skipping, sequence reframing, sequence knock-in, or any combination thereof.

A splicing disruption may be a modification that disrupts the splicing of a target nucleic acid or splicing of a sequence that is transcribed from a target nucleic acid relative to a target nucleic acid without the splicing disruption.

A frameshift mutation may be a modification that alters the reading frame of a target nucleic acid relative to a target nucleic acid without the frameshift mutation. In some embodiments, a frameshift mutation may be a +2 frameshift mutation wherein a reading frame is modified by 2 bases. In some embodiments, a frameshift mutation may be a +1 frameshift mutation wherein a reading frame is modified by 1 base. In some embodiments, a frameshift mutation is a modification that alters the number of bases in a target nucleic acid so that it is not divisible by three. In some embodiments, a frameshift mutation may be a modification that is not a splicing disruption.

A sequence as described in reference to a sequence deletion, sequence skipping, sequence reframing, and sequence knock-in may be a DNA sequence, a RNA sequence, a modified DNA or RNA sequence, a mutated sequence, a wild-type sequence, a coding sequence, a non-coding sequence, an exonic sequence (exon), an intronic sequence (intron), or any combination thereof. Such a sequence may be a sequence that is associated with a disease as described herein, such as DMD.

In some embodiments, sequence deletion is a modification where one or more sequences in a target nucleic acid are deleted relative to a target nucleic acid without the sequence deletion. In some embodiments, a sequence deletion may result in or effect a splicing disruption or a frameshift mutation. In some embodiments, a sequence deletion result in or effect a splicing disruption.

In some embodiments, sequence skipping is a modification where one or more sequences in a target nucleic acid are skipped upon transcription or translation of the target nucleic acid relative to a target nucleic acid without the sequence skipping. In some embodiments, sequence skipping may result in or effect a splicing disruption or a frameshift mutation. In some embodiments, sequence skipping may result in or effect a splicing disruption.

In some embodiments, sequence reframing is a modification where one or more bases in a target are modified so that the reading frame of the sequence is reframed relative to a target nucleic acid without the sequence reframing. In some embodiments, sequence reframing may result in or effect a splicing disruption or a frameshift mutation. In some embodiments, sequence reframing may result in or effect a frameshift mutation.

In some embodiments, sequence knock-in is a modification where one or more sequences is inserted into a target nucleic acid relative to a target nucleic acid without the sequence knock-in. In some embodiments, sequence knock-in may result in or effect a splicing disruption or a frameshift mutation. In some embodiments, sequence knock-in may result in or effect a splicing disruption.

In some embodiments, editing or modification of a target nucleic acid may be locus specific, wherein compositions, systems, and methods described herein may edit or modify a target nucleic acid at one or more specific loci to effect one or more specific mutations comprising splicing disruption mutations, frameshift mutations, sequence deletion, sequence skipping, sequence reframing, sequence knock-in, or any combination thereof. For example, editing or modification of a specific locus may effect any one of a splicing disruption, frameshift (e.g., 1+ or 2+frameshift), sequence deletion, sequence skipping, sequence reframing, sequence knock-in, or any combination thereof. In some embodiments, editing or modification of a target nucleic acid may be locus specific, modification specific, or both. In some embodiments, editing or modification of a target nucleic acid may be locus specific, modification specific, or both, wherein compositions, systems, and methods described herein comprise an effector protein described herein and a guide nucleic acid described herein.

Methods of editing a target nucleic acid or modulating the expression of a target nucleic acid may be performed in vivo. Methods of editing a target nucleic acid or modulating the expression of a target nucleic acid may be performed in vitro. For example, a plasmid may be modified in vitro using a composition described herein and introduced into a cell or organism. Methods of editing a target nucleic acid or modulating the expression of a target nucleic acid may be performed ex vivo. For example, methods may comprise obtaining a cell from a subject, modifying a target nucleic acid in the cell with methods described herein, and returning the cell to the subject.

Also described herein is a method of modifying a target nucleic acid within a human gene, or associated with expression of a human gene, the method comprising contacting the target nucleic acid with any one of the compositions described herein, any one of the nucleic acid expression vectors or libraries described herein, any one of the pharmaceutical compositions described herein, or any one of the systems described herein, thereby modifying the target nucleic acid.

In some embodiments, the method of modifying a target nucleic acid comprises insertion or deletion of an exon, intron, exon fragment, intron fragment, gene regulatory region, gene regulatory region fragment, or any combinations thereof. In some embodiments, the modifying of the target nucleic acid comprises insertion of an exon, intron, exon fragment, intron fragment, gene regulatory region, gene regulatory region fragment, or combinations thereof. In some embodiments, the method of modifying further comprising contacting the target nucleic acid with a guide nucleic acid. In some embodiments, the method of modifying is performed in a cell. In some embodiments, the method of modifying is performed in vivo. In some embodiments, the method of modifying as described herein, wherein the target nucleic acid comprises a mutation associated with a disease, and optionally wherein the disease is any one of the diseases recited in TABLE 5 and/or wherein the target nucleic acid is encoded by a gene recited in TABLE 4. In some embodiments, the disease is a genetic disorder. In some embodiments, the genetic disorder is a neurological disorder. In some embodiments, the target nucleic acid is encoded by a gene recited in TABLE 4. In some embodiments, the gene comprises one or more mutations. In some embodiments, the one or more mutations comprise a point mutation, a single nucleotide polymorphism (SNP), a chromosomal mutation, a copy number mutation, or any combination thereof. In some embodiments, the disease is any one of the diseases recited in TABLE 5.

In some embodiments, the modification of the target nucleic acid comprises insertion of a donor nucleic acid, deletion of a target nucleic acid, insertion of a donor nucleic acid fragment, deletion of a target nucleic acid fragment, or combinations thereof. In some embodiments, the modification of the target nucleic acid comprises insertion of a donor nucleic acid or donor nucleic acid fragment into the target nucleic acid. In some embodiments, the modification of the target nucleic acid comprises deletion of a target nucleic acid or a target nucleic acid fragment from the target nucleic acid.

Donor Nucleic Acids

In some embodiments, a donor nucleic acid comprises a nucleic acid that is incorporated into a target nucleic acid or target sequence. In some embodiments, a donor nucleic acid may be incorporated into an insertion site in a target nucleic acid. Exemplary insertion sites are described herein. In some embodiments, a donor nucleic acid comprises a structural motif that is recognized by polypeptides and/or partner polypeptides described herein. In some embodiments, the structural motif is an intermolecular recombination motif. In some embodiments, the structural motif is one or more inverted repeats, inverted terminal repeats, or combinations thereof.

In reference to a viral vector, the term donor nucleic acid refers to a sequence of nucleotides that will be or has been introduced into a cell following transfection of the viral vector. The donor nucleic acid may be introduced into the cell by any mechanism of the transfecting viral vector, including, but not limited to, integration into the genome of the cell or introduction of an episomal plasmid or viral genome. As another example, when used in reference to the activity of an effector protein, the term donor nucleic acid refers to a sequence of nucleotides that will be or has been inserted at the site of cleavage by the effector protein (cleaving (hydrolysis of a phosphodiester bond) of a nucleic acid resulting in a nick or double strand break—nuclease activity). As yet another example, when used in reference to homologous recombination, the term donor nucleic acid refers to a sequence of DNA that serves as a template in the process of homologous recombination, which may carry the modification that is to be or has been introduced into the target nucleic acid. By using this donor nucleic acid as a template, the genetic information, including the modification, is copied into the target nucleic acid by way of homologous recombination.

Donor nucleic acids of any suitable size may be integrated into a target nucleic acid or genome. In some embodiments, the donor polynucleotide integrated into a genome is less than 3, about 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 16, 17, 18, 19, 20, 25, 30, 35, 40, 45, 50, 100, 150, 200, 250, 300, 350, 400, 450, 500 kilobases in length. In some embodiments, donor nucleic acids are more than 500 kilobases (kb) in length.

The donor nucleic acid may comprise a sequence that is derived from a plant, bacteria, virus or an animal. The animal may be human. The animal may be a non-human animal, such as, by way of non-limiting example, a mouse, rat, hamster, rabbit, pig, bovine, deer, sheep, goat, chicken, cat, dog, ferret, a bird, non-human primate (e.g., marmoset, rhesus monkey). The non-human animal may be a domesticated mammal or an agricultural mammal.

Also described herein is a composition comprising an effector protein and an effector partner combination as described in TABLE 6. In some embodiments, the composition comprises the nucleic acid that is a donor nucleic acid. In some embodiments, the donor nucleic acid is linear double-stranded DNA or not linear double-stranded DNA. In some embodiments, the donor nucleic acid comprises a structural motif that is recognized by the polypeptide. In some embodiments, the structural motif is an intermolecular recombination motif, and optionally wherein the structural motif is one or more inverted repeats, inverted terminal repeats, or combinations thereof.

Genetically Modified Cells and Organisms

Methods of editing described herein may be employed to generate a genetically modified cell. The cell may be a eukaryotic cell (e.g., a mammalian cell) or a prokaryotic cell (e.g., an archaeal cell). The cell may be derived from a multicellular organism and cultured as a unicellular entity. The cell may comprise a heritable genetic modification, such that progeny cells derived therefrom comprise the heritable genetic mutation. The cell may be progeny of a genetically modified cell comprising a genetic modification of the genetically modified parent cell. A genetically modified cell may comprise a deletion, insertion, mutation, or non-native sequence relative to a wild-type version of the cell or the organism from which the cell was derived.

Methods may comprise contacting a cell with a nucleic acid (e.g., a plasmid or mRNA) comprising a nucleotide sequence encoding an effector protein, wherein the effector protein comprises an amino acid sequence that is at least 65%, at least 70%, at least 75%, at least 80%, at least 85%, at least 90%, at least 95%, at least 97%, at least 98%, or at least 99% identical to any one of the sequences of TABLE 1.

Methods may comprise contacting a cell with a nucleic acid (e.g., a plasmid or mRNA) comprising a nucleotide sequence encoding an effector partner, wherein the effector partner comprises an amino acid sequence that is at least 65%, at least 70%, at least 75%, at least 80%, at least 85%, at least 90%, at least 95%, at least 97%, at least 98%, or at least 99% identical to any one of the sequences of TABLE 1.1.

Methods may comprise contacting cells with a nucleic acid (e.g., a plasmid or RNA) comprising a a guide nucleic acid described herein or a nucleotide sequence, when transcribed, produces a guide nucleic acid described herein. Contacting may comprise electroporation, acoustic poration, optoporation, viral vector-based delivery, iTOP, nanoparticle delivery (e.g., lipid or gold nanoparticle delivery), cell-penetrating peptide (CPP) delivery, DNA nanostructure delivery, or any combination thereof.

Methods may comprise contacting a cell with one or more of an effector protein, an effector partner, or a multimeric complex thereof, wherein each effector protein comprises an amino acid sequence that is at least 65%, at least 70%, at least 75%, at least 80%, at least 85%, at least 90%, at least 95%, at least 97%, at least 98%, or at least 99% identical to any one of the sequences of TABLE 1, and wherein each effector partner comprises an amino acid sequence that is at least 65%, at least 70%, at least 75%, at least 80%, at least 85%, at least 90%, at least 95%, at least 97%, at least 98%, or at least 99% identical to any one of the sequences of TABLE 1.1.

Methods may comprise contacting a cell with an effector protein, or a nucleic acid (e.g., a plasmid or mRNA) encoding the effector protein, an effector partner, or a nucleic acid (e.g., a plasmid or mRNA) encoding the effector partner, and a guide nucleic acid or a nucleic acid (e.g., a plasmid or RNA) comprising a nucleotide sequence that encodes a guide nucleic acid described herein, wherein the effector protein comprises an amino acid sequence that is at least 65%, at least 70%, at least 75%, at least 80%, at least 85%, at least 90%, at least 95%, at least 97%, at least 98%, or at least 99% identical to any one of the sequences of TABLE 1, and the effector partner comprises an amino acid sequence that is at least 65%, at least 70%, at least 75%, at least 80%, at least 85%, at least 90%, at least 95%, at least 97%, at least 98%, or at least 99% identical to any one of the sequences of TABLE 1.1. Such methods include contacting a cell with an RNP complex as described herein.

Methods of the disclosure may be performed in a subject. Compositions of the disclosure may be administered to a subject. A subject may be a human. A subject may be a mammal (e.g., rat, mouse, cow, dog, pig, sheep, horse). A subject may be a vertebrate or an invertebrate. A subject may be a laboratory animal. A subject may be a patient. A subject may be at risk of developing, suffering from, or displaying symptoms a disease or disorder as set forth in herein. The subject may have a mutation associated with a gene described herein. The subject may display symptoms associated with a mutation of a gene described herein. In some embodiments, a mutation comprises a point mutation or single nucleotide polymorphism (SNP), a chromosomal mutation, a copy number mutation, or any combination thereof. A point mutation optionally comprises a substitution, insertion, or deletion. In some embodiments, a mutation comprises a chromosomal mutation. A chromosomal mutation may comprise an inversion, a deletion, a duplication, or a translocation. In some embodiments, a mutation comprises a copy number variation. A copy number variation may comprise a gene amplification or an expanding trinucleotide repeat. In some embodiments, mutations may be as described herein.

Methods of the disclosure may be performed in a cell. A cell may be in vitro. A cell may be in vivo. A cell may be ex vivo. A cell may be an isolated cell. A cell may be a cell inside of an organism. A cell may be an organism. A cell may be a cell in a cell culture. A cell may be one of a collection of cells. A cell may be a mammalian cell or derived from a mammalian cell. A cell may be a rodent cell or derived from a rodent cell. A cell may be a human cell or derived from a human cell. A cell may be a eukaryotic cell or derived from a eukaryotic cell. A cell may be a pluripotent stem cell. A cell may be a plant cell or derived from a plant cell. A cell may be an animal cell or derived from an animal cell. A cell may be an invertebrate cell or derived from an invertebrate cell. A cell may be a vertebrate cell or derived from a vertebrate cell.

A cell may be from a specific organ or tissue. The tissue may be muscle. The muscle may be skeletal muscle. In some embodiments, skeletal muscles include the following: abductor digiti minimi (foot), abductor digiti minimi (hand), abductor hallucis, abductor pollicis brevis, abductor pollicis longus, adductor brevis, adductor hallucis, adductor longus, adductor magnus, adductor pollicis, anconeus, articularis cubiti, articularis genu, aryepiglotticus, auricularis, biceps brachii, biceps femoris, brachialis, brachioradialis, buccinator, bulbospongiosus, constrictor of pharynx-inferior, constrictor of pharynx-middle, constrictor of pharynx-superior, coracobrachialis, corrugator supercilii, cremaster, cricothyroid, dartos, deep transverse perinei, deltoid, depressor anguli oris, depressor labii inferioris, diaphragm, digastric, digastric (anterior view), erector spinae—spinalis, erector spinae—iliocostalis, erector spinae—longissimus, extensor carpi radialis brevis, extensor carpi radialis longus, extensor carpi ulnaris, extensor digiti minimi (hand), extensor digitorum (hand), extensor digitorum brevis (foot), extensor digitorum longus (foot), extensor hallucis brevis, extensor hallucis longus, extensor indicis, extensor pollicis brevis, extensor pollicis longus, external oblique abdominis, flexor carpi radialis, flexor carpi ulnaris, flexor digiti minimi brevis (foot), flexor digiti minimi brevis (hand), flexor digitorum brevis, flexor digitorum longus (foot), flexor digitorum profundus, flexor digitorum superficialis, flexor hallucis brevis, flexor hallucis longus, flexor pollicis brevis, flexor pollicis longus, frontalis, gastrocnemius, gemellus inferior, gemellus superior, genioglossus, geniohyoid, gluteus maximus, gluteus medius, gluteus minimus, gracilis, hyoglossus, iliacus, inferior oblique, inferior rectus, infraspinatus, intercostals external, intercostals innermost, intercostals internal, internal oblique abdominis, interossei—dorsal of hand, interossei-dorsal of foot, interossei-palmar of hand, interossei—plantar of foot, interspinales, intertransversarii, intrinsic muscles of tongue, ishiocavernosus, lateral cricoarytenoid, lateral pterygoid, lateral rectus, latissimus dorsi, levator anguli oris, levator ani-coccygeus, levator ani—iliococcygeus, levator ani-pubococcygeus, levator ani-puborectalis, levator ani-pubovaginalis, levator labii superioris, levator labii superioris, alaeque nasi, levator palpebrae superioris, levator scapulae, levator veli palatini, levatores costarum, longus capitis, longus colli, lumbricals of foot, lumbricals of hand, masseter, medial pterygoid, medial rectus, mentalis, m. uvulae, mylohyoid, nasalis, oblique arytenoid, obliquus capitis inferior, obliquus capitis superior, obturator externus, obturator internus (A), obturator internus (B), omohyoid, opponens digiti minimi (hand), opponens pollicis, orbicularis oculi, orbicularis oris, palatoglossus, palatopharyngeus, palmaris brevis, palmaris longus, pectineus, pectoralis major, pectoralis minor, peroneus brevis, peroneus longus, peroneus tertius, piriformis (A), piriformis (B), plantaris, platysma, popliteus, posterior cricoarytenoid, procerus, pronator quadratus, pronator teres, psoas major, psoas minor, pyramidalis, quadratus femoris, quadratus lumborum, quadratus plantae, rectus abdominis, rectus capitus anterior, rectus capitus lateralis, rectus capitus posterior major, rectus capitus posterior minor, rectus femoris, rhomboid major, rhomboid minor, risorius, salpingopharyngeus, sartorius, scalenus anterior, scalenus medius, scalenus minimus, scalenus posterior, semimembranosus, semitendinosus, serratus anterior, serratus posterior inferior, serratus posterior superior, soleus, sphincter ani, sphincter urethrae, splenius capitis, splenius cervicis, stapedius, sternocleidomastoid, sternohyoid, sternothyroid, styloglossus, stylohyoid, stylohyoid (anterior view), stylopharyngeus, subclavius, subcostalis, subscapularis, superficial transverse perinei, superior oblique, superior rectus, supinator, supraspinatus, temporalis, temporoparietalis, tensor fasciae lata, tensor tympani, tensor veli palatini, teres major, teres minor, thyro-arytenoid & vocalis, thyro-epiglotticus, thyrohyoid, tibialis anterior, tibialis posterior, transverse arytenoid, transversospinalis-multifidus, transversospinalis-rotatores, transversospinalis-semispinalis, transversus abdominis, transversus thoracis, trapezius, triceps, vastus intermedius, vastus lateralis, vastus medialis, zygomaticus major, or zygomaticus minor. In some embodiments, the cell is a myocyte. In some embodiments, the cell is a muscle cell. In some embodiments, the muscle cell is a skeletal muscle cell. In some embodiments, the skeletal muscle cell is a red (slow) skeletal muscle cell, a white (fast) skeletal muscle cell or an intermediate skeletal muscle cell.

The tissue may be the subject's blood, bone marrow, or cord blood. The tissue may be heterologous donor blood, cord blood, or bone marrow. The tissue may be allogenic blood, cord blood, or bone marrow. In some embodiments, the cell is a: a stem cell, muscle satellite cell, muscle stem cell, myoblast, muscle progenitor cell, a pluripotent stem cell or a cell derived from a pluripotent stem cell.

### XIII. METHODS OF DETECTING A TARGET NUCLEIC ACID

Provided herein are methods of detecting target nucleic acids. Methods may comprise detecting target nucleic acids with compositions or systems described herein. Methods may comprise detecting a target nucleic acid in a sample, e.g., a cell lysate, a biological fluid, or environmental sample. Methods may comprise detecting a target nucleic acid in a cell. In some embodiments, methods of detecting a target nucleic acid in a sample or cell comprises contacting the sample or cell with an effector protein or a multimeric complex thereof, a guide nucleic acid, wherein at least a portion of the guide nucleic acid is complementary to at least a portion of the target nucleic acid, and a reporter nucleic acid that is cleaved in the presence of the effector protein, the guide nucleic acid, and the target nucleic acid, and detecting a signal produced by cleavage of the reporter nucleic acid, thereby detecting the target nucleic acid in the sample. In some embodiments, methods result in trans cleavage of the reporter nucleic acid. In some embodiments, methods result in cis cleavage of the reporter nucleic acid.

In some embodiments, methods of detecting comprise contacting a target nucleic acid, a cell comprising the target nucleic acid, or a sample comprising a target nucleic acid with an effector protein that comprises an amino acid sequence that is at least is at least 65%, at least 70%, at least 75%, at least 80%, at least 85%, at least 90%, at least 92%, at least 95%, at least 97%, at least 98%, at least 99%, or 100% identical to any one of the sequences of TABLE 1.

Methods may comprise contacting the sample to a complex comprising a guide nucleic acid comprising a segment that is reverse complementary to a segment of the target nucleic acid and an effector protein that exhibits sequence independent cleavage upon forming a complex comprising the segment of the guide nucleic acid binding to the segment of the target nucleic acid; and assaying for a signal indicating cleavage of at least some protein-nucleic acids of a population of protein-nucleic acids, wherein the signal indicates a presence of the target nucleic acid in the sample and wherein absence of the signal indicates an absence of the target nucleic acid in the sample.

Methods may comprise contacting the sample comprising the target nucleic acid with a guide nucleic acid targeting a target nucleic acid segment, an effector protein capable of being activated when complexed with the guide nucleic acid and the target nucleic acid segment, a single stranded nucleic acid of a reporter comprising a detection moiety, wherein the nucleic acid of a reporter is capable of being cleaved by the activated effector protein, thereby generating a first detectable signal, cleaving the single stranded nucleic acid of a reporter using the effector protein that cleaves as measured by a change in color, and measuring the first detectable signal on the support medium.

Methods may comprise contacting the sample or cell with an effector protein or a multimeric complex thereof and a guide nucleic acid at a temperature of at least about 25° C., at least about 30° C., at least about 35° C., at least about 40° C., at least about 50° C., or at least about 65° C. In some embodiments, the temperature is not greater than 80° C. In some embodiments, the temperature is about 25° C., about 30° C., about 35° C., about 40° C., about 45° C., about 50° C., about 55° C., about 60° C., about 65° C., or about 70° C. In some embodiments, the temperature is about 25° C. to about 45° C., about 35° C. to about 55° C., or about 55° C. to about 65° C.

Methods may comprise cleaving a strand of a single-stranded target nucleic acid with an effector protein or a multimeric complex thereof, as assessed with an in vitro cis-cleavage assay. A cleavage assay may comprise an assay designed to visualize, quantitate or identify cleavage of a nucleic acid. In some embodiments, the cleavage activity may be cis-cleavage activity. In some embodiments, the cleavage activity may be trans-cleavage activity. An example of such an assay (an in vitro cis-cleavage assay). An example of such an assay may follow a procedure comprising: (i) providing equimolar amounts of an effector protein comprising at least 70% sequence identity to any one of the sequences set forth in TABLE 1, optionally an effector partner comprising at least 70% sequence identity to any one of the sequences set forth in TABLE 1.1, and a guide nucleic acid, under conditions to form a ribonucleoprotein complex; (ii) adding linear dsDNA comprising a nucleic acid sequence targeted by the guide nucleic acid and adjacent to a PAM (iii) incubating the mixture under conditions to enable cleavage of the plasmid; (iv) quenching the reaction with EDTA and a protease; and (v) analyzing the reaction products (e.g., viewing the cleaved and uncleaved linear dsDNA with gel electrophoresis).

In some embodiments, there is a threshold of detection for methods of detecting target nucleic acids. In some embodiments, methods are not capable of detecting target nucleic acids that are present in a sample or solution at a concentration less than or equal to 10 nM. The term “threshold of detection” is used herein to describe the minimal amount of target nucleic acid that must be present in a sample in order for detection to occur. For example, when a threshold of detection is 10 nM, then a signal may be detected when a target nucleic acid is present in the sample at a concentration of 10 nM or more. In some embodiments, the threshold of detection is less than or equal to 5 nM, 1 nM, 0.5 nM, 0.1 nM, 0.05 nM, 0.01 nM, 0.005 nM, 0.001 nM, 0.0005 nM, 0.0001 nM, 0.00005 nM, 0.00001 nM, 10 pM, 1 pM, 500 fM, 250 fM, 100 fM, 50 fM, 10 fM, 5 fM, 1 fM, 500 attomole (aM), 100 aM, 50 aM, 10 aM, or 1 aM. In some embodiments, the threshold of detection is in a range of from 1 aM to 1 nM, 1 aM to 500 pM, 1 aM to 200 pM, 1 aM to 100 pM, 1 aM to 10 pM, 1 aM to 1 pM, 1 aM to 500 fM, 1 aM to 100 fM, 1 aM to 1 fM, 1 aM to 500 aM, 1 aM to 100 aM, 1 aM to 50 aM, 1 aM to 10 aM, 10 aM to 1 nM, 10 aM to 500 pM, 10 aM to 200 pM, 10 aM to 100 pM, 10 aM to 10 pM, 10 aM to 1 pM, 10 aM to 500 fM, 10 aM to 100 fM, 10 aM to 1 fM, 10 aM to 500 aM, 10 aM to 100 aM, 10 aM to 50 aM, 100 aM to 1 nM, 100 aM to 500 pM, 100 pM to 200 pM, 100 aM to 100 pM, 100 aM to 10 pM, 100 aM to 1 pM, 100 aM to 500 fM, 100 aM to 100 fM, 100 aM to 1 fM, 100 aM to 500 aM, 500 aM to 1 nM, 500 aM to 500 pM, 500 aM to 200 pM, 500 aM to 100 pM, 500 aM to 10 pM, 500 aM to 1 pM, 500 aM to 500 fM, 500 aM to 100 fM, 500 aM to 1 fM, 1 fM to 1 nM, 1 fM to 500 pM, 1 fM to 200 pM, 1 fM to 100 pM, 1 fM to 10 pM, 1 fM to 1 pM, 10 fM to 1 nM, 10 fM to 500 pM, 10 fM to 200 pM, 10 fM to 100 pM, 10 fM to 10 pM, 10 fM to 1 pM, 500 fM to 1 nM, 500 fM to 500 pM, 500 fM to 200 pM, 500 fM to 100 pM, 500 fM to 10 pM, 500 fM to 1 pM, 800 fM to 1 nM, 800 fM to 500 pM, 800 fM to 200 pM, 800 fM to 100 pM, 800 fM to 10 pM, 800 fM to 1 pM, 1 pM to 1 nM, 1 pM to 500 pM, 1 pM to 200 pM, 1 pM to 100 pM, or 1 pM to 10 pM. In some embodiments, the threshold of detection in a range of from 800 fM to 100 pM, 1 pM to 10 pM, 10 fM to 500 fM, 10 fM to 50 fM, 50 fM to 100 fM, 100 fM to 250 fM, or 250 fM to 500 fM. In some embodiments, the threshold of detection is in a range of from 2 aM to 100 pM, from 20 aM to 50 pM, from 50 aM to 20 pM, from 200 aM to 5 pM, or from 500 aM to 2 pM. In some embodiments, the minimum concentration at which a target nucleic acid is detected in a sample is in a range of from 1 aM to 1 nM, 10 aM to 1 nM, 100 aM to 1 nM, 500 aM to 1 nM, 1 fM to 1 nM, 1 fM to 500 pM, 1 fM to 200 pM, 1 fM to 100 pM, 1 fM to 10 pM, 1 fM to 1 pM, 10 fM to 1 nM, 10 fM to 500 pM, 10 fM to 200 pM, 10 fM to 100 pM, 10 fM to 10 pM, 10 fM to 1 pM, 500 fM to 1 nM, 500 fM to 500 pM, 500 fM to 200 pM, 500 fM to 100 pM, 500 fM to 10 pM, 500 fM to 1 pM, 800 fM to 1 nM, 800 fM to 500 pM, 800 fM to 200 pM, 800 fM to 100 pM, 800 fM to 10 pM, 800 fM to 1 pM, 1 pM to 1 nM, 1 pM to 500 pM, from 1 pM to 200 pM, 1 pM to 100 pM, or 1 pM to 10 pM. In some embodiments, the minimum concentration at which a target nucleic acid is detected in a sample is in a range of from 2 aM to 100 pM, from 20 aM to 50 pM, from 50 aM to 20 pM, from 200 aM to 5 pM, or from 500 aM to 2 pM. In some embodiments, the minimum concentration at which a single stranded target nucleic acid may be detected in a sample is in a range of from 1 aM to 100 pM. In some embodiments, the minimum concentration at which a target nucleic acid may be detected in a sample is in a range of from 1 fM to 100 pM. In some embodiments, the minimum concentration at which a single stranded target nucleic acid may be detected in a sample is in a range of from 10 fM to 100 pM. In some embodiments, the minimum concentration at which a single stranded target nucleic acid may be detected in a sample is in a range of from 800 fM to 100 pM. In some embodiments, the minimum concentration at which a single stranded target nucleic acid may be detected in a sample is in a range of from 1 pM to 10 pM. In some embodiments, the devices, systems, fluidic devices, kits, and methods described herein detect a target single-stranded nucleic acid in a sample comprising a plurality of nucleic acids such as a plurality of non-target nucleic acids, where the target single-stranded nucleic acid is present at a concentration as low as 1 aM, 10 aM, 100 aM, 500 aM, 1 fM, 10 fM, 500 fM, 800 fM, 1 pM, 10 pM, 100 pM, or 1 pM.

In some embodiments, the target nucleic acid is present in a cleavage reaction at a concentration of about 10 nM, about 20 nM, about 30 nM, about 40 nM, about 50 nM, about 60 nM, about 70 nM, about 80 nM, about 90 nM, about 100 nM, about 200 nM, about 300 nM, about 400 nM, about 500 nM, about 600 nM, about 700 nM, about 800 nM, about 900 nM, about 1 μM, about 10 μM, or about 100 μM. In some embodiments, the target nucleic acid is present in the cleavage reaction at a concentration of from 10 nM to 20 nM, from 20 nM to 30 nM, from 30 nM to 40 nM, from 40 nM to 50 nM, from 50 nM to 60 nM, from 60 nM to 70 nM, from 70 nM to 80 nM, from 80 nM to 90 nM, from 90 nM to 100 nM, from 100 nM to 200 nM, from 200 nM to 300 nM, from 300 nM to 400 nM, from 400 nM to 500 nM, from 500 nM to 600 nM, from 600 nM to 700 nM, from 700 nM to 800 nM, from 800 nM to 900 nM, from 900 nM to 1 μM, from 1 μM to 10 μM, from 10 μM to 100 μM, from 10 nM to 100 nM, from 10 nM to 1 μM, from 10 nM to 10 μM, from 10 nM to 100 μM, from 100 nM to 1 μM, from 100 nM to 10 μM, from 100 nM to 100 μM, or from 1 μM to 100 μM. In some embodiments, the target nucleic acid is present in the cleavage reaction at a concentration of from 20 nM to 50 μM, from 50 nM to 20 μM, or from 200 nM to 5 μM.

In some embodiments, methods detect a target nucleic acid in less than 60 minutes. In some embodiments, methods detect a target nucleic acid in less than about 120 minutes, less than about 110 minutes, less than about 100 minutes, less than about 90 minutes, less than about 80 minutes, less than about 70 minutes, less than about 60 minutes, less than about 55 minutes, less than about 50 minutes, less than about 45 minutes, less than about 40 minutes, less than about 35 minutes, less than about 30 minutes, less than about 25 minutes, less than about 20 minutes, less than about 15 minutes, less than about 10 minutes, less than about 5 minutes, less than about 4 minutes, less than about 3 minutes, less than about 2 minutes, or less than about 1 minute.

In some embodiments, methods require at least about 120 minutes, at least about 110 minutes, at least about 100 minutes, at least about 90 minutes, at least about 80 minutes, at least about 70 minutes, at least about 60 minutes, at least about 55 minutes, at least about 50 minutes, at least about 45 minutes, at least about 40 minutes, at least about 35 minutes, at least about 30 minutes, at least about 25 minutes, at least about 20 minutes, at least about 15 minutes, at least about 10 minutes, or at least about 5 minutes to detect a target nucleic acid. In some embodiments, the sample is contacted with the reagents for from 5 minutes to 120 minutes, from 5 minutes to 100 minutes, from 10 minutes to 90 minutes, from 15 minutes to 45 minutes, or from 20 minutes to 35 minutes.

In some embodiments, methods of detecting are performed in less than 10 hours, less than 9 hours, less than 8 hours, less than 7 hours, less than 6 hours, less than 5 hours, less than 4 hours, less than 3 hours, less than 2 hours, less than 1 hour, less than 50 minutes, less than 45 minutes, less than 40 minutes, less than 35 minutes, less than 30 minutes, less than 25 minutes, less than 20 minutes, less than 15 minutes, less than 10 minutes, less than 9 minutes, less than 8 minutes, less than 7 minutes, less than 6 minutes, or less than 5 minutes. In some embodiments, methods of detecting are performed in about 5 minutes to about 10 hours, about 10 minutes to about 8 hours, about 15 minutes to about 6 hours, about 20 minutes to about 5 hours, about 30 minutes to about 2 hours, or about 45 minutes to about 1 hour.

Methods may comprise detecting a detectable signal within 5 minutes of contacting the sample and/or the target nucleic acid with the guide nucleic acid and/or the effector protein. In some embodiments, detecting occurs within 1, 2, 3, 4, 5, 6, 7, 8, 9, 10, 11, 12, 13, 14, 15, 20, 25, 30, 35, 40, 45, 50, 55, 60, 70, 80, 90, 100, 110, or 120 minutes of contacting the target nucleic acid. In some embodiments, detecting occurs within 1 to 120, 5 to 100, 10 to 90, 15 to 80, 20 to 60, or 30 to 45 minutes of contacting the target nucleic acid.

Amplification

Methods of detecting may comprise amplifying a target nucleic acid for detection using any of the compositions or systems described herein. Amplifying may comprise changing the temperature of the amplification reaction, also known as thermal amplification (e.g., PCR). Amplifying may be performed at essentially one temperature, also known as isothermal amplification. Amplifying may improve at least one of sensitivity, specificity, or accuracy of the detection of the target nucleic acid.

Amplifying may comprise subjecting a target nucleic acid to an amplification reaction selected from transcription mediated amplification (TMA), helicase dependent amplification (HDA), or circular helicase dependent amplification (cHDA), strand displacement amplification (SDA), recombinase polymerase amplification (RPA), loop mediated amplification (LAMP), exponential amplification reaction (EXPAR), rolling circle amplification (RCA), ligase chain reaction (LCR), simple method amplifying RNA targets (SMART), single primer isothermal amplification (SPIA), multiple displacement amplification (MDA), nucleic acid sequence based amplification (NASBA), hinge-initiated primer-dependent amplification of nucleic acids (HIP), nicking enzyme amplification reaction (NEAR), and improved multiple displacement amplification (IMDA).

In some embodiments, amplification of the target nucleic acid comprises modifying the sequence of the target nucleic acid. For example, amplification may be used to insert a PAM sequence into a target nucleic acid that lacks a PAM sequence. In some embodiments, amplification may be used to increase the homogeneity of a target nucleic acid in a sample. For example, amplification may be used to remove a nucleic acid variation that is not of interest in the target nucleic acid.

Amplifying may take 1, 2, 3, 4, 5, 6, 7, 8, 9, 10, 11, 12, 13, 14, 15, 16, 17, 18, 19, 20, 25, 30, 40, 50, or 60 minutes. Amplifying may be performed at a temperature of around 20-45° C. Amplifying may be performed at a temperature of less than about 20° C., less than about 25° C., less than about 30° C., 35° C., less than about 37° C., less than about 40° C., or less than about 45° C. The nucleic acid amplification reaction may be performed at a temperature of at least about 20° C., at least about 25° C., at least about 30° C., at least about 35° C., at least about 37° C., at least about 40° C., or at least about 45° C.

### XIV. METHODS OF TREATING A DISORDER

Described herein are compositions, systems and methods for treating a disease in a subject by modifying a target nucleic acid associated with a gene or expression of a gene related to the disease. In some embodiments, methods comprise administering a composition or cell described herein to a subject. By way of non-limiting example, the disease may be a cancer, an ophthalmological disorder, a neurological disorder, a neurodegenerative disease, a blood disorder, or a metabolic disorder, or a combination thereof. The disease may be an inherited disorder, also referred to as a genetic disorder. The disease may be the result of an infection or associated with an infection.

The compositions, systems and methods described herein may be used to treat, prevent, or inhibit a disease or syndrome in a subject. In some embodiments, the disease is a liver disease, a lung disease, an eye disease, or a muscle disease. Exemplary diseases and syndromes include, but are not limited to the diseases and syndromes listed in TABLE 5. In some embodiments, the disease is a genetic disorder. In some embodiments, the genetic disorder is a neurological disorder. In some embodiments, gene is a human gene. In some embodiments, the human gene is a gene recited in TABLE 4.

In some embodiments, compositions, systems and methods modify at least one gene associated with the disease or the expression thereof. In some embodiments, the disease is Alzheimer's disease and the gene is selected from APP, BACE-1, PSD95, MAPT, PSEN1, PSEN2, and APOEc4. In some embodiments, the disease is Parkinson's disease and the gene is selected from SNCA, GDNF, and LRRK2. In some embodiments, the disease comprises Centronuclear myopathy and the gene is DNM2. In some embodiments, the disease is Huntington's disease and the gene is HTT. In some embodiments, the disease is Alpha-1 antitrypsin deficiency (AATD) and the gene is SERPINAl. In some embodiments, the disease is amyotrophic lateral sclerosis (ALS) and the gene is selected from SOD1, FUS, C9ORF72, ATXN2, TARDBP, and CHCHD10. In some embodiments, the disease comprises Alexander Disease and the gene is GFAP. In some embodiments, the disease comprises anaplastic large cell lymphoma and the gene is CD30. In some embodiments, the disease comprises Angelman Syndrome and the gene is UBE3A. In some embodiments, the disease comprises calcific aortic stenosis and the gene is Apo(a). In some embodiments, the disease comprises CD3Z-associated primary T-cell immunodeficiency and the gene is CD3Z or CD247. In some embodiments, the disease comprises CD18 deficiency and the gene is ITGB2. In some embodiments, the disease comprises CD40L deficiency and the gene is CD40L. In some embodiments, the disease is congenital adrenal hyperplasia and the gene is CAH1. In some embodiments, the disease comprises CNS trauma and the gene is VEGF. In some embodiments, the disease comprises coronary heart disease and the gene is selected from FGA, FGB, and FGG. In some embodiments, the disease comprises MECP2 Duplication syndrome and Rett syndrome and the gene is MECP2. In some embodiments, the disease comprises a bleeding disorder (coagulation) and the gene is FXI. In some embodiments, the disease comprises fragile X syndrome and the gene is FMR1. In some embodiments, the disease comprises Fuchs corneal dystrophy and the gene is selected from ZEB1, SLC4A11, and LOXHD1. In some embodiments, the disease comprises GM2-Gangliosidoses (e.g., Tay Sachs Disease, Sandhoff disease) and the gene is selected from HEXA and HEXB. In some embodiments, the disease comprises Hearing loss disorders and the gene is DFNA36. In some embodiments, the disease is Pompe disease, including infantile onset Pompe disease (IOPD) and late onset Pompe disease (LOPD) and the gene is GAA. In some embodiments, the disease is Retinitis pigmentosa and the gene is selected from PDE6B, RHO, RP1, RP2, RPGR, PRPH2, IMPDH1, PRPF31, CRB1, PRPF8, TULP1, CA4, HPRPF3, ABCA4, EYS, CERKL, FSCN2, TOPORS, SNRNP200, PRCD, NR2E3, MERTK, USH2A, PROM1, KLHL7, CNGB1, TTC8, ARL6, DHDDS, BEST1, LRAT, SPARA7, CRX, CLRN1, RPE65, and WDR19. In some embodiments, the disease comprises Leber Congenital Amaurosis Type 10 and the gene is CEP290. In some embodiments, the disease is cardiovascular disease and/or lipodystrophies and the gene is selected from ABCG5, ABCG8, AGT, ANGPTL3, APOCIII, APOA1, APOL1, ARH, CDKN2B, CFB, CXCL12, FXI, FXII, GATA-4, MIA3, MKL2, MTHFD1L, MYH7, NKX2-5, NOTCH1, PKK, PCSK9, PSRC1, SMAD3, and TTR. In some embodiments, the disease comprises acromegaly and the gene is GHR. In some embodiments, the disease comprises acute myeloid leukemia and the gene is CD22. In some embodiments, the disease is diabetes and the gene is GCGR. In some embodiments, the disease is NAFLD/NASH and the gene is selected from DGAT2 and PNPLA3. In some embodiments, the disease is cancer and the gene is selected from STAT3, YAP1, FOXP3, AR (Prostate cancer), and IRF4 (multiple myeloma). In some embodiments, the disease is cystic fibrosis and the gene is CFTR. In some embodiments, the disease is Duchenne muscular dystrophy and the gene is DMD. In some embodiments, the disease is ornithine transcarbamylase deficiency and the gene is OTC. In some embodiments, the disease comprises angioedema and the gene is PKK. In some embodiments, the disease comprises thalassemia and the gene is TMPRSS6. In some embodiments, the disease comprises achondroplasia and the gene is FGFR3. In some embodiments, the disease comprises Cri du chat syndrome and the gene is selected from CTNND2. In some embodiments, the disease comprises sickle cell anemia and the gene is Beta globin gene. In some embodiments, the disease comprises Alagille Syndrome and the gene is selected from JAG1 and NOTCH2. In some embodiments, the disease comprises Charcot-Marie-Tooth disease and the gene is selected from PMP22 and MFN2. In some embodiments, the disease comprises Crouzon syndrome and the gene is selected from FGFR2, FGFR3, and FGFR3. In some embodiments, the disease comprises Dravet Syndrome and the gene is selected from SCN1A and SCN2A. In some embodiments, the disease comprises Emery-Dreifuss syndrome and the gene is selected from EMD, LMNA, SYNE1, SYNE2, FHL1, and TMEM43. In some embodiments, the disease comprises Factor V Leiden thrombophilia and the gene is F5. In some embodiments, the disease is fabry disease and the gene is GLA. In some embodiments, the disease is facioscapulohumeral muscular dystrophy and the gene is FSHDJ. In some embodiments, the disease comprises Fanconi anemia and the gene is selected from FANCA, FANCB, FANCC, FANCD1, FANCD2, FANCE, FANCF, FANCG, FANCI, FANCJ, FANCL, FANCM, FANCN, FANCP, FANCS, RAD51C, and XPF. In some embodiments, the disease comprises Familial Creutzfeld—Jakob disease and the gene is PRNP. In some embodiments, the disease comprises Familial Mediterranean Fever and the gene is MEFV. In some embodiments, the disease comprises Friedreich's ataxia and the gene is FXN. In some embodiments, the disease comprises Gaucher disease and the gene is GBA. In some embodiments, the disease comprises human papilloma virus (HPV) infection and the gene is HPV E7. In some embodiments, the disease comprises hemochromatosis and the gene is HFE, optionally comprising a C282Y mutation. In some embodiments, the disease comprises Hemophilia A and the gene is FVIII. In some embodiments, the disease is hereditary angioedema and the gene is SERPING1. In some embodiments, the disease comprises histiocytosis and the gene is CD1. In some embodiments, the disease comprises immunodeficiency 17 and the gene is CD3D. In some embodiments, the disease comprises immunodeficiency 13 and the gene is CD4. In some embodiments, the disease comprises Common Variable Immunodeficiency and the gene is selected from CD19 and CD81. In some embodiments, the disease comprises Joubert syndrome and the gene is selected from INPP5E, TMEM216, AHI1, NPHP1, CEP290, TMEM67, RPGRIP1L, ARL13B, CC2D2A, OFD1, TMEM138, TCTN3, ZNF423, and AMRC9. In some embodiments, the disease comprises leukocyte adhesion deficiency and the gene is CD18. In some embodiments, the disease comprises Li—Fraumeni syndrome and the gene is TP53. In some embodiments, the disease comprises lymphoproliferative syndrome and the gene is CD27. In some embodiments, the disease comprises Lynch syndrome and the gene is selected from MSH2, MLH1, MSH6, PMS2, PMS1, TGFBR2, and MLH3. In some embodiments, the disease comprises mantle cell lymphoma and the gene is CD5. In some embodiments, the disease comprises Marfan syndrome and the gene is FBN1. In some embodiments, the disease comprises mastocytosis and the gene is CD2. In some embodiments, the disease comprises methylmalonic acidemia and the gene is selected from MMAA, MMAB, and MUT. In some embodiments, the disease is mycosis fungoides and the gene is CD7. In some embodiments, the disease is myotonic dystrophy and the gene is selected from CNBP and DMPK. In some embodiments, the disease comprises neurofibromatosis and the gene is selected from NF1, and NF2. In some embodiments, the disease comprises osteogenesis imperfecta and the gene is selected from COL1A1, COL1A2, and IFITM5. In some embodiments, the disease is non-small cell lung cancer and the gene is selected from KRAS, EGFR, ALK, METex14, BRAF V600E, ROS1, RET, and NTRK. In some embodiments, the disease comprises Peutz—Jeghers syndrome and the gene is STK11. In some embodiments, the disease comprises polycystic kidney disease and the gene is selected from PKD1 and PKD2. In some embodiments, the disease comprises Severe Combined Immune Deficiency and the gene is selected from IL7R, RAG1, and JAK3. In some embodiments, the disease comprises PRKAG2 cardiac syndrome and the gene is PRKAG2. In some embodiments, the disease comprises spinocerebellar ataxia and the gene is selected from ATXN1, ATXN2, ATXN3, PLEKHG4, SPTBN2, CACNA1A, ATXN7, ATXN8OS, ATXN10, TTBK2, PPP2R2B, KCNC3, PRKCG, ITPR1, TBP, KCND3, and FGF14. In some embodiments, the disease is thrombophilia due to antithrombin III deficiency and the gene is SERPINC1. In some embodiments the disease is spinal muscular atrophy and the gene is SMNJ. In some embodiments, the disease comprises Usher Syndrome and the gene is selected from MYO7A, USH1C, CDH23, PCDH15, USH1G, USH2A, GPR98, DFNB31, and CLRN1. In some embodiments, the disease comprises von Willebrand disease and the gene is VWF. In some embodiments, the disease comprises Waardenburg syndrome and the gene is selected from PAX3, MITF, WS2B, WS2C, SNAI2, EDNRB, EDN3, and SOX10. In some embodiments, the disease comprises Wiskott-Aldrich Syndrome and the gene is WAS. In some embodiments, the disease comprises von Hippel—Lindau disease and the gene is VHL. In some embodiments, the disease comprises Wilson disease and the gene is ATP7B. In some embodiments, the disease comprises Zellweger syndrome and the gene is selected from PEX1, PEX2, PEX3, PEXS, PEX6, PEX10, PEX12, PEX13, PEX14, PEX16, PEX19, and PEX26. In some embodiments, the disease comprises infantile myofibromatosis and the gene is CD34. In some embodiments, the disease comprises platelet glycoprotein IV deficiency and the gene is CD36. In some embodiments, the disease comprises immunodeficiency with hyper-IgM type 3 and the gene is CD40. In some embodiments, the disease comprises hemolytic uremic syndrome and the gene is CD46.

In some embodiments, the disease comprises complement hyperactivation, angiopathic thrombosis, or protein-losing enteropathy and the gene is CD55. In some embodiments, the disease comprises hemolytic anemia and the gene is CD59. In some embodiments, the disease comprises calcification of joints and arteries and the gene is CD73. In some embodiments, the disease comprises immunoglobulin alpha deficiency and the gene is CD79A. In some embodiments, the disease comprises C syndrome and the gene is CD96. In some embodiments, the disease comprises hairy cell leukemia and the gene is CD123. In some embodiments, the disease comprises histiocytic sarcoma and the gene is CD163. In some embodiments, the disease comprises autosomal dominant deafness and the gene is CD164. In some embodiments, the disease comprises immunodeficiency 25 and the gene is CD247. In some embodiments, the disease comprises methymalonic acidemia due to transcobalamin receptor defect and the gene is CD320.

Cancer

In some embodiments, the disease is cancer. Non-limiting examples of cancers include: acute lymphoblastic leukemia; acute lymphoblastic lymphoma; acute lymphocytic leukemia; acute myelogenous leukemia; acute myeloid leukemia (adult/childhood); adrenocortical carcinoma; anal cancer; appendix cancer; astrocytoma; atypical teratoid/rhabdoid tumor; basal-cell carcinoma; bile duct cancer; bladder cancer; bone osteosarcoma; brain cancer; brain tumor; brainstem glioma; breast cancer; bronchial adenoma, carcinoid, or tumor; Burkitt lymphoma; carcinomacervical cancer; chronic lymphocytic leukemia; chronic myelogenous leukemia; chronic myeloid leukemia; colon cancer; colorectal cancer; emphysema; endometrial cancer; esophageal cancer; Ewing sarcoma; gallbladder cancer; gastric (stomach) cancer; gastrointestinal tumor; gliomahairy cell leukemia; head and neck cancer; liver cancer; Hodgkin's lymphoma; hypopharyngeal cancer; Kaposi Sarcoma; kidney cancer lip and oral cavity cancer; liposarcoma; lung cancer, non-small cell lung cancer; WaldenstrOm; melanoma; mesotheliomamyelogenous leukemia; myeloid leukemia; myeloma; nasopharyngeal carcinoma; neuroblastoma; non-Hodgkin's lymphoma; ovarian cancer; pancreatic cancer; pineal cancer; pituitary tumor; prostate cancer; rectal cancer; renal cell carcinomaretinoblastoma; spinal cord tumor; squamous cell carcinoma; squamous neck cancer; T-cell lymphoma, cutaneous (Mycosis Fungoides and Sézary syndrome); testicular cancer; throat cancer; thyroid cancer; urethral cancer; uterine cancervaginal cancer; and Wilms Tumor. In some embodiments, the cancer is a solid cancer (i.e., a tumor). In some embodiments, the cancer is selected from a blood cell cancer, a leukemia, and a lymphoma. The cancer may be a leukemia, such as, by way of non-limiting example, acute myeloid (or myelogenous) leukemia (AML), chronic myeloid (or myelogenous) leukemia (CML), acute lymphocytic (or lymphoblastic) leukemia (ALL), and chronic lymphocytic leukemia (CLL). In some embodiments, the cancer is any one of colon cancer, rectal cancer, renal-cell carcinoma, liver cancer, bladder cancer, cancer of the kidney or ureter, lung cancer, non-small cell lung cancer, cancer of the small intestine, esophageal cancer, melanoma, bone cancer, pancreatic cancer, skin cancer, brain cancer (e.g., glioblastoma), cancer of the head or neck, melanoma, uterine cancer, ovarian cancer, breast cancer, testicular cancer, cervical cancer, stomach cancer, Hodgkin's Disease, non-Hodgkin's lymphoma, and thyroid cancer.

In some embodiments, mutations are associated with cancer or are causative of cancer. The target nucleic acid, in some embodiments, comprises a portion of a gene comprising a mutation associated with a disease, such as cancer, a gene whose overexpression is associated with cancer, a tumor suppressor gene, an oncogene, a checkpoint inhibitor gene, a gene associated with cellular growth, a gene associated with cellular metabolism, a gene associated with cell cycle, or a combination thereof. Non-limiting examples of genes comprising a mutation associated with a disease such as cancer are ABL, ACE, AF4/HRX, AKT-2, ALK, ALK/NPM, AML1, AML1/MTG8, APC, ATM, AXIN2, AXL, BAP1, BARD1, BCL-2, BCL-3, BCL-6, BCR/ABL, BLM, BMPR1A, BRCA1, BRCA2, BRIP1, c-MYC, CASR, CCR5, CDC73, CDH1, CDK4, CDKN1B, CDKN1C, CDKN2A, CEBPA, CHEK2, CREBBP, CTNNA1, DBL, DEK/CAN, DICER1, DIS3L2, E2A/PBX1, EGFR, ENL/HRX, EPCAM, ERG/TLS, ERBB, ERBB-2, ETS-1, EWS/FLI-1, FH, FKRP, FLCN, FMS, FOS, FPS, GATA2, GCG, GLI, GPC3, GPGSP, GREM1, HER2/neu, HOX11, HOXB13, HRAS, HST, IL-3, INT-2, JAK1, JUN, KIT, KS3, K-SAM, LBC, LCK, LMO1, LMO2, L-MYC, LYL-1, LYT-10, LYT-10/Ca 1, MAS, MAX, MDM-2, MEN1, MET, MITF, MLH1, MLL, MOS, MSH1, MSH2, MSH3, MSH6, MTG8/AML1, MUTYH, MYB, MYH11/CBFB, NBN, NEU, NF1, NF2, N-MYC, NTHL1, OST, PALB2, PAX-5, PBX1/E2A, PCDCl, PDGFRA, PHOX2B, PIM-1, PM S2, POLD1, POLE, POT1, PPARG, PRAD-1, PRKAR1A, PTCH1, PTEN, RAD 50, RAD51C, RAD51D, RAF, RAR/PML, RAS-H, RAS-K, RAS-N, RB1, RECQL4, REL/NRG, RET, RHOM1, RHOM2, ROS, RUNX1, SDHA, SDHAF, SDHAF2, SDHB, SDHC, SDHD, SET/CAN, SIS, SKI, SMAD4, SMARCA4, SMARCB1, SMARCE1, SRC, STK11, SUFU, TAL1, TAL2, TAN-1, TIAM1, TERC, TERT, TIMP3, TMEM127, TNF, TP53, TRAC, TSC1, TSC2, TRK, VHL, WRN, and WT1. Non-limiting examples of oncogenes are KRAS, NRAS, BRAF, MYC, CTNNB1, and EGFR. In some embodiments, the oncogene is a gene that encodes a cyclin dependent kinase (CDK). Non-limiting examples of CDKs are Cdkl, Cdk4, Cdk5, Cdk7, Cdk8, Cdk9, Cdk11 and CDK20. Non-limiting examples of tumor suppressor genes are TP53, RB1, and PTEN.

Infections

Described herein are compositions and methods for treating an infection in a subject. Infections may be caused by a pathogen, e.g., bacteria, viruses, fungi, and parasites. Compositions and methods may modify a target nucleic acid associated with the pathogen or parasite causing the infection. In some embodiments, the target nucleic acid may be in the pathogen or parasite itself or in a cell, tissue or organ of the subject that the pathogen or parasite infects. In some embodiments, the methods described herein include treating an infection caused by one or more bacterial pathogens. Non-limiting examples of bacterial pathogens include Acholeplasma laidlawii, Brucella abortus, Chlamydia psittaci, Chlamydia trachomatis, Cryptococcus neoformans, Escherichia coli, Legionella pneumophila, Lyme disease spirochetes, methicillin-resistant Staphylococcus aureus, Mycobacterium leprae, Mycobacterium tuberculosis, Mycoplasma arginini, Mycoplasma arthritidis, Mycoplasma genitalium, Mycoplasma hyorhinis, Mycoplasma orale, Mycoplasma pneumoniae, Mycoplasma salivarium, Neisseria gonorrhoeae, Neisseria meningitidis, Pneumococcus, Pseudomonas aeruginosa, sexually transmitted infection, Streptococcus agalactiae, Streptococcus pyogenes, and Treponema pallidum.

In some embodiments, methods described herein include treating an infection caused by one or more viral pathogens. Non-limiting examples of viral pathogens include adenovirus, blue tongue virus, chikungunya, coronavirus (e.g., SARS-CoV-2), cytomegalovirus, Dengue virus, Ebola, Epstein-Barr virus, feline leukemia virus, Hemophilus influenzae B, Hepatitis virus A, Hepatitis virus B, Hepatitis virus C, herpes simplex virus I, herpes simplex virus II, human papillomavirus (HPV) including HPV16 and HPV18, human serum parvo-like virus, human T-cell leukemia viruses, immunodeficiency virus (e.g., HIV), influenza virus, lymphocytic choriomeningitis virus, measles virus, mouse mammary tumor virus, mumps virus, murine leukemia virus, polio virus, rabies virus, Reovirus, respiratory syncytial virus (RSV), rubella virus, Sendai virus, simian virus 40, Sindbis virus, varicella-zoster virus, vesicular stomatitis virus, wart virus, West Nile virus, yellow fever virus, or any combination thereof.

In some embodiments, methods described herein include treating an infection caused by one or more parasites. Non-limiting examples of parasites include helminths, annelids, platyhelminthes, nematodes, and thorny-headed worms. In some embodiments, parasitic pathogens comprise, without limitation, Babesia bovis, Echinococcus granulosus, Eimeria tenella, Leishmania tropica, Mesocestoides corti, Onchocerca volvulus, Plasmodium falciparum, Plasmodium vivax, Schistosoma japonicum, Schistosoma mansoni, Schistosoma spp., Taenia hydatigena, Taenia ovis, Taenia saginata, Theileria parva, Toxoplasma gondii, Toxoplasma spp., Trichinella spiralis, Trichomonas vaginalis, Trypanosoma brucei, Trypanosoma cruzi, Trypanosoma rangeli, Trypanosoma rhodesiense, Balantidium coli, Entamoeba histolytica, Giardia spp., Isospora spp., Trichomonas spp., or any combination thereof.

### ILLUSTRATIVE EMBODIMENTS

The present disclosure provides the following illustrative embodiments.

Embodiment 1. A composition that comprises an isolated polypeptide, or a recombinant nucleic acid encoding the isolated polypeptide, wherein the isolated polypeptide comprises an amino acid sequence that is at least 75% identical to any one of the sequences set forth in TABLE 1.

Embodiment 2. A composition that comprises:
(i) a polypeptide, or a nucleic acid encoding the polypeptide, wherein the polypeptide comprises an amino acid sequence that is at least 75% identical to any one of the sequences set forth in TABLE 1; and
(ii) an engineered guide nucleic acid or a DNA molecule that encodes the engineered guide nucleic acid.

Embodiment 3. A composition that comprises:
(i) a polypeptide, or a nucleic acid encoding the polypeptide, wherein the polypeptide comprises an amino acid sequence that is at least 75% identical to any one of the sequences set forth in TABLE 1; and
(ii) a donor nucleic acid; and
(iii) an engineered guide nucleic acid or a nucleic acid that encodes the engineered guide nucleic acid.

Embodiment 4. A composition that comprises:
(i) an isolated polypeptide, or a recombinant nucleic acid encoding the isolated polypeptide, wherein the isolated polypeptide comprises an amino acid sequence that is at least 75% identical to any one of the sequences set forth in TABLE 1; and
(ii) one or more partner polypeptides or isolated partner polypeptides, or one or more recombinant nucleic acids encoding the one or more partner polypeptides or isolated partner polypeptides.

Embodiment 5. A composition that comprises:
(i) a polypeptide, or a nucleic acid encoding the polypeptide, wherein the polypeptide comprises an amino acid sequence that is at least 75% identical to any one of the sequences set forth in TABLE 1;
(ii) one or more partner polypeptides, or one or more nucleic acids encoding the one or more partner polypeptides; and
(iii) a nucleic acid, wherein the nucleic acid is a donor nucleic acid, or an engineered guide nucleic acid or a nucleic acid that encodes the engineered guide nucleic acid.

Embodiment 6. The composition of embodiment 5 wherein the nucleic acid is a donor nucleic acid.

Embodiment 7. The composition of embodiment 5, wherein the nucleic acid is an engineered guide nucleic acid or a nucleic acid that encodes the engineered guide nucleic acid.

Embodiment 8. A composition that comprises:
(i) a polypeptide, or a nucleic acid encoding the polypeptide, wherein the polypeptide comprises an amino acid sequence that is at least 75% identical to any one of the sequences set forth in TABLE 1;
(ii) one or more partner polypeptides, or one or more nucleic acids encoding the one or more partner polypeptides;
(iii) a donor nucleic acid; and
(iv) an engineered guide nucleic acid or a nucleic acid that encodes the engineered guide nucleic acid.

Embodiment 9. The composition of any one of embodiments 4-8, wherein the one or more partner polypeptide comprises an amino acid sequence that is at least 75% identical to any one of the sequences set forth in TABLE 1.1.

Embodiment 10. A composition that comprises one or more isolated partner polypeptides or one or more recombinant nucleic acids encoding the one or more isolated partner polypeptides wherein the one or more isolated partner polypeptide comprises an amino acid sequence that is at least 75% identical to any one of the sequences set forth in TABLE 1.1.

Embodiment 11. A composition that comprises:
(i) one or more partner polypeptides, or one or more nucleic acids encoding the one or more partner polypeptides, wherein the one or more partner polypeptide comprises an amino acid sequence that is at least 75% identical to any one of the sequences set forth in TABLE 1.1; and
(ii) a nucleic acid, wherein the nucleic acid is a donor nucleic acid, or an engineered guide nucleic acid or a nucleic acid that encodes the engineered guide nucleic acid.

Embodiment 12. The composition of embodiment 11, wherein the nucleic acid is a donor nucleic acid.

Embodiment 13. The composition of embodiment 11, wherein the nucleic acid is an engineered guide nucleic acid or a nucleic acid that encodes the engineered guide nucleic acid.

Embodiment 14. A composition that comprises:
(i) one or more partner polypeptides, or one or more nucleic acids encoding the one or more partner polypeptides, wherein the one or more partner polypeptide comprises an amino acid sequence that is at least 75% identical to any one of the sequences set forth in TABLE 1.1; and
(ii) a donor nucleic acid and
(iii) an engineered guide nucleic acid or a nucleic acid that encodes the engineered guide nucleic acid.

Embodiment 15. The composition of any one of embodiments 1-8, wherein the polypeptide comprises an amino acid sequence that is at least 80% identical to any one of the sequences set forth in TABLE 1.

Embodiment 16. The composition of any one of embodiments 1-8, wherein the polypeptide comprises an amino acid sequence that is at least 85% identical to any one of the sequences set forth in TABLE 1.

Embodiment 17. The composition of any one of embodiments 1-8, wherein the polypeptide comprises an amino acid sequence that is at least 90% identical to any one of the sequences set forth in TABLE 1.

Embodiment 18. The composition of any one of embodiments 1-8, wherein the polypeptide comprises an amino acid sequence that is at least 95% identical to any one of the sequences set forth in TABLE 1.

Embodiment 19. The composition of any one of embodiments 1-8, wherein the polypeptide comprises an amino acid sequence that is 100% identical to any one of the sequences set forth in TABLE 1.

Embodiment 20. The composition of any one of embodiments 1-8, wherein the composition comprises one or more, two or more, three or more, four or more, five or more partner polypeptides, or one or more nucleic acids encoding the one or more, two or more, three or more, four or more, five or more partner polypeptides.

Embodiment 21. The composition of embodiment 20, wherein the one or more partner polypeptides comprises an amino acid sequence that is at least 75% identical to any one of the sequences set forth in TABLE 1.1.

Embodiment 22. The composition of embodiment 20, wherein the one or more partner polypeptides comprises an amino acid sequence that is at least 80% identical to any one of the sequences set forth in TABLE 1.1.

Embodiment 23. The composition of embodiment 20, wherein the one or more partner polypeptides comprises an amino acid sequence that is at least 85% identical to any one of the sequences set forth in TABLE 1.1.

Embodiment 24. The composition of embodiment 20, wherein the one or more partner polypeptides comprises an amino acid sequence that is at least 90% identical to any one of the sequences set forth in TABLE 1.1.

Embodiment 25. The composition of embodiment 20, wherein the one or more partner polypeptides comprises an amino acid sequence that is at least 95% identical to any one of the sequences set forth in TABLE 1.1.

Embodiment 26. The composition of embodiment 20, wherein the one or more partner polypeptides comprises an amino acid sequence that is identical to any one of the sequences set forth in TABLE 1.1.

Embodiment 27. The composition of embodiment 20, wherein the composition comprises two or more partner polypeptides, or one or more nucleic acids encoding the two or more partner polypeptides.

Embodiment 28. The composition of embodiment 20, wherein the composition comprises three or more partner polypeptides, or one or more nucleic acids encoding the three or more partner polypeptides.

Embodiment 29. The composition of embodiment 20, wherein the composition comprises four or more partner polypeptides, or one or more nucleic acids encoding the four or more partner polypeptides.

Embodiment 30. The composition of embodiment 20, wherein the composition comprises five or more partner polypeptides, or one or more nucleic acids encoding the five or more partner polypeptides.

Embodiment 31. The composition of any one of embodiments 26-30, wherein each partner polypeptide independently comprises an amino acid sequence that is 75% identical to any one of the sequences set forth in TABLE 1.1.

Embodiment 32. The composition of any one of embodiments 26-30, wherein each partner polypeptide independently comprises an amino acid sequence that is 80% identical to any one of the sequences set forth in TABLE 1.1.

Embodiment 33. The composition of any one of embodiments 26-30, wherein each partner polypeptide independently comprises an amino acid sequence that is 85% identical to any one of the sequences set forth in TABLE 1.1.

Embodiment 34. The composition of any one of embodiments 26-30, wherein each partner polypeptide independently comprises an amino acid sequence that is 90% identical to any one of the sequences set forth in TABLE 1.1.

Embodiment 35. The composition of any one of embodiments 26-30, wherein each partner polypeptide independently comprises an amino acid sequence that is 95% identical to any one of the sequences set forth in TABLE 1.1.

Embodiment 36. The composition of any one of embodiments 26-30, wherein each partner polypeptide independently comprises an amino acid sequence that is identical to any one of the sequences set forth in TABLE 1.1.

Embodiment 37. The composition of embodiment 20, wherein the composition comprises a polypeptide and a partner polypeptide combination as described in TABLE 6.

Embodiment 38. The composition of any one of embodiments 1-37, wherein the composition comprises the nucleic acid that is a donor nucleic acid.

Embodiment 39. The composition of embodiment 38, wherein the donor nucleic acid is linear double-stranded DNA.

Embodiment 40. The composition of embodiment 38, wherein the donor nucleic acid is not linear double-stranded DNA.

Embodiment 41. The composition of any one of embodiments 38-40, wherein the donor nucleic acid comprises a structural motif that is recognized by the polypeptide.

Embodiment 42. The composition of embodiments 41, wherein the structural motif is an intermolecular recombination motif.

Embodiment 43. The composition of any one of embodiments 41 or 42, wherein the structural motif is one or more inverted repeats, inverted terminal repeats, or combinations thereof.

Embodiment 44. The composition of any one of embodiments 1-43, wherein the composition modifies a target sequence in a target nucleic acid.

Embodiment 45. The composition of embodiment 44, wherein the target sequence is downstream to a protospacer adjacent motif (PAM).

Embodiment 46. The composition of embodiment 44, wherein the target nucleic acid comprises an insertion site.

Embodiment 47. The composition of embodiment 46, wherein the insertion site is recognized by a polypeptide or partner polypeptide.

Embodiment 48. The composition of any one of embodiments 44-47, wherein the composition comprises the nucleic acid encoding an engineered guide nucleic acid or the engineered guide nucleic acid, wherein the engineered guide nucleic acid comprises a first region and a second region comprising a nucleic acid sequence that is complementary to the target sequence in the target nucleic acid, wherein the first region and the second region are heterologous to each other.

Embodiment 49. The composition of embodiment 48, wherein the first region, at least partially, interacts with the polypeptide.

Embodiment 50. The composition of embodiment 48, wherein the first region, at least partially, interacts with the polypeptide, or partner polypeptide, or both.

Embodiment 51. The composition of any one of embodiments 48-50, wherein the engineered guide nucleic acid comprises at least 10 contiguous nucleotides that are complementary to a eukaryotic sequence.

Embodiment 52. The composition of any one of embodiments 48-50, wherein the engineered guide nucleic acid comprises one or more phosphorothioate (PS) backbone modifications, 2′-fluoro (2′-F) sugar modifications, or 2′-O-Methyl (2′ OMe) sugar modifications.

Embodiment 53. The composition of any one of embodiments 48-52, wherein the composition further comprises an additional engineered guide nucleic acid that binds a different loci of the target nucleic acid than the engineered guide nucleic acid.

Embodiment 54. The composition of embodiment 20, wherein the polypeptide or the partner polypeptide, or both, is fused to one or more heterologous polypeptide.

Embodiment 55. The composition of embodiment 54, wherein the polypeptide or the partner polypeptide, or both, is fused to one or more heterologous polypeptide, wherein the heterologous polypeptide is a nuclear localization signal (NLS).

Embodiment 56. The composition of any one of embodiments 54-55, wherein the polypeptide comprises a RuvC domain that is capable of cleaving a target nucleic acid.

Embodiment 57. The composition of any one of embodiments 54-55, wherein the polypeptide is a nuclease that is capable of cleaving at least one strand of a target nucleic acid.

Embodiment 58. The composition of any one of embodiments 54-55, wherein the polypeptide is a nuclease that is capable of modification of at least one strand of a target nucleic acid.

Embodiment 59. The composition of embodiment 58, wherein the modification of the target nucleic acid comprises cleaving the target nucleic acid, deleting a nucleic acid of the target nucleic acid, inserting a nucleic acid into the target nucleic acid, substituting a nucleic acid of the target nucleic acid with an alternative nucleic acid, more than one of the foregoing, or combinations thereof.

Embodiment 60. The composition of any one of embodiments 58-59, wherein the modification of the target nucleic acid comprises insertion of a nucleic acid into the target nucleic acid.

Embodiment 61. The composition of any one of embodiments 58-59, wherein the modification of the target nucleic acid comprises insertion of a donor nucleic acid, deletion of a target nucleic acid, insertion of a donor nucleic acid fragment, deletion of a target nucleic acid fragment, or combinations thereof.

Embodiment 62. The composition of any one of embodiments 58-59, wherein the modification of the target nucleic acid comprises insertion of a donor nucleic acid or donor nucleic acid fragment into the target nucleic acid.

Embodiment 63. The composition of any one of embodiments 58-59, wherein the modification of the target nucleic acid comprises deletion of a target nucleic acid or a target nucleic acid fragment from the target nucleic acid.

Embodiment 64. The composition of any one of embodiments 58-59, wherein a donor nucleic acid can be a nucleotide, a nucleotide sequence, a coding sequence, a gene, a gene fragment, an exon, an intron, an exon fragment, an intron fragment, a gene regulatory region, a gene regulatory region fragment, coding sequences thereof, or combinations thereof.

Embodiment 65. The composition of any one of embodiments 58-59, wherein the modification of the target nucleic acid comprises site-specific recombinase activity.

Embodiment 66. The composition of any one of embodiments 58-59, wherein the modification of the target nucleic acid comprises transposase or transposase-like activity.

Embodiment 67. The composition of any one of embodiments 58-59, wherein the modification of the target nucleic acid comprises modification of a length of about 100 base pairs to about 500 base pairs of the target nucleic acid.

Embodiment 68. The composition of any one of embodiments 44-67, wherein the target sequence is within a human gene.

Embodiment 69. A nucleic acid expression vector that encodes a polypeptide, wherein the polypeptide comprises an amino acid sequence that is at least 75% identical to any one of the sequences set forth in TABLE 1.

Embodiment 70. A library of nucleic acid expression vectors comprising the nucleic acid expression vector of embodiment 69, wherein the nucleic acid expression vector encoding the polypeptide further encodes a partner polypeptide or wherein the library further comprises a separate nucleic acid expression vector encoding the partner polypeptide, and wherein the partner polypeptide comprises an amino acid sequence that is at least 75% identical to any one of the sequences set forth in TABLE 1.1.

Embodiment 71. The library of embodiment 70, wherein the nucleic acid expression vector of embodiment 69 or 70 encoding the polypeptide and/or the partner polypeptide further encodes a donor nucleic acid, or wherein the library further comprises a separate nucleic acid expression vector encoding the donor nucleic acid.

Embodiment 72. The library of embodiment 70, wherein the nucleic acid expression vector of any one of embodiments 69-71 encoding the polypeptide, the partner polypeptide, and/or a donor nucleic acid further encodes a target nucleic acid or wherein the library further comprises a separate nucleic acid expression vector encoding the target nucleic acid.

Embodiment 73. The nucleic acid expression vector or library of nucleic acid expression vectors of any one of embodiments 69 to 72, wherein at least one nucleic acid expression vector is a viral vector.

Embodiment 74. The nucleic acid expression vector or library of nucleic acid expression vectors of embodiment 73, wherein the viral vector is an adeno associated viral (AAV) vector.

Embodiment 75. The nucleic acid expression vector or library of nucleic acid expression vectors of any one of embodiments 69 to 72, wherein at least one nucleic acid expression vector is a lipid or a lipid nanoparticle.

Embodiment 76. A pharmaceutical composition, comprising the composition of any one of embodiments 1-68 or the nucleic acid expression vector or library of any one of embodiments 69-75; and a pharmaceutically acceptable excipient.

Embodiment 77. A system comprising the composition of any one of embodiments 1-68 or the nucleic acid expression vector or library of any one of embodiments 69-75.

Embodiment 78. The system of embodiment 77, comprising at least one detection reagent for detecting a target nucleic acid.

Embodiment 79. The system of embodiment 78, wherein the at least one detection reagent is selected from a reporter nucleic acid, a detection moiety, an additional polypeptide, or a combination thereof, optionally wherein the reporter nucleic acid comprises a fluorophore, a quencher, or a combination thereof.

Embodiment 80. The system of any one of embodiments 78-79, wherein the at least one detection reagent is operably linked to a polypeptide or partner polypeptide, such that a detection event occurs upon contacting the system with a target nucleic acid.

Embodiment 81. The system of any one of embodiments 77-80, comprising at least one amplification reagent for amplifying a target nucleic acid.

Embodiment 82. The system of embodiment 81, wherein the at least one amplification reagent is selected from the group consisting of a primer, an activator, a dNTP, an rNTP, and combinations thereof.

Embodiment 83. A method of modifying a target nucleic acid within a human gene, or associated with expression of a human gene, the method comprising contacting the target nucleic acid with the composition of any one of embodiments 1-68, the nucleic acid expression vector or library of any one of embodiments 69-75, the pharmaceutical composition of embodiment 76, or the system of any one of embodiments 77-82, thereby modifying the target nucleic acid.

Embodiment 84. The method of embodiment 83, wherein the modifying of the target nucleic acid comprises insertion or deletion of an exon, intron, exon fragment, intron fragment, gene regulatory region, gene regulatory region fragment, or any combinations thereof.

Embodiment 85. The method of embodiment 83, wherein the modifying of the target nucleic acid comprises insertion of an exon, intron, exon fragment, intron fragment, gene regulatory region, gene regulatory region fragment, or combinations thereof.

Embodiment 86. The method of any one of embodiments 83-85, further comprising contacting the target nucleic acid with a guide nucleic acid.

Embodiment 87. The method of any one of embodiments 83-86, wherein the method is performed in a cell.

Embodiment 88. The method of embodiment 87, wherein the method is performed in vivo.

Embodiment 89. The method of any one of embodiments 83-88, wherein the target nucleic acid comprises a mutation associated with a disease.

Embodiment 90. The method of embodiment 89, wherein the disease is a genetic disorder.

Embodiment 91. The method of embodiment 90, wherein the genetic disorder is a neurological disorder.

Embodiment 92. The method of any one of embodiments 83-91, wherein the target nucleic acid is encoded by a gene recited in TABLE 4.

Embodiment 93. The method of embodiment 92, wherein the gene comprises one or more mutations.

Embodiment 94. The method of embodiment 93, wherein the one or more mutations comprise a point mutation, a single nucleotide polymorphism (SNP), a chromosomal mutation, a copy number mutation, or any combination thereof.

Embodiment 95. The method of embodiment 90, wherein the disease is any one of the diseases recited in TABLE 5.

Embodiment 96. A cell comprising the composition of any one of embodiments 1-68 or the nucleic acid expression vector or library of any one of embodiments 69-75.

Embodiment 97. A cell that comprises a target nucleic acid modified by the composition of any one of embodiments 1-68 or the nucleic acid expression vector or library of any one of embodiments 69-75.

Embodiment 98. The cell of embodiment 96 or 97, wherein the cell is a eukaryotic cell.

Embodiment 99. The cell of any one of embodiments 96-98, wherein the cell is a mammalian cell.

Embodiment 100. The cell of any one of embodiments 96-99, wherein the cell is a human cell.

Embodiment 101. A population of cells that comprises at least one cell of any one of embodiments 95-100.

Embodiment 102. A method of treating a disease associated with a mutation or aberrant expression of a human gene in a subject in need thereof, the method comprising administering to the subject the pharmaceutical composition of embodiment 76.

Embodiment 103. The method of embodiment 102, wherein the disease is a genetic disorder.

Embodiment 104. The method of embodiment 103, wherein the genetic disorder is a neurological disorder.

Embodiment 105. The method of embodiment 102, wherein the human gene is a gene recited in TABLE 4.

Embodiment 106. The method of embodiment 102, wherein the disease is any one of the diseases recited in TABLE 5.

### Sequences and Tables

TABLE 1 provides illustrative amino acid sequences of effector proteins that are useful in the compositions, systems and methods described herein. (SEQ ID corresponds to Effector ID in column to its immediate right).

TABLE 1
EXEMPLARY AMINO ACID SEQUENCE(S)
OF EFFECTOR PROTEIN(S)

| SEQ ID NO: | Effector ID |
|---|---|
| 1 | 2456001 |
| 2 | 2456475 |
| 3 | 2456558 |
| 4 | 2457490 |
| 5 | 2457658 |
| 6 | 2457689 |
| 7 | 2457793 |
| 8 | 2457935 |
| 9 | 2458067 |
| 10 | 2459252 |
| 11 | 2460727 |
| 12 | 2460813 |
| 13 | 2460841 |
| 14 | 2460904 |
| 15 | 2460927 |
| 16 | 2460977 |
| 17 | 2462434 |
| 18 | 2462454 |
| 19 | 2463018 |
| 20 | 2463119 |
| 21 | 2464186 |
| 22 | 2464978 |
| 23 | 2465492 |
| 24 | 2465502 |
| 25 | 2465535 |
| 26 | 2465684 |
| 27 | 2465703 |
| 28 | 2466615 |
| 29 | 2466640 |
| 30 | 2466645 |
| 31 | 2466655 |
| 32 | 2466697 |
| 33 | 2466980 |
| 34 | 2467447 |
| 35 | 2467918 |
| 36 | 2467960 |
| 37 | 2467986 |
| 38 | 2468305 |
| 39 | 2468372 |
| 40 | 2468693 |
| 41 | 2468823 |
| 42 | 2468912 |
| 43 | 2468988 |
| 44 | 2469268 |
| 45 | 2469304 |
| 46 | 2469439 |
| 47 | 2469808 |
| 48 | 2469819 |
| 49 | 2469824 |
| 50 | 2470354 |
| 51 | 2473512 |
| 52 | 2482511 |
| 53 | 2483526 |
| 54 | 2483741 |
| 55 | 2489259 |
| 56 | 2490524 |
| 57 | 2490801 |
| 58 | 2490995 |
| 59 | 2491088 |
| 60 | 2504283 |
| 61 | 2504313 |
| 62 | 2504384 |
| 63 | 2504398 |
| 64 | 2504480 |
| 65 | 2504486 |
| 66 | 2504537 |
| 67 | 2504664 |
| 68 | 2504681 |
| 69 | 2504885 |
| 70 | 2505104 |
| 71 | 2505192 |
| 72 | 2505203 |
| 73 | 2505234 |
| 74 | 2505278 |
| 75 | 2505666 |
| 76 | 2505670 |
| 77 | 2505886 |
| 78 | 2505958 |
| 79 | 2506634 |
| 80 | 2506662 |
| 81 | 2506867 |
| 82 | 2507107 |
| 83 | 2507193 |
| 84 | 2507535 |
| 85 | 2507558 |
| 86 | 2507740 |
| 87 | 2507920 |
| 88 | 2508016 |
| 89 | 2508076 |
| 90 | 2508184 |
| 91 | 2508454 |
| 92 | 2509122 |
| 93 | 2509421 |
| 94 | 2509689 |
| 95 | 2509812 |
| 96 | 2510622 |
| 97 | 2512817 |
| 98 | 2513039 |
| 99 | 2513437 |
| 100 | 2514574 |
| 101 | 2514639 |
| 102 | 2514917 |
| 103 | 2514993 |
| 104 | 2515125 |
| 105 | 2519365 |
| 106 | 2520309 |
| 107 | 2520321 |
| 108 | 2520815 |
| 109 | 2520872 |
| 110 | 2521221 |
| 111 | 2521365 |
| 112 | 2521462 |
| 113 | 2521488 |
| 114 | 2521520 |
| 115 | 2521566 |
| 116 | 2521693 |
| 117 | 2521795 |
| 118 | 2521836 |
| 119 | 2521914 |
| 120 | 2522106 |
| 121 | 2522156 |
| 122 | 2522180 |
| 123 | 2522205 |
| 124 | 2522208 |
| 125 | 2522283 |
| 126 | 2522388 |
| 127 | 2522406 |
| 128 | 2522427 |
| 129 | 2522444 |
| 130 | 2522739 |
| 131 | 2522792 |
| 132 | 2522843 |
| 133 | 2523900 |
| 134 | 2526395 |
| 135 | 2526520 |
| 136 | 2532645 |
| 137 | 2532927 |
| 138 | 2533225 |
| 139 | 2533240 |
| 140 | 2533877 |
| 141 | 2534949 |
| 142 | 2535292 |
| 143 | 2536836 |
| 144 | 2537132 |
| 145 | 2537146 |
| 146 | 2537379 |
| 147 | 2538576 |
| 148 | 2538582 |
| 149 | 2538893 |
| 150 | 2538912 |
| 151 | 2539582 |
| 152 | 2540760 |
| 153 | 2542176 |
| 154 | 2542314 |
| 155 | 2542321 |
| 156 | 2542504 |
| 157 | 2542546 |
| 158 | 2542603 |
| 159 | 2542660 |
| 160 | 2542678 |
| 161 | 2542856 |
| 162 | 2542961 |
| 163 | 2542982 |
| 164 | 2542993 |
| 165 | 2543049 |
| 166 | 2543336 |
| 167 | 2543415 |
| 168 | 2543456 |
| 169 | 2543553 |
| 170 | 2543727 |
| 171 | 2544565 |
| 172 | 2545576 |
| 173 | 2545774 |
| 174 | 2549059 |
| 175 | 2549426 |
| 176 | 2549542 |
| 177 | 2549749 |
| 178 | 2551318 |
| 179 | 2552263 |
| 180 | 2553740 |
| 181 | 2555300 |
| 182 | 2555429 |
| 183 | 2555732 |
| 184 | 2555743 |
| 185 | 2557277 |
| 186 | 2559207 |
| 187 | 2559356 |
| 188 | 2559367 |
| 189 | 2559541 |
| 190 | 2559544 |
| 191 | 2559747 |
| 192 | 2560387 |
| 193 | 2560437 |
| 194 | 2560621 |
| 195 | 2561184 |
| 196 | 2563225 |
| 197 | 2563834 |
| 198 | 2564858 |
| 199 | 2566876 |
| 200 | 2568378 |
| 201 | 2569008 |
| 202 | 2569140 |
| 203 | 2570277 |
| 204 | 2570395 |
| 205 | 2570998 |
| 206 | 2572578 |
| 207 | 2572822 |
| 208 | 2572874 |
| 209 | 2572904 |
| 210 | 2573626 |
| 211 | 2573858 |
| 212 | 2573979 |
| 213 | 2574339 |
| 214 | 2574480 |
| 215 | 2575022 |
| 216 | 2575248 |
| 217 | 2575975 |
| 218 | 2576124 |
| 219 | 2576604 |
| 220 | 2576783 |
| 221 | 2578899 |
| 222 | 2579280 |
| 223 | 2580408 |
| 224 | 2580412 |
| 225 | 2581095 |
| 226 | 2581145 |
| 227 | 2581162 |
| 228 | 2581239 |
| 229 | 2581436 |
| 230 | 2581467 |
| 231 | 2581554 |
| 232 | 2581611 |
| 233 | 2581622 |
| 234 | 2581692 |
| 235 | 2581801 |
| 236 | 2581900 |
| 237 | 2582748 |
| 238 | 2585153 |
| 239 | 2585490 |
| 240 | 2585740 |
| 241 | 2585756 |
| 242 | 2585858 |
| 243 | 2585862 |
| 244 | 2585963 |
| 245 | 2585975 |
| 246 | 2587058 |
| 247 | 2594512 |
| 248 | 2595285 |
| 249 | 2595296 |
| 250 | 2596238 |
| 251 | 2607761 |
| 252 | 2610314 |
| 253 | 2618595 |
| 254 | 2618809 |
| 255 | 2619117 |
| 256 | 2619261 |
| 257 | 2621890 |
| 258 | 2621901 |
| 259 | 2628024 |
| 260 | 2629316 |
| 261 | 2631654 |
| 262 | 2631725 |
| 263 | 2631733 |
| 264 | 2631899 |
| 265 | 2631942 |
| 266 | 2635243 |
| 267 | 2636967 |
| 268 | 2637525 |
| 269 | 2639392 |
| 270 | 2639486 |
| 271 | 2639844 |
| 272 | 2640111 |
| 273 | 2640390 |
| 274 | 2640394 |
| 275 | 2640521 |
| 276 | 2640532 |
| 277 | 2641112 |
| 278 | 2641808 |
| 279 | 2641851 |
| 280 | 2642080 |
| 281 | 2642178 |
| 282 | 2642301 |
| 283 | 2642927 |
| 284 | 2643487 |
| 285 | 2643718 |
| 286 | 2643741 |
| 287 | 2643749 |
| 288 | 2643777 |
| 289 | 2645072 |
| 290 | 2645389 |
| 291 | 2645407 |
| 292 | 2645582 |
| 293 | 2645604 |
| 294 | 2645609 |
| 295 | 2645629 |
| 296 | 2645965 |
| 297 | 2645994 |
| 298 | 2646041 |
| 299 | 2646098 |
| 300 | 2646113 |
| 301 | 2646119 |
| 302 | 2646278 |
| 303 | 2646295 |
| 304 | 2646303 |
| 305 | 2646334 |
| 306 | 2646355 |
| 307 | 2646358 |
| 308 | 2646390 |
| 309 | 2646408 |
| 310 | 2646830 |
| 311 | 2646934 |
| 312 | 2646962 |
| 313 | 2647076 |
| 314 | 2647091 |
| 315 | 2647113 |
| 316 | 2647159 |
| 317 | 2647165 |
| 318 | 2647204 |
| 319 | 2647896 |
| 320 | 2648017 |
| 321 | 2648219 |
| 322 | 2648249 |
| 323 | 2648260 |
| 324 | 2648589 |
| 325 | 2648617 |
| 326 | 2648628 |
| 327 | 2648641 |
| 328 | 2648953 |
| 329 | 2649138 |
| 330 | 2649178 |
| 331 | 2649330 |
| 332 | 2649348 |
| 333 | 2649388 |
| 334 | 2649407 |
| 335 | 2649418 |
| 336 | 2649881 |
| 337 | 2650573 |
| 338 | 2650642 |
| 339 | 2650650 |
| 340 | 2650663 |
| 341 | 2650676 |
| 342 | 2652498 |
| 343 | 2655342 |
| 344 | 2655877 |
| 345 | 2656012 |
| 346 | 2656041 |
| 347 | 2656102 |
| 348 | 2656122 |
| 349 | 2656637 |
| 350 | 2656683 |
| 351 | 2656732 |
| 352 | 2656745 |
| 353 | 2656872 |
| 354 | 2656881 |
| 355 | 2657264 |
| 356 | 2657281 |
| 357 | 2658718 |
| 358 | 2658736 |
| 359 | 2659684 |
| 360 | 2660855 |
| 361 | 2661138 |
| 362 | 2661149 |
| 363 | 2661203 |
| 364 | 2661243 |
| 365 | 2661723 |
| 366 | 2661753 |
| 367 | 2662365 |
| 368 | 2663028 |
| 369 | 2663104 |
| 370 | 2663367 |
| 371 | 2663380 |
| 372 | 2663400 |
| 373 | 2663750 |
| 374 | 2663760 |
| 375 | 2663780 |
| 376 | 2663815 |
| 377 | 2663833 |
| 378 | 2663974 |
| 379 | 2664009 |
| 380 | 2664025 |
| 381 | 2664156 |
| 382 | 2664342 |
| 383 | 2664593 |
| 384 | 2664914 |
| 385 | 2664957 |
| 386 | 2664986 |
| 387 | 2665006 |
| 388 | 2665109 |
| 389 | 2665325 |
| 390 | 2665379 |
| 391 | 2665417 |
| 392 | 2665447 |
| 393 | 2666085 |
| 394 | 2666163 |
| 395 | 2666290 |
| 396 | 2666471 |
| 397 | 2666830 |
| 398 | 2667583 |
| 399 | 2667596 |
| 400 | 2667609 |
| 401 | 2667637 |
| 402 | 2667659 |
| 403 | 2667740 |
| 404 | 2667863 |
| 405 | 2667874 |
| 406 | 2668940 |
| 407 | 2669158 |
| 408 | 2669252 |
| 409 | 2669317 |
| 410 | 2669455 |
| 411 | 2670262 |
| 412 | 2670431 |
| 413 | 2670948 |
| 414 | 2671509 |
| 415 | 2671561 |
| 416 | 2673019 |
| 417 | 2673030 |
| 418 | 2673085 |
| 419 | 2673122 |
| 420 | 2673750 |
| 421 | 2674873 |
| 422 | 2674903 |
| 423 | 2674920 |
| 424 | 2675029 |
| 425 | 2675057 |
| 426 | 2675141 |
| 427 | 2675820 |
| 428 | 2675905 |
| 429 | 2678009 |
| 430 | 2678104 |
| 431 | 2678129 |
| 432 | 2680715 |
| 433 | 2680787 |
| 434 | 2680813 |
| 435 | 2681033 |
| 436 | 2681040 |
| 437 | 2681339 |
| 438 | 2682278 |
| 439 | 2682344 |
| 440 | 2682347 |
| 441 | 2684692 |
| 442 | 2684983 |
| 443 | 2685267 |
| 444 | 2687981 |
| 445 | 2687986 |
| 446 | 2687998 |
| 447 | 2688086 |
| 448 | 2688136 |
| 449 | 2688183 |
| 450 | 2688225 |
| 451 | 2688430 |
| 452 | 2689663 |
| 453 | 2690183 |
| 454 | 2690859 |

TABLE 1.1 provides illustrative amino acid sequences of effector partner proteins that are useful in the compositions, systems and methods described herein.

TABLE 2.1
EXEMPLARY AMINO ACID SEQUENCE(S)
OF EFFECTOR PARTNER PROTEIN(S)

| SEQ ID NO: | Partner ID |
|---|---|
| 455 | 2464187 |
| 456 | 2466643 |
| 457 | 2482514 |
| 458 | 2504538 |
| 459 | 2505190 |
| 460 | 2509417 |
| 461 | 2533878 |
| 462 | 2542964 |
| 463 | 2533879 |
| 464 | 2543050 |
| 465 | 2551321 |
| 466 | 2575246 |
| 467 | 2635246 |
| 468 | 2646099 |
| 469 | 2646116 |
| 470 | 2646356 |
| 471 | 2648954 |
| 472 | 2650569 |
| 473 | 2646104 |
| 474 | 2650646 |
| 475 | 2650661 |
| 476 | 2650672 |
| 477 | 2658714 |
| 478 | 2658739 |
| 479 | 2660856 |
| 480 | 2663365 |
| 481 | 2663378 |
| 482 | 2663398 |
| 483 | 2664026 |
| 484 | 2664917 |
| 485 | 2665105 |
| 486 | 2666088 |
| 487 | 2667599 |
| 488 | 2673120 |
| 489 | 2674906 |
| 490 | 2663377 |
| 491 | 2673119 |
| 492 | 2681036 |
| 493 | 2585758 |
| 494 | 2463120 |
| 495 | 2468301 |
| 496 | 2468820 |
| 497 | 2468908 |
| 498 | 2468985 |
| 499 | 2504660 |
| 500 | 2537141 |
| 501 | 2539580 |
| 502 | 2542319 |
| 503 | 2542604 |
| 504 | 2640108 |
| 505 | 2585757 |
| 506 | 2631650 |
| 507 | 2643716 |
| 508 | 2646274 |
| 509 | 2646298 |
| 510 | 2647205 |
| 511 | 2648613 |
| 512 | 2648626 |
| 513 | 2641805 |
| 514 | 2456002 |
| 515 | 2465685 |
| 516 | 2466638 |
| 517 | 2466656 |
| 518 | 2466698 |
| 519 | 2504281 |
| 520 | 2504385 |
| 521 | 2505204 |
| 522 | 2507556 |
| 523 | 2540761 |
| 524 | 2642175 |
| 525 | 2569006 |
| 526 | 2572875 |
| 527 | 2646391 |
| 528 | 2646406 |
| 529 | 2647077 |
| 530 | 2647111 |
| 531 | 2656010 |
| 532 | 2656743 |
| 533 | 2661139 |
| 534 | 2661150 |
| 535 | 2663751 |
| 536 | 2663831 |
| 537 | 2663972 |
| 538 | 2664157 |
| 539 | 2664594 |
| 540 | 2666828 |
| 541 | 2505279 |
| 542 | 2538574 |
| 543 | 2542502 |
| 544 | 2568379 |
| 545 | 2581237 |
| 546 | 2641110 |
| 547 | 2645070 |
| 548 | 2646304 |
| 549 | 2460978 |
| 550 | 2462455 |
| 551 | 2463019 |
| 552 | 2508182 |
| 553 | 2515126 |
| 554 | 2535290 |
| 555 | 2543457 |
| 556 | 2545577 |
| 557 | 2585863 |
| 558 | 2587059 |
| 559 | 2619115 |
| 560 | 2621888 |
| 561 | 2640392 |
| 562 | 2640530 |
| 563 | 2646353 |
| 564 | 2655343 |
| 565 | 2656638 |
| 566 | 2656870 |
| 567 | 2657279 |
| 568 | 2661204 |
| 569 | 2663105 |
| 570 | 2664958 |
| 571 | 2665323 |
| 572 | 2665380 |
| 573 | 2665418 |
| 574 | 2666288 |
| 575 | 2667638 |
| 576 | 2668938 |
| 577 | 2671559 |
| 578 | 2682345 |
| 579 | 2682348 |
| 580 | 2684690 |
| 581 | 2688226 |
| 582 | 2690860 |
| 583 | 2466978 |
| 584 | 2507105 |
| 585 | 2509810 |
| 586 | 2513037 |
| 587 | 2581093 |
| 588 | 2581146 |
| 589 | 2581465 |
| 590 | 2582746 |
| 591 | 2585738 |
| 592 | 2631897 |
| 593 | 2642081 |
| 594 | 2648590 |
| 595 | 2456476 |
| 596 | 2457488 |
| 597 | 2457659 |
| 598 | 2457687 |
| 599 | 2460728 |
| 600 | 2465490 |
| 601 | 2465500 |
| 602 | 2465533 |
| 603 | 2469266 |
| 604 | 2469302 |
| 605 | 2469820 |
| 606 | 2483742 |
| 607 | 2489257 |
| 608 | 2490525 |
| 609 | 2490993 |
| 610 | 2504396 |
| 611 | 2504886 |
| 612 | 2505102 |
| 613 | 2505235 |
| 614 | 2505668 |
| 615 | 2505959 |
| 616 | 2506635 |
| 617 | 2507533 |
| 618 | 2512818 |
| 619 | 2522844 |
| 620 | 2523898 |
| 621 | 2526396 |
| 622 | 2526521 |
| 623 | 2534950 |
| 624 | 2538580 |
| 625 | 2542658 |
| 626 | 2549427 |
| 627 | 2549750 |
| 628 | 2552264 |
| 629 | 2553741 |
| 630 | 2555741 |
| 631 | 2557278 |
| 632 | 2559205 |
| 633 | 2559354 |
| 634 | 2560438 |
| 635 | 2560622 |
| 636 | 2564856 |
| 637 | 2570278 |
| 638 | 2570396 |
| 639 | 2570999 |
| 640 | 2573856 |
| 641 | 2576781 |
| 642 | 2578900 |
| 643 | 2580409 |
| 644 | 2581163 |
| 645 | 2581434 |
| 646 | 2581609 |
| 647 | 2581620 |
| 648 | 2581693 |
| 649 | 2581799 |
| 650 | 2581898 |
| 651 | 2585859 |
| 652 | 2585964 |
| 653 | 2585976 |
| 654 | 2595286 |
| 655 | 2595297 |
| 656 | 2619262 |
| 657 | 2636968 |
| 658 | 2640109 |
| 659 | 2641806 |
| 660 | 2641852 |
| 661 | 2642176 |
| 662 | 2643775 |
| 663 | 2645583 |
| 664 | 2645605 |
| 665 | 2645607 |
| 666 | 2645630 |
| 667 | 2646117 |
| 668 | 2646932 |
| 669 | 2646963 |
| 670 | 2647160 |
| 671 | 2647163 |
| 672 | 2648220 |
| 673 | 2648250 |
| 674 | 2649176 |
| 675 | 2649328 |
| 676 | 2649349 |
| 677 | 2649386 |
| 678 | 2649408 |
| 679 | 2649882 |
| 680 | 2655878 |
| 681 | 2656681 |
| 682 | 2656733 |
| 683 | 2662363 |
| 684 | 2663758 |
| 685 | 2663781 |
| 686 | 2663813 |
| 687 | 2664010 |
| 688 | 2664987 |
| 689 | 2665007 |
| 690 | 2665448 |
| 691 | 2666469 |
| 692 | 2667584 |
| 693 | 2667864 |
| 694 | 2667875 |
| 695 | 2669250 |
| 696 | 2670429 |
| 697 | 2671510 |
| 698 | 2673017 |
| 699 | 2673028 |
| 700 | 2673086 |
| 701 | 2674874 |
| 702 | 2675058 |
| 703 | 2675139 |
| 704 | 2675822 |
| 705 | 2675906 |
| 706 | 2678127 |
| 707 | 2680814 |
| 708 | 2681034 |
| 709 | 2681340 |
| 710 | 2684984 |
| 711 | 2685265 |
| 712 | 2687984 |
| 713 | 2687996 |
| 714 | 2689664 |
| 715 | 2457936 |
| 716 | 2460928 |
| 717 | 2464976 |
| 718 | 2467448 |
| 719 | 2469809 |
| 720 | 2469822 |
| 721 | 2505667 |
| 722 | 2505887 |
| 723 | 2506660 |
| 724 | 2509687 |
| 725 | 2510620 |
| 726 | 2514640 |
| 727 | 2514918 |
| 728 | 2532646 |
| 729 | 2533223 |
| 730 | 2542857 |
| 731 | 2543334 |
| 732 | 2544566 |
| 733 | 2559748 |
| 734 | 2560388 |
| 735 | 2618810 |
| 736 | 2639845 |
| 737 | 2642928 |
| 738 | 2645387 |
| 739 | 2645405 |
| 740 | 2648261 |
| 741 | 2649419 |
| 742 | 2456555 |
| 743 | 2458064 |
| 744 | 2460811 |
| 745 | 2462435 |
| 746 | 2504310 |
| 747 | 2506868 |
| 748 | 2520310 |
| 749 | 2520322 |
| 750 | 2520870 |
| 751 | 2537380 |
| 752 | 2542674 |
| 753 | 2549056 |
| 754 | 2555301 |
| 755 | 2561182 |
| 756 | 2563226 |
| 757 | 2566873 |
| 758 | 2572905 |
| 759 | 2573624 |
| 760 | 2574478 |
| 761 | 2585488 |
| 762 | 2618591 |
| 763 | 2621908 |
| 764 | 2639393 |
| 765 | 2642302 |
| 766 | 2645992 |
| 767 | 2646335 |
| 768 | 2646831 |
| 769 | 2652499 |
| 770 | 2656038 |
| 771 | 2656119 |
| 772 | 2656882 |
| 773 | 2657265 |
| 774 | 2659685 |
| 775 | 2661240 |
| 776 | 2661724 |
| 777 | 2664339 |
| 778 | 2667741 |
| 779 | 2669159 |
| 780 | 2669318 |
| 781 | 2670263 |
| 782 | 2670945 |
| 783 | 2674917 |
| 784 | 2680716 |
| 785 | 2682279 |
| 786 | 2688083 |
| 787 | 2688137 |
| 788 | 2688184 |
| 789 | 2460843 |
| 790 | 2460905 |
| 791 | 2468691 |
| 792 | 2483527 |
| 793 | 2504481 |
| 794 | 2504484 |
| 795 | 2507190 |
| 796 | 2508017 |
| 797 | 2508077 |
| 798 | 2520813 |
| 799 | 2542315 |
| 800 | 2542544 |
| 801 | 2543412 |
| 802 | 2579281 |
| 803 | 2581551 |
| 804 | 2628025 |
| 805 | 2631726 |
| 806 | 2631730 |
| 807 | 2640528 |
| 808 | 2643488 |
| 809 | 2643738 |
| 810 | 2643750 |
| 811 | 2645962 |
| 812 | 2646042 |
| 813 | 2647893 |
| 814 | 2466613 |
| 815 | 2467961 |
| 816 | 2467984 |
| 817 | 2470355 |
| 818 | 2514991 |
| 819 | 2521464 |
| 820 | 2521489 |
| 821 | 2521518 |
| 822 | 2521567 |
| 823 | 2521691 |
| 824 | 2522386 |
| 825 | 2522737 |
| 826 | 2522793 |
| 827 | 2536837 |
| 828 | 2542177 |
| 829 | 2543728 |
| 830 | 2545772 |
| 831 | 2559365 |
| 832 | 2563835 |
| 833 | 2576125 |
| 834 | 2576602 |
| 835 | 2631940 |
| 836 | 2639484 |
| 837 | 2656100 |
| 838 | 2663029 |
| 839 | 2666164 |
| 840 | 2667661 |
| 841 | 2669452 |
| 842 | 2467919 |
| 843 | 2468373 |
| 844 | 2490802 |
| 845 | 2504679 |
| 846 | 2521222 |
| 847 | 2521363 |
| 848 | 2521796 |
| 849 | 2521834 |
| 850 | 2521915 |
| 851 | 2522104 |
| 852 | 2522154 |
| 853 | 2522182 |
| 854 | 2522203 |
| 855 | 2522206 |
| 856 | 2522281 |
| 857 | 2522407 |
| 858 | 2522425 |
| 859 | 2522442 |
| 860 | 2559542 |
| 861 | 2559545 |
| 862 | 2573977 |
| 863 | 2575020 |
| 864 | 2580413 |
| 865 | 2637523 |
| 866 | 2457794 |
| 867 | 2469440 |
| 868 | 2473513 |
| 869 | 2491086 |
| 870 | 2507738 |
| 871 | 2508455 |
| 872 | 2509123 |
| 873 | 2513438 |
| 874 | 2537130 |
| 875 | 2538891 |
| 876 | 2543551 |
| 877 | 2572576 |
| 878 | 2574337 |
| 879 | 2594510 |
| 880 | 2629314 |
| 881 | 2648018 |
| 882 | 2661751 |
| 883 | 2667610 |
| 884 | 2673751 |
| 885 | 2678007 |
| 886 | 2678102 |
| 887 | 2687982 |
| 888 | 2690184 |
| 889 | 2459261 |
| 890 | 2465701 |
| 891 | 2514575 |
| 892 | 2519363 |
| 893 | 2532928 |
| 894 | 2533241 |
| 895 | 2538913 |
| 896 | 2549543 |
| 897 | 2569141 |
| 898 | 2572823 |
| 899 | 2575976 |
| 900 | 2585154 |
| 901 | 2596236 |
| 902 | 2607759 |
| 903 | 2610312 |
| 904 | 2640391 |
| 905 | 2649136 |
| 906 | 2455999 |
| 907 | 2465682 |
| 908 | 2466642 |
| 909 | 2466653 |
| 910 | 2504285 |
| 911 | 2505201 |
| 912 | 2505276 |
| 913 | 2507560 |
| 914 | 2538578 |
| 915 | 2540758 |
| 916 | 2542506 |
| 917 | 2555427 |
| 918 | 2555730 |
| 919 | 2568376 |
| 920 | 2569010 |
| 921 | 2572872 |
| 922 | 2581241 |
| 923 | 2645074 |
| 924 | 2646301 |
| 925 | 2646388 |
| 926 | 2647115 |
| 927 | 2656014 |
| 928 | 2656747 |
| 929 | 2661136 |
| 930 | 2661147 |
| 931 | 2663748 |
| 932 | 2663835 |
| 933 | 2663976 |
| 934 | 2664154 |
| 935 | 2664591 |
| 936 | 2666832 |
| 937 | 2468303 |
| 938 | 2468910 |
| 939 | 2482513 |
| 940 | 2504662 |
| 941 | 2507918 |
| 942 | 2509419 |
| 943 | 2537144 |
| 944 | 2542963 |
| 945 | 2542984 |
| 946 | 2542995 |
| 947 | 2551320 |
| 948 | 2631652 |
| 949 | 2635245 |
| 950 | 2646115 |
| 951 | 2646276 |
| 952 | 2646297 |
| 953 | 2648615 |
| 954 | 2648639 |
| 955 | 2650571 |
| 956 | 2650644 |
| 957 | 2650648 |
| 958 | 2650674 |
| 959 | 2658716 |
| 960 | 2658738 |
| 961 | 2664916 |
| 962 | 2665107 |
| 963 | 2666087 |
| 964 | 2667598 |
| 965 | 2674905 |
| 966 | 2675031 |
| 967 | 2680785 |
| 968 | 2681038 |
| 969 | 2688428 |

TABLE 2 provides illustrative sequences of exemplary heterologous polypeptide modifications of effector protein(s) that are useful in the compositions, systems and methods described herein.

TABLE 2
SEQUENCES OF EXEMPLARY HETEROLOGOUS POLYPEPTIDE
MODIFICATIONS OF EFFECTOR PROTEIN(S)

| SEQ | | |
|---|---|---|
| ID | | |
| NO: | Description | Sequence* |
| | NLS | KR(K/R)R |
| | NLS | (P/R)XXKR(^{∧}D/E)(K/R) |
| 972 | NLS | KRX(W/F/Y)XXAF |
| | NLS | (R/P)XXKR(K/R)(^{∧}D/E) |
| 974 | NLS | LGKR(K/R)(W/F/Y) |
| | NLS | KRX₁₀K(K/R)(K/R) |
| 976 | EEP | GLFXALLXLLXSLWXLLLXA |
| | NLS | K(K/R)RK |
| | NLS | KRX₁₁K(K/R)(K/R) |
| | NLS | KRX₁₂K(K/R)(K/R) |
| | NLS | KRX₁₀K(K/R)X(K/R) |
| | NLS | KRX₁₁K(K/R)X(K/R) |
| | NLS | KRX₁₂K(K/R)X(K/R) |
| 983 | NLS | APKKKRKVGIHGVPAA |

*wherein X is independently any amino acid; and ^{∧}D/E is any naturally occurring amino acid except Asp or Glu

TABLE 3 provides illustrative repeat sequences for use in guide nucleic acids that are useful in the compositions, systems and methods described herein.

TABLE 3
EXEMPLARY REPEAT SEQUENCES FOR USE IN GUIDE NUCLEIC ACIDS

| Effector | Effector | Repeat | |
|---|---|---|---|
| Protein | Partner | Sequence | |
| SEQ ID NO: | SEQ ID NO: | SEQ ID NO: | Repeat Sequence (5′→3′), shown as RNA |
| 42 | 938 | 985 | GUCGCGCGCGACUCCCCGAUGUGAAGGGGACUGAAAC |
| 300 | 950 | 986 | GAUUGUAAAGAACAACUUGGCUAU |
| 308 | 925 | 987 | UGCUUGCAAAAAUGGACGCG |
| 16 | 549 | 988 | CGGAUCAUCCCCGCGCGGGGGGGAACAC |
| 82 | 584 | 989 | GUCCGCCCCGCGCAUGCGGGGAUCGACC |
| 280 | 593 | 990 | GGUUCCUCCGCGUGCGCGGAGAUAGACC |
| 353 | 566 | 991 | GGCUUCAUCCCCGCUGAGCGGGGCCACC |
| 390 | 572 | 992 | CGGUUCAUCCCCGCGCACGCGGGGAACAC |
| 198 | 636 | 993 | GACUGAAUCACAGACCUGAAUAAGAAGGGAUUAAGAC |
| 235 | 649 | 994 | UGCGUUACAAGAUCUCUUAACGGUUCAACAU |
| 254 | 735 | 995 | GCUUCAAUGGGGCCGCGCUUUCGGAAGCGCGGAAAC |
| 174 | 753 | 996 | AAUUUCAAUCCACGCCCCCCGCGAGGGGAGCGAC |
| 40 | 791 | 997 | GUCGCUCCCCGCGUGGGGGCGUGGAUUGAAAC |
| 88 | 796 | 998 | GUUUUCCCCACACGCGUGGGGGUGGACCG |
| 106 | 748 | 999 | GUUUUCCCCGCGAGUGCGGGGGUGAGGCG |
| 222 | 802 | 1000 | AUCGUCCCCACGGGCGUGGGGGUGAACCG |
| 124 | 855 | 1001 | GUUUCAACCCUCACCCAACCUCAAGGCCGGGUGCAAC |
| 125 | 856 | 1002 | GUUUCAAUCUCAACCGAGAUGCCAGGCCCCUGGCGAC |
| 173 | 830 | 1003 | UUUCAAUCUCUGCUGAGCUUCCCUGACCACAUAGAC |
| 188 | 831 | 1004 | GUUUCAAUCUCAGACGAGAUGCCAGGCCGCUUGAGCC |
| 394 | 839 | 1005 | GUUUCAAUUCUCAAUGCCCCCGAGAAGGCAUUGCUAC |
| 51 | 868 | 1006 | UUUCCGGAGCAUACCCGUUCA |
| 252 | 903 | 1007 | AUUUCAAUUCUACUUAGUUCUAUUAAUAC |
| 329 | 905 | 1008 | GUUGUAUCUUAUCAAACAUCUACAUCAAACCACAAC |

TABLE 4 provides illustrative target nucleic acids that are useful in the compositions, systems and methods described herein.

TABLE 4
EXEMPLARY TARGET NUCLEIC ACIDS
Exemplary targets

AAVS1, ABCA4, ABCB11, ABCC8, ABCD1, ABCG5, ABCG8, ACAD9, ACADM, ACADVL, ACAT1, ACTA1, ACOX1, ACSF3, ADA,
ADAMTS2, ADGRG1, AGA, AGL, AGPS, AGXT, AHI1, AIRE, ALDH3A2, ALDOB, ALG6, ALK, ALKBH5, ALMS1, ALPL, AMRC9, AMT,
ANAPC10, ANAPC11, ANGPTL3, ANGPTL4, ANGPTL7, APC, Apo(a), APOCIII, APOE, APOEε4, APOL1, APP, AQP2, AR, ARFRP1, ARG1,
ARH, ARL13B, ARL6, ARSA, ARSB, ASL, ASNS, ASPA, ASS1, ATM, ATP6V1B1, ATP7A, ATP7B, ATRX, ATXN1, ATXN10, ATXN2, ATXN3,
ATXN7, ATXN8OS, AXIN1, AXIN2, B2M, BACE-1, BAK1, BAP1, BARD1, BAX2, BBS1, BBS10, BBS12, BBS2, BCKDHA, BCKDHB, BCL2L2,
BCS1L, BEST1, Betaglobin gene, BLM, BMPR1A, BRAF, BRAFV600E, BRCA1, BRCA2, BRIP1, BSND, C9orf72, CA4, CACNA1A, CAH1,
CAPN3, CASR, CBS, CCNB1 CC2D2A, CCR5, CD1, CD2, CD3, CD3D, CD3Z, CD4, CD5, CD6, CD7, CD8A, CD8B, CD9, CD14, CD18,
CD19, CD21, CD22, CD23, CD27, CD28, CD30, CD33, CD34, CD36, CD38, CD40, CD40L, CD44, CD46, CD47, CD48, CD52, CD55,
CD57, CD58, CD59, CD68, CD69, CD72, CD73, CD74, CD79A, CD80, CD81, CD83, CD84, CD86, CD90, CD93, CD96, CD99, CD100,
CD123, CD160, CD163, CD164, CD164L2, CD166, CD200, CD204, CD207, CD209, CD226, CD244, CD247, CD274, CD276, CD300,
CD320, CDC73, CDH1, CDH23, CDK11, CDK4, CDKN1A, CDKN1B, CDKN1C, CDKN2A, CDKN2B, CEBPA, CELA3B, CEP 290, CERKL,
CFB, CFTR, CHCHD10, CHEK2, CHM, CHRNE, CIDEB, CIITA, CLN3, CLN5, CLN6, CLN8, CLRN1, CLTA, CMT1A, CNBP, CNGB1,
CNGB3, COL1A1, COL1A2, COL27A1, COL4A3, COL4A4, COL4A5, COL6A1, COL6A2, COL6A3, COL7A1, CPSI, CPT1A, CPT2, CRB1,
CREBBP, CRX, CRYAA, CTNNA1, CTNNB1, CTNND2, CTNS, CTSK, CXCL12, CYBA, CYBB, CYP11B1, CYP11B2, CYP17A1, CYP19A1,
CYP21A2, CYP27A1, DBT, DCC, DCLREIC, DERL2, DFNA36, DFNB31, DGAT2, DHCR7, DHDDS, DICER1, DIS3L2, DLD, DMD, DMPK,
DNAH5, DNAI1, DNAI2, DNM2, DNMT1, DPC4, DUX4, DYSF, EDA, EDN3, EDNRB, EGFR, EIF2B5, EMC2, EMC3, EMD, EMX1, EN1,
EPCAM, ERCC6, ERCC8, ESCO2, ETFA, ETFDH, ETHE1, EVC, EVC2, EYS, F5, F9, FXI, FAH, FAM161A, FANCA, FANCB, FANCC,
FANCD1, FANCD2, FANCE, FANCF, FANCG, FANCI, FANCJ, FANCL, FANCM, FANCN, FANCP, FANCS, FBN1, FGF14, FGFR2,
FGFR3, FGA, FGB, FGG, FH, FHL1, FIX, FKRP, FKTN, FLCN, FMR1, FOXP 3, FSCN2, FSHD1, FUS, FUT8, FVIII, FXII, FXN, G6PC,
GAA, GALC, GALK1, GALT, GAMT, GATA2, GATA-4, GBA, GBE1, GCDH, GCGR, GDNF, GFAP, GFM1, GHR, GJB1, GJB2, GLA, GLB1,
GLDC, GLE1, GNE, GNPTAB, GNPTG, GNS, GPAM, GPC3, GPR98, GREM1, GRHPR, GRIN2B, H2AFX, H2AX, HADHA, HAX1, HBA1,
HBA2, HBB, HBV cccDNA, HER2, HEXA, HEXB, HFE, HGSNAT, HLCS, HMGCL, HAO1, HOGA1, HOXB13, HPRPF3, HPRT1, HPS1,
HPS3, HRAS, HRD1, HSD3B2, HSD17B4, HSD17B13, HTT, HUS1, HYAL1, HYLS1, IDS, IDUA, IFITM5, IFN, IFN-γ, IKBKAP, IL2RG, IL7R,
IMPDH1, INPP5E, IRF4, ITGB2, ITPR1, IVD, JAG1, JAK1, JAK3, KCNC3, KCND3, KCNJ11, KLKB1, KLHL7, KRAS, LAMA1, LAMA2,
LAMA3, LAMB3, LAMC2, LCA5, LDHA, LDLR, LDLRAP1, LHX3, LIFR, LIPA, LMNA, LMOD3, LOR, LOXHD1, LPA, LPL, LRAT, LRP6,
LRPPRC, LRRK2, MADR2, MAN2B1, MAPT, MARC1, MAX, MCM6, MCOLN1, MECP2, MED17, MEFV, MEN1, MERTK, MESP2, MET,
METex14, MFN2, MFSD8, MIA3, MITF, MKL2, MKS1, MLC1, MLH1, MLH3, MMAA, MMAB, MMACHC, MMADHC, MMD, MPI, MPL,
MPV17, MSH2, MSH3, MSH6, MTHFD1L, MTHFR, MTM1, MTRR, MTTP, MUT, MUTYH, MYC, MYH7, MYO7A, MYOC, NAGLU, NAGS,
NAV1.7, NBN, NDRG1, NDUFAF5, NDUFS6, NEB, NF1, NF2, NKX2-5, NOG, NOTCH1, NOTCH2, NPC1, NPC2, NPHP1, NPHS1, NPHS2,
NRAS, NR2E3, NTHL1, NTRK, NTRK1, OAT, OCT4, OFD1, OPA3, OTC, PAH, PALB2, PAQR8, PAX3, PC, PCCA, PCCB, PCDH15, PCSK9,
PD1, PDCD1, PDE6B, PDGFRA, PDHA1, PDHB, PEX1, PEX10, PEX12, PEX13, PEX14, PEX16, PEX19, PEX2, PEX26, PEX3, PEX5,
PEX6, PEX7, PFKM, PHGDH, PHOX2B, PKD1, PKD2, PKHD1, PKK, PLEKHG4, PMM2, PMP22, PMS1, PMS2, PNPLA3, POLD1, POLE,
POMGNT1, POT1, POU5F1, PPM1A, PPP2R2B, PPT1, PRCD, PRKAG2, PRKAR1A, PRKCG, PRNP, PROM1, PROP1, PRPF31, PRPF8,
PRPH2, PRPS1, PSAP, PSD3, PSD95, PSEN1, PSEN2, PSRC1, PTCH1, PTEN, PTS, PUS1, PYGM, RAB23, RAD50, RAD51C, RAD51D,
RAG1, RAG2, RAPSN, RARS2, RB1, RDH12, RECQL4, RET, RHO, RICTOR, RMRP, ROS1, RP1, RP2, RPE65, RPGR, RPGRIP1L, RPL32P3,
RPTOR, RS1, RTCA, RTEL1, RUNX1, SACS, SAMHD1, SCN1A, SCN2A, SDHA, SDHAF2, SDHB, SDHC, SDHD, SEL1L, SEPSECS,
SERPINA1, SERPINC1, SERPING1, SGCA, SGCB, SGCG, SGSH, SIRT1, SLC12A3, SLC12A6, SLC17A5, SLC22A5, SLC25A13, SLC25A15,
SLC26A2, SLC26A4, SLC35A3, SLC35B4, SLC37A4, SLC39A4, SLC4A11, SLC6A8, SLC7A7, SMAD3, SMAD4, SMARCA4, SMARCAL1,
SMARCB1, SMARCE1, SMN1, SMPD1, SNAI2, SNCA, SNRNP200, SOD1, SOX10, SPARA7, SPTBN2, STAR, STAT3, STK11, SUFU, SUMF1,
SYNE1, SYNE2, SYS1, TARDBP, TAT, TBK1, TBP, TCF4, TCIRG1, TCTN3, TECPR2, TERC, TERT, TFR2, TGFBR2, TGM1, TH, TLE3,
TMEM127, TMEM138, TMEM216, TMEM43, TMEM67, TMPRSS6, TNNI2, TNNT1, TNNT3, TOP1, TOPORS, TP53, TPM2, TPM3, TPP1,
TRAC, TRMU, TSC1, TSC2, TSFM, TSPAN14, TTBK2, TTC8, TTPA, TTR, TULP1, TYMP, UBE2G2, UBE2J1, UBE3A, USH1C, USH1G,
USH2A, VEGF, VHL, VPS13A, VPS13B, VPS35, VPS45, VRK1, VSX2, VWF, WAS, WDR19, WDR48, WNT10A, WRN, WS2B, WS2C, WT1,
XPA, XPC, XPF, XRCC3, YAP1, ZAC1, ZEB1, ZFYVE26, and ZNF423

TABLE 5 provides illustrative diseases and syndromes for compositions, systems and methods described herein.

TABLE 5
DISEASES AND SYNDROMES
Exemplary Diseases and Syndromes

11-hydroxylase deficiency; 17, 20-desmolase deficiency; 17-hydroxylase deficiency; 3-hydroxyisobutyrate aciduria; 3-hydroxysteroid
dehydrogenase deficiency; 46, XY gonadal dysgenesis; AAA syndrome; ABCA3 deficiency; ABCC8-associated hyperinsulinism;
aceruloplasminemia; acromegaly; achondrogenesis type 2; acral peeling skin syndrome; acrodermatitis enteropathica; acute bacterial infection;
adrenocortical micronodular hyperplasia; adrenoleukodystrophies; adrenomyeloneuropathies; Aicardi-Goutieres syndrome; AIDS; Alagille
disease (also called Alagille Syndrome); Alexander Disease; Alpers syndrome; alpha-1 antitrypsin deficiency (AATD); alpha-mannosidosis;
Alstrom syndrome; Alzheimer's disease; amebic dysentery; amelogenesis imperfecta; amish type microcephaly; amyotrophic lateral sclerosis
(ALS); anaplastic large cell lymphoma; anauxetic dysplasia; androgen insensitivity syndrome; angiopathic thrombosis; antiphospholipid
syndrome; Antley-Bixler syndrome; APECED; Apert syndrome; aplasia of lacrimal and salivary glands; arginase-1 deficiency; argininosuccinic
aciduria; argininemia; arrhythmogenic right ventricular dysplasia; Arts syndrome; ARVD2; arylsulfatase deficiency type metachromatic
leukodystrophy; ataxia telangiectasia; atherosclerotic cardiovascular disease; autoimmune lymphoproliferative syndrome; autoimmune
polyglandular syndrome type 1; autosomal dominant anhidrotic ectodermal dysplasia; autosomal dominant deafness; autosomal dominant
polycystic kidney disease; autosomal recessive microtia; autosomal recessive renal glucosuria; autosomal visceral heterotaxy; babesiosis;
bacterial vaginosis; balantidial dysentery; Bardet-Biedl syndrome; Bartter syndrome; basal cell nevus syndrome; Batten disease; benign
recurrent intrahepatic cholestasis; beta-mannosidosis; β-thalassemia; Bethlem myopathy; Blackfan-Diamond anemia; bleeding disorder
(coagulation); blepharophimosis; Byler disease; C syndrome; CADASIL; calcific aortic stenosis; calcification of joints and arteries; carbamoyl
phosphate synthetase I deficiency; carcinoid syndrome diarrhea; cardiofaciocutaneous syndrome; cardiovascular disease (CVD); Carney triad;
carnitine palmitoyltransferase deficiencies; cartilage-hair hypoplasia; cblC type of combined methylmalonic aciduria; CD18 deficiency; CD3Z-
associated primary T-cell immunodeficiency; CD40L deficiency; CDAGS syndrome; CDG1A; CDG1B; CDG1M; CDG2C; CEDN1K
syndrome; central core disease; centronuclear myopathy; cerebral capillary malformation; cerebrooculofacioskeletal syndrome type 4;
cerebrooculogacioskeletal syndrome; cerebrotendinous xanthomatosis; Chagas' Disease; Charcot Marie Tooth Disesase; chemotherapy;
cherubism; CHILD syndrome; chronic granulomatous disease; chronic recurrent multifocal osteomyelitis; cirrhosis; citrin deficiency;
citrullinemia type I; citrullinemia type II; classic hemochromatosis; CNPPB syndrome; cobalamin C disease; Cockayne syndrome; coenzyme
Q10 deficiency; Coffin-Lowry syndrome; Cohen syndrome; combined deficiency of coagulation factors V; common variable immune
deficiency 3; complement hyperactivation; complete androgen insentivity; cone rod dystrophies; conformational diseases; congenital adrenal
hyperplasia (CAH); congenital bile acid synthesis defect type 1; congenital bile acid synthesis defect type 2; congenital defect in bile acid
synthesis type; congenital erythropoietic porphyria; congenital generalized osteosclerosis; congenital muscular dystrophy 1A (MDC1A);
Cornelia de Lange syndrome; coronary heart disease; Cousin syndrome; Cowden disease; COX deficiency; Cri du chat syndrome; Crigler-
Najjar disease; Crigler-Najjar syndrome type 1; Crisponi syndrome; Crouzon syndrome; Currarino syndrome; Curth-Macklin type ichthyosis
hystrix; cutaneous T-cell lymphoma; cutis laxa; cystic fibrosis; cystinosis; d-2-hydroxyglutaric aciduria; DDP syndrome; Dejerine-Sottas
disease; dementia; Denys-Drash syndrome; Dercum disease; desmin cardiomyopathy; desmin myopathy; DGUOK-associated mitochondrial
DNA depletion; diabetes Type I; diabetes Type II; disorders of glutamate metabolism; distal spinal muscular atrophy type 5; DNA repair
diseases; dominant optic atrophy; Doyne honeycomb retinal dystrophy; Dravet Syndrome; Duchenne muscular dystrophy; dyskeratosis
congenita; Ehlers-Danlos syndrome type 4; Ehlers-Danlos syndromes; Elejalde disease; Ellis-van Creveld disease; Emery-Dreifuss muscular
dystrophies; encephalomyopathic mtDNA depletion syndrome; encephalitis; enzymatic diseases; EPCAM-associated congenital tufting
enteropathy; epidermolysis bullosa with pyloric atresia; epilepsy; fabry disease; facioscapulohumeral muscular dystrophy; Factor V Leiden
thrombophilia; Faisalabad histiocytosis; familial atypical mycobacteriosis; familial capillary malformation-arteriovenous; Familial Creutzfeld-
Jakob disease; familial esophageal achalasia; familial glomuvenous malformation; familial hemophagocytic lymphohistiocytosis; familial
mediterranean fever; familial megacalyces; familial schwannomatosis; familial spina bifida; familial splenic asplenia/hypoplasia; familial
thrombotic thrombocytopenia purpura; Fanconi disease (Fanconi anemia); Feingold syndrome; FENIB; fibrodysplasia ossificans progressiva;
FKTN; Fragile X syndrome; Francois-Neetens fleck corneal dystrophy; Frasier syndrome; Friedreich's ataxia; FTDP-17; Fuchs corneal
dystrophy; fucosidosis; G6PD deficiency; galactosialidosis; Galloway syndrome; Gardner syndrome; Gaucher disease; Gitelman syndrome;
glaucoma; GLUT1 deficiency; GM2- Gangliosidoses (e.g., Tay Sachs Disease, Sandhoff Disease) glycogen storage disease type 1b; glycogen
storage disease type 2; glycogen storage disease type 3; glycogen storage disease type 4; glycogen storage disease type 9a; glycogen storage
diseases; GM1-gangliosidosis; Greenberg syndrome; Greig cephalopolysyndactyly syndrome; hair genetic diseases; hairy cell leukemia;
HANAC syndrome; harlequin type ichtyosis congenita; HDR syndrome; hearing loss; heart failure; hemochromatosis type 3; hemochromatosis
type 4; hemolytic anemia; hemolytic uremic syndrome; hemophilia A; hemophilia B; hepatitis C infection; hereditary angioedema type 3;
hereditary angioedemas; hereditary hemorrhagic telangiectasia; hereditary hypofibrinogenemia; hereditary intraosseous vascular malformation;
hereditary leiomyomatosis and renal cell cancer; hereditary neuralgic amyotrophy; hereditary orotic aciduria; hereditary sensory and autonomic
neuropathy type; Hermansky-Pudlak disease; HHH syndrome; HHT2; hidrotic ectodermal dysplasia type 1; hidrotic ectodermal dysplasias;
histiocytic sarcoma; HNF4A-associated hyperinsulinism; HNPCC; homozygous familial hypercholesterolemia; hormone refractory prostate
cancer; human immunodeficiency with microcephaly; Human monkeypox (MPX); human papilloma virus (HPV) infection; Huntington's
disease; hyper-IgD syndrome; hyperinsulinism-hyperammonemia syndrome; hypercholesterolemia; hypertension; hypertrophy of the retinal
pigment epithelium; hypochondrogenesis; hypohidrotic ectodermal dysplasia; hypotension; ICF syndrome; idiopathic congenital intestinal
pseudo-obstruction; immunodeficiency 13; immunodeficiency 17; immunodeficiency 25; immunodeficiency with hyper-IgM type 1;
immunodeficiency with hyper-IgM type 3; immunodeficiency with hyper-IgM type 4; immunodeficiency with hyper-IgM type 5;
immunoglobulin alpha deficiency; inborn errors of thyroid metabolism; infantile myofibromatosis; infantile visceral myopathy; infantile X-
linked spinal muscular atrophy; influenza A; influenza B; insulin resistance; intradialytic hypotension; intrahepatic cholestasis of pregnancy;
invasive aspergillosis; invasive mucormycosis; IPEX syndrome; IRAK4 deficiency; isolated congenital asplenia; Jeune syndrome; Johanson-
Blizzard syndrome; Joubert syndrome; JP-HHT syndrome; juvenile hemochromatosis; juvenile hyalin fibromatosis; juvenile nephronophthisis;
Kabuki mask syndrome; Kallmann syndromes; Kartagener syndrome; KCNJ11-associated hyperinsulinism; Kearns-Sayre syndrome; Kostmann
disease; Kozlowski type of spondylometaphyseal dysplasia; Krabbe disease; LADD syndrome; late infantile-onset neuronal ceroid
lipofuscinosis; LCK deficiency; LDHCP syndrome; Leber Congenital Amaurosis Teyp 10; Legius syndrome; Leigh syndrome; lethal congenital
contracture syndrome 2; lethal congenital contracture syndromes; lethal contractural syndrome type 3; lethal neonatal CPT deficiency type 2;
lethal osteosclerotic bone dysplasia; leukocyte adhesion deficiency; Li Fraumeni syndrome; LIG4 syndrome; limb girdle muscular dystrophies
(LGMD1B, LGMD2A, LGMD2B); lipodystrophy; lissencephaly type 1; lissencephaly type 3; Loeys-Dietz syndrome; low phospholipid-
associated cholelithiasis; Lynch Syndrome; lysinuric protein intolerance; a lysosomal storage disease (e.g., Hunter syndrome, Hurler syndrome);
macular dystrophy; Maffucci syndrome; Majeed syndrome; malaria; mannose-binding protein deficiency; mantle cell lymphoma; Marfan
disease; Marshall syndrome; MASA syndrome; mastocytosis; MCAD deficiency; McCune-Albright syndrome; MCKD2; Meckel syndrome;
MECP2 Duplication Syndrome; Meesmann corneal dystrophy; megacystis-microcolon-intestinal hypoperistalsis; megaloblastic anemia type 1;
MEHMO; MELAS; Melnick-Needles syndrome; MEN2s; meningitis; Menkes disease; metachromatic leukodystrophies; methymalonic
acidemia due to transcobalamin receptor defect; methylmalonic acidurias; methylvalonic aciduria; microcoria-congenital nephrosis syndrome;
microvillous atrophy; migraine; mitochondrial neurogastrointestinal encephalomyopathy; monilethrix; monosomy X; mosaic trisomy 9
syndrome; Mowat-Wilson syndrome; mucolipidosis type 2; mucolipidosis type Ma; mucolipidosis type IV; mucopolysaccharidoses;
mucopolysaccharidosis type 3A; mucopolysaccharidosis type 3C; mucopolysaccharidosis type 4B; multiminicore disease; multiple acyl-CoA
dehydrogenation deficiency; multiple cutaneous and mucosal venous malformations; multiple endocrine neoplasia type 1; multiple myeloma;
multiple sclerosis; multiple sulfatase deficiency; mycosis fungoides; myotonic dystrophy; NAIC; nail-patella syndrome; nemaline myopathies;
neonatal diabetes mellitus; neonatal surfactant deficiency; nephronophtisis; Netherton disease; neurofibromatoses; neurofibromatosis type 1;
Niemann-Pick disease type A; Niemann-Pick disease type B; Niemann-Pick disease type C; NKX2E; non-alcoholic fatty liver disease
(NAFLD); non-alcoholic steatohepatitis (NASH); Noonan syndrome; North American Indian childhood cirrhosis; NROB1 duplication-
associated DSD; ocular genetic diseases; oculo-auricular syndrome; OLEDAID; oligomeganephronia; oligomeganephronic renal hypolasia;
Ollier disease; Opitz-Kaveggia syndrome; ornithine transcarbamylase deficiency (OTCD); orofaciodigital syndrome type 1; orofaciodigital
syndrome type 2; osseous Paget disease; osteogenesis imperfecta; otopalatodigital syndrome type 2; orthostatic hypotension; overactive bladder;
OXPHOS diseases; pain; palmoplantar hyperkeratosis; panlobar nephroblastomatosis; Parkes-Weber syndrome; Parkinson's disease; partial
deletion of 21q22.2-q22.3; Pearson syndrome; Pelizaeus-Merzbacher disease; Pendred syndrome; pentalogy of Cantrell; peroxisomal acyl-CoA-
oxidase deficiency; Peutz-Jeghers syndrome; Pfeiffer syndrome; Pierson syndrome; pigmented nodular adrenocortical disease; pipecolic
acidemia; Pitt-Hopkins syndrome; plasmalogens deficiency; platelet glycoprotein IV deficiency; pleuropulmonary blastoma and cystic
nephroma; pneumonia; polycystic kidney disease; polycystic ovarian disease; polycystic lipomembranous osteodysplasia; Pompe disease;
including infantile onset Pompe disease (IOPD) and late onset Pompe disease (LOPD); porphyrias; post-herpetic neuralgia; PRKAG2 cardiac
syndrome; premature ovarian failure; primary erythermalgia; primary hemochromatoses; primary hyperoxaluria; progressive familial
intrahepatic cholestasis; propionic acidemia; prostate cancer; protein-losing enteropathy; pulmonary arterial hypertension; pyruvate
decarboxylase deficiency; RAPADILINO syndrome; renal cystinosis; restless leg syndrome; retinitis pigmentosa; Rett Syndrome; rhabdoid
tumor predisposition syndrome; Rieger syndrome; ring chromosome 4; Roberts syndrome; Robinow-Sorauf syndrome; Rothmund-Thomson
syndrome; severe combined immunodeficiency disorder (SCID); Saethre-Chotzen syndrome; Sandhoff disease; SC phocomelia syndrome;
SCAS; Schinzel phocomelia syndrome; schizophrenia; severe hypertriglyceridemia; short rib-polydactyly syndrome type 1; short rib-
polydactyly syndrome type 4; short-rib polydactyly syndrome type 2; short-rib polydactyly syndrome type 3; Shwachman disease; Shwachman-
Diamond disease; sickle cell anemia; Silver-Russell syndrome; Simpson-Golabi-Behmel syndrome; skin infection; Smith-Lemli-Opitz
syndrome; SPG7-associated hereditary spastic paraplegia; spherocytosis; spinocerebellar ataxia; spinal muscular atrophy; split-hand/foot
malformation with long bone deficiencies; spondylocostal dysostosis; sporadic amyotrophic lateral sclerosis; sporadic visceral myopathy with
inclusion bodies; storage diseases; Stargardt macular dystrophy; STRA6-associated syndrome; stroke; subependymal glioma; tardive
dyskinesia; Tay-Sachs disease; thanatophoric dysplasia; thromboembolism; thrombosis; thrombophilia due to antithrombin III deficiency;
thyroid metabolism diseases; Tourette syndrome; transcarbamylase deficiency; transthyretin-associated amyloidosis; trisomy 13; trisomy 22;
trisomy 2p syndrome; tuberous sclerosis; tufting enteropathy; ullrich congenital muscular dystrophy (UCMD); urea cycle diseases; Usher
Syndrome; Van Den Ende-Gupta syndrome; Van der Woude syndrome; variegated mosaic aneuploidy syndrome; VLCAD deficiency; von
Hippel-Lindau disease; von Willebrand disease; Waardenburg syndrome; WAGR syndrome; Walker-Warburg syndrome; Werner syndrome;
Wilson's disease; Wiskott-Aldrich Syndrome; Wolcott-Rallison syndrome; Wolfram syndrome; X-linked agammaglobulinemia; X-linked
chronic idiopathic intestinal pseudo-obstruction; X-linked cleft palate with ankyloglossia; X-linked dominant chondrodysplasia punctata; X-
linked ectodermal dysplasia; X-linked Emery-Dreifuss muscular dystrophy; X-linked lissencephaly; X-linked lymphoproliferative disease; X-
linked visceral heterotaxy; xanthinuria type 1; xanthinuria type 2; xeroderma pigmentosum; XPV; and Zellweger disease.

### EXAMPLES

The following examples are included for illustrative purposes only and are not intended to limit the scope of the invention.

### Example 1: Metagenomic Identification of Effector Proteins and Effector Partners

Genes encoding effector proteins and effector partners were identified by sequence homology and structural analyses of potential CRISPR arrays and cognate proteins. Two groups of proteins emerged: a first group of proteins were identified close to the CRISPR arrays and a second group of proteins were identified close to the first group of proteins. All proteins were sorted by structural similarity into clusters. Through BLAST and HEIPred analysis, the identified proteins were found to be structurally similar to the IS family of transposases. Specifically, the first group of proteins were structurally similar to Ist21 transposases of the IS family of transposases (e.g., as encoded by istA) and identified as effector proteins of interest, and the second group of proteins were structurally similar to the helper proteins (e.g., as encoded by istB) of the IS family of transposases. Of the second group of proteins, two-subgroups of proteins emerged: a first subgroup that was generally found downstream of and typically shared an overlapping ORF with the first group of proteins; and a second subgroup that was found in varying locations. When a protein from the second subgroup was found downstream of the first subgroup, it was typically found to be ˜150 bp away and may have some overlapping ORFs. There were some exceptions, for example, when a protein from the second subgroup was found far downstream, such a protein was found to be about −3.5 kb away. When a protein from the second subgroup was found upstream, the ORF overlapped with the first group of proteins about half the time, otherwise the proteins were found to be within 50 bp of one another. Furthermore, if the ORF of a first subgroup does not overlap with the first group, then the two proteins were found within 50 bp of each other.

Without being bound by theory, it is contemplated that the identified effector proteins function as RNA-guided transposases. Also, without being bound by theory, it is contemplated that the second group of proteins, like the IS helper proteins, are also helper or partner proteins for the identified effector proteins of interest.

Effector Protein Library: In total, 454 effector proteins (SEQ ID NO: 1 to SEQ ID NO: 454), as set forth in TABLE 1 were selected as candidates.

Effector Partner Protein Library: In total, 515 effector partners (SEQ ID NO: 455 to SEQ ID NO: 969), as set forth in TABLE 1.1 were selected as candidates.

TABLE 6 describes effector protein and partner combinations identified in the above-described metagenomic analysis.

TABLE 6
COMPOSITIONS COMPRISING EFFECTOR PROTEIN AND
EFFECTOR PARTNER EXEMPLARY COMBINATIONS

| | Effector | | Effector | | Effector | | Location |
|---|---|---|---|---|---|---|---|
| | Protein | | Partner 1 | Effector | Partner 2 | Effector | Relative to |
| | (SEQ ID | Effector | (SEQ ID | Partner 1 | (SEQ ID | Partner 2 | Effector |
| # | NO) | Protein ID | NO) | ID | NO) | ID | Partner 1 |
| 1 | 1 | 2456001 | 906 | 2455999 | 514 | 2456002 | upstream |
| 2 | 2 | 2456475 | 595 | 2456476 | | | |
| 3 | 3 | 2456558 | 742 | 2456555 | | | |
| 4 | 4 | 2457490 | 596 | 2457488 | | | |
| 5 | 5 | 2457658 | 597 | 2457659 | | | |
| 6 | 6 | 2457689 | 598 | 2457687 | | | |
| 7 | 7 | 2457793 | 866 | 2457794 | | | |
| 8 | 8 | 2457935 | 715 | 2457936 | | | |
| 9 | 9 | 2458067 | 743 | 2458064 | | | |
| 10 | 10 | 2459252 | 889 | 2459261 | | | |
| 11 | 11 | 2460727 | 599 | 2460728 | | | |
| 12 | 12 | 2460813 | 744 | 2460811 | | | |
| 13 | 13 | 2460841 | 789 | 2460843 | | | |
| 14 | 14 | 2460904 | 790 | 2460905 | | | |
| 15 | 15 | 2460927 | 716 | 2460928 | | | |
| 16 | 16 | 2460977 | 549 | 2460978 | | | |
| 17 | 17 | 2462434 | 745 | 2462435 | | | |
| 18 | 18 | 2462454 | 550 | 2462455 | | | |
| 19 | 19 | 2463018 | 551 | 2463019 | | | |
| 20 | 20 | 2463119 | 494 | 2463120 | | | |
| 21 | 21 | 2464186 | 455 | 2464187 | | | |
| 22 | 22 | 2464978 | 717 | 2464976 | | | |
| 23 | 23 | 2465492 | 600 | 2465490 | | | |
| 24 | 24 | 2465502 | 601 | 2465500 | | | |
| 25 | 25 | 2465535 | 602 | 2465533 | | | |
| 26 | 26 | 2465684 | 907 | 2465682 | 515 | 2465685 | upstream |
| 27 | 27 | 2465703 | 890 | 2465701 | | | |
| 28 | 28 | 2466615 | 814 | 2466613 | | | |
| 29 | 29 | 2466640 | 908 | 2466642 | 516 | 2466638 | upstream |
| 30 | 30 | 2466645 | 456 | 2466643 | | | |
| 31 | 31 | 2466655 | 909 | 2466653 | 517 | 2466656 | upstream |
| 32 | 32 | 2466697 | | 2466695 | 518 | 2466698 | upstream |
| 33 | 33 | 2466980 | 583 | 2466978 | | | |
| 34 | 34 | 2467447 | 718 | 2467448 | | | |
| 35 | 35 | 2467918 | 842 | 2467919 | | | |
| 36 | 36 | 2467960 | 815 | 2467961 | | | |
| 37 | 37 | 2467986 | 816 | 2467984 | | | |
| 38 | 38 | 2468305 | 937 | 2468303 | 495 | 2468301 | downstream |
| 39 | 39 | 2468372 | 843 | 2468373 | | | |
| 40 | 40 | 2468693 | 791 | 2468691 | | | |
| 41 | 41 | 2468823 | 496 | 2468820 | | | |
| 42 | 42 | 2468912 | 938 | 2468910 | 497 | 2468908 | downstream |
| 43 | 43 | 2468988 | 498 | 2468985 | | | |
| 44 | 44 | 2469268 | 603 | 2469266 | | | |
| 45 | 45 | 2469304 | 604 | 2469302 | | | |
| 46 | 46 | 2469439 | 867 | 2469440 | | | |
| 47 | 47 | 2469808 | 719 | 2469809 | | | |
| 48 | 48 | 2469819 | 605 | 2469820 | | | |
| 49 | 49 | 2469824 | 720 | 2469822 | | | |
| 50 | 50 | 2470354 | 817 | 2470355 | | | |
| 51 | 51 | 2473512 | 868 | 2473513 | | | |
| 52 | 52 | 2482511 | 939 | 2482513 | 457 | 2482514 | downstream |
| 53 | 53 | 2483526 | 792 | 2483527 | | | |
| 54 | 54 | 2483741 | 606 | 2483742 | | | |
| 55 | 55 | 2489259 | 607 | 2489257 | | | |
| 56 | 56 | 2490524 | 608 | 2490525 | | | |
| 57 | 57 | 2490801 | 844 | 2490802 | | | |
| 58 | 58 | 2490995 | 609 | 2490993 | | | |
| 59 | 59 | 2491088 | 869 | 2491086 | | | |
| 60 | 60 | 2504283 | 910 | 2504285 | 519 | 2504281 | upstream |
| 61 | 61 | 2504313 | 746 | 2504310 | | | |
| 62 | 62 | 2504384 | 520 | 2504385 | | | |
| 63 | 63 | 2504398 | 610 | 2504396 | | | |
| 64 | 64 | 2504480 | 793 | 2504481 | | | |
| 65 | 65 | 2504486 | 794 | 2504484 | | | |
| 66 | 66 | 2504537 | 458 | 2504538 | | | |
| 67 | 67 | 2504664 | 940 | 2504662 | 499 | 2504660 | downstream |
| 68 | 68 | 2504681 | 845 | 2504679 | | | |
| 69 | 69 | 2504885 | 611 | 2504886 | | | |
| 70 | 70 | 2505104 | 612 | 2505102 | | | |
| 71 | 71 | 2505192 | 459 | 2505190 | | | |
| 72 | 72 | 2505203 | 911 | 2505201 | 521 | 2505204 | upstream |
| 73 | 73 | 2505234 | 613 | 2505235 | | | |
| 74 | 74 | 2505278 | 912 | 2505276 | 541 | 2505279 | upstream |
| 75 | 75 | 2505666 | 721 | 2505667 | | | |
| 76 | 76 | 2505670 | 614 | 2505668 | | | |
| 77 | 77 | 2505886 | 722 | 2505887 | | | |
| 78 | 78 | 2505958 | 615 | 2505959 | | | |
| 79 | 79 | 2506634 | 616 | 2506635 | | | |
| 80 | 80 | 2506662 | 723 | 2506660 | | | |
| 81 | 81 | 2506867 | 747 | 2506868 | | | |
| 82 | 82 | 2507107 | 584 | 2507105 | | | |
| 83 | 83 | 2507193 | 795 | 2507190 | | | |
| 84 | 84 | 2507535 | 617 | 2507533 | | | |
| 85 | 85 | 2507558 | 913 | 2507560 | 522 | 2507556 | upstream |
| 86 | 86 | 2507740 | 870 | 2507738 | | | |
| 87 | 87 | 2507920 | 941 | 2507918 | | | |
| 88 | 88 | 2508016 | 796 | 2508017 | | | |
| 89 | 89 | 2508076 | 797 | 2508077 | | | |
| 90 | 90 | 2508184 | 552 | 2508182 | | | |
| 91 | 91 | 2508454 | 871 | 2508455 | | | |
| 92 | 92 | 2509122 | 872 | 2509123 | | | |
| 93 | 93 | 2509421 | 942 | 2509419 | 460 | 2509417 | downstream |
| 94 | 94 | 2509689 | 724 | 2509687 | | | |
| 95 | 95 | 2509812 | 585 | 2509810 | | | |
| 96 | 96 | 2510622 | 725 | 2510620 | | | |
| 97 | 97 | 2512817 | 618 | 2512818 | | | |
| 98 | 98 | 2513039 | 586 | 2513037 | | | |
| 99 | 99 | 2513437 | 873 | 2513438 | | | |
| 100 | 100 | 2514574 | 891 | 2514575 | | | |
| 101 | 101 | 2514639 | 726 | 2514640 | | | |
| 102 | 102 | 2514917 | 727 | 2514918 | | | |
| 103 | 103 | 2514993 | 818 | 2514991 | | | |
| 104 | 104 | 2515125 | 553 | 2515126 | | | |
| 105 | 105 | 2519365 | 892 | 2519363 | | | |
| 106 | 106 | 2520309 | 748 | 2520310 | | | |
| 107 | 107 | 2520321 | 749 | 2520322 | | | |
| 108 | 108 | 2520815 | 798 | 2520813 | | | |
| 109 | 109 | 2520872 | 750 | 2520870 | | | |
| 110 | 110 | 2521221 | 846 | 2521222 | | | |
| 111 | 111 | 2521365 | 847 | 2521363 | | | |
| 112 | 112 | 2521462 | 819 | 2521464 | | | |
| 113 | 113 | 2521488 | 820 | 2521489 | | | |
| 114 | 114 | 2521520 | 821 | 2521518 | | | |
| 115 | 115 | 2521566 | 822 | 2521567 | | | |
| 116 | 116 | 2521693 | 823 | 2521691 | | | |
| 117 | 117 | 2521795 | 848 | 2521796 | | | |
| 118 | 118 | 2521836 | 849 | 2521834 | | | |
| 119 | 119 | 2521914 | 850 | 2521915 | | | |
| 120 | 120 | 2522106 | 851 | 2522104 | | | |
| 121 | 121 | 2522156 | 852 | 2522154 | | | |
| 122 | 122 | 2522180 | 853 | 2522182 | | | |
| 123 | 123 | 2522205 | 854 | 2522203 | | | |
| 124 | 124 | 2522208 | 855 | 2522206 | | | |
| 125 | 125 | 2522283 | 856 | 2522281 | | | |
| 126 | 126 | 2522388 | 824 | 2522386 | | | |
| 127 | 127 | 2522406 | 857 | 2522407 | | | |
| 128 | 128 | 2522427 | 858 | 2522425 | | | |
| 129 | 129 | 2522444 | 859 | 2522442 | | | |
| 130 | 130 | 2522739 | 825 | 2522737 | | | |
| 131 | 131 | 2522792 | 826 | 2522793 | | | |
| 132 | 132 | 2522843 | 619 | 2522844 | | | |
| 133 | 133 | 2523900 | 620 | 2523898 | | | |
| 134 | 134 | 2526395 | 621 | 2526396 | | | |
| 135 | 135 | 2526520 | 622 | 2526521 | | | |
| 136 | 136 | 2532645 | 728 | 2532646 | | | |
| 137 | 137 | 2532927 | 893 | 2532928 | | | |
| 138 | 138 | 2533225 | 729 | 2533223 | | | |
| 139 | 139 | 2533240 | 894 | 2533241 | | | |
| 140 | 140 | 2533877 | 461 | 2533878 | 463 | 2533879 | downstream |
| 141 | 141 | 2534949 | 623 | 2534950 | | | |
| 142 | 142 | 2535292 | 554 | 2535290 | | | |
| 143 | 143 | 2536836 | 827 | 2536837 | | | |
| 144 | 144 | 2537132 | 874 | 2537130 | | | |
| 145 | 145 | 2537146 | 943 | 2537144 | 500 | 2537141 | downstream |
| 146 | 146 | 2537379 | 751 | 2537380 | | | |
| 147 | 147 | 2538576 | 914 | 2538578 | 542 | 2538574 | upstream |
| 148 | 148 | 2538582 | 624 | 2538580 | | | |
| 149 | 149 | 2538893 | 875 | 2538891 | | | |
| 150 | 150 | 2538912 | 895 | 2538913 | | | |
| 151 | 151 | 2539582 | 501 | 2539580 | | | |
| 152 | 152 | 2540760 | 915 | 2540758 | 523 | 2540761 | upstream |
| 153 | 153 | 2542176 | 828 | 2542177 | | | |
| 154 | 154 | 2542314 | 799 | 2542315 | | | |
| 155 | 155 | 2542321 | 502 | 2542319 | | | |
| 156 | 156 | 2542504 | 916 | 2542506 | 543 | 2542502 | upstream |
| 157 | 157 | 2542546 | 800 | 2542544 | | | |
| 158 | 158 | 2542603 | 503 | 2542604 | | | |
| 159 | 159 | 2542660 | 625 | 2542658 | | | |
| 160 | 160 | 2542678 | 752 | 2542674 | | | |
| 161 | 161 | 2542856 | 730 | 2542857 | | | |
| 162 | 162 | 2542961 | 944 | 2542963 | 462 | 2542964 | downstream |
| 163 | 163 | 2542982 | 945 | 2542984 | | | |
| 164 | 164 | 2542993 | 946 | 2542995 | | | |
| 165 | 165 | 2543049 | 464 | 2543050 | | | |
| 166 | 166 | 2543336 | 731 | 2543334 | | | |
| 167 | 167 | 2543415 | 801 | 2543412 | | | |
| 168 | 168 | 2543456 | 555 | 2543457 | | | |
| 169 | 169 | 2543553 | 876 | 2543551 | | | |
| 170 | 170 | 2543727 | 829 | 2543728 | | | |
| 171 | 171 | 2544565 | 732 | 2544566 | | | |
| 172 | 172 | 2545576 | 556 | 2545577 | | | |
| 173 | 173 | 2545774 | 830 | 2545772 | | | |
| 174 | 174 | 2549059 | 753 | 2549056 | | | |
| 175 | 175 | 2549426 | 626 | 2549427 | | | |
| 176 | 176 | 2549542 | 896 | 2549543 | | | |
| 177 | 177 | 2549749 | 627 | 2549750 | | | |
| 178 | 178 | 2551318 | 947 | 2551320 | 465 | 2551321 | downstream |
| 179 | 179 | 2552263 | 628 | 2552264 | | | |
| 180 | 180 | 2553740 | 629 | 2553741 | | | |
| 181 | 181 | 2555300 | 754 | 2555301 | | | |
| 182 | 182 | 2555429 | 917 | 2555427 | | | |
| 183 | 183 | 2555732 | 918 | 2555730 | | | |
| 184 | 184 | 2555743 | 630 | 2555741 | | | |
| 185 | 185 | 2557277 | 631 | 2557278 | | | |
| 186 | 186 | 2559207 | 632 | 2559205 | | | |
| 187 | 187 | 2559356 | 633 | 2559354 | | | |
| 188 | 188 | 2559367 | 831 | 2559365 | | | |
| 189 | 189 | 2559541 | 860 | 2559542 | | | |
| 190 | 190 | 2559544 | 861 | 2559545 | | | |
| 191 | 191 | 2559747 | 733 | 2559748 | | | |
| 192 | 192 | 2560387 | 734 | 2560388 | | | |
| 193 | 193 | 2560437 | 634 | 2560438 | | | |
| 194 | 194 | 2560621 | 635 | 2560622 | | | |
| 195 | 195 | 2561184 | 755 | 2561182 | | | |
| 196 | 196 | 2563225 | 756 | 2563226 | | | |
| 197 | 197 | 2563834 | 832 | 2563835 | | | |
| 198 | 198 | 2564858 | 636 | 2564856 | | | |
| 199 | 199 | 2566876 | 757 | 2566873 | | | |
| 200 | 200 | 2568378 | 919 | 2568376 | 544 | 2568379 | upstream |
| 201 | 201 | 2569008 | 920 | 2569010 | 525 | 2569006 | upstream |
| 202 | 202 | 2569140 | 897 | 2569141 | | | |
| 203 | 203 | 2570277 | 637 | 2570278 | | | |
| 204 | 204 | 2570395 | 638 | 2570396 | | | |
| 205 | 205 | 2570998 | 639 | 2570999 | | | |
| 206 | 206 | 2572578 | 877 | 2572576 | | | |
| 207 | 207 | 2572822 | 898 | 2572823 | | | |
| 208 | 208 | 2572874 | 921 | 2572872 | 526 | 2572875 | upstream |
| 209 | 209 | 2572904 | 758 | 2572905 | | | |
| 210 | 210 | 2573626 | 759 | 2573624 | | | |
| 211 | 211 | 2573858 | 640 | 2573856 | | | |
| 212 | 212 | 2573979 | 862 | 2573977 | | | |
| 213 | 213 | 2574339 | 878 | 2574337 | | | |
| 214 | 214 | 2574480 | 760 | 2574478 | | | |
| 215 | 215 | 2575022 | 863 | 2575020 | | | |
| 216 | 216 | 2575248 | 466 | 2575246 | | | |
| 217 | 217 | 2575975 | 899 | 2575976 | | | |
| 218 | 218 | 2576124 | 833 | 2576125 | | | |
| 219 | 219 | 2576604 | 834 | 2576602 | | | |
| 220 | 220 | 2576783 | 641 | 2576781 | | | |
| 221 | 221 | 2578899 | 642 | 2578900 | | | |
| 222 | 222 | 2579280 | 802 | 2579281 | | | |
| 223 | 223 | 2580408 | 643 | 2580409 | | | |
| 224 | 224 | 2580412 | 864 | 2580413 | | | |
| 225 | 225 | 2581095 | 587 | 2581093 | | | |
| 226 | 226 | 2581145 | 588 | 2581146 | | | |
| 227 | 227 | 2581162 | 644 | 2581163 | | | |
| 228 | 228 | 2581239 | 922 | 2581241 | 545 | 2581237 | upstream |
| 229 | 229 | 2581436 | 645 | 2581434 | | | |
| 230 | 230 | 2581467 | 589 | 2581465 | | | |
| 231 | 231 | 2581554 | 803 | 2581551 | | | |
| 232 | 232 | 2581611 | 646 | 2581609 | | | |
| 233 | 233 | 2581622 | 647 | 2581620 | | | |
| 234 | 234 | 2581692 | 648 | 2581693 | | | |
| 235 | 235 | 2581801 | 649 | 2581799 | | | |
| 236 | 236 | 2581900 | 650 | 2581898 | | | |
| 237 | 237 | 2582748 | 590 | 2582746 | | | |
| 238 | 238 | 2585153 | 900 | 2585154 | | | |
| 239 | 239 | 2585490 | 761 | 2585488 | | | |
| 240 | 240 | 2585740 | 591 | 2585738 | | | |
| 241 | 241 | 2585756 | 505 | 2585757 | 493 | 2585758 | downstream |
| 242 | 242 | 2585858 | 651 | 2585859 | | | |
| 243 | 243 | 2585862 | 557 | 2585863 | | | |
| 244 | 244 | 2585963 | 652 | 2585964 | | | |
| 245 | 245 | 2585975 | 653 | 2585976 | | | |
| 246 | 246 | 2587058 | 558 | 2587059 | | | |
| 247 | 247 | 2594512 | 879 | 2594510 | | | |
| 248 | 248 | 2595285 | 654 | 2595286 | | | |
| 249 | 249 | 2595296 | 655 | 2595297 | | | |
| 250 | 250 | 2596238 | 901 | 2596236 | | | |
| 251 | 251 | 2607761 | 902 | 2607759 | | | |
| 252 | 252 | 2610314 | 903 | 2610312 | | | |
| 253 | 253 | 2618595 | 762 | 2618591 | | | |
| 254 | 254 | 2618809 | 735 | 2618810 | | | |
| 255 | 255 | 2619117 | 559 | 2619115 | | | |
| 256 | 256 | 2619261 | 656 | 2619262 | | | |
| 257 | 257 | 2621890 | 560 | 2621888 | | | |
| 258 | 258 | 2621901 | 763 | 2621908 | | | |
| 259 | 259 | 2628024 | 804 | 2628025 | | | |
| 260 | 260 | 2629316 | 880 | 2629314 | | | |
| 261 | 261 | 2631654 | 948 | 2631652 | 506 | 2631650 | downstream |
| 262 | 262 | 2631725 | 805 | 2631726 | | | |
| 263 | 263 | 2631733 | 806 | 2631730 | | | |
| 264 | 264 | 2631899 | 592 | 2631897 | | | |
| 265 | 265 | 2631942 | 835 | 2631940 | | | |
| 266 | 266 | 2635243 | 949 | 2635245 | 467 | 2635246 | downstream |
| 267 | 267 | 2636967 | 657 | 2636968 | | | |
| 268 | 268 | 2637525 | 865 | 2637523 | | | |
| 269 | 269 | 2639392 | 764 | 2639393 | | | |
| 270 | 270 | 2639486 | 836 | 2639484 | | | |
| 271 | 271 | 2639844 | 736 | 2639845 | | | |
| 272 | 272 | 2640111 | 658 | 2640109 | 504 | 2640108 | downstream |
| 273 | 273 | 2640390 | 904 | 2640391 | | | |
| 274 | 274 | 2640394 | 561 | 2640392 | | | |
| 275 | 275 | 2640521 | 807 | 2640528 | | | |
| 276 | 276 | 2640532 | 562 | 2640530 | | | |
| 277 | 277 | 2641112 | 546 | 2641110 | | | |
| 278 | 278 | 2641808 | 659 | 2641806 | 513 | 2641805 | downstream |
| 279 | 279 | 2641851 | 660 | 2641852 | | | |
| 280 | 280 | 2642080 | 593 | 2642081 | | | |
| 281 | 281 | 2642178 | 661 | 2642176 | 524 | 2642175 | downstream |
| 282 | 282 | 2642301 | 765 | 2642302 | | | |
| 283 | 283 | 2642927 | 737 | 2642928 | | | |
| 284 | 284 | 2643487 | 808 | 2643488 | | | |
| 285 | 285 | 2643718 | 507 | 2643716 | | | |
| 286 | 286 | 2643741 | 809 | 2643738 | | | |
| 287 | 287 | 2643749 | 810 | 2643750 | | | |
| 288 | 288 | 2643777 | 662 | 2643775 | | | |
| 289 | 289 | 2645072 | 923 | 2645074 | 547 | 2645070 | upstream |
| 290 | 290 | 2645389 | 738 | 2645387 | | | |
| 291 | 291 | 2645407 | 739 | 2645405 | | | |
| 292 | 292 | 2645582 | 663 | 2645583 | | | |
| 293 | 293 | 2645604 | 664 | 2645605 | | | |
| 294 | 294 | 2645609 | 665 | 2645607 | | | |
| 295 | 295 | 2645629 | 666 | 2645630 | | | |
| 296 | 296 | 2645965 | 811 | 2645962 | | | |
| 297 | 297 | 2645994 | 766 | 2645992 | | | |
| 298 | 298 | 2646041 | 812 | 2646042 | | | |
| 299 | 299 | 2646098 | 468 | 2646099 | 473 | 2646104 | far |
| | | | | | | | downstream |
| 300 | 300 | 2646113 | 950 | 2646115 | 469 | 2646116 | downstream |
| 301 | 301 | 2646119 | 667 | 2646117 | | | |
| 302 | 302 | 2646278 | 951 | 2646276 | 508 | 2646274 | downstream |
| 303 | 303 | 2646295 | 952 | 2646297 | 509 | 2646298 | downstream |
| 304 | 304 | 2646303 | 924 | 2646301 | 548 | 2646304 | upstream |
| 305 | 305 | 2646334 | 767 | 2646335 | | | |
| 306 | 306 | 2646355 | 563 | 2646353 | | | |
| 307 | 307 | 2646358 | 470 | 2646356 | | | |
| 308 | 308 | 2646390 | 925 | 2646388 | 527 | 2646391 | upstream |
| 309 | 309 | 2646408 | 528 | 2646406 | | | |
| 310 | 310 | 2646830 | 768 | 2646831 | | | |
| 311 | 311 | 2646934 | 668 | 2646932 | | | |
| 312 | 312 | 2646962 | 669 | 2646963 | | | |
| 313 | 313 | 2647076 | 529 | 2647077 | | | |
| 314 | 314 | 2647091 | | 2647092 | | | |
| 315 | 315 | 2647113 | 926 | 2647115 | 530 | 2647111 | upstream |
| 316 | 316 | 2647159 | 670 | 2647160 | | | |
| 317 | 317 | 2647165 | 671 | 2647163 | | | |
| 318 | 318 | 2647204 | 510 | 2647205 | | | |
| 319 | 319 | 2647896 | 813 | 2647893 | | | |
| 320 | 320 | 2648017 | 881 | 2648018 | | | |
| 321 | 321 | 2648219 | 672 | 2648220 | | | |
| 322 | 322 | 2648249 | 673 | 2648250 | | | |
| 323 | 323 | 2648260 | 740 | 2648261 | | | |
| 324 | 324 | 2648589 | 594 | 2648590 | | | |
| 325 | 325 | 2648617 | 953 | 2648615 | 511 | 2648613 | downstream |
| 326 | 326 | 2648628 | 512 | 2648626 | | | |
| 327 | 327 | 2648641 | 954 | 2648639 | | | |
| 328 | 328 | 2648953 | 471 | 2648954 | | | |
| 329 | 329 | 2649138 | 905 | 2649136 | | | |
| 330 | 330 | 2649178 | 674 | 2649176 | | | |
| 331 | 331 | 2649330 | 675 | 2649328 | | | |
| 332 | 332 | 2649348 | 676 | 2649349 | | | |
| 333 | 333 | 2649388 | 677 | 2649386 | | | |
| 334 | 334 | 2649407 | 678 | 2649408 | | | |
| 335 | 335 | 2649418 | 741 | 2649419 | | | |
| 336 | 336 | 2649881 | 679 | 2649882 | | | |
| 337 | 337 | 2650573 | 955 | 2650571 | 472 | 2650569 | downstream |
| 338 | 338 | 2650642 | 956 | 2650644 | | | |
| 339 | 339 | 2650650 | 957 | 2650648 | 474 | 2650646 | downstream |
| 340 | 340 | 2650663 | 475 | 2650661 | | | |
| 341 | 341 | 2650676 | 958 | 2650674 | 476 | 2650672 | downstream |
| 342 | 342 | 2652498 | 769 | 2652499 | | | |
| 343 | 343 | 2655342 | 564 | 2655343 | | | |
| 344 | 344 | 2655877 | 680 | 2655878 | | | |
| 345 | 345 | 2656012 | 927 | 2656014 | 531 | 2656010 | upstream |
| 346 | 346 | 2656041 | 770 | 2656038 | | | |
| 347 | 347 | 2656102 | 837 | 2656100 | | | |
| 348 | 348 | 2656122 | 771 | 2656119 | | | |
| 349 | 349 | 2656637 | 565 | 2656638 | | | |
| 350 | 350 | 2656683 | 681 | 2656681 | | | |
| 351 | 351 | 2656732 | 682 | 2656733 | | | |
| 352 | 352 | 2656745 | 928 | 2656747 | 532 | 2656743 | upstream |
| 353 | 353 | 2656872 | 566 | 2656870 | | | |
| 354 | 354 | 2656881 | 772 | 2656882 | | | |
| 355 | 355 | 2657264 | 773 | 2657265 | | | |
| 356 | 356 | 2657281 | 567 | 2657279 | | | |
| 357 | 357 | 2658718 | 959 | 2658716 | 477 | 2658714 | downstream |
| 358 | 358 | 2658736 | 960 | 2658738 | 478 | 2658739 | downstream |
| 359 | 359 | 2659684 | 774 | 2659685 | | | |
| 360 | 360 | 2660855 | 479 | 2660856 | | | |
| 361 | 361 | 2661138 | 929 | 2661136 | 533 | 2661139 | upstream |
| 362 | 362 | 2661149 | 930 | 2661147 | 534 | 2661150 | upstream |
| 363 | 363 | 2661203 | 568 | 2661204 | | | |
| 364 | 364 | 2661243 | 775 | 2661240 | | | |
| 365 | 365 | 2661723 | 776 | 2661724 | | | |
| 366 | 366 | 2661753 | 882 | 2661751 | | | |
| 367 | 367 | 2662365 | 683 | 2662363 | | | |
| 368 | 368 | 2663028 | 838 | 2663029 | | | |
| 369 | 369 | 2663104 | 569 | 2663105 | | | |
| 370 | 370 | 2663367 | 480 | 2663365 | | | |
| 371 | 371 | 2663380 | 481 | 2663378 | 490 | 2663377 | downstream |
| 372 | 372 | 2663400 | 482 | 2663398 | | | |
| 373 | 373 | 2663750 | 931 | 2663748 | 535 | 2663751 | upstream |
| 374 | 374 | 2663760 | 684 | 2663758 | | | |
| 375 | 375 | 2663780 | 685 | 2663781 | | | |
| 376 | 376 | 2663815 | 686 | 2663813 | | | |
| 377 | 377 | 2663833 | 932 | 2663835 | 536 | 2663831 | upstream |
| 378 | 378 | 2663974 | 933 | 2663976 | 537 | 2663972 | upstream |
| 379 | 379 | 2664009 | 687 | 2664010 | | | |
| 380 | 380 | 2664025 | 483 | 2664026 | | | |
| 381 | 381 | 2664156 | 934 | 2664154 | 538 | 2664157 | upstream |
| 382 | 382 | 2664342 | 777 | 2664339 | | | |
| 383 | 383 | 2664593 | 935 | 2664591 | 539 | 2664594 | upstream |
| 384 | 384 | 2664914 | 961 | 2664916 | 484 | 2664917 | downstream |
| 385 | 385 | 2664957 | 570 | 2664958 | | | |
| 386 | 386 | 2664986 | 688 | 2664987 | | | |
| 387 | 387 | 2665006 | 689 | 2665007 | | | |
| 388 | 388 | 2665109 | 962 | 2665107 | 485 | 2665105 | downstream |
| 389 | 389 | 2665325 | 571 | 2665323 | | | |
| 390 | 390 | 2665379 | 572 | 2665380 | | | |
| 391 | 391 | 2665417 | 573 | 2665418 | | | |
| 392 | 392 | 2665447 | 690 | 2665448 | | | |
| 393 | 393 | 2666085 | 963 | 2666087 | 486 | 2666088 | downstream |
| 394 | 394 | 2666163 | 839 | 2666164 | | | |
| 395 | 395 | 2666290 | 574 | 2666288 | | | |
| 396 | 396 | 2666471 | 691 | 2666469 | | | |
| 397 | 397 | 2666830 | 936 | 2666832 | 540 | 2666828 | upstream |
| 398 | 398 | 2667583 | 692 | 2667584 | | | |
| 399 | 399 | 2667596 | 964 | 2667598 | 487 | 2667599 | downstream |
| 400 | 400 | 2667609 | 883 | 2667610 | | | |
| 401 | 401 | 2667637 | 575 | 2667638 | | | |
| 402 | 402 | 2667659 | 840 | 2667661 | | | |
| 403 | 403 | 2667740 | 778 | 2667741 | | | |
| 404 | 404 | 2667863 | 693 | 2667864 | | | |
| 405 | 405 | 2667874 | 694 | 2667875 | | | |
| 406 | 406 | 2668940 | 576 | 2668938 | | | |
| 407 | 407 | 2669158 | 779 | 2669159 | | | |
| 408 | 408 | 2669252 | 695 | 2669250 | | | |
| 409 | 409 | 2669317 | 780 | 2669318 | | | |
| 410 | 410 | 2669455 | 841 | 2669452 | | | |
| 411 | 411 | 2670262 | 781 | 2670263 | | | |
| 412 | 412 | 2670431 | 696 | 2670429 | | | |
| 413 | 413 | 2670948 | 782 | 2670945 | | | |
| 414 | 414 | 2671509 | 697 | 2671510 | | | |
| 415 | 415 | 2671561 | 577 | 2671559 | | | |
| 416 | 416 | 2673019 | 698 | 2673017 | | | |
| 417 | 417 | 2673030 | 699 | 2673028 | | | |
| 418 | 418 | 2673085 | 700 | 2673086 | | | |
| 419 | 419 | 2673122 | 488 | 2673120 | 491 | 2673119 | downstream |
| 420 | 420 | 2673750 | 884 | 2673751 | | | |
| 421 | 421 | 2674873 | 701 | 2674874 | | | |
| 422 | 422 | 2674903 | 965 | 2674905 | 489 | 2674906 | downstream |
| 423 | 423 | 2674920 | 783 | 2674917 | | | |
| 424 | 424 | 2675029 | 966 | 2675031 | | | |
| 425 | 425 | 2675057 | 702 | 2675058 | | | |
| 426 | 426 | 2675141 | 703 | 2675139 | | | |
| 427 | 427 | 2675820 | 704 | 2675822 | | | |
| 428 | 428 | 2675905 | 705 | 2675906 | | | |
| 429 | 429 | 2678009 | 885 | 2678007 | | | |
| 430 | 430 | 2678104 | 886 | 2678102 | | | |
| 431 | 431 | 2678129 | 706 | 2678127 | | | |
| 432 | 432 | 2680715 | 784 | 2680716 | | | |
| 433 | 433 | 2680787 | 967 | 2680785 | | | |
| 434 | 434 | 2680813 | 707 | 2680814 | | | |
| 435 | 435 | 2681033 | 708 | 2681034 | | | |
| 436 | 436 | 2681040 | 968 | 2681038 | 492 | 2681036 | downstream |
| 437 | 437 | 2681339 | 709 | 2681340 | | | |
| 438 | 438 | 2682278 | 785 | 2682279 | | | |
| 439 | 439 | 2682344 | 578 | 2682345 | | | |
| 440 | 440 | 2682347 | 579 | 2682348 | | | |
| 441 | 441 | 2684692 | 580 | 2684690 | | | |
| 442 | 442 | 2684983 | 710 | 2684984 | | | |
| 443 | 443 | 2685267 | 711 | 2685265 | | | |
| 444 | 444 | 2687981 | 887 | 2687982 | | | |
| 445 | 445 | 2687986 | 712 | 2687984 | | | |
| 446 | 446 | 2687998 | 713 | 2687996 | | | |
| 447 | 447 | 2688086 | 786 | 2688083 | | | |
| 448 | 448 | 2688136 | 787 | 2688137 | | | |
| 449 | 449 | 2688183 | 788 | 2688184 | | | |
| 450 | 450 | 2688225 | 581 | 2688226 | | | |
| 451 | 451 | 2688430 | 969 | 2688428 | | | |
| 452 | 452 | 2689663 | 714 | 2689664 | | | |
| 453 | 453 | 2690183 | 888 | 2690184 | | | |
| 454 | 454 | 2690859 | 582 | 2690860 | | | |

In total, 454 exemplary effector protein and effector partner(s) combinations were identified and are set forth in TABLE 6. Some combinations (e.g., composition no. 454) identify an effector protein but do not identify any effector partners. For compositions listed without effector partners, it is envisioned that an effector partner candidate may be found by further genomic analysis.

### Example 2: Activity of Effector Protein and Effector Partner In Vitro

Effector proteins and effector partner combinations are tested for their ability to guide the direct transposition of a donor nucleic acid into a target plasmid in an in vitro assay. A first plasmid encoding an effector protein, a second plasmid encoding an effector partner, and a third target plasmid. Donor DNA can be generated from a plasmid or a linear double-stranded DNA molecule. The donor DNA contains the spectinomycin resistance gene with a structural motif, inverted terminal repeats (ITRs) that a transposase can recognize. Where more than one effector partner is identified for an effector protein (e.g., comp. no. 436 in TABLE 11 above), the second plasmid encoding the effector partner can further include one or more nucleotide sequences encoding the additional effector partners or the additional effector partners can be encoded by a fourth plasmid. Plasmids encoding the effector protein and effector partner(s) are bacterial nuc-doc expression vectors. A target plasmid containing an 51 spacer (5′-TATTAAATACTCGTATTGCTGTTCGATTAT-3′ (SEQ ID NO: 984) and an ampicillin resistance gene are also generated.

To test for transposase activity, plasmids encoding the effector proteins and effector partners are contacted with a guide RNA, in combinations for example, as set forth in TABLE 3, along with the donor DNA and the target template. The composition is incubated for a sufficient amount of time to allow the effector protein and/or effector partners to recognize the ITRs and direct transposition of the donor nucleic acid to the 51 spacer in the target plasmid. The insertion of the donor DNA into the target plasmid demonstrates the transposase activity of the effector protein and effector partner combinations. After transposition, the target plasmid will contain the spectinomycin and ampicillin resistance genes. The target plasmid is then transformed into E. coli and screened using spectinomycin and ampicillin. Only target plasmids that have successfully been transposed into will allow transformed bacteria to survive both antibiotics. Next, plasmids from the surviving colonies are sequenced by next generation sequencing (NGS) of PCR amplicons to assess whether the donor DNA was integrated in the target site as directed by the guide RNA, as well as PAM requirements of the same. Controls can include gene products of MuA, MuB, IstA, and IstB.

### Example 3: Indel Activity of Effector Proteins and Effector Partners in Lipofected Eukaryotic Cells

Effector proteins and/or effector partners combinations as described in Example 1 are tested for their ability to form indels within a target nucleic acid (e.g., genomic DNA) in eukaryotic cells (e.g., immune cell, T cell, HEK29 cell, or any other eukaryotic cell). Plasmid pairs co-expressing the effector protein, effector partner(s), and gRNA (1 plasmid/target) are delivered to eukaryotic cells via lipofection using a lipofection reagent. Lipofected cells are incubated to allow for indel formation. Indels are detected by next generation sequencing (NGS) of PCR amplicons at the targeted loci, and indel percentage is calculated as the fraction of sequencing reads containing insertions or deletions relative to an unedited reference sequence.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1.-60. (canceled)

61. A composition that comprises:
(i) a polypeptide, or a nucleic acid encoding the polypeptide, wherein the polypeptide comprises an amino acid sequence that is at least 75% identical to any one of the sequences set forth in TABLE 1; and
(ii) an engineered guide nucleic acid or a DNA molecule that encodes the engineered guide nucleic acid.

62. The composition of claim 1, wherein the composition comprises one or more partner polypeptides, wherein the one or more partner polypeptides comprises an amino acid sequence that is at least 75% identical to any one of the sequences set forth in TABLE 1.1.

63. The composition of claim 2, wherein the composition comprises a polypeptide and a partner polypeptide combination as described in TABLE 6.

64. The composition of claim 1, wherein the composition comprises a donor nucleic acid.

65. The composition of claim 1, wherein the polypeptide has site-specific recombinase activity.

66. The composition of claim 1, wherein the polypeptide has transposase activity.

67. A nucleic acid expression vector that encodes a polypeptide, wherein the polypeptide comprises an amino acid sequence that is at least 75% identical to any one of the sequences set forth in TABLE 1.

68. A system comprising:
(i) a polypeptide, or a nucleic acid encoding the polypeptide, wherein the polypeptide comprises an amino acid sequence that is at least 75% identical to any one of the sequences set forth in TABLE 1; and
(ii) an engineered guide nucleic acid or a DNA molecule that encodes the engineered guide nucleic acid.

69. A method of modifying a target nucleic acid within a human gene, or associated with expression of a human gene, the method comprising contacting the target nucleic acid with the composition of claim 1.

70. A cell that comprises a target nucleic acid modified by the composition of claim 1.

71. A method of treating a disease associated with a mutation or aberrant expression of a human gene in a subject in need thereof, the method comprising administering to the subject the composition of claim 1.

72. The method of claim 71, wherein the disease is any one of the diseases recited in TABLE 5 and/or wherein the human gene is a gene recited in TABLE 4.
